SEQUENCE LISTING (1 sequence)

SEQ ID NO: 1 - protein, 1068 aa, Homo sapiens
MPPRPSSGELWGIHLMPPRILVECLLPNGMIVTLECLREATLITIKHELFKEARKYPLHQLLQDESSYIFVSVTQEAEREEFFDETRRLCDLRLFQPFLKVIEPVGNREEKILNREIGFAIGMPVCEFDMVKDPEVQDFRRNILNVCKEAVDLRDLNSPHSRAMYVYPPNVESSPELPKHIYNKLDKGQIIVVIWVIVSPNNDKQKYTLKINHDCVPEQVIAEAIRKKTRSMLLSSEQLKLCVLEYQGKYILKVCGCDEYFLEKYPLSQYKYIRSCIMLGRMPNLMLMAKESLYSQLPMDCFTMPSYSRRISTATPYMNGETSTKSLWVINSALRIKILCATYVNVNIRDIDKIYVRTGIYHGGEPLCDNVNTQRVPCSNPRWNEWLNYDIYIPDLPRAARLCLSICSVKGRKGAKEEHCPLAWGNINLFDYTDTLVSGKMALNLWPVPHGLEDLLNPIGVTGSNPNKETPCLELEFDWFSSVVKFPDMSVIEEHANWSVSREAGFSYSHAGLSNRLARDNELRENDKEQLKAISTRDPLSEITEQEKDFLWSHRHYCVTIPEILPKLLLSVKWNSRDEVAQMYCLVKDWPPIKPEQAMELLDCNYPDPMVRGFAVRCLEKYLTDDKLSQYLIQLVQVLKYEQYLDNLLVRFLLKKALTNQRIGHFFFWHLKSEMHNKTVSQRFGLLLESYCRACGMYLKHLNRQVEAMEKLINLTDILKQEKKDETQKVQMKFLVEQMRRPDFMDALQGFLSPLNPAHQLGNLRLEECRIMSSAKRPLWLNWENPDIMSELLFQNNEIIFKNGDDLRQDMLTLQIIRIMENIWQNQGLDLRMLPYGCLSIGDCVGLIEVVRNSHTIMQIQCKGGLKGALQFNSHTLHQWLKDKNKGEIYDAAIDLFTRSCAGYCVATFILGIGDRHNSNIMVKDDGQLFHIDFGHFLDHKKKKFGYKRERVPFVLTQDFLIVISKGAQECTKTREFERFQEMCYKAYLAIRQHANLFINLFSMMLGSGMPELQSFDDIAYIRKTLALDKTEQEALEYFMKQMNDAHHGGWTTKMDWIFHTIKQHALN

(19) United States
(12) Patent Application Publication
(10) Pub. No.: US 2026/0130925 A1
(43) Pub. Date: May 14, 2026

(54) METHODS FOR TREATING CANCER

(71) Applicant: Genesis Molecular AI, Inc., Burlingame, CA (US)

(72) Inventors: Brett Busch, San Diego, CA (US); Nicholas Simon Stock, Encinitas, CA (US); Advait Nagle, San Diego, CA (US); Evan Nathaniel Feinberg, Palo Alto, CA (US); Martin Indarte, Krakow (PL)

(21) Appl. No.: 19/118,605

(22) PCT Filed: Oct. 13, 2023
(86) PCT No.: PCT/US2023/076876

§ 371 Date: Apr. 4, 2025

Related U.S. Application Data
(60) Provisional application No. 63/416,253, filed on Oct. 14, 2022.
(60) Provisional application No. 63/416,177, filed on Oct. 14, 2022.
(60) Provisional application No. 63/416,169, filed on Oct. 14, 2022.

(51) Int. Cl. A61K 31/635; A61K 31/352; A61K 31/4025; A61K 31/4035; A61K 31/444; A61K 31/453; A61K 31/4545; A61K 31/4709; A61K 31/4725; A61K 31/501; A61K 31/517; A61K 31/5377; A61K 31/541; C07D 217/14; C07D 217/24; C07D 217/26; C07D 239/91; C07D 311/28; C07D 401/04; C07D 403/04; C07D 403/14; C07D 405/04; C07D 405/12; C07D 405/14; C07D 407/04; C07D 409/04; C07D 409/14; C07D 413/04; C07D 413/14; C07D 417/04; C07D 471/04; C07D 491/052

(52) U.S. Cl. CPC A61K 31/635; A61K 31/352; A61K 31/4025; A61K 31/4035; A61K 31/444; A61K 31/453; A61K 31/4545; A61K 31/4709; A61K 31/4725; A61K 31/501; A61K 31/517; A61K 31/5377; A61K 31/541; C07D 217/14; C07D 217/24; C07D 217/26; C07D 239/91; C07D 311/28; C07D 401/04; C07D 403/04; C07D 403/14; C07D 405/04; C07D 405/12; C07D 405/14; C07D 407/04; C07D 409/04; C07D 409/14; C07D 413/04; C07D 413/14; C07D 417/04; C07D 471/04; C07D 491/052

(57) ABSTRACT

This disclosure provides compounds of Formula (I), Formula (II), Formula (III), and pharmaceutically acceptable salts of any of the foregoing, that inhibit PI3Kα. These compounds are useful for treating diseases such as cancer in a subject.

### CROSS-REFERENCE TO RELATED APPLICATIONS

This application claims the benefit of priority to U.S. Application No. 63/416,177, filed on Oct. 14, 2022, U.S. Application No. 63/416,253 filed on Oct. 14, 2022, U.S. Application No. 63/416,169, filed on Oct. 14, 2022, the contents of which are hereby incorporated by reference in their entireties.

### SEQUENCE LISTING

This application contains a Sequence Listing that has been submitted electronically as an XML file named 49366-0038WO1_ST.26_SL.XML.” The XML file, created on Oct. 13, 2023, is 2,933 bytes in size. The material in the XML file is hereby incorporated by reference in its entirety.

### TECHNICAL FIELD

This disclosure provides compounds of Formula (I), Formula (II), Formula (III), and pharmaceutically acceptable salts of any of the foregoing, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### BACKGROUND

Phosphatidylinositol 4,5-bisphosphate 3-kinase (PI3K) isoform alpha (PI3Kα), encoded by the PIK3CA gene is a part of the PI3K/AKT/TOR signaling network and is altered in several human cancers.

Activation of the PI3K pathway occurs in approximately 30-50% human cancers and contributes to resistance to various anti-cancer therapies. (See, Bauer, T. M. et al., Pharmacol. Ther. 2015, 146, 53-60.) However, development of PI3K inhibitors has been problematic for several reasons, in particular, inability to specifically inhibit signaling by mutant PI3Kα while sparing wild-type PI3Kα, and the related dose-limiting toxicities that prevent sustained PI3K pathway suppression. (See, Hanker et al., Cancer Discovery, April 2019; 9: 482-491.) For example, alpelisib is a PI3K inhibitor that is equipotent against wild-type and mutant forms of PI3Kα, which results in dose-limiting toxicities and hyperglycemia.

Thus, selectively targeting PI3Kα represents an approach for the treatment of proliferative disorders such as cancer.

### SUMMARY

Some embodiments provide a compound of Formula (I):

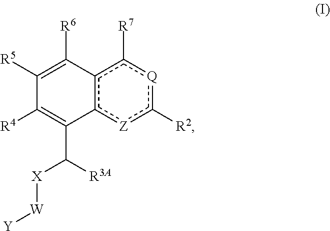

or a pharmaceutically acceptable salt of any of the foregoing, wherein:
Q is NR^{1A} or CR¹;
Z is NR^{Z1} or CR^{Z2}, wherein one or both of Q and Z is N;
each [symbol] represents a single bond or a double bond;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1A} is absent, hydrogen, C1-C6 alkyl, C1-C6 haloalkyl, C1-C6 thioalkyl, C1-C6 haloalkyl, C1-C6 alkoxy, or C1-C6 alkoxyalkyl;
R^{Z1} is absent, hydrogen, cyano, C1-C6 alkyl, or C1-C6 alkyl;
R^{Z2} is hydrogen, C1-C6 alkyl, or C1-C6 haloalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

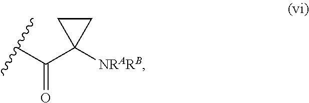

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═NR^{7A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

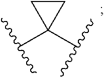 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (II):

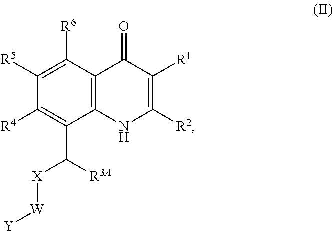

or a pharmaceutically acceptable salt of any of the foregoing, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

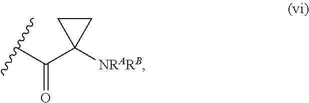

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{T1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R³ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═NR^{7A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

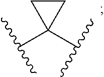 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (III):

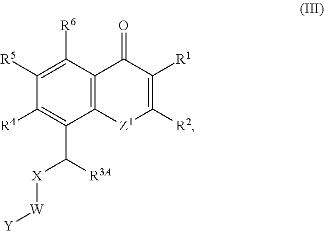

or a pharmaceutically acceptable salt of any of the foregoing, wherein:
Z¹ is S, —S(O₂)—, or O;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B}

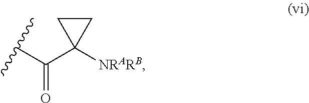

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═R^{A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

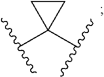 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (I-A):

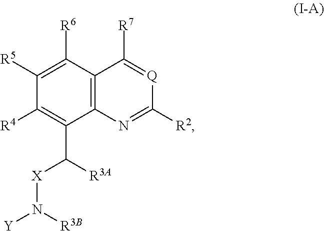

or a pharmaceutically acceptable salt thereof, wherein:
Q is N or CR¹;
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

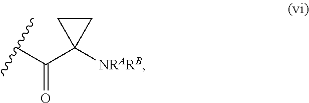

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, —NR^{7A}R^{7B}, —NR^{7A}C(═O)R^{7B}, or —C(═O)NR^{7A}R^{7B};
R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

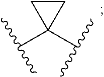 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (II-A):

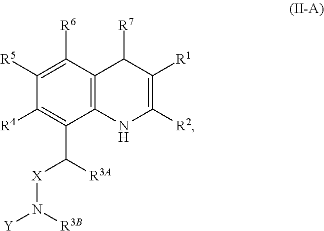

or a pharmaceutically acceptable salt thereof, wherein:
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-3 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

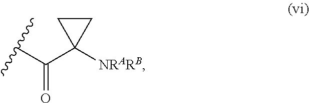

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

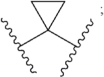 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R¹;
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (III-A):

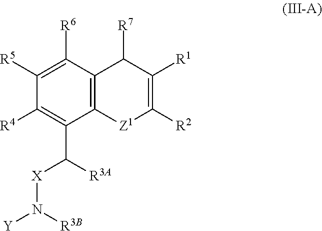

or a pharmaceutically acceptable salt thereof, wherein:
Z is S, —S(O₂)—, or O;
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

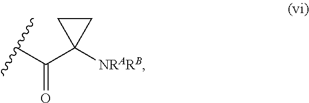

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;

X is a bond, CH₂, CH(CH₃), C(CH₃)₂, or

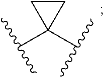 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R¹;
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Also provided herein is a pharmaceutical composition comprising a compound of Formula (I), Formula (II), or Formula (III), or a pharmaceutically acceptable salt of any of the foregoing, and one or more pharmaceutically acceptable excipients.

Provided herein is a method for treating cancer in a subject in need thereof, comprising administering to the subject a therapeutically effective amount of a compound of Formula (I), Formula (II), or Formula (III), or a pharmaceutically acceptable salt of any of the foregoing, or a pharmaceutical composition as provided herein.

Also provided herein is a method for treating cancer in a subject in need thereof, comprising (a) determining that the cancer is associated with a dysregulation of a PIK3CA gene, a PI3Kα protein, or expression or activity or level of any of the same; and (b) administering to the subject a therapeutically effective amount of a compound of Formula (I), Formula (II), or Formula (III), or a pharmaceutically acceptable salt of any of the foregoing, or a pharmaceutical composition as provided herein.

Provided herein is a method of treating a PI3Kα-associated disease in a subject, comprising administering to a subject identified or diagnosed as having a PI3Kα-associated disease a therapeutically effective amount of a compound of Formula (I), Formula (II), or Formula (III), or a pharmaceutically acceptable salt of any of the foregoing, or a pharmaceutical composition as provided herein.

This disclosure also provides a method of treating a PI3Kα-associated disease in a subject, comprising: determining that the cancer in the subject is a PI3Kα-associated disease; and administering to the subject a therapeutically effective amount of a compound of Formula (I), Formula (II), or Formula (III), or a pharmaceutically acceptable salt of any of the foregoing, or a pharmaceutical composition as provided herein.

Further provided herein is a method of treating a PI3Kα-associated cancer in a subject, comprising administering to a subject identified or diagnosed as having a PI3Kα-associated cancer a therapeutically effective amount of a compound of Formula (I), Formula (II), or Formula (III), or a pharmaceutically acceptable salt of any of the foregoing, or a pharmaceutical composition as provided herein.

This disclosure also provides a method of treating a PI3Kα-associated cancer in a subject, comprising: determining that the cancer in the subject is a PI3Kα-associated cancer; and administering to the subject a therapeutically effective amount of a compound of Formula (I), Formula (II), or Formula (III), or a pharmaceutically acceptable salt of any of the foregoing, or a pharmaceutical composition as provided herein.

Provided herein is a method of treating a subject, comprising administering a therapeutically effective amount of a compound of Formula (I), Formula (II), or Formula (III), or a pharmaceutically acceptable salt of any of the foregoing, or a pharmaceutical composition as provided herein, to a subject having a clinical record that indicates that the subject has a dysregulation of a PIK3CA gene, a PI3Kα protein, or expression or activity or level of any of the same.

This disclosure also provides a method for inhibiting PI3Kα in a mammalian cell, comprising contacting the mammalian cell with an effective amount of a compound of Formula (I), Formula (II), or Formula (III), or a pharmaceutically acceptable salt of any of the foregoing.

Other embodiments include those described in the Detailed Description and/or in the claims.

### Additional Definitions

To facilitate understanding of the disclosure set forth herein, a number of additional terms are defined below. Generally, the nomenclature used herein and the laboratory procedures in organic chemistry, medicinal chemistry, and pharmacology described herein are those well-known and commonly employed in the art. Unless defined otherwise, all technical and scientific terms used herein generally have the same meaning as commonly understood by one of ordinary skill in the art to which this disclosure belongs. Each of the patents, applications, published applications, and other publications that are mentioned throughout the specification and the attached appendices are incorporated herein by reference in their entireties.

The term “about” when referring to a number or a numerical range means that the number or numerical range referred to is an approximation, for example, within experimental variability and/or statistical experimental error, and thus the number or numerical range may vary up to ±10% of the stated number or numerical range.

The term “acceptable” with respect to a formulation, composition or ingredient, as used herein, means having no persistent detrimental effect on the general health of the subject being treated.

The term “inhibit” or “inhibition of” means to reduce by a measurable amount, or to prevent entirely (e.g., 100% inhibition).

The phrase “therapeutically effective amount” means an amount of compound that, when administered to a subject in need of such treatment, is sufficient to (i) treat a PI3Kα protein-associated disease, (ii) attenuate, ameliorate, or eliminate one or more symptoms of the particular disease, or (iii) delay the onset of one or more symptoms of the particular disease described herein.

The term “pharmaceutically acceptable salt” refers to a formulation of a compound that does not cause significant irritation to an organism to which it is administered and does not abrogate the biological activity and properties of the compound.

As used herein, the term “subject” refer to any animal, including mammals such as primates (e.g., humans), mice, rats, other rodents, rabbits, dogs, cats, swine, cattle, sheep, horses, primates, and humans. In some embodiments, the subject is a human. In some embodiments, the subject has experienced and/or exhibited at least one symptom of the disease to be treated and/or prevented.

As used herein, terms “treat” or “treatment” refer to therapeutic or palliative measures. Beneficial or desired clinical results include, but are not limited to, alleviation, in whole or in part, of symptoms associated with a disease, diminishment of the extent of disease, stabilized (i.e., not worsening) state of disease, delay or slowing of disease progression, amelioration or palliation of the disease state (e.g., one or more symptoms of the disease), and remission (whether partial or total), whether detectable or undetectable. “Treatment” can also mean prolonging survival as compared to expected survival if not receiving treatment.

The term “halogen” refers to fluoro (F), chloro (Cl), bromo (Br), or iodo (I).

The term “oxo” refers to a divalent doubly bonded oxygen atom (i.e., “=O”). As used herein, oxo groups are attached to carbon atoms to form carbonyls.

The term “hydroxyl” refers to an —OH radical.

The term “cyano” refers to a —CN radical.

The term “alkyl” refers to a saturated acyclic hydrocarbon radical that may be a straight chain or branched chain, containing the indicated number of carbon atoms. For example, C₁₋₁₀ indicates that the group may have from 1 to 10 (inclusive) carbon atoms in it. Non-limiting examples include methyl, ethyl, iso-propyl, tert-butyl, n-hexyl. The term “saturated” as used in this context means only single bonds present between constituent carbon atoms and other available valences occupied by hydrogen and/or other substituents as defined herein.

The term “haloalkyl” refers to an alkyl, in which one or more hydrogen atoms is/are replaced with an independently selected halogen.

The term “alkoxy” refers to an —O-alkyl radical (e.g., —OCH₃).

The term “alkoxyalkyl” refers to an -alkyl-O-alkyl radical (e.g., —CH₂CH₂OCH₃).

The term “thioalkyl” refers to an —S-alkyl radical (e.g., —SCH₃) or an -alkyl-S-alkyl radical (e.g., —CH₂CH₂SCH₃).

The term “hydroxyalkyl” refers to an alkyl, in which one or more hydrogen atoms is/are replaced with hydroxyl.

The term “aryl” refers to a 6-20 membered all carbon ring system wherein at least one ring in the system is aromatic (e.g., 6-carbon monocyclic, 10-carbon bicyclic, or 14-carbon tricyclic aromatic ring system). Examples of aryl groups include phenyl, naphthyl, tetrahydronaphthyl, and the like.

The term “cycloalkyl” as used herein refers to cyclic saturated hydrocarbon groups having, e.g., 3 to 20 ring carbons, preferably 3 to 16 ring carbons, and more preferably 3 to 12 ring carbons or 3-10 ring carbons or 3-6 ring carbons. Examples of cycloalkyl groups include, without limitation, cyclopropyl, cyclobutyl, cyclopentyl, cyclohexyl, cycloheptyl, and cyclooctyl. Cycloalkyl may include multiple fused and/or bridged rings. Non-limiting examples of fused/bridged cycloalkyl includes: bicyclo[1.1.0]butane, bicyclo[2.1.0]pentane, bicyclo[1.1.1]pentane, bicyclo[3.1.0]hexane, bicyclo[2.1.1]hexane, bicyclo[3.2.0]heptane, bicyclo[4.1.0]heptane, bicyclo[2.2.1]heptane, bicyclo[3.1.1]heptane, bicyclo[4.2.0]octane, bicyclo[3.2.1]octane, bicyclo[2.2.2]octane, and the like. Cycloalkyl also includes spirocyclic rings (e.g., spirocyclic bicycle wherein two rings are connected through just one atom). Non-limiting examples of spirocyclic cycloalkyls include spiro[2.2]pentane, spiro[2.5]octane, spiro[3.5]nonane, spiro[3.5]nonane, spiro[3.5]nonane, spiro[4.4]nonane, spiro[2.6]nonane, spiro[4.5]decane, spiro[3.6]decane, spiro[5.5]undecane, and the like. The term “saturated” as used in this context means only single bonds present between constituent carbon atoms.

The term “heteroaryl”, as used herein, refers to a ring system having 5 to 20 ring atoms, such as 5, 6, 9, 10, or 14 ring atoms; wherein at least one ring in the system contains one or more heteroatoms independently selected from the group consisting of N, O, S, Si, and B, and at least one ring in the system is aromatic (but does not have to be a ring which contains a heteroatom, e.g. tetrahydroisoquinolinyl, e.g., tetrahydroquinolinyl). Heteroaryl groups can include monocyclic, bridged, fused, and spiro ring systems, so long as one ring in the system is aromatic. Examples of heteroaryl include thienyl, pyridinyl, furyl, oxazolyl, oxadiazolyl, pyrrolyl, imidazolyl, triazolyl, thiodiazolyl, pyrazolyl, isoxazolyl, thiadiazolyl, pyranyl, pyrazinyl, pyrimidinyl, pyridazinyl, triazinyl, thiazolyl benzothienyl, benzoxadiazolyl, benzofuranyl, benzimidazolyl, benzotriazolyl, cinnolinyl, indazolyl, indolyl, isoquinolinyl, isothiazolyl, naphthyridinyl, purinyl, thienopyridinyl, pyrido[2,3-d]pyrimidinyl, pyrrolo[2,3-b]pyridinyl, quinazolinyl, quinolinyl, thieno[2,3-c]pyridinyl, pyrazolo[3,4-b]pyridinyl, pyrazolo[3,4-c]pyridinyl, pyrazolo[4,3-c]pyridine, pyrazolo[4,3-b]pyridinyl, tetrazolyl, chromane, 2,3-dihydrobenzo[b][1,4]dioxine, benzo[d][1,3]dioxole, 2,3-dihydrobenzofuran, tetrahydroquinoline, 2,3-dihydrobenzo[b][1,4]oxathiine, isoindoline, and others. In some embodiments, the heteroaryl is selected from thienyl, pyridinyl, furyl, pyrazolyl, imidazolyl, isoindolinyl, pyranyl, pyrazinyl, and pyrimidinyl. For purposes of clarification, heteroaryl also includes aromatic lactams, aromatic cyclic ureas, or vinylogous analogs thereof, in which each ring nitrogen adjacent to a carbonyl is tertiary (i.e., all three valences are occupied by non-hydrogen substituents), such as one or more of

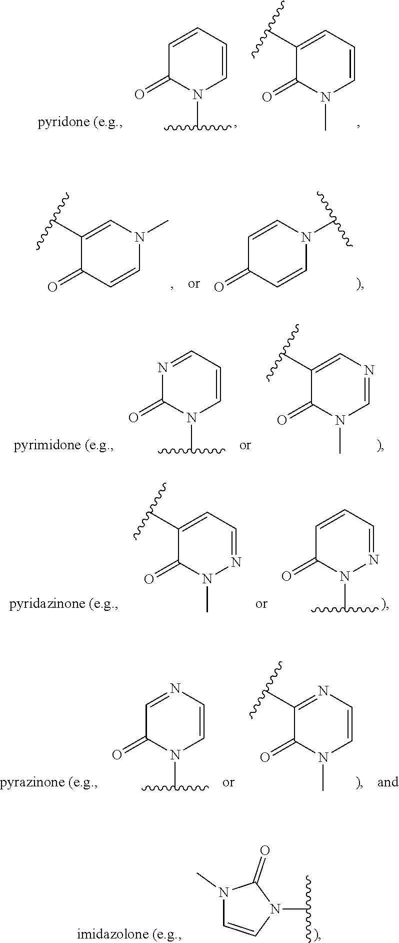

wherein each ring nitrogen adjacent to a carbonyl is tertiary (i.e., the oxo group (i.e., “=O”) herein is a constituent part of the heteroaryl ring).

The term “heterocyclyl” refers to a saturated or partially unsaturated ring systems with 3-16 ring atoms (e.g., 3-8 membered monocyclic, 5-12 membered bicyclic, or 10-14 membered tricyclic ring system) having at least one heteroatom selected from O, N, S, Si, and B, wherein one or more ring atoms may be substituted by 1-3 oxo (forming, e.g., a lactam) and one or more N or S atoms may be substituted by 1-2 oxido (forming, e.g., an N-oxide, an S-oxide, or an S,S-dioxide), valence permitting Heterocyclyl groups include monocyclic, bridged, fused, and spiro ring systems. Heterocyclyl groups include monocyclic, bridged, fused, and spiro ring systems. Examples of heterocyclyl groups include piperazinyl, pyrrolidinyl, dioxanyl, morpholinyl, tetrahydrofuranyl, tetrahydropyridyl, dihydropyrazinyl, dihydropyridyl, dihydropyrrolyl, dihydrofuranyl, dihydrothiophenyl, and the like. Heterocyclyl may include multiple fused and bridged rings. Non-limiting examples of fused/bridged heterocyclyl includes: 2-azabicyclo[1.1.0]butane, 2-azabicyclo[2.1.0]pentane, 2-azabicyclo[1.1.1]pentane, 3-azabicyclo[3.1.0]hexane, 5-azabicyclo[2.1.1]hexane, 3-azabicyclo[3.2.0]heptane, octahydrocyclopenta[c]pyrrole, 3-azabicyclo[4.1.0]heptane, 7-azabicyclo[2.2.1]heptane, 6-azabicyclo[3.1.1]heptane, 7-azabicyclo[4.2.0]octane, 2-azabicyclo[2.2.2]octane, 3-azabicyclo[3.2.1]octane, 2-oxabicyclo[1.1.0]butane, 2-oxabicyclo[2.1.0]pentane, 2-oxabicyclo[1.1.1]pentane, 3-oxabicyclo[3.1.0]hexane, 5-oxabicyclo[2.1.1]hexane, 3-oxabicyclo[3.2.0]heptane, 3-oxabicyclo[4.1.0]heptane, 7-oxabicyclo[2.2.1]heptane, 6-oxabicyclo[3.1.1]heptane, 7-oxabicyclo[4.2.0]octane, 2-oxabicyclo[2.2.2]octane, 3-oxabicyclo[3.2.1]octane, and the like. Heterocyclyl also includes spirocyclic rings (e.g., spirocyclic bicycle wherein two rings are connected through just one atom). Non-limiting examples of spirocyclic heterocyclyls include 2-azaspiro[2.2]pentane, 4-azaspiro[2.5]octane, 1-azaspiro[3.5]nonane, 2-azaspiro[3.5]nonane, 7-azaspiro[3.5]nonane, 2-azaspiro[4.4]nonane, 6-azaspiro[2.6]nonane, 1,7-diazaspiro[4.5]decane, 7-azaspiro[4.5]decane 2,5-diazaspiro[3.6]decane, 3-azaspiro[5.5]undecane, 2-oxaspiro[2.2]pentane, 4-oxaspiro[2.5]octane, 1-oxaspiro[3.5]nonane, 2-oxaspiro[3.5]nonane, 7-oxaspiro[3.5]nonane, 2-oxaspiro[4.4]nonane, 6-oxaspiro[2.6]nonane, 1,7-dioxaspiro[4.5]decane, 2,5-dioxaspiro[3.6]decane, 1-oxaspiro[5.5]undecane, 3-oxaspiro[5.5]undecane, 3-oxa-9-azaspiro[5.5]undecane and the like.

An “aralkyl” refers to an aryl group, as defined herein, connected to the remainder of the molecule via a divalent C1-C6 alkyl group, as described herein. Non-limiting examples of an aralkyl group are benzyl, ethylphenyl, methylnaphthyl, and the like.

A “heteroaralkyl” refers to a heteroaryl group, as defined herein, connected to the remainder of the molecule via a divalent C1-C6 alkyl group, as described herein. Non-limiting examples of an aralkyl group are methylpyridyl, ethylpyrimidinyl, methylimidazolyl, and the like.

As used herein, examples of aromatic rings include: benzene, pyridine, pyrimidine, pyrazine, pyridazine, pyridone, pyrrole, pyrazole, oxazole, thioazole, isoxazole, isothiazole, and the like.

As used herein, when a ring is described as being “partially unsaturated”, it means said ring has one or more additional degrees of unsaturation (in addition to the degree of unsaturation attributed to the ring itself, e.g., one or more double or triple bonds between constituent ring atoms), provided that the ring is not aromatic. Examples of such rings include: cyclopentene, cyclohexene, cycloheptene, dihydropyridine, tetrahydropyridine, dihydropyrrole, dihydrofuran, dihydrothiophene, and the like.

For the avoidance of doubt, and unless otherwise specified, for rings and cyclic groups (e.g., aryl, heteroaryl, heterocyclyl, cycloalkyl, and the like described herein) containing a sufficient number of ring atoms to form bicyclic or higher order ring systems (e.g., tricyclic, polycyclic ring systems), it is understood that such rings and cyclic groups encompass those having fused rings, including those in which the points of fusion are located (i) on adjacent ring atoms (e.g., [x.x.0] ring systems, in which 0 represents a zero atom bridge

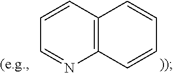

(ii) a single ring atom (spiro-fused ring systems)

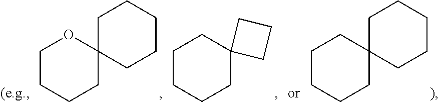

or (iii) a contiguous array of ring atoms (bridged ring systems having all bridge lengths>0)

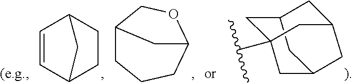

In addition, atoms making up the compounds of the present embodiments are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In addition, the compounds generically or specifically disclosed herein are intended to include all tautomeric forms. Thus, by way of example, a compound containing the moiety:

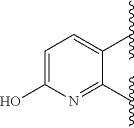 [SMILES: *c1ccc(O)nc1*]

encompasses the tautomeric form containing the moiety:

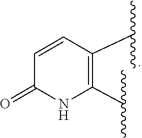 [SMILES: *c1ccc(=O)[nH]c1*]

Similarly, a pyridinyl or pyrimidinyl moiety that is described to be optionally substituted with hydroxyl encompasses pyridone or pyrimidone tautomeric forms.

The compounds provided herein may encompass various stereochemical forms. The compounds also encompass enantiomers (e.g., R and S isomers), diastereomers, as well as mixtures of enantiomers (e.g., R and S isomers) including racemic mixtures and mixtures of diastereomers, as well as individual enantiomers and diastereomers, which arise as a consequence of structural asymmetry in certain compounds. Unless otherwise indicated, when a disclosed compound is named or depicted by a structure without specifying the stereochemistry (e.g., a “flat” structure) and has one or more chiral centers, it is understood to represent all possible stereoisomers of the compound. Likewise, unless otherwise indicated, when a disclosed compound is named or depicted by a structure that specifies the stereochemistry (e.g., a structure with “wedge” and/or “dashed” bonds) and has one or more chiral centers, it is understood to represent the indicated stereoisomer of the compound.

To the extent that the compounds described herein (e.g., compounds of Formula (I), Formula (II), and Formula (III)) share substituent numbering, the substituent disclosure applies to all formulae having a particular substituent.

The details of one or more embodiments of this disclosure are set forth in the accompanying drawings and the description below. Other features and advantages of the present disclosure will be apparent from the description and drawings, and from the claims.

### DETAILED DESCRIPTION

This disclosure provides compounds of Formula (I), Formula (II), or Formula (III), and pharmaceutically acceptable salts of any of the foregoing, that inhibit phosphatidylinositol 4,5-bisphosphate 3-kinase (PI3K) isoform alpha (PI3Kα). These compounds are useful for treating a disease in which increased PI3Kα activation contributes to the pathology, symptoms, and/or progression of the disease (e.g., cancer) in a subject.

### Formulae (I), Formula (II), and Formula (III) Compounds

Some embodiments provide a compound of Formula (I):

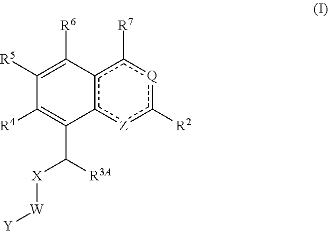

or a pharmaceutically acceptable salt thereof, wherein:
Q is NR^{1A} or CR¹;
Z is NR^{Z1} or CR^{Z2}, wherein one or both of Q and Z is N;
each [symbol] represents a single bond or a double bond;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1A} is absent, hydrogen, C1-C6 alkyl, C1-C6 haloalkyl, C1-C6 thioalkyl, C1-C6 haloalkyl, C1-C6 alkoxy, or C1-C6 alkoxyalkyl;
R^{Z1} is absent, hydrogen, cyano, C1-C6 alkyl, or C1-C6 alkyl;
R^{Z2} is hydrogen, C1-C6 alkyl, or C1-C6 haloalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B2},

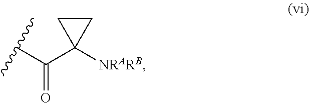

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═R^{A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

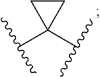 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, Q is NR^{1A}.

In some embodiments Q is CR¹.

In some embodiments, Z is NR^{Z1}.

In some embodiments, Z is CR^{Z2}.

In some embodiments, Q is NR^{1A} and Z is NR^{Z1}.

Some embodiments provide a compound of Formula (I-A):

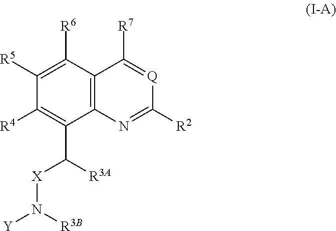

or a pharmaceutically acceptable salt thereof, wherein:
Q is N or CR¹;
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

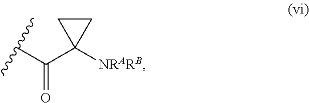

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, —NR^{7A}R^{7B}, —NR^{7A}C(═O)R^{7B}, or —C(═O)NR^{7A}R^{7B};
R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

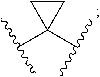 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, Q is N.

In some embodiments Q is CR¹.

Some embodiments provide a compound of Formula (II):

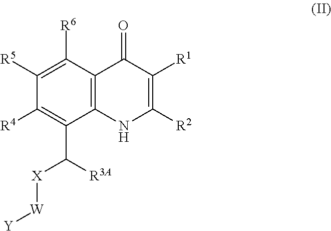

or a pharmaceutically acceptable salt thereof, wherein:
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

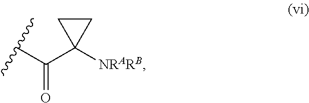

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

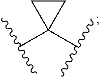 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (II-A):

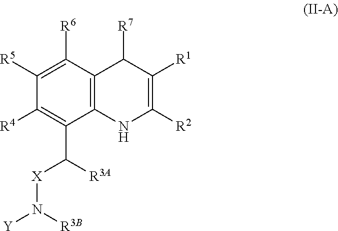

or a pharmaceutically acceptable salt thereof, wherein:
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-3 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

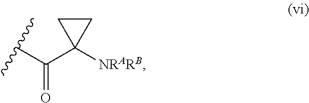

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{SB}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

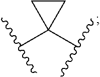 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R^{Y1};
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (III):

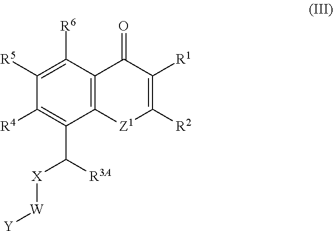

or a pharmaceutically acceptable salt thereof, wherein:
Z¹ is S, —S(O₂)—, or O;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, C1-C6 alkoxyalkyl optionally substituted with —C(═O)NR^{A}R^{C}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

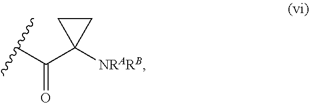

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, —SO₂(C1-C6 alkyl), and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;

X is a bond, CH₂, CH(CH₃), C(CH₃)₂, or

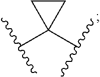 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 4-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

Some embodiments provide a compound of Formula (III-A):

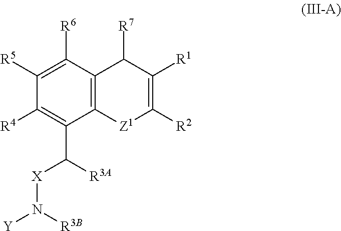

or a pharmaceutically acceptable salt thereof, wherein:
Z is S, —S(O₂)—, or O;
R¹ is hydrogen, cyano, C3-C6 cycloalkyl, or C1-C6 alkyl optionally substituted with phenyl optionally substituted with halogen;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

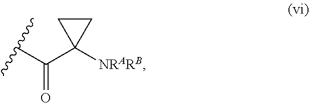

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen and C1-C6 alkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
X is a bond, CH₂, CH(CH₃), C(CH₃)₂, or

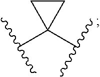 [SMILES: *C1(*)CC1]

Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, and C1-C6 alkyl optionally substituted with —CO₂R^{A} or 5-6 membered heteroaryl optionally substituted with R¹;
R^{Y1} is —SO₂(C1-C6 alkyl) or C1-C6 alkyl optionally substituted with oxo; and
each R^{F} and R^{G} is independently selected from hydrogen, phenyl, and C1-C6 alkyl optionally substituted with oxo or —NR^{A}R^{B}.

In some embodiments, Z¹ is S.

In some embodiments, Z¹ is —S(O₂)—.

In some embodiments, Z¹ is O.

In some embodiments, R¹ is hydrogen.

In some embodiments, R¹ is cyano.

In some embodiments, R¹¹ is C3-C6 cycloalkyl. In some embodiments, R¹ is cyclopropyl or cyclobutyl.

In some embodiments, R¹ is C1-C6 alkyl optionally substituted with phenyl optionally substituted with halogen. In some embodiments, R¹ is C1-C6 alkyl substituted with phenyl optionally substituted with halogen. In some embodiments, R¹¹ is C1-C6 alkyl substituted with phenyl substituted with halogen. In some embodiments, R¹ is para-fluorobenzyl. In some embodiments, R¹¹ is C1-C6 alkyl substituted with phenyl. In some embodiments, R¹ is benzyl. In some embodiments, R¹ is ethyl-1-phenyl or ethyl-2-phenyl. In some embodiments, R¹¹ is C1-C6 alkyl. In some embodiments, R¹ is methyl, ethyl, or isopropyl. In some embodiments, R¹ is methyl.

In some embodiments, R¹ is C1-C6 thioalkyl. In some embodiments, R¹ is C1-C3 thioalkyl. In some embodiments, R¹ is thiomethyl, thioethyl, or thiopropyl. In some embodiments, R¹ is methyl-thiomethyl, methyl-thioethyl, or ethyl-thiomethyl. In some embodiments, R¹ is thiomethyl.

In some embodiments, R¹ is C1-C6 haloalkyl. In some embodiments, R¹ is C1-C3 haloalkyl. In some embodiments, R¹ is C1-C3 fluoroalkyl. In some embodiments, R¹ is CF₃. In some embodiments, R¹ is CHF₂.

In some embodiments, R¹ is C1-C6 alkoxy. In some embodiments, R¹ is C1-C3 alkoxy. In some embodiments, R¹ is —OCH₃, —OCH₂CH₃, or —OCH₂CH₂CH₃. In some embodiments, R¹ is —OCH₃.

In some embodiments, R¹ is C1-C6 alkoxyalkyl. In some embodiments, R¹ is C1-C3 alkoxyalkyl. In some embodiments, R¹ is —CH₂OCH₃, —CH₂OCH₂CH₃, or —CH₂CH₂OCH₃. In some embodiments, R¹ is —CH₂OCH₃.

In some embodiments, R^{1A} is absent.

In some embodiments, R^{1A} is hydrogen.

In some embodiments, R^{1A} is C1-C6 alkyl. In some embodiments, R^{1A} is methyl, ethyl, or isopropyl. In some embodiments, R^{1A} is methyl.

In some embodiments, R^{1A} is C1-C6 haloalkyl. In some embodiments, R^{1A} is C1-C3 haloalkyl. In some embodiments, R^{1A} is C1-C3 fluoroalkyl. In some embodiments, R^{1A} is trifluoromethyl. In some embodiments, R^{1A} is CHF₂.

In some embodiments, R^{1A} is C1-C6 thioalkyl. In some embodiments, R^{1A} is C1-C3 thioalkyl. In some embodiments, R^{1A} is thiomethyl, thioethyl, or thiopropyl. In some embodiments, R^{1A} is methyl-thiomethyl, methyl-thioethyl, or ethyl-thiomethyl. In some embodiments, R^{1A} is thiomethyl.

In some embodiments, R^{1A} is C1-C6 alkoxy. In some embodiments, R^{1A} is C1-C3 alkoxy.

In some embodiments, R^{1A} is —OCH₃, —OCH₂CH₃, or —OCH₂CH₂CH₃. In some embodiments, R^{1A} is —OCH₃.

In some embodiments, R^{1A} is C1-C6 alkoxyalkyl. In some embodiments, R^{1A} is C1-C3 alkoxyalkyl. In some embodiments, R^{1A} is —CH₂OCH₃, —CH₂OCH₂CH₃, or —CH₂CH₂OCH₃. In some embodiments, R^{1A} is —CH₂OCH₃.

In some embodiments, R^{Z1} is absent.

In some embodiments, R^{Z1} is hydrogen.

In some embodiments, R^{Z1} is cyano.

In some embodiments, R^{Z1} is C1-C6 alkyl. In some embodiments, R^{Z1} is methyl, ethyl, or isopropyl. In some embodiments, R^{Z1} is methyl.

In some embodiments, R^{Z1} is C1-C6 haloalkyl. In some embodiments, R^{Z1} is C1-C3 haloalkyl. In some embodiments, R^{Z1} is trifluoromethyl.

In some embodiments, R^{Z2} is hydrogen.

In some embodiments, R^{Z2} is C1-C6 alkyl. In some embodiments, R^{Z2} is methyl, ethyl, or isopropyl. In some embodiments, R^{Z2} is methyl.

In some embodiments, R^{Z2} is C1-C6 haloalkyl. In some embodiments, R^{Z2} is C1-C3 haloalkyl. In some embodiments, R^{Z2} is trifluoromethyl.

In some embodiments, R² is phenyl optionally substituted with 1-4 independently selected R^{2A}. In some embodiments, R² is phenyl substituted with 1 or 2 independently selected R^{2A}. In some embodiments, R² is phenyl substituted with 1 R^{2A}. In some embodiments, R² is phenyl substituted with 2 independently selected R^{2A}. In some embodiments, R² is phenyl optionally substituted with 3 independently selected R^{2A}. In some embodiments, R² is phenyl.

In some embodiments, R² is 5-10 membered heteroaryl optionally substituted with 1-4 independently selected R^{2A}. In some embodiments, R² is 5-10 membered heteroaryl substituted with 1 or 2 independently selected R^{2A}. In some embodiments, R² is 5-10 membered heteroaryl substituted with 1 R^{2A}. In some embodiments, R² is 5-10 membered heteroaryl substituted with 2 independently selected R^{2A}. In some embodiments, R² is 5-10 membered heteroaryl optionally substituted with 3 independently selected R^{2A}. In some embodiments, R² is 5-10 membered heteroaryl.

In some embodiments, R² is 6 membered heteroaryl optionally substituted with 1-4 independently selected R^{2A}. In some embodiments, R² is 6 membered heteroaryl substituted with 1 or 2 independently selected R^{2A}. In some embodiments, R² is 6 membered heteroaryl substituted with 1 R^{2A}. In some embodiments, R² is 6 membered heteroaryl substituted with 2 independently selected R^{2A}. In some embodiments, R² is 6 membered heteroaryl optionally substituted with 3 independently selected R^{2A}. In some embodiments, R² is 6 membered heteroaryl.

In some embodiments, R² is 9 membered heteroaryl optionally substituted with 1-4 independently selected R^{2A}. In some embodiments, R² is 9 membered heteroaryl substituted with 1 or 2 independently selected R^{2A}. In some embodiments, R² is 9 membered heteroaryl substituted with 1 R^{2A}. In some embodiments, R² is 9 membered heteroaryl substituted with 2 independently selected R^{2A}. In some embodiments, R² is 9 membered heteroaryl optionally substituted with 3 independently selected R^{2A}. In some embodiments, R² is 9 membered heteroaryl.

In some embodiments, the heteroaryl of R² is pyridinyl, pyrimidinyl, pyridazinyl, indole, indazole, azaindole, azaindazole, indoline, azaindoline, isoindoline, azaisoindoline, benzimidazolyl, benzoxazolyl, benzothiazolyl, benzoisoxazolyl, benzisothiazolyl, quinolinyl, or isoquinolinyl. In some embodiments, the heteroaryl of R² is pyridinyl or pyrimidinyl. In some embodiments, the heteroaryl of R² is indole, indazole, azaindole, azaindazole, indoline, azaindoline, isoindoline, or azaisoindoline.

In some embodiments, R² is 4-10 membered heterocyclyl optionally substituted with 1-4 independently selected R^{2A}. In some embodiments, R² is 4-10 membered heterocyclyl substituted with 1 or 2 independently selected R^{2A}. In some embodiments, R² is 4-10 membered heterocyclyl substituted with 1 R^{2A}. In some embodiments, R² is 4-10 membered heterocyclyl substituted with 2 independently selected R^{2A}. In some embodiments, R² is 4-10 membered heterocyclyl optionally substituted with 3 independently selected R^{2A}. In some embodiments, R² is 4-10 membered heterocyclyl.

In some embodiments, R² is 5-8 membered heterocyclyl optionally substituted with 1-4 independently selected R^{2A}. In some embodiments, R² is 5-8 membered heterocyclyl substituted with 1 or 2 independently selected R^{2A}. In some embodiments, R² is 5-8 membered heterocyclyl substituted with 1 R^{2A}. In some embodiments, R² is 5-8 membered heterocyclyl substituted with 2 independently selected R^{2A}. In some embodiments, R² is 5-8 membered heterocyclyl optionally substituted with 3 independently selected R^{2A}. In some embodiments, R² is 5-8 membered heterocyclyl.

In some embodiments, the heterocyclyl of R² is piperidinyl, piperazinyl, or morpholinyl.

In some embodiments, R² is 4-10 membered cycloalkyl optionally substituted with 1-4 independently selected R^{2A}. In some embodiments, R² is 4-10 membered cycloalkyl substituted with 1 or 2 independently selected R^{2A}. In some embodiments, R² is 4-10 membered cycloalkyl substituted with 1 R^{2A}. In some embodiments, R² is 4-10 membered cycloalkyl substituted with 2 independently selected R^{2A}. In some embodiments, R² is 4-10 membered cycloalkyl optionally substituted with 3 independently selected R^{2A}. In some embodiments, R² is 4-10 membered cycloalkyl.

In some embodiments, R² is 5-7 membered cycloalkyl optionally substituted with 1-4 independently selected R^{2A}. In some embodiments, R² is 5-7 membered cycloalkyl substituted with 1 or 2 independently selected R^{2A}. In some embodiments, R² is 5-7 membered cycloalkyl substituted with 1 R^{2A}. In some embodiments, R² is 5-7 membered cycloalkyl substituted with 2 independently selected R^{2A}. In some embodiments, R² is 5-7 membered cycloalkyl optionally substituted with 3 independently selected R^{2A}. In some embodiments, R² is 5-7 membered cycloalkyl.

In some embodiments, the cycloalkyl of R² is cyclopentyl, [1.1.1]bicyclopentyl, or cyclohexyl.

In some embodiments, R² is C1-C6 alkoxyalkyl optionally substituted with —C(═O)NR^{A}R^{C}. In some embodiments, R² is C1-C6 alkoxyalkyl. In some embodiments, R² is C1-C3 alkoxyalkyl. In some embodiments, R² is —CH₂OCH₃, —CH₂OCH₂CH₃, or —CH₂CH₂OCH₃. In some embodiments, R² is —CH₂OCH₃.

In some embodiments, 1, 2, 3, or 4 of R^{2A} are independently halogen. In some embodiments, 1, 2, or 3 of R^{2A} are independently fluoro or chloro. In some embodiments, 1 or 2 of R^{2A} are independently fluoro or chloro.

In some embodiments, 1, 2, 3, or 4 of R^{2A} are independently cyano. In some embodiments, 1 or 2 of R^{2A} are cyano.

In some embodiments, 1, 2, 3, or 4 of R^{2A} are independently hydroxyl. In some embodiments, 1 or 2 of R^{2A} are hydroxyl.

In some embodiments, 1, 2, 3, or 4 of R^{2A} are independently —NR^{A}R^{B}. In some embodiments, 1 or 2 of R^{2A} are independently —NR^{A}R^{B}.

In some embodiments, 1, 2, 3, or 4 of R^{2A} are independently —C(═O)NR^{A}R^{B}. In some embodiments, 1 or 2 of R^{2A} are independently —C(═O)NR^{A}R^{B}.

In some embodiments, 1, 2, 3, or 4 of R^{2A} are independently

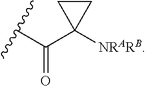

In some embodiments, 1 or 2 of R^{2A} are independently

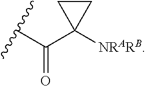

In some embodiments, 1, 2, 3, or 4 of R^{2A} are independently —NHC(═O)R^{C}. In some embodiments, 1 or 2 of R^{2A} are independently —NHC(═O)R^{C}.

In some embodiments, 1, 2, 3, or 4 of R^{2A} are independently —C(═O)NR^{D}R^{E}. In some embodiments, 1 or 2 of R^{2A} are independently —C(═O)NR^{D}R^{E}.

In some embodiments, 1, 2, 3, or 4 of R^{2A} are independently —C(═O)OR^{F}. In some embodiments, 1 or 2 of R^{2A} are independently —C(═O)OR^{F}.

In some embodiments, 1, 2, 3, or 4 of R^{2A} are independently —SO₂R^{F}. In some embodiments, 1 or 2 of R^{2A} are independently —SO₂R^{F}.

In some embodiments, 1, 2, 3, or 4 of R^{2A} are independently —NHSO₂R^{F}. In some embodiments, 1 or 2 of R^{2A} are independently —NHSO₂R^{F}.

In some embodiments, 1, 2, 3, or 4 of R^{2A} are independently —SO₂NR^{F}R^{G}. In some embodiments, 1 or 2 of R^{2A} are independently —SO₂NR^{F}R^{G}.

In some embodiments, 1, 2, 3, or 4 of R^{2A} are independently —NHC(═O)C1-C6 alkyl optionally substituted with NR^{A}R^{B}. In some embodiments, 1, 2, or 3 of R^{2A} are independently —NHC(═O)C1-C6 alkyl substituted with NR^{A}R^{B}. In some embodiments, 1, 2, or 3 of R^{2A} are independently —NHC(═O)C1-C6 alkyl. In some embodiments, 1 or 2 of R^{2A} are independently —NHC(═O)C1-C6 alkyl optionally substituted with NR^{A}R^{B}. In some embodiments, 1 or 2 of R^{2A} are independently —NHC(═O)C1-C6 alkyl substituted with NR^{A}R^{B}. In some embodiments, 1 or 2 of R^{2A} are independently —NHC(═O)C1-C6 alkyl.

In some embodiments, 1, 2, 3, or 4 of R^{2A} are independently C1-C6 haloalkyl. In some embodiments, 1 or 2 of R^{2A} are independently C1-C3 haloalkyl. In some embodiments, 1 or 2 of R^{2A} are trifluoromethyl.

In some embodiments, 1, 2, 3, or 4 of R^{2A} are independently C1-C6 hydroxyalkyl. In some embodiments, 1 or 2 of R^{2A} are independently C1-C3 hydroxyalkyl.

In some embodiments, 1, 2, 3, or 4 of R^{2A} are independently 5-10 membered heteroaryl optionally substituted with 1-3 substituents independently selected from C1-C6 alkyl and —NR^{A}R^{B}. In some embodiments, 1, 2, 3, or 4 of R^{2A} are independently 5-10 membered heteroaryl substituted with 1-3 substituents independently selected from C1-C6 alkyl and —NR^{A}R^{B}. In some embodiments, 1, 2, 3, or 4 of R^{2A} are independently 5-10 membered heteroaryl.

In some embodiments, 1 of R^{2A} is 5-6 membered heteroaryl optionally substituted with 1-3 substituents independently selected from C1-C6 alkyl and —NR^{A}R^{B}. In some embodiments, 1 of R^{2A} is 5-6 membered heteroaryl substituted with 1-3 substituents independently selected from C1-C6 alkyl and —NR^{A}R^{B}. In some embodiments, 1 of R^{2A} is 5-6 membered heteroaryl.

In some embodiments, 1, 2, 3, or 4 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, 1 of R^{2A} is 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl substituted with C1-C6 alkoxy, —C(═O)NR^{A}R^{B}, or —NHC(═O)C1-C6 alkyl substituted with —NR^{A}R^{B}.

In some embodiments, 1, 2, 3, or 4 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl. In some embodiments, 1 of R^{2A} is 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl.

In some embodiments, 1, 2, 3, or 4 of R^{2A} are independently 4-10 membered heterocyclyl.

In some embodiments, 1 of R^{2A} is 4-10 membered heterocyclyl.

In some embodiments, 1, 2, or 3 of R^{2A} are independently C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

In some embodiments, 1, 2, 3, or 4 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl.

In some embodiments, 1, 2, 3, or 4 of R^{2A} are independently C1-C6 alkyl. In some embodiments, 1, 2, or 3 of R^{2A} are independently C1-C3 alkyl. In some embodiments, 1, 2, or 3 of R^{2A} are methyl. In some embodiments, 1 or 2 of R^{2A} are independently C1-C6 alkyl. In some embodiments, 1 or 2 of R^{2A} are independently C1-C3 alkyl. In some embodiments, 1 or 2 of R^{2A} are methyl.

In some embodiments, 1, 2, 3, or 4 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl optionally substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl substituted with C1-C6 alkyl, aralkyl, heteroaralkyl, or —C(═O)C3-C6 cycloalkyl.

In some embodiments, 1, 2, 3, or 4 of R^{2A} are independently C1-C6 alkoxy substituted with —NR^{A}R^{B} or 4-10 membered heterocyclyl.

In some embodiments, 1, 2, 3, or 4 of R^{2A} are independently C1-C6 alkoxy.

In some embodiments, 1, 2, 3, or 4 of R^{2A} are independently C3-C6 cycloalkyl optionally substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl optionally substituted with C1-C6 alkyl.

In some embodiments, 1, 2, 3, or 4 of R^{2A} are independently C3-C6 cycloalkyl substituted with 4-10 membered heterocyclyl substituted with C1-C6 alkyl.

In some embodiments, 1, 2, 3, or 4 of R^{2A} are independently C3-C6 cycloalkyl substituted with 4-10 membered heterocyclyl.

In some embodiments, 1, 2, 3, or 4 of R^{2A} are independently C3-C6 cycloalkyl. In some embodiments, 1 or 2 of R^{2A} are independently C3-C6 cycloalkyl.

In some embodiments, R² is piperidinyl substituted with 1-2 independently selected R^{2A}.

In some embodiments, R² is piperidinyl substituted with 2-4 independently selected R^{2A}. In some embodiments, R² is morpholinyl substituted with 2-4 independently selected R^{2A}. In some embodiments, R² is morpholinyl. In some embodiments, R² is piperazinyl substituted with 1-2 independently selected R^{2A}.

In some embodiments, R² is phenyl substituted with 1-2 independently selected R^{2A}. In some embodiments, R² is pyridinyl substituted with 1-2 independently selected R^{2A}.

In some embodiments, R² is indolyl substituted with 1-2 independently selected R^{2A}. In some embodiments, R² is indazolyl substituted with 1-2 independently selected R^{2A}. In some embodiments, R² is 7-azaindolyl substituted with 1-2 independently selected R^{2A}. In some embodiments, R² is 7-azaindazolyl substituted with 1-2 independently selected R^{2A}. In some embodiments, R² is 1,2-dihydro-3H-indazol-3-one substituted with 1-2 independently selected R^{2A}. In some embodiments, R² is isoindolinyl substituted with 1-2 independently selected R^{2A}. In some embodiments, R² is isoindolinyl. In some embodiments, R² is 2-indolinone substituted with 1-2 independently selected R^{2A}. In some embodiments, R² is benzimidazolyl substituted with 1-2 independently selected R^{2A}. In some embodiments, R² is imidazopyridinyl substituted with 1-2 independently selected R^{2A}. In some embodiments, R² is 1,3-dihydro-2H-benzo[d]imidazol-2-onyl substituted with 1-2 independently selected R^{2A}. In some embodiments, R² is [1,2,4]triazolo[1,5-a]pyridine substituted with 1-2 independently selected R^{2A}.

In some embodiments, one R^{2A} is C1-C6 alkyl, C1-C6 haloalkyl, C1-C6 hydroxyalkyl, cyano, or hydroxyl, and the other R^{2A} are independently selected from C1-C6 haloalkyl, C1-C6 hydroxyalkyl, cyano, hydroxyl, halogen, —NR^{A}R^{B}, —C(═O)NR^{A}R^{B},

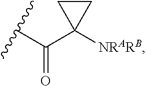

—NHC(═O)R^{C}, —C(═O)NR^{D}R^{E}, —C(═O)OR^{F}, —SO₂R^{F}, —NHSO₂R^{F}, —SO₂NR^{F}R^{G}, —NHC(═O)C1-C6 alkyl optionally substituted with NR^{A}R^{B}, 5-10 membered heteroaryl optionally substituted with 1-3 substituents independently selected from C1-C6 alkyl and —NR^{A}R^{B}, 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B} C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl, C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and C3-C6 cycloalkyl optionally substituted with 4-10 membered heterocyclyl optionally substituted with C1-C6 alkyl.

In some embodiments, each R^{2A} is independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1, 2, or 3 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, 1 or 2 R^{2A} are independently selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, R² is substituted with 1 R^{2A}. In some embodiments, R² is substituted with 2 independently selected R^{2A}. In some embodiments, R² is substituted with 3 independently selected R^{2A}. In some embodiments, R² is substituted with 4 independently selected R^{2A}.

In some embodiments, R² is substituted with 1 R^{2A}. In some embodiments, R^{2A} is halogen. In some embodiments, R^{2A} is fluoro or chloro. In some embodiments, R^{2A} is cyano. In some embodiments, R^{2A} is hydroxyl. In some embodiments, R^{2A} is R^{2A}, —NR^{A}R^{B}. In some embodiments, R^{2A} is —C(═O)NR^{A}R^{B}. In some embodiments, R^{2A} is

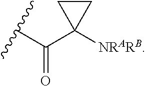

In some embodiments, R^{2A} is —NHC(═O)R^{C}. In some embodiments, —C(═O)NR^{D}R^{B}. In some embodiments, R^{2A} is —C(═O)OR^{F}. In some embodiments, R^{2A} is —SO₂R^{F}. In some embodiments, R^{2A} is —NHSO₂R^{F}. In some embodiments, R^{2A} is —NHC(═O)C1-C6 alkyl optionally substituted with NR^{A}R^{B}. In some embodiments, R^{2A} is C1-C6 haloalkyl. In some embodiments, R^{2A} is C1-C6 hydroxyalkyl. In some embodiments, R^{2A} is 5-10 membered heteroaryl optionally substituted with 1-3 substituents independently selected from C1-C6 alkyl and —NR^{A}R^{B}. In some embodiments, R^{2A} is 5-6 membered heteroaryl optionally substituted with 1-3 substituents independently selected from C1-C6 alkyl and —NR^{A}R^{B}. In some embodiments, R^{2A} is 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B} In some embodiments, R^{2A} is 4-10 membered heterocyclyl. In some embodiments, R^{2A} is C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl. In some embodiments, R^{2A} is C1-C6 alkyl. In some embodiments, R^{2A} is C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl. In some embodiments, R^{2A} is C1-C6 alkoxy.

In some embodiments, R^{2A} is C3-C6 cycloalkyl optionally substituted with 4-10 membered heterocyclyl optionally substituted with C1-C6 alkyl. In some embodiments, R^{2A} is selected from halogen, methyl, ethyl, —NH₂, —NHCH₃, —N(CH₃)₂, —C(═O)NH₂, —C(═O)NHCH₃, —C(═O)N(CH₃)₂, —C(═O)NHOH, —SO₂NH₂, —SO₂NHCH₃, —SO₂N(CH₃)₂, cyclopropyl, cyclobutyl, trifluoromethyl, 2,2,2-trifluoroethyl, and acetyl.

In some embodiments, R² is C1-C6 alkoxy optionally substituted with —C(═O)NR^{A}R^{C}. In some embodiments, R² is C1-C6 alkoxy substituted with —C(═O)NR^{A}R^{C}. In some embodiments, R² is C3-C6 alkoxy substituted with —C(═O)NR^{A}R^{C}. In some embodiments, R² is C1-C6 alkoxy.

In some embodiments, X is a bond.

In some embodiments, X is CH₂.

In some embodiments, X is CH(CH₃).

In some embodiments, X is C(CH₃)₂.

In some embodiments, X is

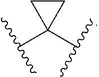 [SMILES: *C1(*)CC1]

In some embodiments, W is O.

In some embodiments, R^{3A} is hydrogen.

In some embodiments, R^{3A} is C1-C6 alkyl. In some embodiments, R^{3A} is methyl or ethyl.

In some embodiments, R^{3A} is methyl.

In some embodiments, R^{3A} is C1-C6 alkoxy. In some embodiments, R^{3A} is C1-C3 alkoxy.

In some embodiments, R^{3A} is —OCH₃, —OCH₂CH₃, or —OCH₂CH₂CH₃. In some embodiments, R^{3A} is —OCH₃.

In some embodiments, R^{3A} is C1-C6 haloalkyl. In some embodiments, R^{3A} is C1-C3 haloalkyl. In some embodiments, R^{3A} is C1-C3 fluoroalkyl. In some embodiments, R^{3A} is CF₃. In some embodiments, R^{3A} is CHF₂.

In some embodiments, W is NR^{3B}.

In some embodiments, one of R^{3A} and R^{3B} is hydrogen and the other of R^{3A} and R^{3B} is C1-C6 alkyl. In some embodiments, one of R^{3A} and R^{3B} is hydrogen and the other of R^{3A} and R^{3B} is methyl. In some embodiments, each of R^{3A} and R^{3B} is hydrogen. In some embodiments, each of R^{3A} and R^{3B} is an independently selected C1-C6 alkyl. In some embodiments, each of R^{3A} and R^{3B} is methyl.

In some embodiments, one of R^{3A} and R^{3B} is hydrogen and the other of R^{3A} and R^{3B} is C1-C6 alkoxy. In some embodiments, one of R^{3A} and R^{3B} is C1-C6 alkyl and the other of R^{3A} and R^{3B} is C1-C6 alkoxy. In some embodiments, R^{3A} is C1-C6 alkoxy. In some embodiments, R^{3A} is C1-C3 alkoxy. In some embodiments, R^{3A} is —OCH₃, —OCH₂CH₃, or —OCH₂CH₂CH₃. In some embodiments, R^{3A} is —OCH₃.

In some embodiments, one of R^{3A} and R^{3B} is hydrogen and the other of R^{3A} and R^{3B} is C1-C6 haloalkyl. In some embodiments, one of R^{3A} and R^{3B} is C1-C6 alkyl and the other of R^{3A} and R^{3B} is C1-C6 haloalkyl. In some embodiments, R^{3A} is C1-C6 haloalkyl. In some embodiments, R^{3A} is C1-C3 haloalkyl. In some embodiments, R^{3A} is C1-C3 fluoroalkyl. In some embodiments, R^{3A} is CF₃. In some embodiments, R^{3A} is CHF₂.

In some embodiments, R^{3A} and R^{3B}, together with the carbon and nitrogen atoms, respectively, to which they are attached together form a 4-8 membered heterocyclyl group. In some embodiments, R^{3A} and R^{3B}, together with the carbon and nitrogen atoms, respectively, to which they are attached together form a 5-6 membered heterocyclyl group.

In some embodiments, Y is phenyl optionally substituted with R^{Y}, naphthyl substituted with R^{Y}, or 5-10 membered heteroaryl substituted with R^{Y}.

In some embodiments, Y is phenyl optionally substituted with 1-3 independently selected R^{Y}. In some embodiments, Y is phenyl substituted with 1 or 2 independently selected R^{Y}.

In some embodiments, Y is phenyl substituted with 1 R^{Y}. In some embodiments, Y is phenyl substituted with 2 independently selected R^{Y}. In some embodiments, Y is phenyl optionally substituted with 3 independently selected R^{Y}. In some embodiments, Y is phenyl.

In some embodiments, Y is naphthyl optionally substituted with 1-3 independently selected R^{Y}. In some embodiments, Y is naphthyl substituted with 1 or 2 independently selected R^{Y}. In some embodiments, Y is naphthyl substituted with 1 R^{Y}. In some embodiments, Y is naphthyl substituted with 2 independently selected R^{Y}. In some embodiments, Y is naphthyl optionally substituted with 3 independently selected R^{Y}. In some embodiments, Y is naphthyl.

In some embodiments, Y is 5-10 membered heteroaryl optionally substituted with 1-3 independently selected R^{Y}. In some embodiments, Y is 5-10 membered heteroaryl substituted with 1 or 2 independently selected R^{Y}. In some embodiments, Y is 5-10 membered heteroaryl substituted with 1 R^{Y}. In some embodiments, Y is 5-10 membered heteroaryl substituted with 2 independently selected R^{Y}. In some embodiments, Y is 5-10 membered heteroaryl optionally substituted with 3 independently selected R^{Y}. In some embodiments, Y is 5-10 membered heteroaryl.

In some embodiments, Y is 6 membered heteroaryl optionally substituted with 1-3 independently selected R^{Y}. In some embodiments, Y is 6 membered heteroaryl substituted with 1 or 2 independently selected R^{Y}. In some embodiments, Y is 6 membered heteroaryl substituted with 1 R^{Y}. In some embodiments, Y is 6 membered heteroaryl substituted with 2 independently selected R^{Y}. In some embodiments, Y is 6 membered heteroaryl optionally substituted with 3 independently selected R^{Y}. In some embodiments, Y is 6 membered heteroaryl.

In some embodiments, Y is 9 membered heteroaryl optionally substituted with 1-3 independently selected R^{Y}. In some embodiments, Y is 9 membered heteroaryl substituted with 1 or 2 independently selected R^{Y}. In some embodiments, Y is 9 membered heteroaryl substituted with 1 R^{Y}. In some embodiments, Y is 9 membered heteroaryl substituted with 2 independently selected R^{Y}. In some embodiments, Y is 9 membered heteroaryl optionally substituted with 3 independently selected R^{Y}. In some embodiments, Y is 9 membered heteroaryl.

In some embodiments, 1, 2, or 3 of R^{Y} is independently halogen. In some embodiments, 1, 2, or 3 of R^{Y} is independently chloro or fluoro. In some embodiments, 1 or 2 of R^{Y} is independently chloro or fluoro.

In some embodiments, 1, 2, or 3 of R^{Y} is hydroxyl. In some embodiments, 1 or 2 of R^{Y} is hydroxyl.

In some embodiments, 1, 2, or 3 of R^{Y} is cyano. In some embodiments, 1 or 2 of R^{Y} is cyano.

In some embodiments, 1, 2, or 3 of R^{Y} is independently C1-C6 haloalkyl optionally substituted with hydroxyl. In some embodiments, 1, 2, or 3 of R^{Y} is independently C1-C6 haloalkyl substituted with hydroxyl. In some embodiments, 1, 2, or 3 of R^{Y} is independently C1-C6 haloalkyl. In some embodiments, 1 or 2 of R^{Y} is independently C1-C3 haloalkyl. In some embodiments, 1 or 2 of R^{Y} is trifluoromethyl. In some embodiments, 1, 2, or 3 of R^{Y} is independently C1-C6 haloalkyl. In some embodiments, 1 or 2 of R^{Y} is independently C1-C3 haloalkyl. In some embodiments, 1 or 2 of R^{Y} is trifluoromethyl.

In some embodiments, 1, 2, or 3 of R^{Y} is independently C1-C6 alkoxy. In some embodiments, 1 or 2 of R^{Y} is independently C1-C3 alkoxy. In some embodiments, 1 or 2 of R^{Y} is methoxy.

In some embodiments, 1, 2, or 3 of R^{Y} is independently C1-C6 haloalkoxy. In some embodiments, 1 or 2 of R^{Y} is independently C1-C3 haloalkoxy. In some embodiments, 1 or 2 of R^{Y} is trifluoromethoxy.

In some embodiments, 1, 2, or 3 of R^{Y} is independently C1-C6 hydroxyalkyl. In some embodiments, 1 or 2 of R^{Y} is independently C1-C3 hydroxyalkyl. In some embodiments, 1 or 2 of R^{Y} is independently mono-hydroxyl C1-C3 alkyl. In some embodiments, 1 or 2 of R^{Y} is independently di-hydroxyl C2-C3 alkyl.

In some embodiments, 1, 2, or 3 of R^{Y} is independently —NHC(═O)R^{C}. In some embodiments, 1 or 2 of R^{Y} is independently —NHC(═O)R^{C}. In some embodiments, 1 of R^{Y} is —NHC(═O)R^{C}. In some embodiments, Y is substituted with 1 R^{Y}, and R^{Y} is —NHC(═O)R^{C}.

In some embodiments, 1, 2, or 3 of R^{Y} is independently —C(═O)NHR^{Y1}. In some embodiments, 1 or 2 of R^{Y} is independently —C(═O)NR¹. In some embodiments, 1 of R^{Y} is —C(═O)NHR^{Y1}. In some embodiments, Y is substituted with 1 R^{Y}, and R^{Y} is —C(═O)NHR^{Y1}.

In some embodiments, 1, 2, or 3 of R^{Y} is independently —CO₂R^{A}. In some embodiments, 1 or 2 of R^{Y} is independently —CO₂R^{A}. In some embodiments, 1 of R^{Y} is —CO₂R^{A}. In some embodiments, Y is substituted with 1 R^{Y}, and R^{Y} is —CO₂R^{A}. In some embodiments, Y is substituted with 1 R^{Y}, and R^{Y} is —CO₂H.

In some embodiments, 1, 2, or 3 of R^{Y} is independently —SO₂NR^{F}R^{G}. In some embodiments, 1 or 2 of R^{Y} is independently —SO₂NR^{F}R^{G}. In some embodiments, 1 of R^{Y} is —SO₂NR^{F}R^{G}. In some embodiments, Y is substituted with 1 R^{Y}, and R^{Y} is —SO₂NR^{F}R^{G}.

In some embodiments, 1, 2, or 3 of R^{Y} is independently —NHSO₂R^{F}. In some embodiments, 1 or 2 of R^{Y} is independently —NHSO₂R^{F}. In some embodiments, 1 of R^{Y} is —NHSO₂R^{F}. In some embodiments, Y is substituted with 1 R^{Y}, and R^{Y} is —NHSO₂R^{F}.

In some embodiments, 1, 2, or 3 of R^{Y} is independently —S(═O)(═NR^{F})R^{G}. In some embodiments, 1 or 2 of R^{Y} is independently —S(═O)(═NR^{F})R^{G}. In some embodiments, 1 of R^{Y} is —S(═O)(═NR^{F})R^{G}. In some embodiments, Y is substituted with 1 R^{Y}, and R^{Y} is —S(═O)(═NR^{F})R^{G}.

In some embodiments, 1, 2, or 3 of R^{Y} is independently —SO₂(C1-C6 alkyl). In some embodiments, 1 or 2 of R^{Y} is independently —SO₂(C1-C6 alkyl). In some embodiments, 1 of R^{Y} is —SO₂(C1-C6 alkyl). In some embodiments, 1 or 2 of R^{Y} is —SO₂CH₃. In some embodiments, 1 of R^{Y} is —SO₂CH₃. In some embodiments, Y is substituted with 1 R^{Y}, and R^{Y} is —SO₂CH₃.

In some embodiments, 1, 2, or 3 of R^{Y} is independently —C(═O)NR^{A}R^{B}. In some embodiments, 1 or 2 of R^{Y} is independently —C(═O)NR^{A}R^{B}. In some embodiments, 1 of R^{Y} is —C(═O)NR^{A}R^{B}. In some embodiments, Y is substituted with 1 R^{Y}, and R^{Y} is —C(═O)NR^{A}R^{B}.

In some embodiments, 1, 2, or 3 of R^{Y} is independently 4-6 membered heteroaryl. In some embodiments, 1 of R^{Y} is 4-6 membered heteroaryl. In some embodiments, Y is substituted with 1 R^{Y}, and R^{Y} is 4-6 membered heteroaryl.

In some embodiments, 1, 2, or 3 of R^{Y} is independently heteroaralkyl. In some embodiments, 1 of R^{Y} is independently heteroaralkyl.

In some embodiments, 1, 2, or 3 of R^{Y} is independently 4-6 membered heterocyclyl optionally substituted with R^{Y1}. In some embodiments, 1 of R^{Y} is 4-6 membered heterocyclyl. In some embodiments, 1 of R^{Y} is 4-6 membered heterocyclyl substituted with R^{Y1}. In some embodiments, Y is substituted with 1 R^{Y}, and R^{Y} is 4-6 membered heterocyclyl substituted with R^{Y1}, where R^{Y1} is hydroxyl. In some embodiments, R^{Y} is

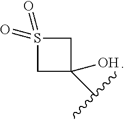 [SMILES: *C1(O)CS(=O)(=O)C1]

In some embodiments, R^{Y} is

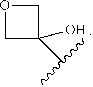 [SMILES: *C1(O)COC1]

In some embodiments, Y is selected from the group consisting of:

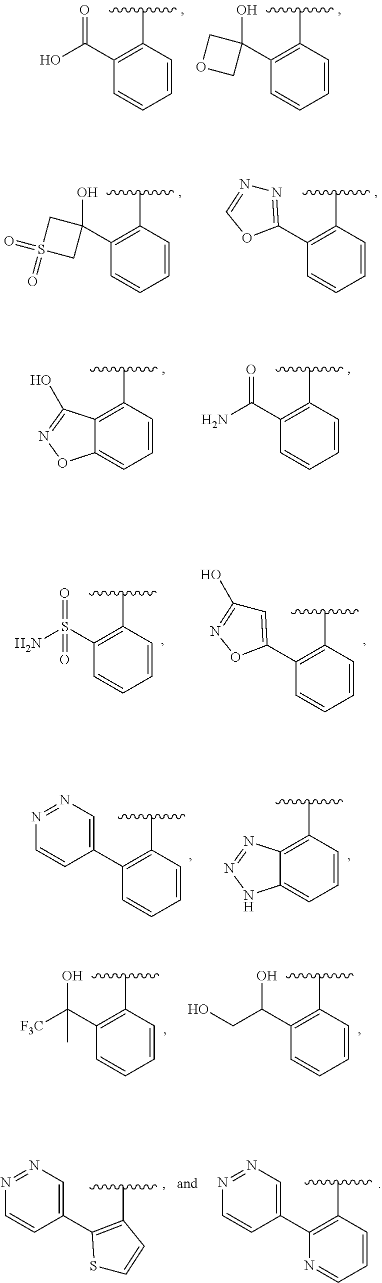

In some embodiments, Y is selected from the group consisting of:

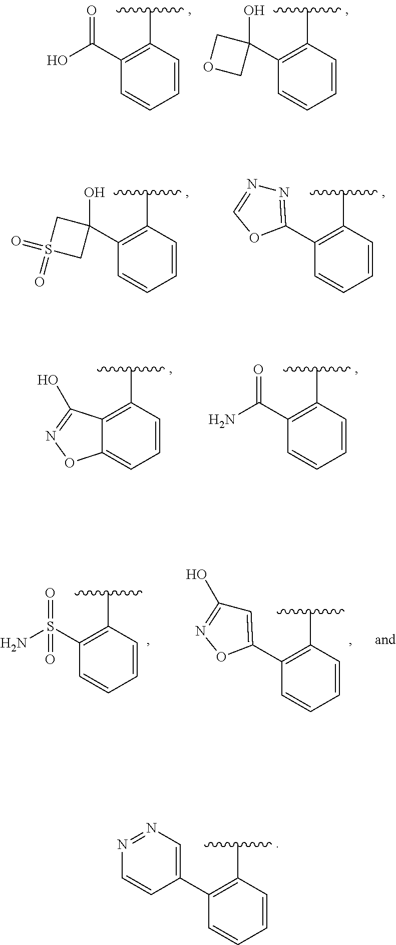

In some embodiments, 1, 2, or 3 of R^{Y} is independently C1-C6 alkyl optionally substituted with —CO₂R^{A} or 4-6 membered heteroaryl optionally substituted with R^{Y1}. In some embodiments, 1, 2, or 3 of R^{Y} is independently C1-C6 alkyl substituted with —CO₂R^{A} or 4-6 membered heteroaryl optionally substituted with R^{Y1}. In some embodiments, 1, 2, or 3 of R^{Y} is independently C1-C6 alkyl substituted with —CO₂R^{A} or 4-6 membered heteroaryl substituted with R^{Y1}. In some embodiments, 1, 2, or 3 of R^{Y} is independently C1-C6 alkyl substituted with —CO₂R^{A} or 4-6 membered heteroaryl. In some embodiments, Y is substituted with 1 R^{Y}, and R^{Y} is C1-C6 alkyl substituted with —CO₂R^{A}. In some embodiments, Y is substituted with 1 R^{Y}, and R^{Y} is C1-C6 alkyl substituted with —CO₂H.

In some embodiments, 1 or 2 of R^{Y} is independently C1-C6 alkyl optionally substituted with —CO₂R^{A} or 4-6 membered heteroaryl optionally substituted with R^{Y1}. In some embodiments, 1 or 2 of R^{Y} is independently C1-C6 alkyl substituted with —CO₂R^{A} or 4-6 membered heteroaryl optionally substituted with R^{Y1}. In some embodiments, 1 or 2 of R^{Y} is independently C1-C6 alkyl substituted with —CO₂R^{A} or 4-6 membered heteroaryl substituted with R^{Y}. In some embodiments, 1 or 2 of R^{Y} is independently C1-C6 alkyl substituted with —CO₂R^{A} or 4-6 membered heteroaryl.

In some embodiments, 1 or 2 of R^{Y} is independently C1-C6 alkyl substituted with —CO₂R^{A}. In some embodiments, 1 or 2 of R^{Y} is independently C1-C6 alkyl substituted 4-6 membered heteroaryl optionally substituted with R^{Y1}. In some embodiments, 1 or 2 of R^{Y} is independently C1-C6 alkyl substituted with 4-6 membered heteroaryl substituted with R^{Y1}. In some embodiments, 1 or 2 of R^{Y} is independently C1-C6 alkyl substituted with 4-6 membered heteroaryl.

In some embodiments, 1, 2, or 3 of R^{Y} is independently 5-6 membered heteroaryl. In some embodiments, 1 of R^{Y} is 5-6 membered heteroaryl. In some embodiments, Y is substituted with 1 R^{Y}, and R^{Y} is 5-6 membered heteroaryl.

In some embodiments, 1, 2, or 3 of R^{Y} is independently heteroaralkyl. In some embodiments, 1 of R^{Y} is independently heteroaralkyl.

In some embodiments, 1, 2, or 3 of R^{Y} is independently C1-C6 alkyl optionally substituted with —CO₂R^{A} or 5-6 membered heteroaryl optionally substituted with R^{Y1}. In some embodiments, 1, 2, or 3 of R^{Y} is independently C1-C6 alkyl substituted with —CO₂R^{A} or 5-6 membered heteroaryl optionally substituted with R^{Y1}. In some embodiments, 1, 2, or 3 of R^{Y} is independently C1-C6 alkyl substituted with —CO₂R^{A} or 5-6 membered heteroaryl substituted with R^{Y1}. In some embodiments, 1, 2, or 3 of R^{Y} is independently C1-C6 alkyl substituted with —CO₂R^{A} or 5-6 membered heteroaryl. In some embodiments, Y is substituted with 1 R^{Y}, and R^{Y} is C1-C6 alkyl substituted with —CO₂R^{A}. In some embodiments, Y is substituted with 1 R^{Y}, and R^{Y} is C1-C6 alkyl substituted with —CO₂H.

In some embodiments, 1 or 2 of R^{Y} is independently C1-C6 alkyl optionally substituted with —CO₂R^{A} or 5-6 membered heteroaryl optionally substituted with R^{Y1}. In some embodiments, 1 or 2 of R^{Y} is independently C1-C6 alkyl substituted with —CO₂R^{A} or 5-6 membered heteroaryl optionally substituted with R^{Y1}. In some embodiments, 1 or 2 of R^{Y} is independently C1-C6 alkyl substituted with —CO₂R^{A} or 5-6 membered heteroaryl substituted with R^{Y}. In some embodiments, 1 or 2 of R^{Y} is independently C1-C6 alkyl substituted with —CO₂R^{A} or 5-6 membered heteroaryl.

In some embodiments, 1 or 2 of R^{Y} is independently C1-C6 alkyl substituted with —CO₂R^{A}. In some embodiments, 1 or 2 of R^{Y} is independently C1-C6 alkyl substituted 5-6 membered heteroaryl optionally substituted with R^{Y1}. In some embodiments, 1 or 2 of R^{Y} is independently C1-C6 alkyl substituted with 5-6 membered heteroaryl substituted with R^{Y1}. In some embodiments, 1 or 2 of R^{Y} is independently C1-C6 alkyl substituted with 5-6 membered heteroaryl.

In some embodiments, 1, 2, or 3 of R^{Y} is independently C1-C6 alkyl. In some embodiments, 1 or 2 of R^{Y} is independently C1-C3 alkyl. In some embodiments, 1, 2, or 3 of R^{Y} is methyl.

In some embodiments, Y is substituted with 1 R^{Y}. In some embodiments, Y is substituted with 2 independently selected R^{Y}. In some embodiments, Y is substituted with 3 independently selected R^{Y}.

In some embodiments, Y is substituted with 1 R^{Y}. In some embodiments, R^{Y} is halogen. In some embodiments, R^{Y} is chloro or fluoro. In some embodiments, R^{Y} is hydroxyl. In some embodiments, R^{Y} is cyano. In some embodiments, R^{Y} is C1-C6 haloalkyl optionally substituted with hydroxyl. In some embodiments, R^{Y} is C1-C6 haloalkyl. In some embodiments, R^{Y} is C1-C6 alkoxy. In some embodiments, R^{Y} is C1-C6 haloalkoxy. In some embodiments, R^{Y} is C1-C6 hydroxyalkyl. In some embodiments, R^{Y} is —NHC(═O)R^{C}. In some embodiments, R^{Y} is —C(═O)NHR^{Y1}. In some embodiments, R^{Y} is —CO₂R^{A}. In some embodiments, R^{Y} is —SO₂NR^{F}R^{G}. In some embodiments, R^{Y} is —NHSO₂R^{F}. In some embodiments, R^{Y} is —S(═O)(═NR^{F})R^{G}. In some embodiments, R^{Y} is —SO₂(C1-C6 alkyl). In some embodiments, R^{Y} is —C(═O)NR^{A}R^{B}. In some embodiments, R^{Y} is 4-6 membered heteroaryl. In some embodiments, R^{Y} is heteroaralkyl. In some embodiments, R^{Y} is 4-6 membered heterocyclyl optionally substituted with R^{Y1}. In some embodiments, R^{Y} is C1-C6 alkyl optionally substituted with —CO₂R^{A} or 4-6 membered heteroaryl optionally substituted with R^{Y1}. In some embodiments, R^{Y} is

In some embodiments, R^{Y1} is —SO₂(C1-C6 alkyl). In some embodiments, R^{Y1} is —SO₂CH₃.

In some embodiments, R^{Y1} is C1-C6 alkyl optionally substituted with oxo. In some embodiments, R^{Y1} is C1-C6 alkyl substituted with oxo. In some embodiments, R^{Y1} is acetyl, 1-oxoethyl, or 1-oxopropyl. In some embodiments, R^{Y1} is C1-C6 alkyl. In some embodiments, R^{Y1} is methyl.

In some embodiments, R⁴ is hydrogen.

In some embodiments, R^{Y1} is hydroxyl.

In some embodiments, R⁴ is C1-C6 alkyl. In some embodiments, R⁴ is methyl or ethyl. In some embodiments, R⁴ is methyl.

In some embodiments, R⁴ is acrylamido.

In some embodiments, R⁵ is hydrogen.

In some embodiments, R⁵ is C1-C6 alkyl. In some embodiments, R⁵ is methyl or ethyl. In some embodiments, R⁵ is methyl.

In some embodiments, R⁵ is cyano.

In some embodiments, R⁵ is —NR^{5A}R^{5B}.

In some embodiments, R⁵ is —NR^{5A}C(═O)R^{5B}.

In some embodiments, R⁵ is —C(═O)NR^{5A}R^{5B}.

In some embodiments, one of R^{5A} and R^{5B} is hydrogen and the other of R^{5A} and R^{5B} is C1-C6 alkyl, C2-C6 alkenyl, or C1-C6 hydroxyalkyl. In some embodiments, one of R^{5A} and R^{5B} is C1-C6 alkyl and the other of R^{5A} and R^{5B} is C1-C6 alkyl, C2-C6 alkenyl, or C1-C6 hydroxyalkyl. In some embodiments, each of R^{5A} and R^{5B} is hydrogen. In some embodiments, each of R^{5A} and R^{5B} is an independently selected C1-C6 alkyl. In some embodiments, each of R^{5A} and R^{5B} is methyl. In some embodiments, the C1-C6 hydroxyalkyl of R^{5A} and R^{5B} is hydroxymethyl, 1-hydroxyethyl, 2-hydroxyethyl, dihydroxypropyl or dihydroxybutyl.

In some embodiments, R⁵ is acrylamido.

In some embodiments, R⁶ is hydrogen.

In some embodiments, R⁶ is halogen. In some embodiments, R⁶ is fluoro. In some embodiments, R⁶ is chloro.

In some embodiments, R⁶ is C1-C6 alkyl. In some embodiments, R⁶ is methyl.

In some embodiments, R⁷ is hydrogen.

In some embodiments, R⁷ is halogen.

In some embodiments, R⁷ is hydroxyl.

In some embodiments, R⁷ is cyano.

In some embodiments, R⁷ is C1-C6 alkyl. In some embodiments, R⁷ is methyl, ethyl, or isopropyl. In some embodiments, R⁷ is methyl.

In some embodiments, R⁷ is C1-C6 alkoxy. In some embodiments, R⁷ is C1-C3 alkoxy. In some embodiments, R⁷ is methoxy.

In some embodiments, R⁷ is C1-C6 alkoxyalkyl. In some embodiments, R⁷ is C1-C3 alkoxy. In some embodiments, R⁷ is methoxymethyl.

In some embodiments, R⁷ is C1-C6 hydroxyalkyl. In some embodiments, R⁷ is C1-C3 hydroxyalkyl. In some embodiments, R⁷ is mono-hydroxyl C1-C3 alkyl. In some embodiments, R⁷ is di-hydroxyl C2-C3 alkyl.

In some embodiments, R⁷ is C1-C6 cyanoalkyl. In some embodiments, R⁷ is C1-C3 cyanoalkyl. In some embodiments, R⁷ is cyanomethyl.

In some embodiments, R¹ is —C(═O)C1-C6 alkyl. In some embodiments, R¹ is —C(═O)CH₃.

In some embodiments, R¹ is —NH(CN).

In some embodiments, R⁷ is 5-6 membered heteroaryl. In some embodiments, R⁷ is a 5 membered heteroaryl. In some embodiments, R⁷ is a 6 membered heteroaryl.

In some embodiments, R⁷ is —C(═NR^{7A})NR^{7A}R^{7B}.

In some embodiments, when each -̅-̅-̅ adjacent to CR⁷ is a single bond, R⁷ is oxo.

In some embodiments, R⁷ is —NR^{7A}R^{7B}.

In some embodiments, R⁷ is NR^{7A}C(═O)R^{7B}.

In some embodiments, R⁷ is —C(═O)NR^{7A}R^{7B}.

In some embodiments, R⁷ is 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}. In some embodiments, R⁷ is an unsubstituted 4-10 membered heterocyclyl. In some embodiments, R⁷ is 4-10 membered heterocyclyl substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}. In some embodiments, R⁷ is an unsubstituted 4-6 membered heterocyclyl. In some embodiments, R⁷ is 4-6 membered heterocyclyl substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}. In some embodiments, R⁷ is 4-6 membered heterocyclyl substituted with 1 substituent independently selected from hydroxyl, C1-C6 haloalkyl, —C(═O)C1-C6 alkyl, or C1-C6 alkyl optionally substituted with C1-C6 alkoxy.

In some embodiments, one of R^{7A} and R^{7B} is hydrogen and the other of R^{7A} and R^{7B} is C2-C6 alkenyl, C2-C6 haloalkenyl, C1-C6 alkyl optionally substituted with oxo, or C1-C6 haloalkyl optionally substituted with oxo. In some embodiments, one of R^{7A} and R^{7B} is C1-C6 alkyl and the other of R^{7A} and R^{7B} is C2-C6 alkenyl, C2-C6 haloalkenyl, C1-C6 alkyl optionally substituted with oxo, or C1-C6 haloalkyl optionally substituted with oxo. In some embodiments, each of R^{7A} and R^{7B} is hydrogen. In some embodiments, each of R^{7A} and R^{7B} is an independently selected C1-C6 alkyl. In some embodiments, each of R^{7A} and R^{7B} is methyl.

In some embodiments, each of R^{A} and R^{B} are independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl optionally substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

In some embodiments, one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

In some embodiments, one of R^{A} and R^{B} is hydrogen and the other of R^{A} and R^{B} is hydroxyl. In some embodiments, one of R^{A} and R^{B} is hydrogen and the other of R^{A} and R^{B} is C1-C6 alkoxy. In some embodiments, one of R^{A} and R^{B} is hydrogen and the other of R^{A} and R^{B} is C3-C6 cycloalkyl. In some embodiments, one of R^{A} and R^{B} is hydrogen and the other of R^{A} and R^{B} is C2-C6 alkenyl. In some embodiments, one of R^{A} and R^{B} is hydrogen and the other of R^{A} and R^{B} is C1-C6 alkyl optionally substituted with hydroxyl or C1-C6 alkoxy. In some embodiments, one of R^{A} and R^{B} is hydrogen and the other of R^{A} and R^{B} is C1-C6 alkyl substituted with hydroxyl. In some embodiments, one of R^{A} and R^{B} is hydrogen and the other of R^{A} and R^{B} is C1-C6 alkyl substituted with C1-C6 alkoxy.

In some embodiments, one of R^{A} and R^{B} is hydrogen and the other of R^{A} and R^{B} is C1-C6 alkyl. In some embodiments, each of R^{A} and R^{B} are hydrogen. In some embodiments, each of R^{A} and R^{B} are an independently selected C1-C6 alkyl optionally substituted with hydroxyl or C1-C6 alkoxy. In some embodiments, each of R^{A} and R^{B} are an independently selected C1-C6 alkyl. In some embodiments, each of R^{A} and R^{B} are methyl.

In some embodiments, R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl.

In some embodiments, R^{A} and R^{B} together with the nitrogen atom to which they are attached form a 4-10 membered heterocyclyl substituted with 1-2 substituents independently selected from halogen, C1-C6 alkyl, and —C(═O)C1-C6 alkyl.

In some embodiments, R^{A} and R^{B} together with the nitrogen atom to which they are attached form a 4-10 membered heterocyclyl substituted with halogen, C1-C6 alkyl, or —C(═O)C1-C6 alkyl. In some embodiments, R^{A} and R^{B} together with the nitrogen atom to which they are attached form a 4-10 membered heterocyclyl substituted with 1-2 substitutents independently selected from fluoro, methyl, and acetyl.

In some embodiments, R^{A} and R^{B} together with the nitrogen atom to which they are attached form a 4-10 membered heterocyclyl. In some embodiments, R^{A} and R^{B} together with the nitrogen atom to which they are attached form a 4-6 membered heterocyclyl.

In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl optionally substituted with C1-C6 alkyl or with C1-C6 hydroxylalkyl. In some embodiments, each R^{C} is independently C3-C6 cycloalkyl, —C(═O)NR¹, or a C1-C6 alkyl substituted with —NR^{A}R^{B} or with 4-10 membered heterocyclyl substituted with C1-C6 alkyl or with C1-C6 hydroxylalkyl. In some embodiments, each R^{C} is independently C3-C6 cycloalkyl, —C(═O)NHR^{Y1}, or a C1-C6 alkyl substituted with —NR^{A}R^{B} or with 4-10 membered heterocyclyl. In some embodiments, each R^{C} is independently C3-C6 cycloalkyl, —C(═O)NHR^{Y1}, or a C1-C6 alkyl.

In some embodiments, each R^{C} is independently C3-C6 cycloalkyl. In some embodiments, each R^{C} is independently —C(═O)NHR^{Y1}. In some embodiments, each R^{C} is independently C1-C6 alkyl optionally substituted with —NR^{A}R^{B} or with 4-10 membered heterocyclyl optionally substituted with C1-C6 alkyl or with C1-C6 hydroxylalkyl. In some embodiments, each R^{C} is independently C1-C6 alkyl substituted with —NR^{A}R^{B} or with 4-10 membered heterocyclyl optionally substituted with C1-C6 alkyl or with C1-C6 hydroxylalkyl.

In some embodiments, one of R^{D} and R^{E} is hydrogen and the other of R^{D} and R^{E} is hydroxyl, C1-C6 alkyl, or C1-C6 alkoxy. In some embodiments, one of R^{D} and R^{E} is hydrogen and the other of R^{D} and R^{E} is hydroxyl. In some embodiments, one of R^{D} and R^{E} is hydrogen and the other of R^{D} and R^{E} is C1-C6 alkyl. In some embodiments, one of R^{D} and R^{E} is hydrogen and the other of R^{D} and R^{E} is C1-C6 alkoxy. In some embodiments, one of R^{D} and R^{E} is hydrogen and the other of R^{D} and R^{E} is hydroxyl, methyl, or methoxy. In some embodiments, each of R^{D} and R^{E} is hydrogen. In some embodiments, each of R^{D} and R^{E} is an independently selected C1-C6 alkyl. In some embodiments, each of R^{D} and R^{E} is methyl.

In some embodiments, one of R^{F} and R^{G} is hydrogen and the other of R^{F} and R^{G} is phenyl or C1-C6 alkyl optionally substituted with oxo or NR^{A}R^{B}. In some embodiments, one of R^{F} and R^{G} is hydrogen and the other of R^{F} and R^{G} is phenyl or C1-C6 alkyl substituted with oxo or —NR^{A}R^{B}. In some embodiments, one of R^{F} and R^{G} is hydrogen and the other of R^{F} and R^{G} is phenyl or C1-C6 alkyl. In some embodiments, one of R^{F} and R^{G} is hydrogen and the other of R^{F} and R^{G} is phenyl. In some embodiments, one of R^{F} and R^{G} is hydrogen and the other of R^{F} and R^{G} is C1-C6 alkyl optionally substituted with oxo or —NR^{A}R^{B}. In some embodiments, one of R^{F} and R^{G} is hydrogen and the other of R^{F} and R^{G} is C1-C6 alkyl substituted with oxo or —NR^{A}R^{B}. In some embodiments, each of R^{F} and R^{G} is hydrogen. In some embodiments, each of R^{F} and R^{G} is an independently selected C1-C6 alkyl. In some embodiments, each of R^{F} and R^{G} is methyl.

In some embodiments, the compound of Formula (I), or a pharmaceutically acceptable salt thereof, is a compound of Formula (I-A1):

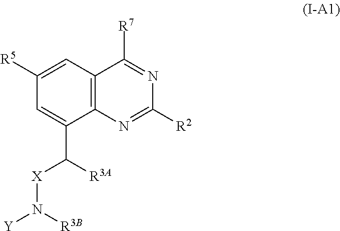

or a pharmaceutically acceptable salt thereof.

In some embodiments, the compound of Formula (I), or a pharmaceutically acceptable salt thereof, is a compound of Formula (I-A2):

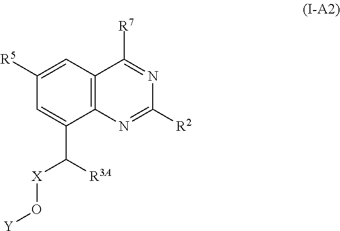

or a pharmaceutically acceptable salt thereof.

In some embodiments, the compound of Formula (I), or a pharmaceutically acceptable salt thereof, is a compound of Formula (I-B1):

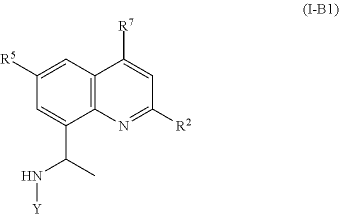

or a pharmaceutically acceptable salt thereof.

In some embodiments, the compound of Formula (I), or a pharmaceutically acceptable salt thereof, is a compound of Formula (I-B2):

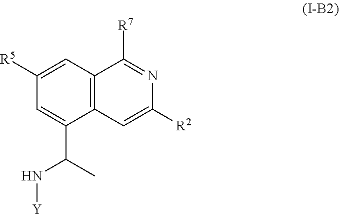

or a pharmaceutically acceptable salt thereof.

In some embodiments, the compound of Formula (I), or a pharmaceutically acceptable salt thereof, is a compound of Formula (I-B3):

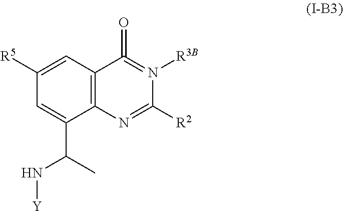

or a pharmaceutically acceptable salt thereof.

In some embodiments, the compound of Formula (I), or a pharmaceutically acceptable salt thereof, is a compound of Formula (I-B4):

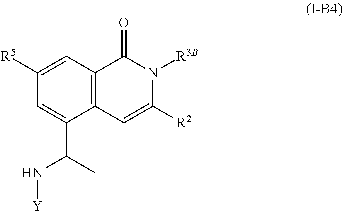

or a pharmaceutically acceptable salt thereof.

In some embodiments, the compound of Formula (I), or a pharmaceutically acceptable salt thereof, is a compound of Formula (I-C):

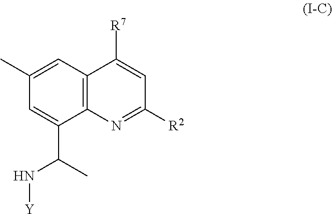

or a pharmaceutically acceptable salt thereof.

In some embodiments, the compound of Formula (I), or a pharmaceutically acceptable salt thereof, is a compound of Formula (I-D):

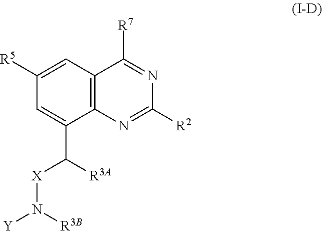

or a pharmaceutically acceptable salt thereof.

In some embodiments, the compound of Formula (I), or a pharmaceutically acceptable salt thereof, is a compound of Formula (I-E):

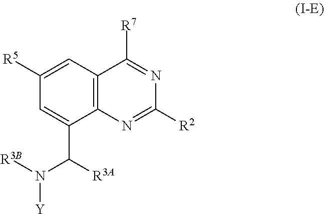

or a pharmaceutically acceptable salt thereof.

In some embodiments, the compound of Formula (I), or a pharmaceutically acceptable salt thereof, is a compound of Formula (I-F):

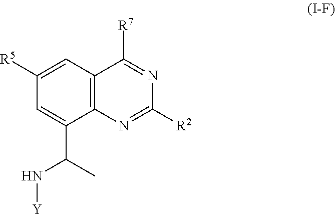

or a pharmaceutically acceptable salt thereof.

In some embodiments, the compound of Formula (I), or a pharmaceutically acceptable salt thereof, is a compound of Formula (I-F1):

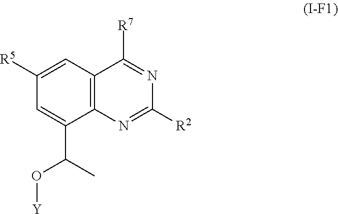

or a pharmaceutically acceptable salt thereof.

In some embodiments, the compound of Formula (I), or a pharmaceutically acceptable salt thereof, is a compound of Formula (I-G):

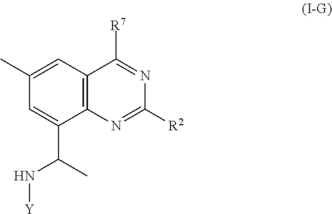

or a pharmaceutically acceptable salt thereof.

In some embodiments, the compound of Formula (I), or a pharmaceutically acceptable salt thereof, is a compound of Formula (I-H1):

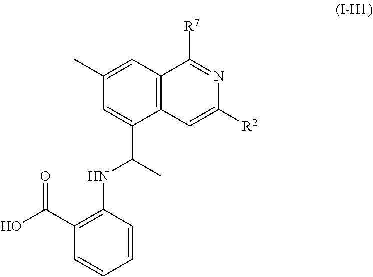

or a pharmaceutically acceptable salt thereof.

In some embodiments, the compound of Formula (I), or a pharmaceutically acceptable salt thereof, is a compound of Formula (I-H2):

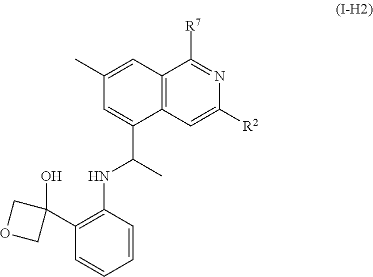

or a pharmaceutically acceptable salt thereof.

In some embodiments, the compound of Formula (I), or a pharmaceutically acceptable salt thereof, is a compound of Formula (I-H3):

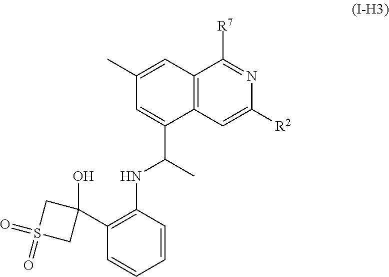

or a pharmaceutically acceptable salt thereof.

In some embodiments, the compound of Formula (I), or a pharmaceutically acceptable salt thereof, is a compound of Formula (I-H4):

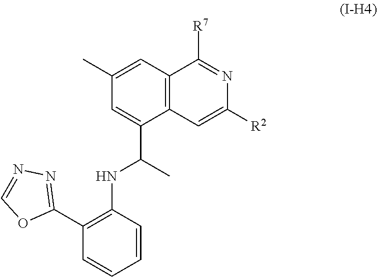

or a pharmaceutically acceptable salt thereof.

In some embodiments, the compound of Formula (I), or a pharmaceutically acceptable salt thereof, is a compound of Formula (I-H5):

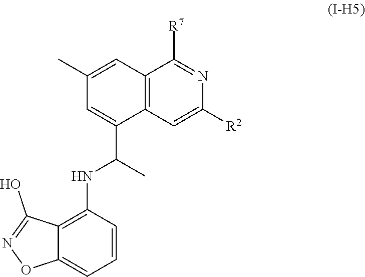

or a pharmaceutically acceptable salt thereof.

In some embodiments, the compound of Formula (I), or a pharmaceutically acceptable salt thereof, is a compound of Formula (I-H6):

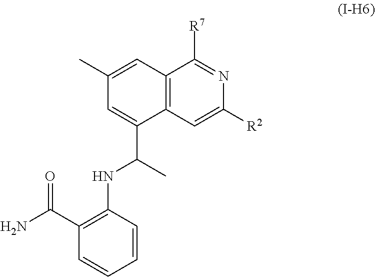

or a pharmaceutically acceptable salt thereof.

In some embodiments, the compound of Formula (I), or a pharmaceutically acceptable salt thereof, is a compound of Formula (I-I1):

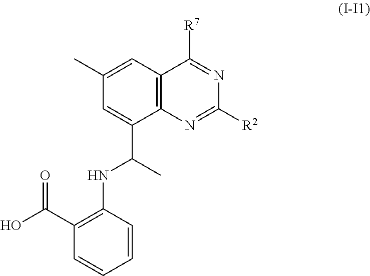

or a pharmaceutically acceptable salt thereof.

In some embodiments, the compound of Formula (I), or a pharmaceutically acceptable salt thereof, is a compound of Formula (I-I2):

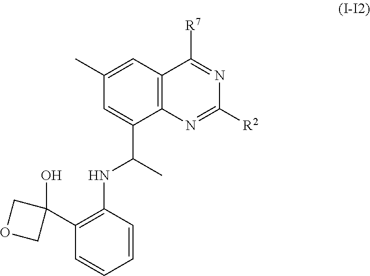

or a pharmaceutically acceptable salt thereof.

In some embodiments, the compound of Formula (I), or a pharmaceutically acceptable salt thereof, is a compound of Formula (I-13):

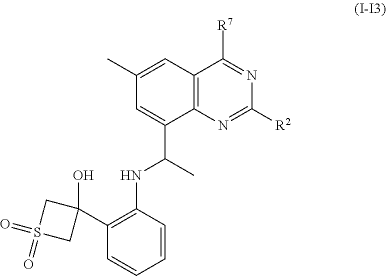

or a pharmaceutically acceptable salt thereof.

In some embodiments, the compound of Formula (I), or a pharmaceutically acceptable salt thereof, is a compound of Formula (I-I4):

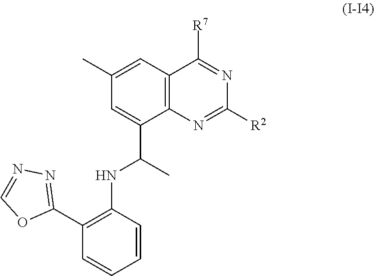

or a pharmaceutically acceptable salt thereof.

In some embodiments, the compound of Formula (I), or a pharmaceutically acceptable salt thereof, is a compound of Formula (I-15):

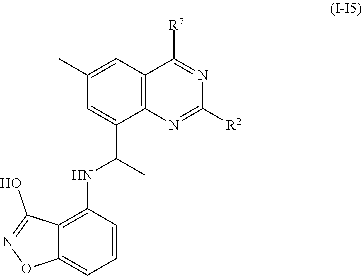

or a pharmaceutically acceptable salt thereof.

In some embodiments, the compound of Formula (I), or a pharmaceutically acceptable salt thereof, is a compound of Formula (I-16):

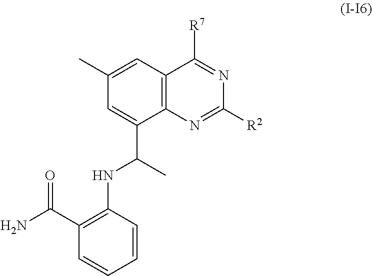

or a pharmaceutically acceptable salt thereof.

In some embodiments, the compound of Formula (II), or a pharmaceutically acceptable salt thereof, is a compound of Formula (II-A1):

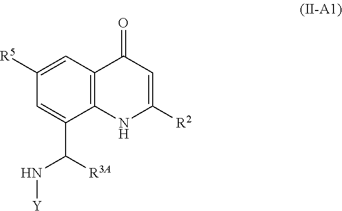

or a pharmaceutically acceptable salt thereof.

In some embodiments, the compound of Formula (II), or a pharmaceutically acceptable salt thereof, is a compound of Formula (II-B):

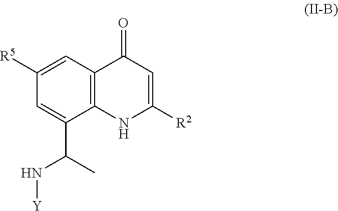

or a pharmaceutically acceptable salt thereof.

In some embodiments, the compound of Formula (II), or a pharmaceutically acceptable salt thereof, is a compound of Formula (II-C):

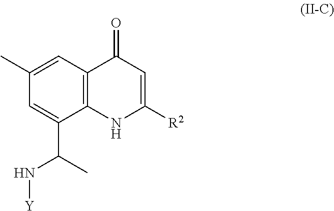

or a pharmaceutically acceptable salt thereof.

In some embodiments, the compound of Formula (III), or a pharmaceutically acceptable salt thereof, is a compound of Formula (III-A1):

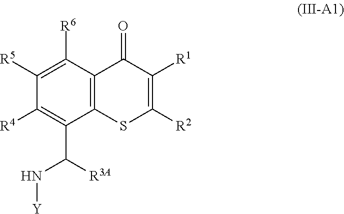

or a pharmaceutically acceptable salt thereof.

In some embodiments, the compound of Formula (III), or a pharmaceutically acceptable salt thereof, is a compound of Formula (III-B):

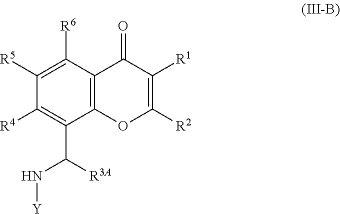

or a pharmaceutically acceptable salt thereof.

In some embodiments, the compound of Formula (III), or a pharmaceutically acceptable salt thereof, is a compound of Formula (III-C):

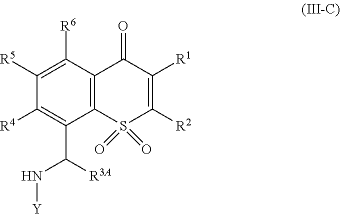

or a pharmaceutically acceptable salt thereof.

In some embodiments, the compound of Formula (III), or a pharmaceutically acceptable salt thereof, is a compound of Formula (III-D):

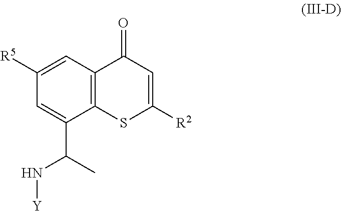

or a pharmaceutically acceptable salt thereof.

In some embodiments, the compound of Formula (III), or a pharmaceutically acceptable salt thereof, is a compound of Formula (III-E):

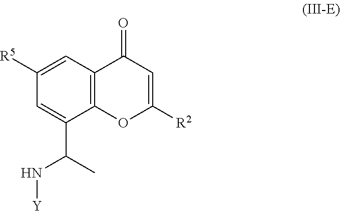

or a pharmaceutically acceptable salt thereof.

In some embodiments, the compound of Formula (III), or a pharmaceutically acceptable salt thereof, is a compound of Formula (III-F):

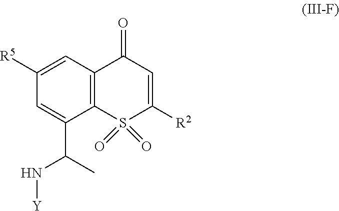

or a pharmaceutically acceptable salt thereof.

### Non-Limiting Exemplary Compounds

In some embodiments, the compound is selected from the group consisting of Compounds 1-147, or a pharmaceutically acceptable salt of any of the foregoing.

In some embodiments, the compound is selected from the group consisting of the compounds delineated in Table A, or a pharmaceutically acceptable salt of any of the foregoing.

TABLE A

| Cmpd | |
|---|---|
| No. | Structure |
| 1 | 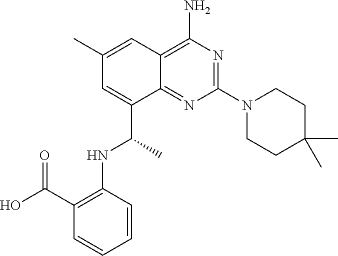 [SMILES: Cc1cc([C@H](C)Nc2ccccc2C(=O)O)c2nc(N3CCC(C)(C)CC3)nc(N)c2c1] |
| 2 | 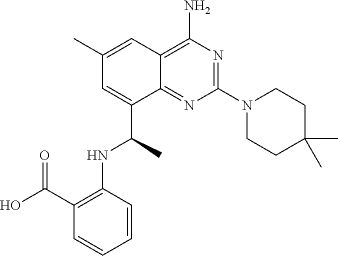 [SMILES: Cc1cc([C@@H](C)Nc2ccccc2C(=O)O)c2nc(N3CCC(C)(C)CC3)nc(N)c2c1] |
| 3 | 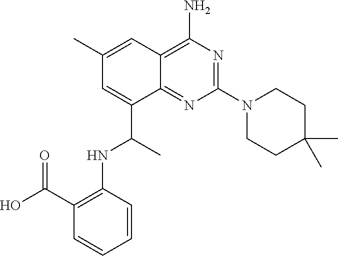 [SMILES: Cc1cc(C(C)Nc2ccccc2C(=O)O)c2nc(N3CCC(C)(C)CC3)nc(N)c2c1] |
| 4 | 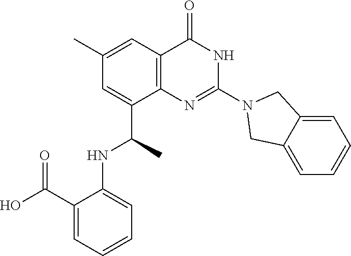 [SMILES: Cc1cc([C@@H](C)Nc2ccccc2C(=O)O)c2nc(N3Cc4ccccc4C3)[nH]c(=O)c2c1] |
| 5 | 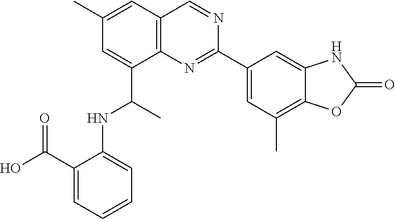 [SMILES: Cc1cc(C(C)Nc2ccccc2C(=O)O)c2nc(-c3cc(C)c4oc(=O)[nH]c4c3)ncc2c1] |
| 6 | 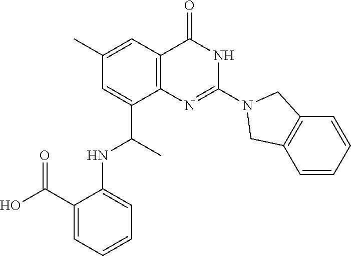 [SMILES: Cc1cc(C(C)Nc2ccccc2C(=O)O)c2nc(N3Cc4ccccc4C3)[nH]c(=O)c2c1] |
| 7 | 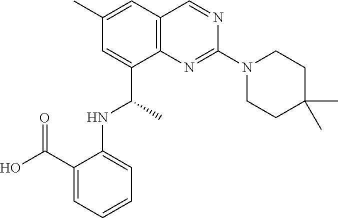 [SMILES: Cc1cc([C@H](C)Nc2ccccc2C(=O)O)c2nc(N3CCC(C)(C)CC3)ncc2c1] |
| 8 | 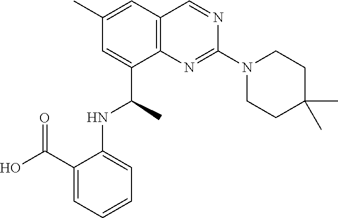 [SMILES: Cc1cc([C@@H](C)Nc2ccccc2C(=O)O)c2nc(N3CCC(C)(C)CC3)ncc2c1] |
| 9 | 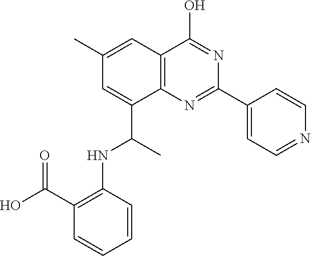 [SMILES: Cc1cc(C(C)Nc2ccccc2C(=O)O)c2nc(-c3ccncc3)nc(O)c2c1] |
| 10 | 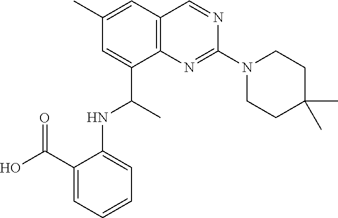 [SMILES: Cc1cc(C(C)Nc2ccccc2C(=O)O)c2nc(N3CCC(C)(C)CC3)ncc2c1] |
| 11 | 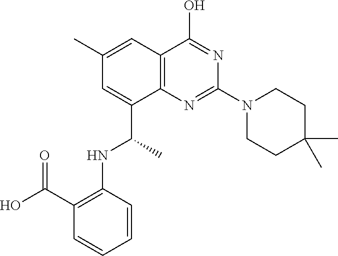 [SMILES: Cc1cc([C@H](C)Nc2ccccc2C(=O)O)c2nc(N3CCC(C)(C)CC3)nc(O)c2c1] |
| 12 | 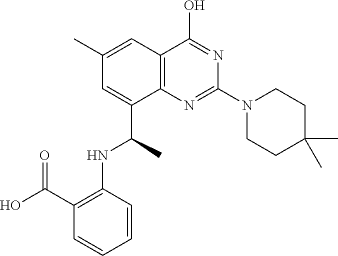 [SMILES: Cc1cc([C@@H](C)Nc2ccccc2C(=O)O)c2nc(N3CCC(C)(C)CC3)nc(O)c2c1] |
| 13 | 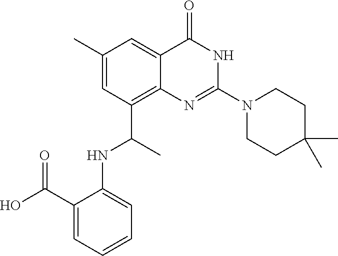 [SMILES: Cc1cc(C(C)Nc2ccccc2C(=O)O)c2nc(N3CCC(C)(C)CC3)[nH]c(=O)c2c1] |
| 14 | 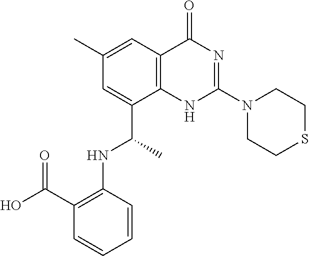 [SMILES: Cc1cc([C@H](C)Nc2ccccc2C(=O)O)c2[nH]c(N3CCSCC3)nc(=O)c2c1] |
| 15 | 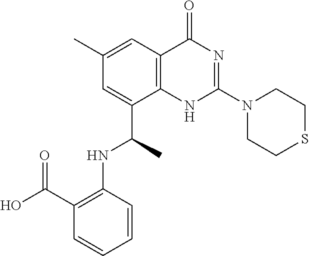 [SMILES: Cc1cc([C@@H](C)Nc2ccccc2C(=O)O)c2[nH]c(N3CCSCC3)nc(=O)c2c1] |
| 16 | 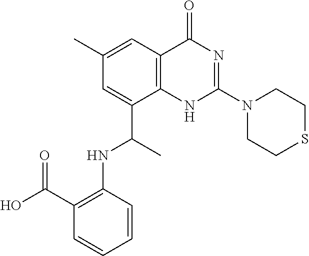 [SMILES: Cc1cc(C(C)Nc2ccccc2C(=O)O)c2[nH]c(N3CCSCC3)nc(=O)c2c1] |
| 17 | 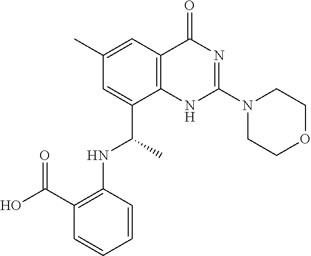 [SMILES: Cc1cc([C@H](C)Nc2ccccc2C(=O)O)c2[nH]c(N3CCOCC3)nc(=O)c2c1] |
| 18 | 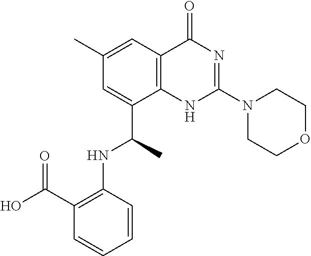 [SMILES: Cc1cc([C@@H](C)Nc2ccccc2C(=O)O)c2[nH]c(N3CCOCC3)nc(=O)c2c1] |
| 19 | 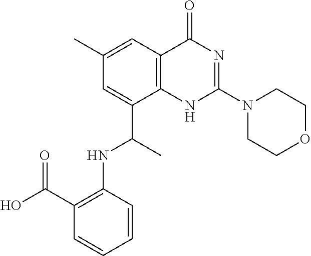 [SMILES: Cc1cc(C(C)Nc2ccccc2C(=O)O)c2[nH]c(N3CCOCC3)nc(=O)c2c1] |
| 20 | 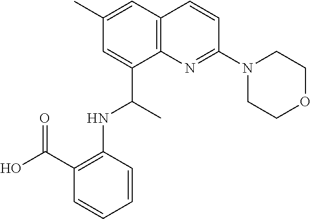 [SMILES: Cc1cc(C(C)Nc2ccccc2C(=O)O)c2nc(N3CCOCC3)ccc2c1] |
| 21 | 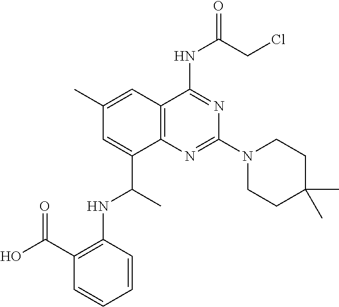 [SMILES: Cc1cc(C(C)Nc2ccccc2C(=O)O)c2nc(N3CCC(C)(C)CC3)nc(NC(=O)CCl)c2c1] |
| 22 | 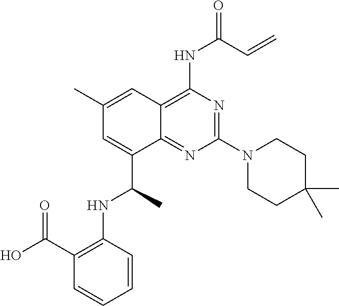 [SMILES: C=CC(=O)Nc1nc(N2CCC(C)(C)CC2)nc2c([C@@H](C)Nc3ccccc3C(=O)O)cc(C)cc12] |
| 23 | 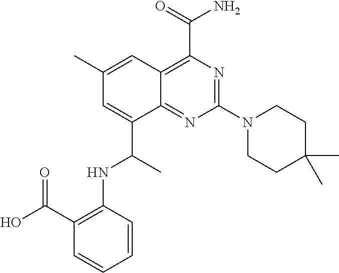 [SMILES: Cc1cc(C(C)Nc2ccccc2C(=O)O)c2nc(N3CCC(C)(C)CC3)nc(C(N)=O)c2c1] |
| 24 | 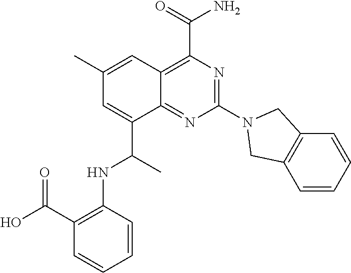 [SMILES: Cc1cc(C(C)Nc2ccccc2C(=O)O)c2nc(N3Cc4ccccc4C3)nc(C(N)=O)c2c1] |
| 25 | 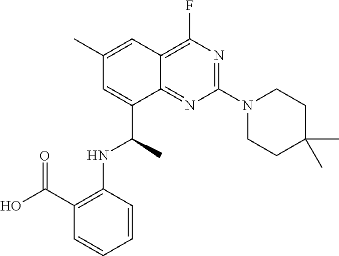 [SMILES: Cc1cc([C@@H](C)Nc2ccccc2C(=O)O)c2nc(N3CCC(C)(C)CC3)nc(F)c2c1] |
| 26 | 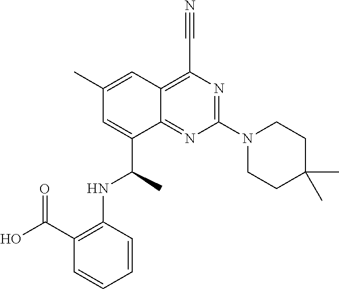 [SMILES: Cc1cc([C@@H](C)Nc2ccccc2C(=O)O)c2nc(N3CCC(C)(C)CC3)nc(C#N)c2c1] |
| 27 | 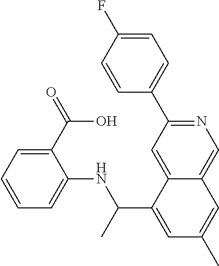 [SMILES: Cc1cc(C(C)Nc2ccccc2C(=O)O)c2cc(-c3ccc(F)cc3)ncc2c1] |
| 28 | 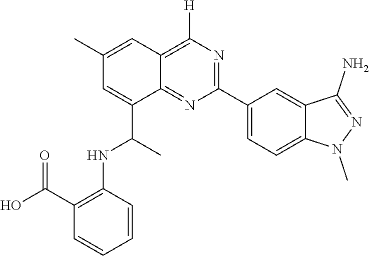 [SMILES: Cc1cc(C(C)Nc2ccccc2C(=O)O)c2nc(-c3ccc4c(c3)c(N)nn4C)ncc2c1] |
| 29 | 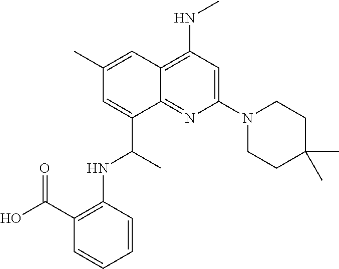 [SMILES: CNc1cc(N2CCC(C)(C)CC2)nc2c(C(C)Nc3ccccc3C(=O)O)cc(C)cc12] |
| 30 | 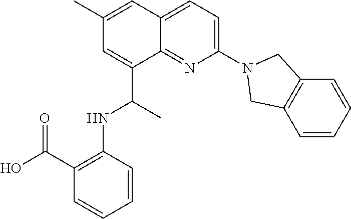 [SMILES: Cc1cc(C(C)Nc2ccccc2C(=O)O)c2nc(N3Cc4ccccc4C3)ccc2c1] |
| 31 | 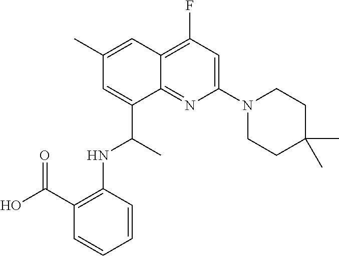 [SMILES: Cc1cc(C(C)Nc2ccccc2C(=O)O)c2nc(N3CCC(C)(C)CC3)cc(F)c2c1] |
| 32 | 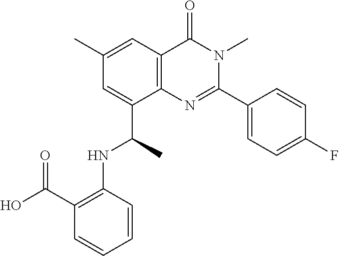 [SMILES: Cc1cc([C@@H](C)Nc2ccccc2C(=O)O)c2nc(-c3ccc(F)cc3)n(C)c(=O)c2c1] |
| 33 | 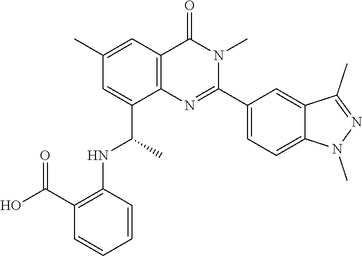 [SMILES: Cc1cc([C@H](C)Nc2ccccc2C(=O)O)c2nc(-c3ccc4c(c3)c(C)nn4C)n(C)c(=O)c2c1] |
| 34 | 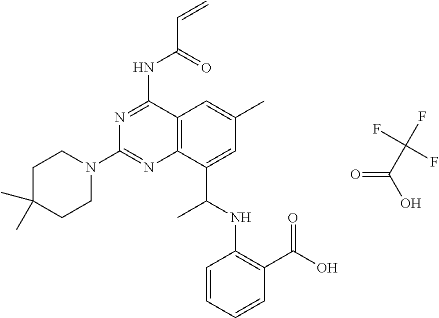 [SMILES: C=CC(=O)Nc1nc(N2CCC(C)(C)CC2)nc2c(C(C)Nc3ccccc3C(=O)O)cc(C)cc12 ; O=C(O)C(F)(F)F] |
| 35 | 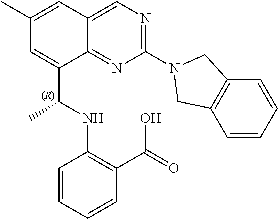 |
| 36 | 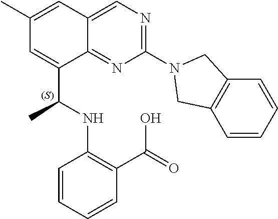 |
| 37 | 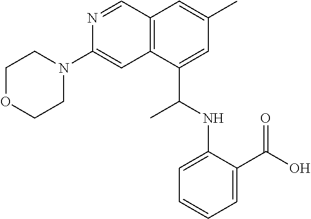 [SMILES: Cc1cc(C(C)Nc2ccccc2C(=O)O)c2cc(N3CCOCC3)ncc2c1] |
| 38 | 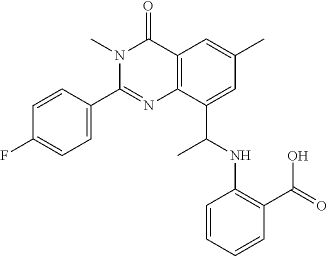 [SMILES: Cc1cc(C(C)Nc2ccccc2C(=O)O)c2nc(-c3ccc(F)cc3)n(C)c(=O)c2c1] |
| 39 | 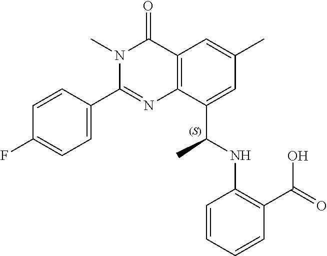 |
| 40 | 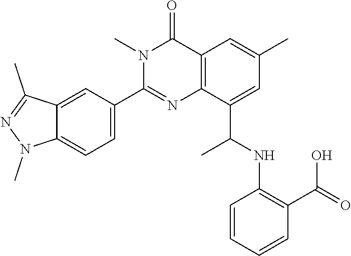 [SMILES: Cc1cc(C(C)Nc2ccccc2C(=O)O)c2nc(-c3ccc4c(c3)c(C)nn4C)n(C)c(=O)c2c1] |
| 41 | 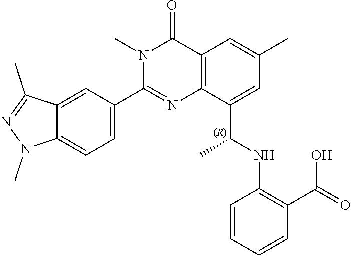 |
| 42 | 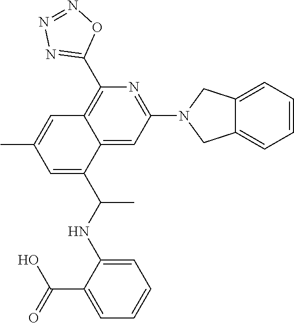 [SMILES: Cc1cc(C(C)Nc2ccccc2C(=O)O)c2cc(N3Cc4ccccc4C3)nc(-c3nnno3)c2c1] |
| 43 | 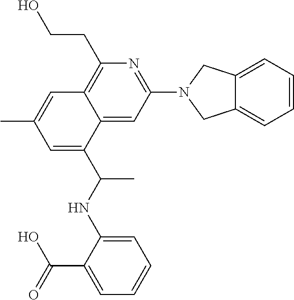 [SMILES: Cc1cc(C(C)Nc2ccccc2C(=O)O)c2cc(N3Cc4ccccc4C3)nc(CCO)c2c1] |
| 44 | 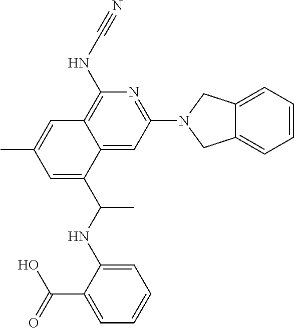 [SMILES: Cc1cc(C(C)Nc2ccccc2C(=O)O)c2cc(N3Cc4ccccc4C3)nc(NC#N)c2c1] |
| 45 | 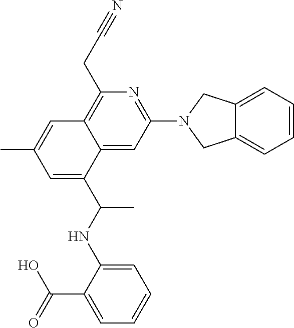 [SMILES: Cc1cc(C(C)Nc2ccccc2C(=O)O)c2cc(N3Cc4ccccc4C3)nc(CC#N)c2c1] |
| 46 | 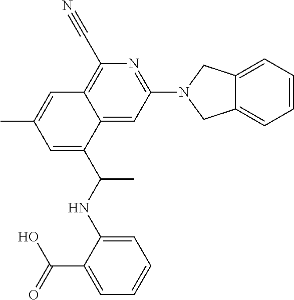 [SMILES: Cc1cc(C(C)Nc2ccccc2C(=O)O)c2cc(N3Cc4ccccc4C3)nc(C#N)c2c1] |
| 47 | 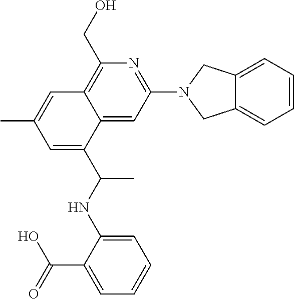 [SMILES: Cc1cc(C(C)Nc2ccccc2C(=O)O)c2cc(N3Cc4ccccc4C3)nc(CO)c2c1] |
| 48 | 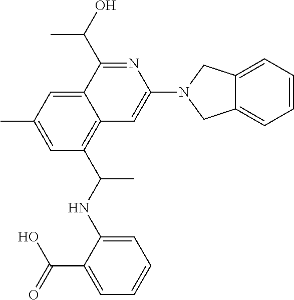 [SMILES: Cc1cc(C(C)Nc2ccccc2C(=O)O)c2cc(N3Cc4ccccc4C3)nc(C(C)O)c2c1] |
| 49 | 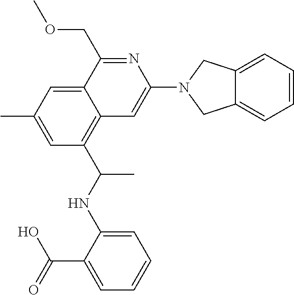 [SMILES: COCc1nc(N2Cc3ccccc3C2)cc2c(C(C)Nc3ccccc3C(=O)O)cc(C)cc12] |
| 50 | 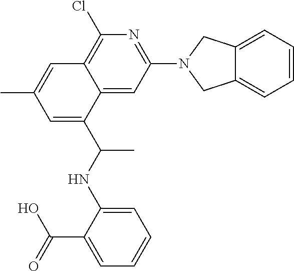 [SMILES: Cc1cc(C(C)Nc2ccccc2C(=O)O)c2cc(N3Cc4ccccc4C3)nc(Cl)c2c1] |
| 51 | 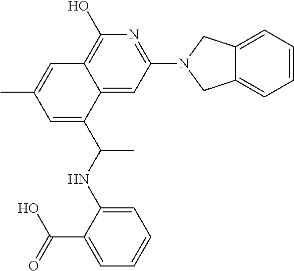 [SMILES: Cc1cc(C(C)Nc2ccccc2C(=O)O)c2cc(N3Cc4ccccc4C3)nc(O)c2c1] |
| 52 | 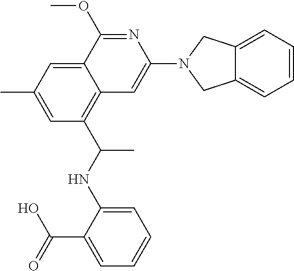 [SMILES: COc1nc(N2Cc3ccccc3C2)cc2c(C(C)Nc3ccccc3C(=O)O)cc(C)cc12] |
| 53 | 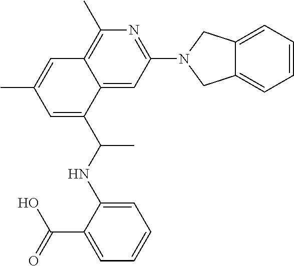 [SMILES: Cc1cc(C(C)Nc2ccccc2C(=O)O)c2cc(N3Cc4ccccc4C3)nc(C)c2c1] |
| 54 | 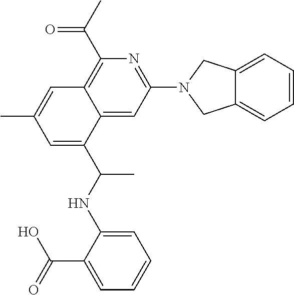 [SMILES: CC(=O)c1nc(N2Cc3ccccc3C2)cc2c(C(C)Nc3ccccc3C(=O)O)cc(C)cc12] |
| 55 | 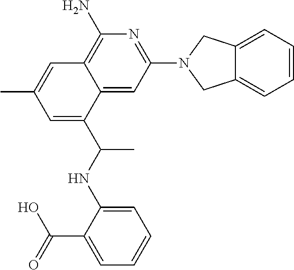 [SMILES: Cc1cc(C(C)Nc2ccccc2C(=O)O)c2cc(N3Cc4ccccc4C3)nc(N)c2c1] |
| 56 | 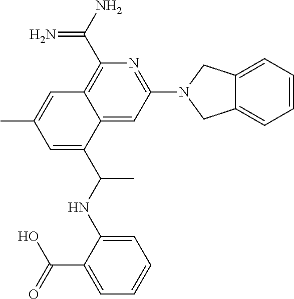 [SMILES: Cc1cc(C(C)Nc2ccccc2C(=O)O)c2cc(N3Cc4ccccc4C3)nc(C(=N)N)c2c1] |
| 57 | 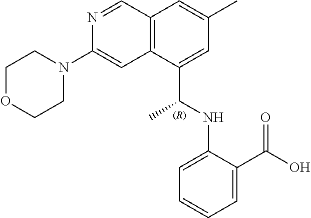 |
| 58 | 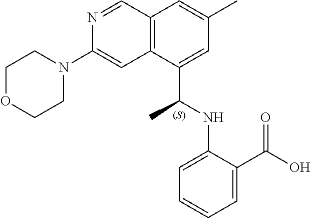 |
| 59 | 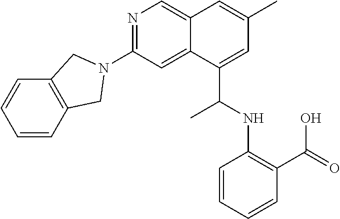 [SMILES: Cc1cc(C(C)Nc2ccccc2C(=O)O)c2cc(N3Cc4ccccc4C3)ncc2c1] |
| 60 | 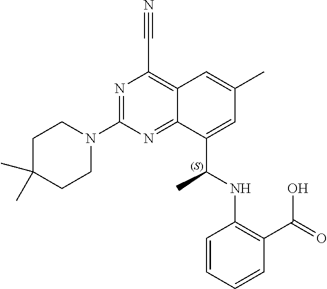 |
| 61 | 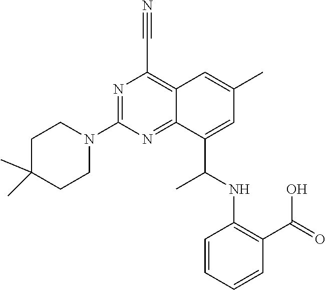 [SMILES: Cc1cc(C(C)Nc2ccccc2C(=O)O)c2nc(N3CCC(C)(C)CC3)nc(C#N)c2c1] |
| 62 | 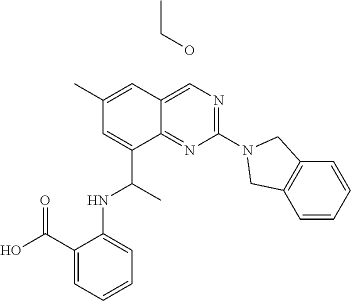 |
| 63 | 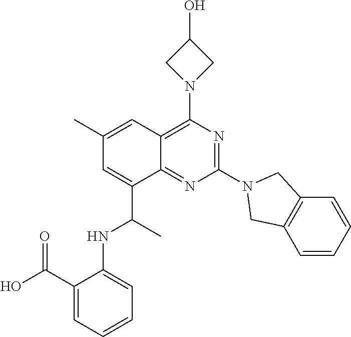 [SMILES: Cc1cc(C(C)Nc2ccccc2C(=O)O)c2nc(N3Cc4ccccc4C3)nc(N3CC(O)C3)c2c1] |
| 64 | 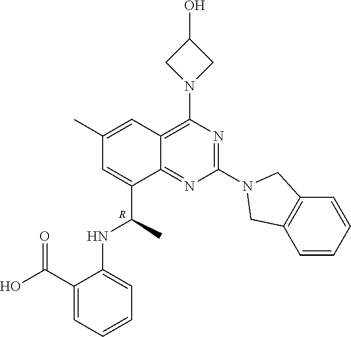 |
| 65 | 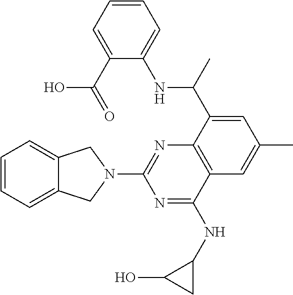 [SMILES: Cc1cc(C(C)Nc2ccccc2C(=O)O)c2nc(N3Cc4ccccc4C3)nc(NC3CC3O)c2c1] |
| 66 | 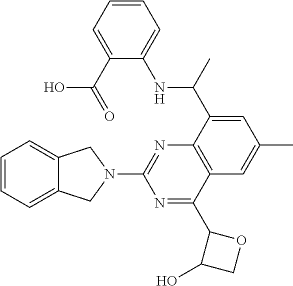 [SMILES: Cc1cc(C(C)Nc2ccccc2C(=O)O)c2nc(N3Cc4ccccc4C3)nc(C3OCC3O)c2c1] |
| 67 | 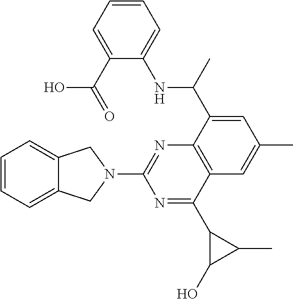 [SMILES: Cc1cc(C(C)Nc2ccccc2C(=O)O)c2nc(N3Cc4ccccc4C3)nc(C3C(C)C3O)c2c1] |
| 68 | 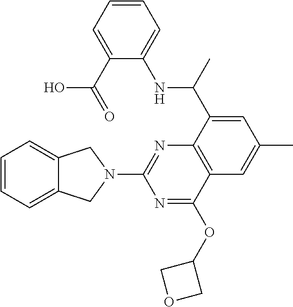 [SMILES: Cc1cc(C(C)Nc2ccccc2C(=O)O)c2nc(N3Cc4ccccc4C3)nc(OC3COC3)c2c1] |
| 69 | 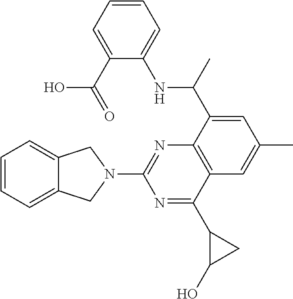 [SMILES: Cc1cc(C(C)Nc2ccccc2C(=O)O)c2nc(N3Cc4ccccc4C3)nc(C3CC3O)c2c1] |
| 70 | 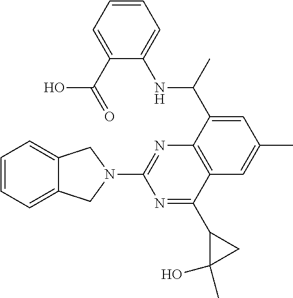 [SMILES: Cc1cc(C(C)Nc2ccccc2C(=O)O)c2nc(N3Cc4ccccc4C3)nc(C3CC3(C)O)c2c1] |
| 71 | 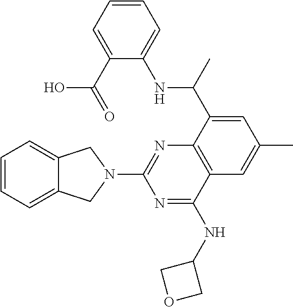 [SMILES: Cc1cc(C(C)Nc2ccccc2C(=O)O)c2nc(N3Cc4ccccc4C3)nc(NC3COC3)c2c1] |
| 72 | 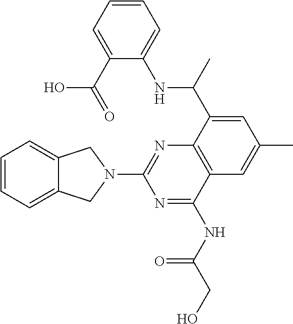 [SMILES: Cc1cc(C(C)Nc2ccccc2C(=O)O)c2nc(N3Cc4ccccc4C3)nc(NC(=O)CO)c2c1] |
| 73 | 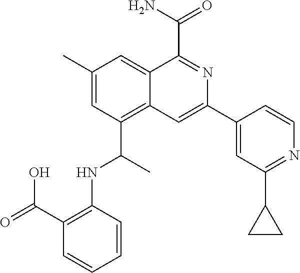 [SMILES: Cc1cc(C(C)Nc2ccccc2C(=O)O)c2cc(-c3ccnc(C4CC4)c3)nc(C(N)=O)c2c1] |
| 74 | 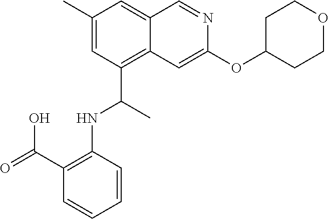 [SMILES: Cc1cc(C(C)Nc2ccccc2C(=O)O)c2cc(OC3CCOCC3)ncc2c1] |
| 75 | 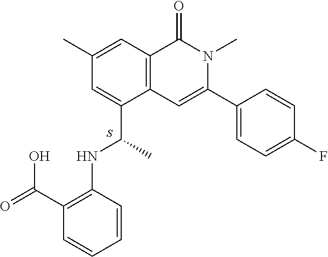 |
| 76 | 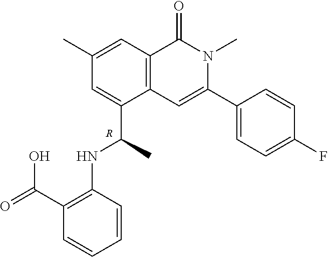 |
| 77 | 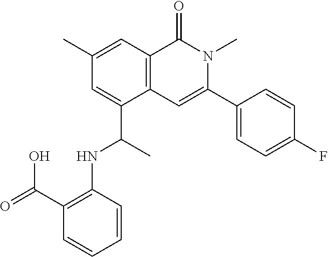 [SMILES: Cc1cc(C(C)Nc2ccccc2C(=O)O)c2cc(-c3ccc(F)cc3)n(C)c(=O)c2c1] |
| 78 | 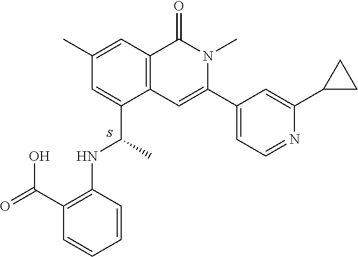 |
| 79 | 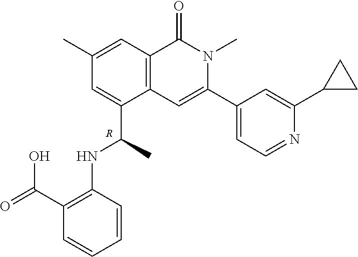 |
| 80 | 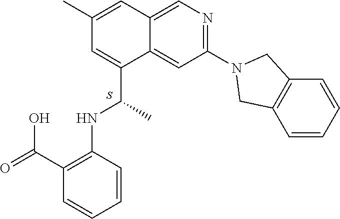 |
| 81 | 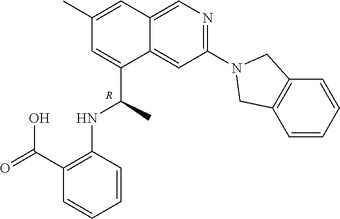 |
| 82 | 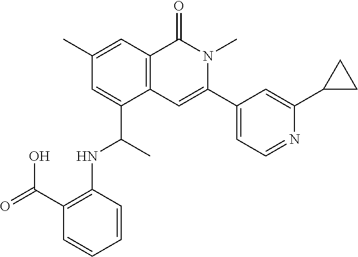 [SMILES: Cc1cc(C(C)Nc2ccccc2C(=O)O)c2cc(-c3ccnc(C4CC4)c3)n(C)c(=O)c2c1] |
| 83 | 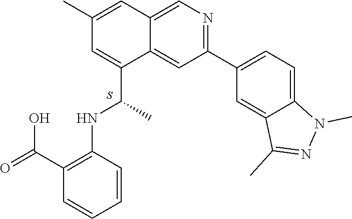 |
| 84 | 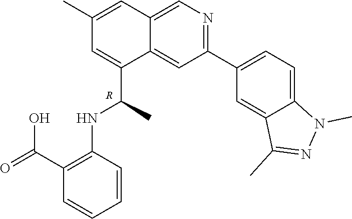 |
| 85 | 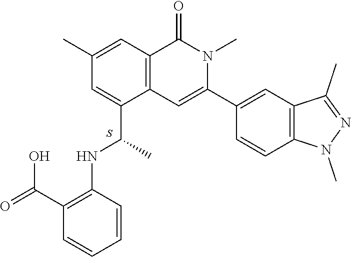 |
| 86 | 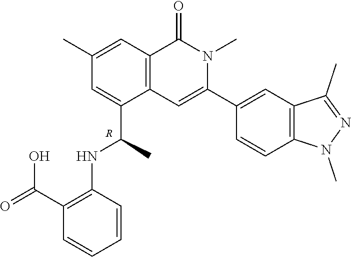 |
| 87 | 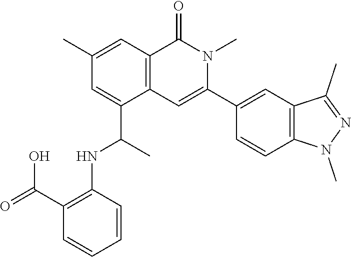 [SMILES: Cc1cc(C(C)Nc2ccccc2C(=O)O)c2cc(-c3ccc4c(c3)c(C)nn4C)n(C)c(=O)c2c1] |
| 88 | 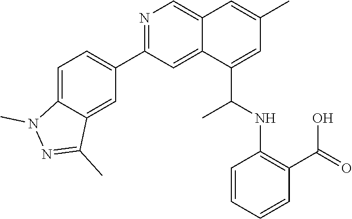 [SMILES: Cc1cc(C(C)Nc2ccccc2C(=O)O)c2cc(-c3ccc4c(c3)c(C)nn4C)ncc2c1] |
| 89 | 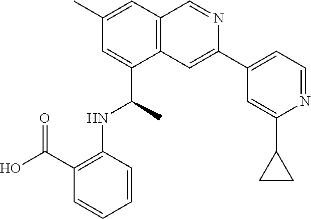 [SMILES: Cc1cc([C@@H](C)Nc2ccccc2C(=O)O)c2cc(-c3ccnc(C4CC4)c3)ncc2c1] |
| 90 | 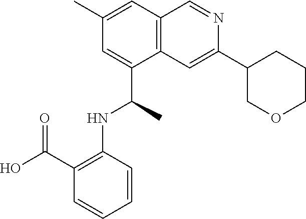 [SMILES: Cc1cc([C@@H](C)Nc2ccccc2C(=O)O)c2cc(C3CCCOC3)ncc2c1] |
| 91 | 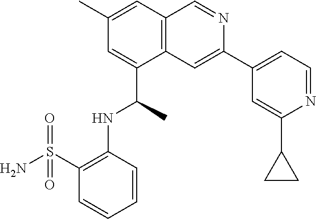 [SMILES: Cc1cc([C@@H](C)Nc2ccccc2S(N)(=O)=O)c2cc(-c3ccnc(C4CC4)c3)ncc2c1] |
| 92 | 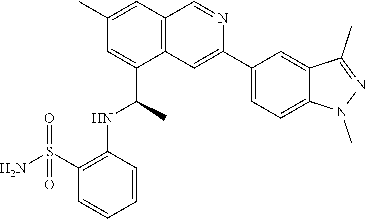 [SMILES: Cc1cc([C@@H](C)Nc2ccccc2S(N)(=O)=O)c2cc(-c3ccc4c(c3)c(C)nn4C)ncc2c1] |
| 93 | 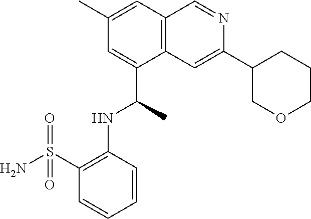 [SMILES: Cc1cc([C@@H](C)Nc2ccccc2S(N)(=O)=O)c2cc(C3CCCOC3)ncc2c1] |
| 94 | 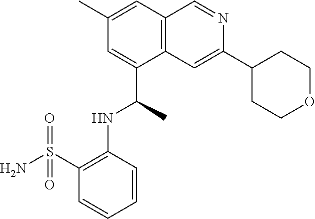 [SMILES: Cc1cc([C@@H](C)Nc2ccccc2S(N)(=O)=O)c2cc(C3CCOCC3)ncc2c1] |
| 95 | 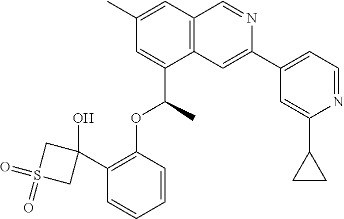 [SMILES: Cc1cc([C@@H](C)Oc2ccccc2C2(O)CS(=O)(=O)C2)c2cc(-c3ccnc(C4CC4)c3)ncc2c1] |
| 96 | 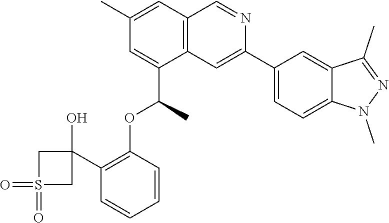 [SMILES: Cc1cc([C@@H](C)Oc2ccccc2C2(O)CS(=O)(=O)C2)c2cc(-c3ccc4c(c3)c(C)nn4C)ncc2c1] |
| 97 | 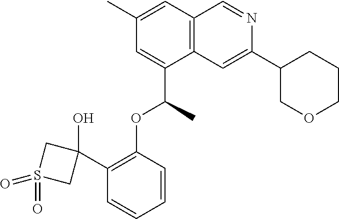 [SMILES: Cc1cc([C@@H](C)Oc2ccccc2C2(O)CS(=O)(=O)C2)c2cc(C3CCCOC3)ncc2c1] |
| 98 | 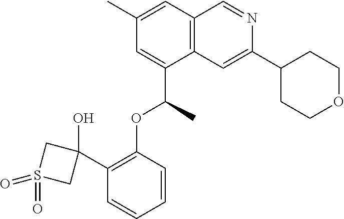 [SMILES: Cc1cc([C@@H](C)Oc2ccccc2C2(O)CS(=O)(=O)C2)c2cc(C3CCOCC3)ncc2c1] |
| 99 | 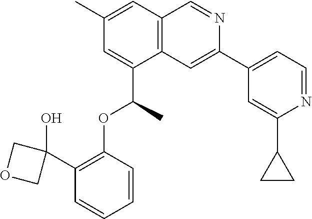 [SMILES: Cc1cc([C@@H](C)Oc2ccccc2C2(O)COC2)c2cc(-c3ccnc(C4CC4)c3)ncc2c1] |
| 100 | 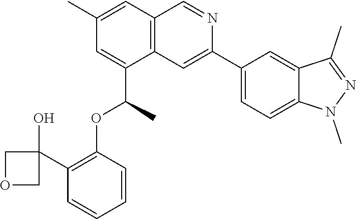 [SMILES: Cc1cc([C@@H](C)Oc2ccccc2C2(O)COC2)c2cc(-c3ccc4c(c3)c(C)nn4C)ncc2c1] |
| 101 | 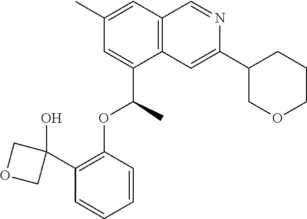 [SMILES: Cc1cc([C@@H](C)Oc2ccccc2C2(O)COC2)c2cc(C3CCCOC3)ncc2c1] |
| 102 | 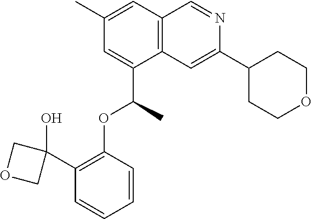 [SMILES: Cc1cc([C@@H](C)Oc2ccccc2C2(O)COC2)c2cc(C3CCOCC3)ncc2c1] |
| 103 | 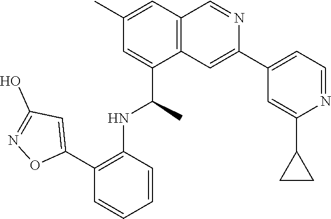 [SMILES: Cc1cc([C@@H](C)Nc2ccccc2-c2cc(O)no2)c2cc(-c3ccnc(C4CC4)c3)ncc2c1] |
| 104 | 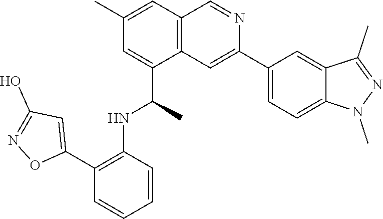 [SMILES: Cc1cc([C@@H](C)Nc2ccccc2-c2cc(O)no2)c2cc(-c3ccc4c(c3)c(C)nn4C)ncc2c1] |
| 105 | 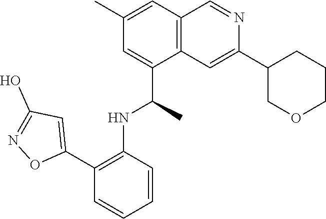 [SMILES: Cc1cc([C@@H](C)Nc2ccccc2-c2cc(O)no2)c2cc(C3CCCOC3)ncc2c1] |
| 106 | 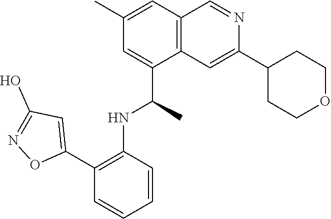 [SMILES: Cc1cc([C@@H](C)Nc2ccccc2-c2cc(O)no2)c2cc(C3CCOCC3)ncc2c1] |
| 107 | 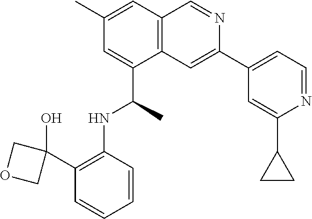 [SMILES: Cc1cc([C@@H](C)Nc2ccccc2C2(O)COC2)c2cc(-c3ccnc(C4CC4)c3)ncc2c1] |
| 108 | 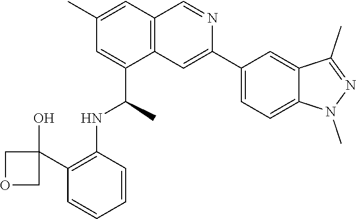 [SMILES: Cc1cc([C@@H](C)Nc2ccccc2C2(O)COC2)c2cc(-c3ccc4c(c3)c(C)nn4C)ncc2c1] |
| 109 | 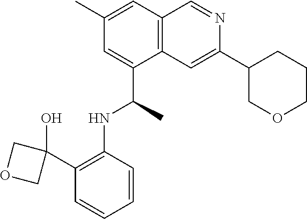 [SMILES: Cc1cc([C@@H](C)Nc2ccccc2C2(O)COC2)c2cc(C3CCCOC3)ncc2c1] |
| 110 | 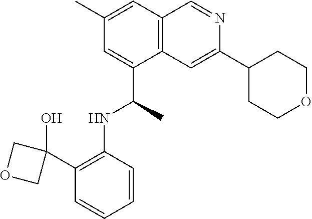 [SMILES: Cc1cc([C@@H](C)Nc2ccccc2C2(O)COC2)c2cc(C3CCOCC3)ncc2c1] |
| 111 | 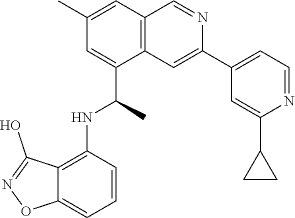 [SMILES: Cc1cc([C@@H](C)Nc2cccc3onc(O)c23)c2cc(-c3ccnc(C4CC4)c3)ncc2c1] |
| 112 | 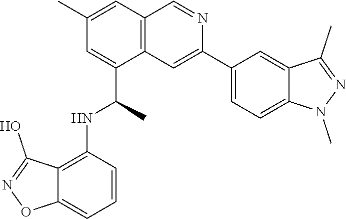 [SMILES: Cc1cc([C@@H](C)Nc2cccc3onc(O)c23)c2cc(-c3ccc4c(c3)c(C)nn4C)ncc2c1] |
| 113 | 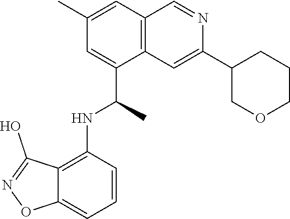 [SMILES: Cc1cc([C@@H](C)Nc2cccc3onc(O)c23)c2cc(C3CCCOC3)ncc2c1] |
| 114 | 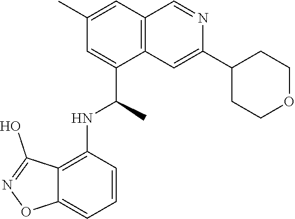 [SMILES: Cc1cc([C@@H](C)Nc2cccc3onc(O)c23)c2cc(C3CCOCC3)ncc2c1] |
| 115 | 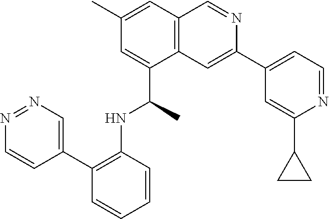 [SMILES: Cc1cc([C@@H](C)Nc2ccccc2-c2ccnnc2)c2cc(-c3ccnc(C4CC4)c3)ncc2c1] |
| 116 | 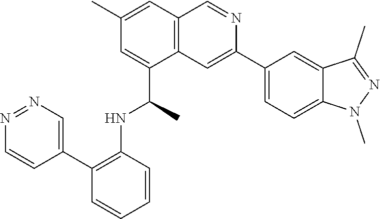 [SMILES: Cc1cc([C@@H](C)Nc2ccccc2-c2ccnnc2)c2cc(-c3ccc4c(c3)c(C)nn4C)ncc2c1] |
| 117 | 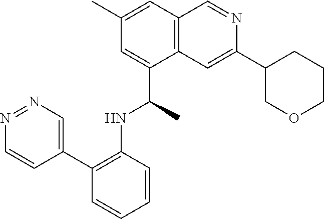 [SMILES: Cc1cc([C@@H](C)Nc2ccccc2-c2ccnnc2)c2cc(C3CCCOC3)ncc2c1] |
| 118 | 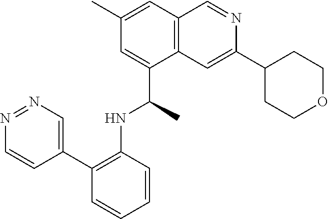 [SMILES: Cc1cc([C@@H](C)Nc2ccccc2-c2ccnnc2)c2cc(C3CCOCC3)ncc2c1] |
| 119 | 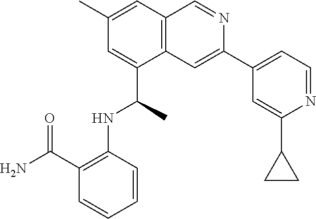 [SMILES: Cc1cc([C@@H](C)Nc2ccccc2C(N)=O)c2cc(-c3ccnc(C4CC4)c3)ncc2c1] |
| 120 | 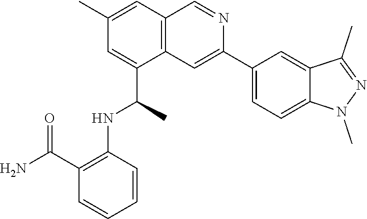 [SMILES: Cc1cc([C@@H](C)Nc2ccccc2C(N)=O)c2cc(-c3ccc4c(c3)c(C)nn4C)ncc2c1] |
| 121 | 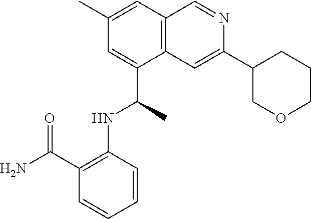 [SMILES: Cc1cc([C@@H](C)Nc2ccccc2C(N)=O)c2cc(C3CCCOC3)ncc2c1] |
| 122 | 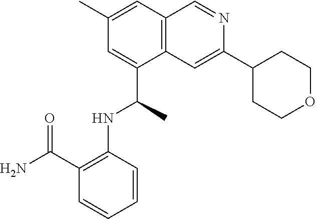 [SMILES: Cc1cc([C@@H](C)Nc2ccccc2C(N)=O)c2cc(C3CCOCC3)ncc2c1] |
| 123 | 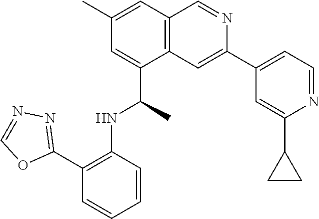 [SMILES: Cc1cc([C@@H](C)Nc2ccccc2-c2nnco2)c2cc(-c3ccnc(C4CC4)c3)ncc2c1] |
| 124 | 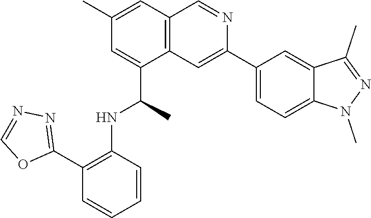 [SMILES: Cc1cc([C@@H](C)Nc2ccccc2-c2nnco2)c2cc(-c3ccc4c(c3)c(C)nn4C)ncc2c1] |
| 125 | 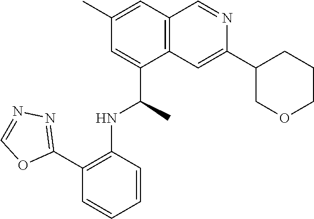 [SMILES: Cc1cc([C@@H](C)Nc2ccccc2-c2nnco2)c2cc(C3CCCOC3)ncc2c1] |
| 126 | 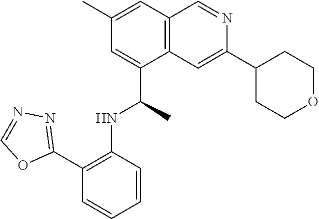 [SMILES: Cc1cc([C@@H](C)Nc2ccccc2-c2nnco2)c2cc(C3CCOCC3)ncc2c1] |
| 127 | 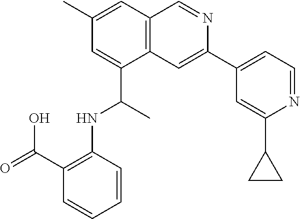 [SMILES: Cc1cc(C(C)Nc2ccccc2C(=O)O)c2cc(-c3ccnc(C4CC4)c3)ncc2c1] |
| 128 | 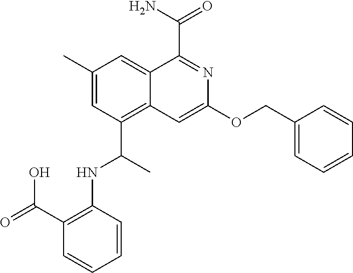 [SMILES: Cc1cc(C(C)Nc2ccccc2C(=O)O)c2cc(OCc3ccccc3)nc(C(N)=O)c2c1] |
| 129 | 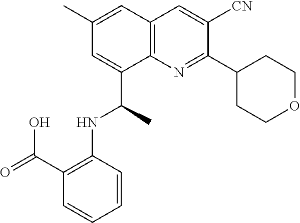 [SMILES: Cc1cc([C@@H](C)Nc2ccccc2C(=O)O)c2nc(C3CCOCC3)c(C#N)cc2c1] |
| 130 | 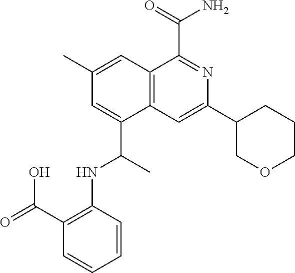 [SMILES: Cc1cc(C(C)Nc2ccccc2C(=O)O)c2cc(C3CCCOC3)nc(C(N)=O)c2c1] |
| 131 | 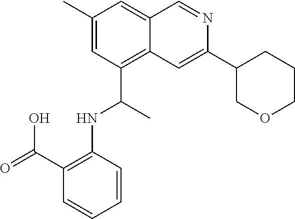 [SMILES: Cc1cc(C(C)Nc2ccccc2C(=O)O)c2cc(C3CCCOC3)ncc2c1] |
| 132 | 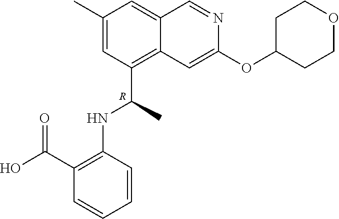 |
| 133 | 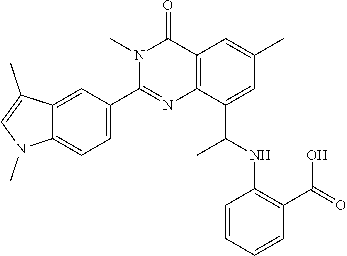 [SMILES: Cc1cc(C(C)Nc2ccccc2C(=O)O)c2nc(-c3ccc4c(c3)c(C)cn4C)n(C)c(=O)c2c1] |
| 134 | 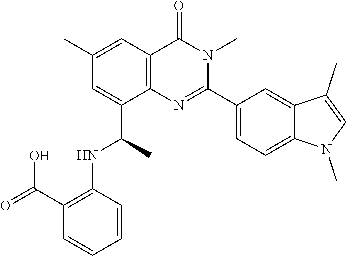 [SMILES: Cc1cc([C@@H](C)Nc2ccccc2C(=O)O)c2nc(-c3ccc4c(c3)c(C)cn4C)n(C)c(=O)c2c1] |
| 135 | 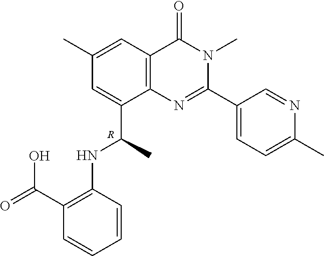 |
| 136 | 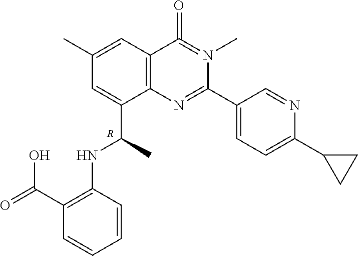 |
| 137 | 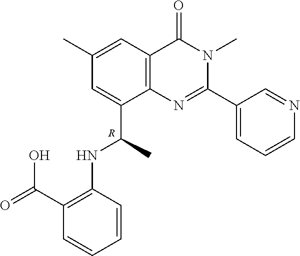 |
| 138 | 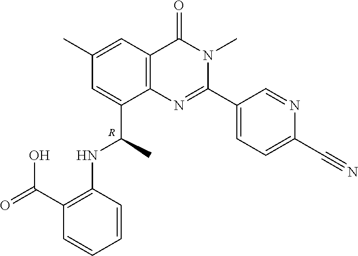 |
| 139 | 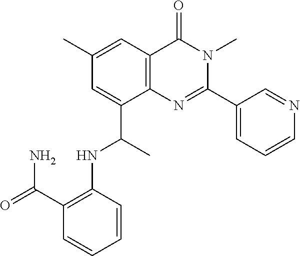 [SMILES: Cc1cc(C(C)Nc2ccccc2C(N)=O)c2nc(-c3cccnc3)n(C)c(=O)c2c1] |
| 140 | 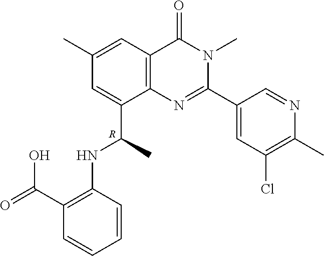 |
| 141 | 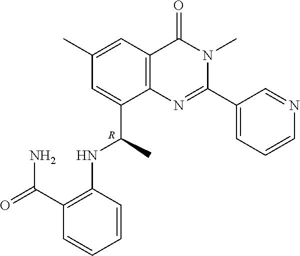 |
| 142 | 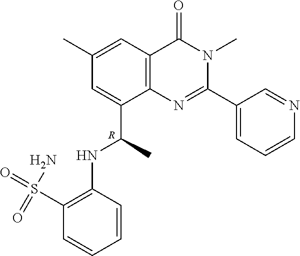 |
| 143 | 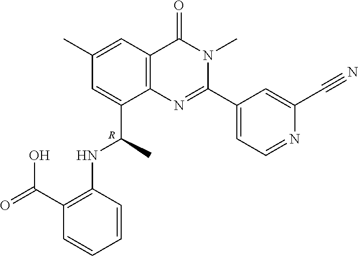 |
| 144 | 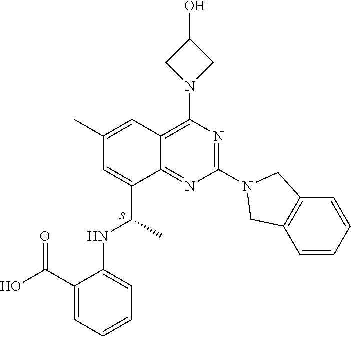 |
| 145 | 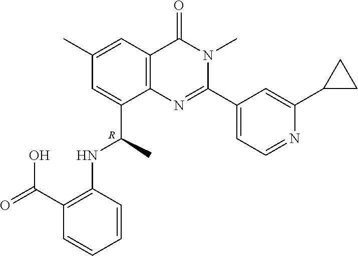 |
| 146 | 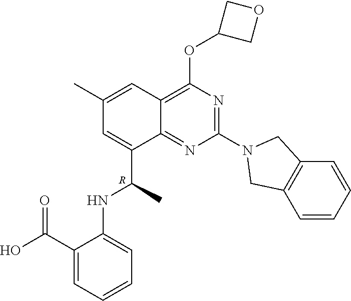 |
| 147 | 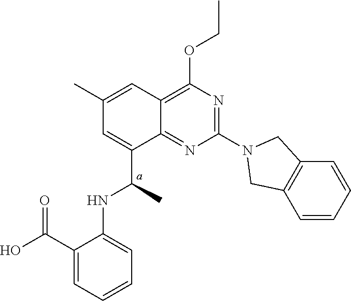 |

In some embodiments, the compound is selected from the group consisting of Compounds 148-139, or a pharmaceutically acceptable salt of any of the foregoing.

In some embodiments, the compound is selected from the group consisting of the compounds delineated in Table B, or a pharmaceutically acceptable salt of any of the foregoing.

TABLE B

| Example | |
|---|---|
| No. | Structure |
| 148 | 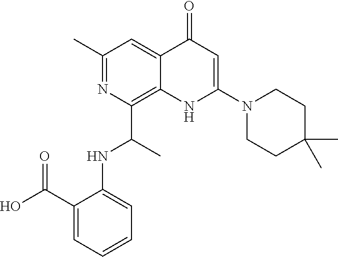 [SMILES: Cc1cc2c(=O)cc(N3CCC(C)(C)CC3)[nH]c2c(C(C)Nc2ccccc2C(=O)O)n1] |
| 149 | 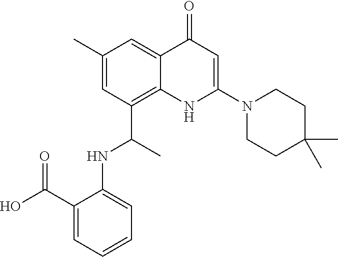 [SMILES: Cc1cc(C(C)Nc2ccccc2C(=O)O)c2[nH]c(N3CCC(C)(C)CC3)cc(=O)c2c1] |
| 150 | 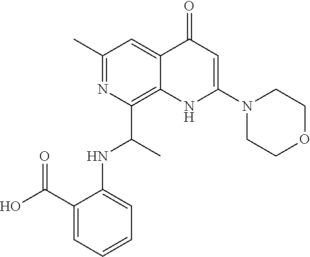 [SMILES: Cc1cc2c(=O)cc(N3CCOCC3)[nH]c2c(C(C)Nc2ccccc2C(=O)O)n1] |
| 151 | 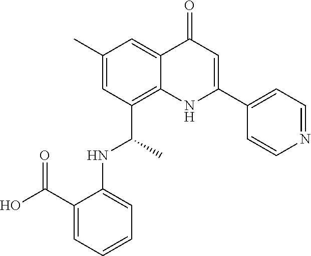 [SMILES: Cc1cc([C@H](C)Nc2ccccc2C(=O)O)c2[nH]c(-c3ccncc3)cc(=O)c2c1] |
| 152 | 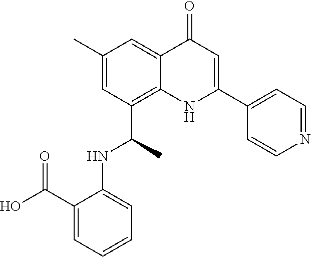 [SMILES: Cc1cc([C@@H](C)Nc2ccccc2C(=O)O)c2[nH]c(-c3ccncc3)cc(=O)c2c1] |
| 153 | 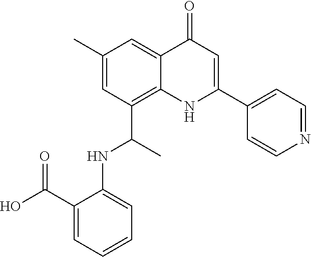 [SMILES: Cc1cc(C(C)Nc2ccccc2C(=O)O)c2[nH]c(-c3ccncc3)cc(=O)c2c1] |
| 154 | 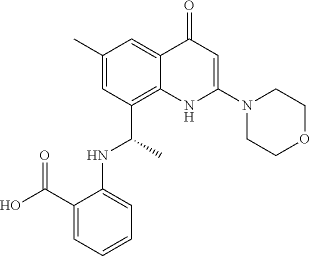 [SMILES: Cc1cc([C@H](C)Nc2ccccc2C(=O)O)c2[nH]c(N3CCOCC3)cc(=O)c2c1] |
| 155 | 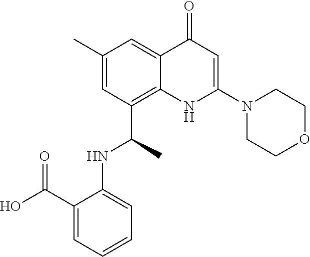 [SMILES: Cc1cc([C@@H](C)Nc2ccccc2C(=O)O)c2[nH]c(N3CCOCC3)cc(=O)c2c1] |
| 156 | 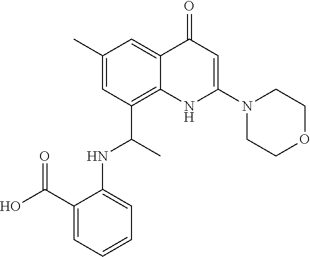 [SMILES: Cc1cc(C(C)Nc2ccccc2C(=O)O)c2[nH]c(N3CCOCC3)cc(=O)c2c1] |

In some embodiments, the compound is selected from the group consisting of the Compounds 140-226, or a pharmaceutically acceptable salt of any of the foregoing.

In some embodiments, the compound is selected from the group consisting of the compounds delineated in Table C, or a pharmaceutically acceptable salt of any of the foregoing.

TABLE C

| Example | |
|---|---|
| No. | Structure |
| 157 | 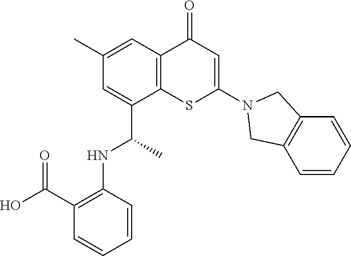 [SMILES: Cc1cc([C@H](C)Nc2ccccc2C(=O)O)c2sc(N3Cc4ccccc4C3)cc(=O)c2c1] |
| 158 | 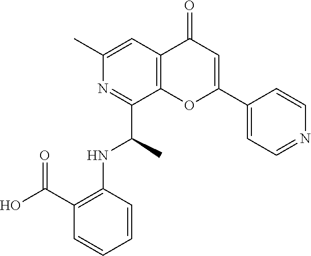 [SMILES: Cc1cc2c(=O)cc(-c3ccncc3)oc2c([C@@H](C)Nc2ccccc2C(=O)O)n1] |
| 159 | 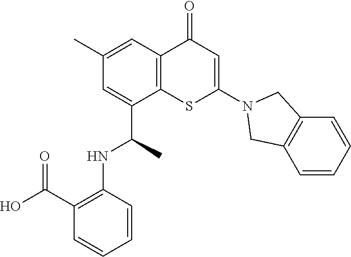 [SMILES: Cc1cc([C@@H](C)Nc2ccccc2C(=O)O)c2sc(N3Cc4ccccc4C3)cc(=O)c2c1] |
| 160 | 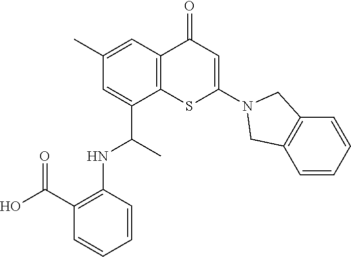 [SMILES: Cc1cc(C(C)Nc2ccccc2C(=O)O)c2sc(N3Cc4ccccc4C3)cc(=O)c2c1] |
| 161 | 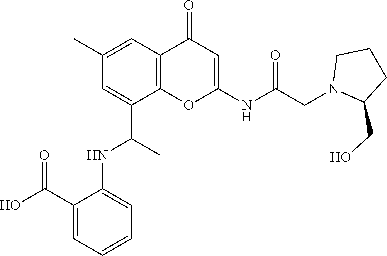 [SMILES: Cc1cc(C(C)Nc2ccccc2C(=O)O)c2oc(NC(=O)CN3CCC[C@H]3CO)cc(=O)c2c1] |
| 162 | 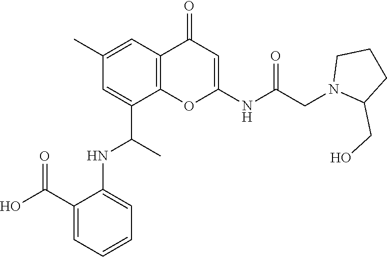 [SMILES: Cc1cc(C(C)Nc2ccccc2C(=O)O)c2oc(NC(=O)CN3CCCC3CO)cc(=O)c2c1] |
| 163 | 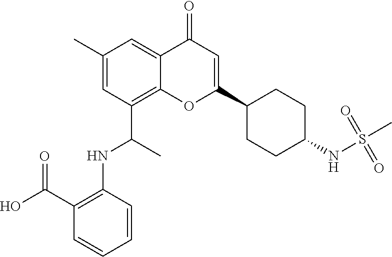 [SMILES: Cc1cc(C(C)Nc2ccccc2C(=O)O)c2oc([C@H]3CC[C@H](NS(C)(=O)=O)CC3)cc(=O)c2c1] |
| 164 | 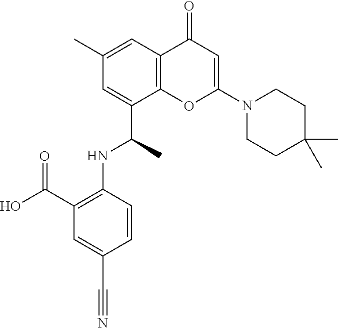 [SMILES: Cc1cc([C@@H](C)Nc2ccc(C#N)cc2C(=O)O)c2oc(N3CCC(C)(C)CC3)cc(=O)c2c1] |
| 165 | 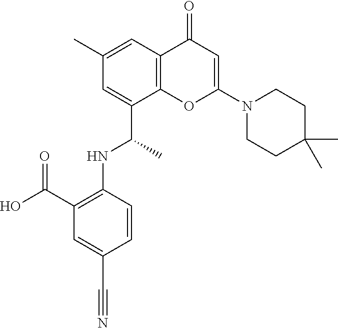 [SMILES: Cc1cc([C@H](C)Nc2ccc(C#N)cc2C(=O)O)c2oc(N3CCC(C)(C)CC3)cc(=O)c2c1] |
| 166 | 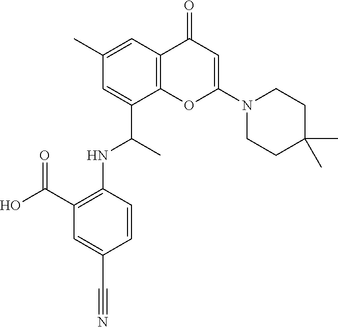 [SMILES: Cc1cc(C(C)Nc2ccc(C#N)cc2C(=O)O)c2oc(N3CCC(C)(C)CC3)cc(=O)c2c1] |
| 167 | 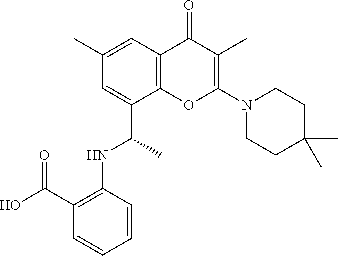 [SMILES: Cc1cc([C@H](C)Nc2ccccc2C(=O)O)c2oc(N3CCC(C)(C)CC3)c(C)c(=O)c2c1] |
| 168 | 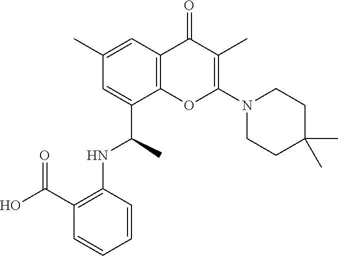 [SMILES: Cc1cc([C@@H](C)Nc2ccccc2C(=O)O)c2oc(N3CCC(C)(C)CC3)c(C)c(=O)c2c1] |
| 169 | 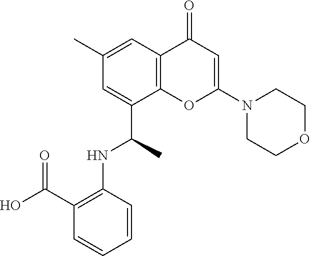 [SMILES: Cc1cc([C@@H](C)Nc2ccccc2C(=O)O)c2oc(N3CCOCC3)cc(=O)c2c1] |
| 170 | 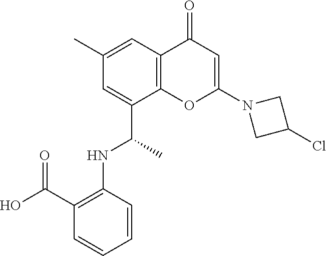 [SMILES: Cc1cc([C@H](C)Nc2ccccc2C(=O)O)c2oc(N3CC(Cl)C3)cc(=O)c2c1] |
| 171 | 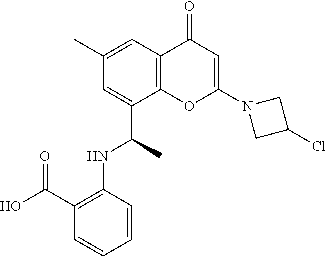 [SMILES: Cc1cc([C@@H](C)Nc2ccccc2C(=O)O)c2oc(N3CC(Cl)C3)cc(=O)c2c1] |
| 172 | 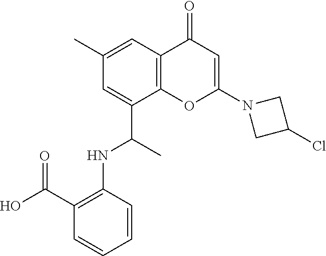 [SMILES: Cc1cc(C(C)Nc2ccccc2C(=O)O)c2oc(N3CC(Cl)C3)cc(=O)c2c1] |
| 173 | 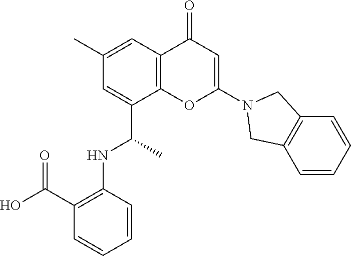 [SMILES: Cc1cc([C@H](C)Nc2ccccc2C(=O)O)c2oc(N3Cc4ccccc4C3)cc(=O)c2c1] |
| 174 | 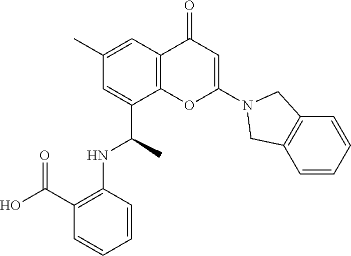 [SMILES: Cc1cc([C@@H](C)Nc2ccccc2C(=O)O)c2oc(N3Cc4ccccc4C3)cc(=O)c2c1] |
| 175 | 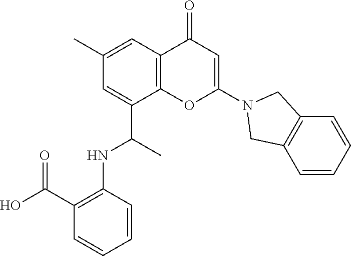 [SMILES: Cc1cc(C(C)Nc2ccccc2C(=O)O)c2oc(N3Cc4ccccc4C3)cc(=O)c2c1] |
| 176 | 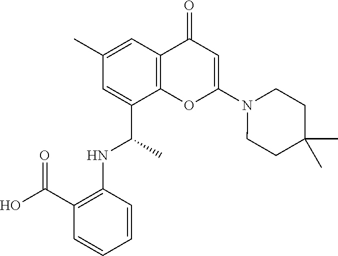 [SMILES: Cc1cc([C@H](C)Nc2ccccc2C(=O)O)c2oc(N3CCC(C)(C)CC3)cc(=O)c2c1] |
| 177 | 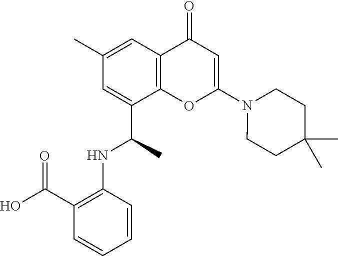 [SMILES: Cc1cc([C@@H](C)Nc2ccccc2C(=O)O)c2oc(N3CCC(C)(C)CC3)cc(=O)c2c1] |
| 178 | 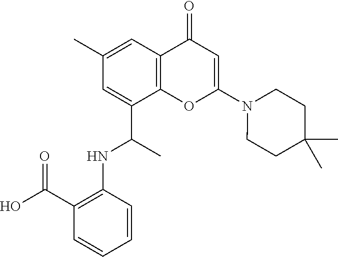 [SMILES: Cc1cc(C(C)Nc2ccccc2C(=O)O)c2oc(N3CCC(C)(C)CC3)cc(=O)c2c1] |
| 179 | 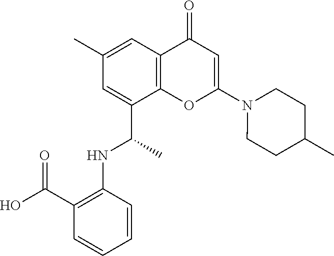 [SMILES: Cc1cc([C@H](C)Nc2ccccc2C(=O)O)c2oc(N3CCC(C)CC3)cc(=O)c2c1] |
| 180 | 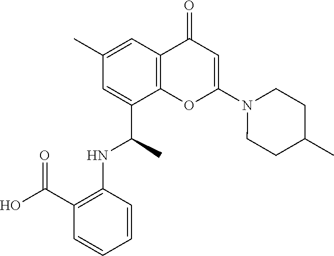 [SMILES: Cc1cc([C@@H](C)Nc2ccccc2C(=O)O)c2oc(N3CCC(C)CC3)cc(=O)c2c1] |
| 181 | 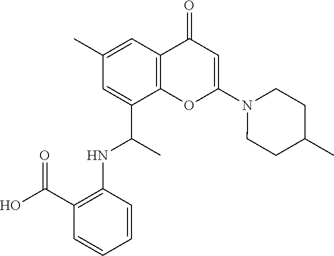 [SMILES: Cc1cc(C(C)Nc2ccccc2C(=O)O)c2oc(N3CCC(C)CC3)cc(=O)c2c1] |
| 182 | 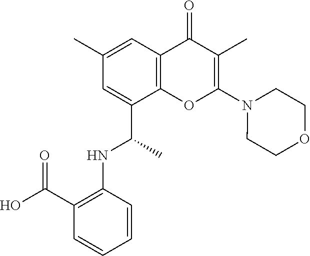 [SMILES: Cc1cc([C@H](C)Nc2ccccc2C(=O)O)c2oc(N3CCOCC3)c(C)c(=O)c2c1] |
| 183 | 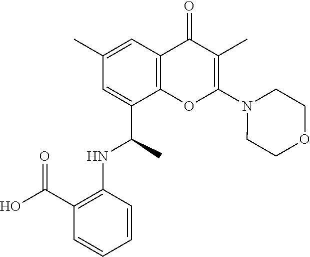 [SMILES: Cc1cc([C@@H](C)Nc2ccccc2C(=O)O)c2oc(N3CCOCC3)c(C)c(=O)c2c1] |
| 184 | 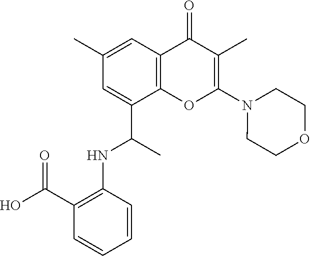 [SMILES: Cc1cc(C(C)Nc2ccccc2C(=O)O)c2oc(N3CCOCC3)c(C)c(=O)c2c1] |
| 185 | 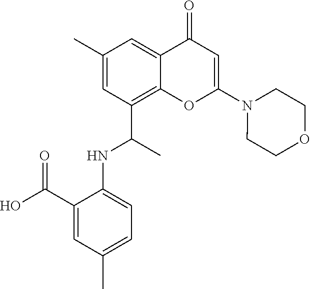 [SMILES: Cc1ccc(NC(C)c2cc(C)cc3c(=O)cc(N4CCOCC4)oc23)c(C(=O)O)c1] |
| 186 | 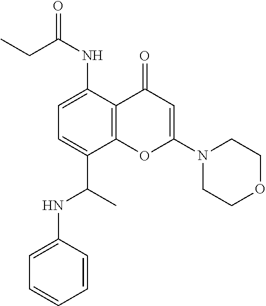 [SMILES: CCC(=O)Nc1ccc(C(C)Nc2ccccc2)c2oc(N3CCOCC3)cc(=O)c12] |
| 187 | 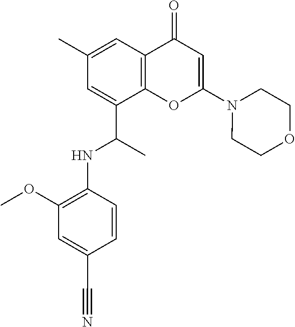 [SMILES: COc1cc(C#N)ccc1NC(C)c1cc(C)cc2c(=O)cc(N3CCOCC3)oc12] |
| 188 | 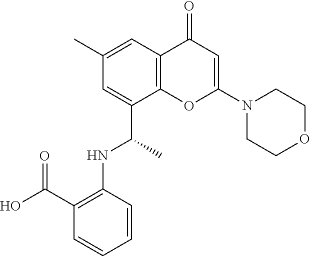 [SMILES: Cc1cc([C@H](C)Nc2ccccc2C(=O)O)c2oc(N3CCOCC3)cc(=O)c2c1] |
| 189 | 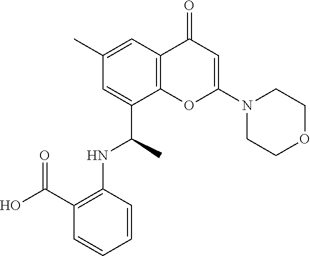 [SMILES: Cc1cc([C@@H](C)Nc2ccccc2C(=O)O)c2oc(N3CCOCC3)cc(=O)c2c1] |
| 190 | 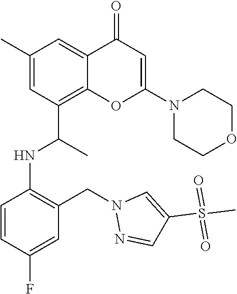 [SMILES: Cc1cc(C(C)Nc2ccc(F)cc2Cn2cc(S(C)(=O)=O)cn2)c2oc(N3CCOCC3)cc(=O)c2c1] |
| 191 | 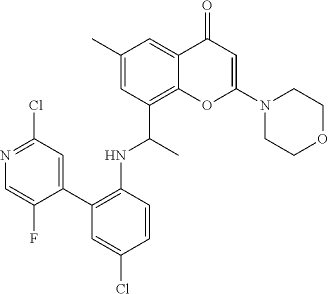 [SMILES: Cc1cc(C(C)Nc2ccc(Cl)cc2-c2cc(Cl)ncc2F)c2oc(N3CCOCC3)cc(=O)c2c1] |
| 192 | 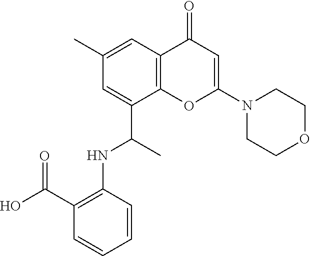 [SMILES: Cc1cc(C(C)Nc2ccccc2C(=O)O)c2oc(N3CCOCC3)cc(=O)c2c1] |
| 193 | 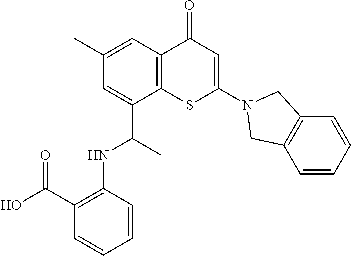 [SMILES: Cc1cc(C(C)Nc2ccccc2C(=O)O)c2sc(N3Cc4ccccc4C3)cc(=O)c2c1] |
| 194 | 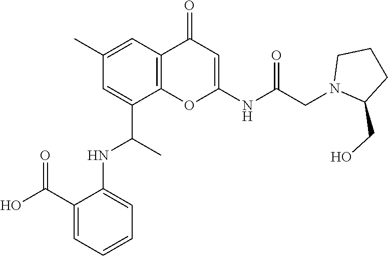 [SMILES: Cc1cc(C(C)Nc2ccccc2C(=O)O)c2oc(NC(=O)CN3CCC[C@H]3CO)cc(=O)c2c1] |
| 195 | 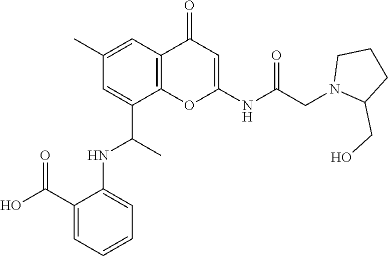 [SMILES: Cc1cc(C(C)Nc2ccccc2C(=O)O)c2oc(NC(=O)CN3CCCC3CO)cc(=O)c2c1] |
| 196 | 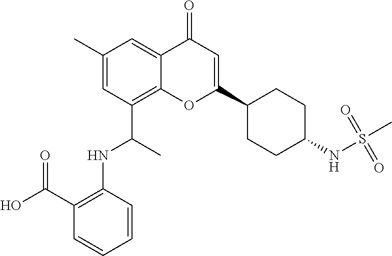 [SMILES: Cc1cc(C(C)Nc2ccccc2C(=O)O)c2oc([C@H]3CC[C@H](NS(C)(=O)=O)CC3)cc(=O)c2c1] |
| 197 | 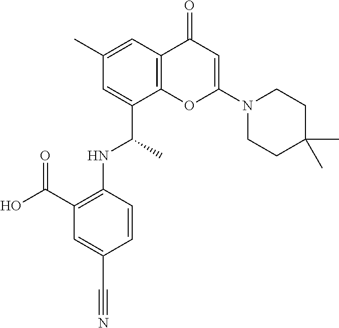 [SMILES: Cc1cc([C@H](C)Nc2ccc(C#N)cc2C(=O)O)c2oc(N3CCC(C)(C)CC3)cc(=O)c2c1] |
| 198 | 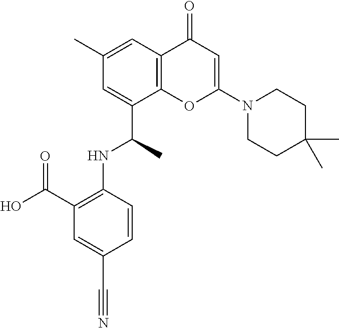 [SMILES: Cc1cc([C@@H](C)Nc2ccc(C#N)cc2C(=O)O)c2oc(N3CCC(C)(C)CC3)cc(=O)c2c1] |
| 199 | 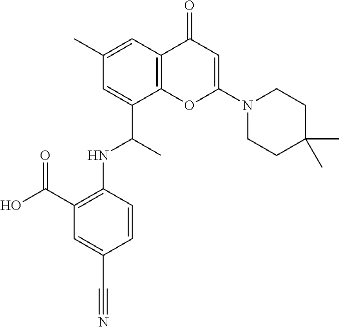 [SMILES: Cc1cc(C(C)Nc2ccc(C#N)cc2C(=O)O)c2oc(N3CCC(C)(C)CC3)cc(=O)c2c1] |
| 200 | 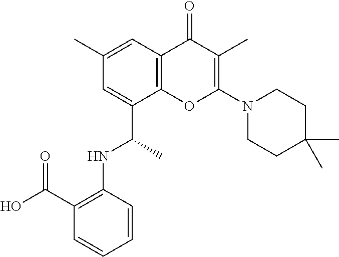 [SMILES: Cc1cc([C@H](C)Nc2ccccc2C(=O)O)c2oc(N3CCC(C)(C)CC3)c(C)c(=O)c2c1] |
| 201 | 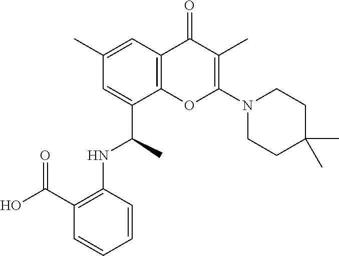 [SMILES: Cc1cc([C@@H](C)Nc2ccccc2C(=O)O)c2oc(N3CCC(C)(C)CC3)c(C)c(=O)c2c1] |
| 202 | 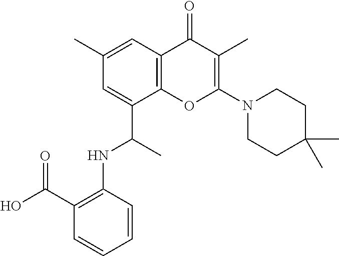 [SMILES: Cc1cc(C(C)Nc2ccccc2C(=O)O)c2oc(N3CCC(C)(C)CC3)c(C)c(=O)c2c1] |
| 203 | 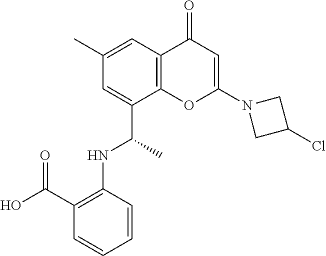 [SMILES: Cc1cc([C@H](C)Nc2ccccc2C(=O)O)c2oc(N3CC(Cl)C3)cc(=O)c2c1] |
| 204 | 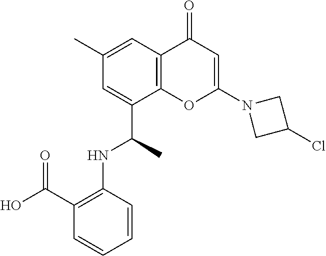 [SMILES: Cc1cc([C@@H](C)Nc2ccccc2C(=O)O)c2oc(N3CC(Cl)C3)cc(=O)c2c1] |
| 205 | 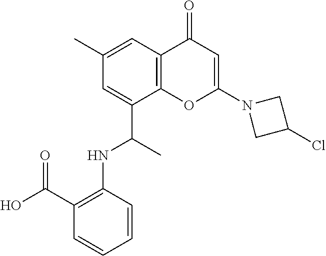 [SMILES: Cc1cc(C(C)Nc2ccccc2C(=O)O)c2oc(N3CC(Cl)C3)cc(=O)c2c1] |
| 206 | 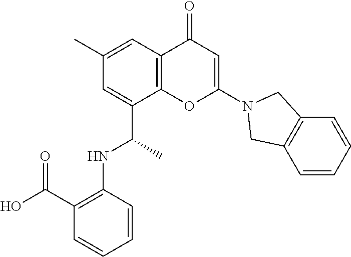 [SMILES: Cc1cc([C@H](C)Nc2ccccc2C(=O)O)c2oc(N3Cc4ccccc4C3)cc(=O)c2c1] |
| 207 | 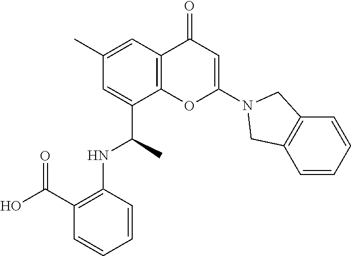 [SMILES: Cc1cc([C@@H](C)Nc2ccccc2C(=O)O)c2oc(N3Cc4ccccc4C3)cc(=O)c2c1] |
| 208 | 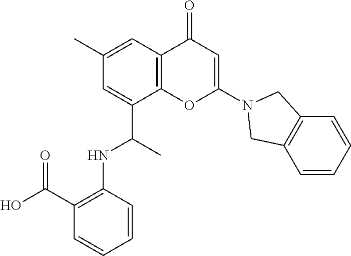 [SMILES: Cc1cc(C(C)Nc2ccccc2C(=O)O)c2oc(N3Cc4ccccc4C3)cc(=O)c2c1] |
| 209 | 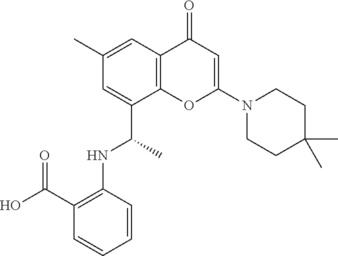 [SMILES: Cc1cc([C@H](C)Nc2ccccc2C(=O)O)c2oc(N3CCC(C)(C)CC3)cc(=O)c2c1] |
| 210 | 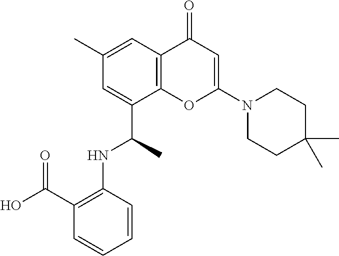 [SMILES: Cc1cc([C@@H](C)Nc2ccccc2C(=O)O)c2oc(N3CCC(C)(C)CC3)cc(=O)c2c1] |
| 211 | 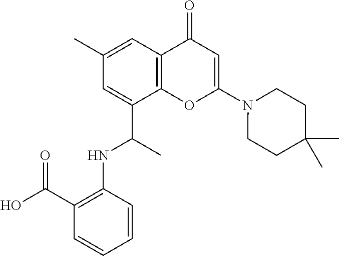 [SMILES: Cc1cc(C(C)Nc2ccccc2C(=O)O)c2oc(N3CCC(C)(C)CC3)cc(=O)c2c1] |
| 212 | 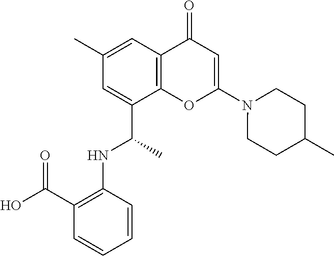 [SMILES: Cc1cc([C@H](C)Nc2ccccc2C(=O)O)c2oc(N3CCC(C)CC3)cc(=O)c2c1] |
| 213 | 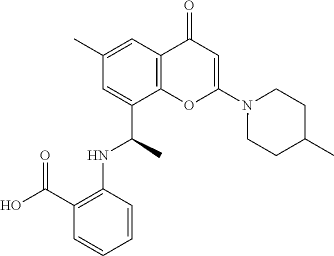 [SMILES: Cc1cc([C@@H](C)Nc2ccccc2C(=O)O)c2oc(N3CCC(C)CC3)cc(=O)c2c1] |
| 214 | 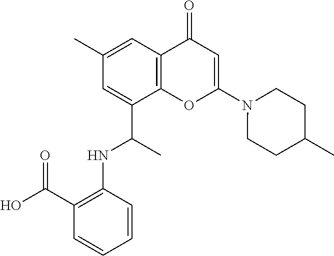 [SMILES: Cc1cc(C(C)Nc2ccccc2C(=O)O)c2oc(N3CCC(C)CC3)cc(=O)c2c1] |
| 215 | 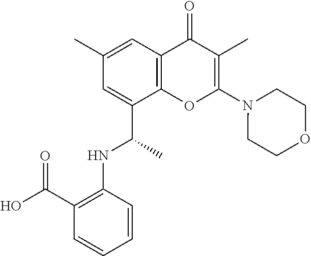 [SMILES: Cc1cc([C@H](C)Nc2ccccc2C(=O)O)c2oc(N3CCOCC3)c(C)c(=O)c2c1] |
| 216 | 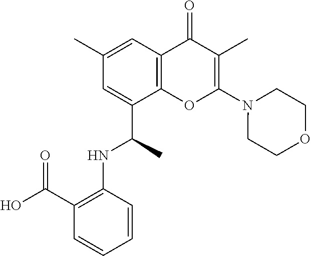 [SMILES: Cc1cc([C@@H](C)Nc2ccccc2C(=O)O)c2oc(N3CCOCC3)c(C)c(=O)c2c1] |
| 217 | 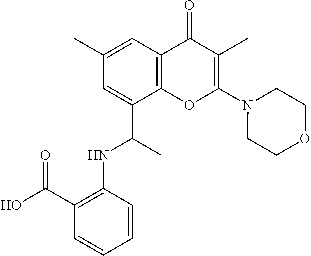 [SMILES: Cc1cc(C(C)Nc2ccccc2C(=O)O)c2oc(N3CCOCC3)c(C)c(=O)c2c1] |
| 218 | 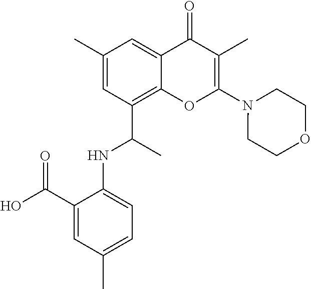 [SMILES: Cc1ccc(NC(C)c2cc(C)cc3c(=O)c(C)c(N4CCOCC4)oc23)c(C(=O)O)c1] |
| 219 | 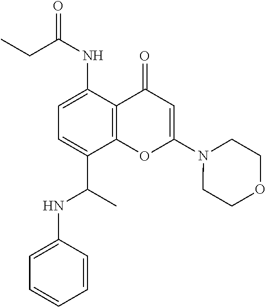 [SMILES: CCC(=O)Nc1ccc(C(C)Nc2ccccc2)c2oc(N3CCOCC3)cc(=O)c12] |
| 220 | 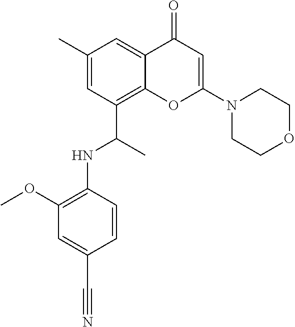 [SMILES: COc1cc(C#N)ccc1NC(C)c1cc(C)cc2c(=O)cc(N3CCOCC3)oc12] |
| 221 | 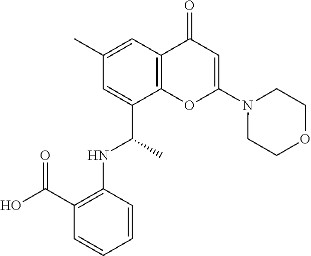 [SMILES: Cc1cc([C@H](C)Nc2ccccc2C(=O)O)c2oc(N3CCOCC3)cc(=O)c2c1] |
| 222 | 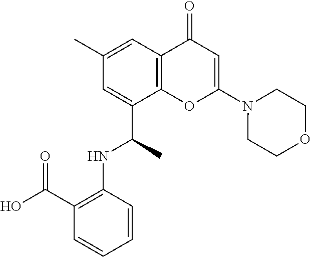 [SMILES: Cc1cc([C@@H](C)Nc2ccccc2C(=O)O)c2oc(N3CCOCC3)cc(=O)c2c1] |
| 223 | 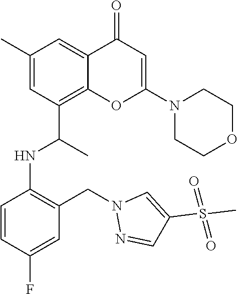 [SMILES: Cc1cc(C(C)Nc2ccc(F)cc2Cn2cc(S(C)(=O)=O)cn2)c2oc(N3CCOCC3)cc(=O)c2c1] |
| 224 | 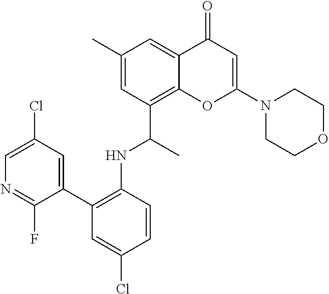 [SMILES: Cc1cc(C(C)Nc2ccc(Cl)cc2-c2cc(Cl)cnc2F)c2oc(N3CCOCC3)cc(=O)c2c1] |
| 225 | 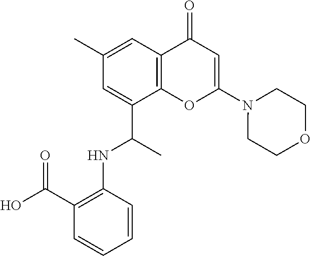 [SMILES: Cc1cc(C(C)Nc2ccccc2C(=O)O)c2oc(N3CCOCC3)cc(=O)c2c1] |
| 226 | 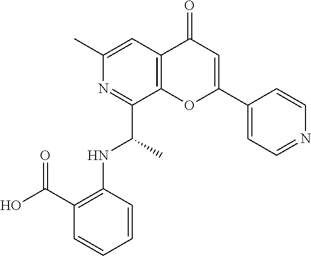 [SMILES: Cc1cc2c(=O)cc(-c3ccncc3)oc2c([C@H](C)Nc2ccccc2C(=O)O)n1] |
| 227 | 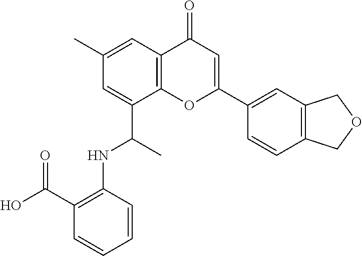 [SMILES: Cc1cc(C(C)Nc2ccccc2C(=O)O)c2oc(-c3ccc4c(c3)COC4)cc(=O)c2c1] |
| 228 | 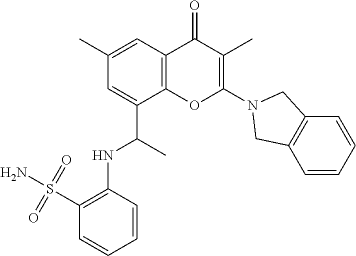 [SMILES: Cc1cc(C(C)Nc2ccccc2S(N)(=O)=O)c2oc(N3Cc4ccccc4C3)c(C)c(=O)c2c1] |
| 229 | 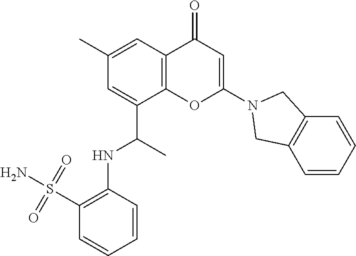 [SMILES: Cc1cc(C(C)Nc2ccccc2S(N)(=O)=O)c2oc(N3Cc4ccccc4C3)cc(=O)c2c1] |
| 230 | 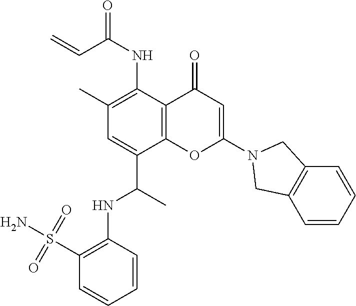 [SMILES: C=CC(=O)Nc1c(C)cc(C(C)Nc2ccccc2S(N)(=O)=O)c2oc(N3Cc4ccccc4C3)cc(=O)c12] |
| 231 | 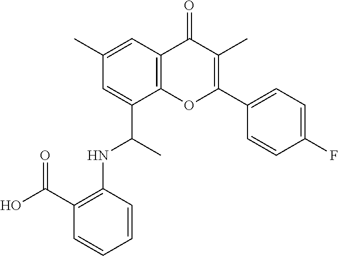 [SMILES: Cc1cc(C(C)Nc2ccccc2C(=O)O)c2oc(-c3ccc(F)cc3)c(C)c(=O)c2c1] |
| 232 | 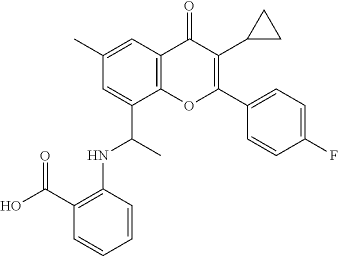 [SMILES: Cc1cc(C(C)Nc2ccccc2C(=O)O)c2oc(-c3ccc(F)cc3)c(C3CC3)c(=O)c2c1] |
| 233 | 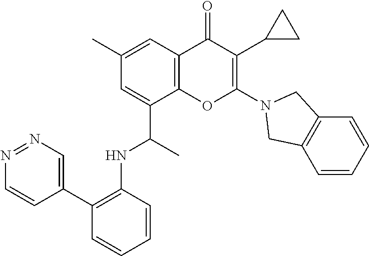 [SMILES: Cc1cc(C(C)Nc2ccccc2-c2ccnnc2)c2oc(N3Cc4ccccc4C3)c(C3CC3)c(=O)c2c1] |
| 234 | 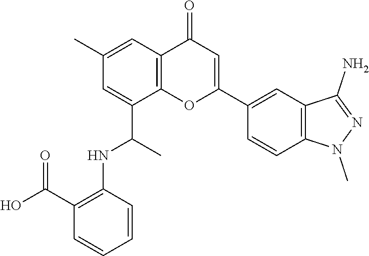 [SMILES: Cc1cc(C(C)Nc2ccccc2C(=O)O)c2oc(-c3ccc4c(c3)c(N)nn4C)cc(=O)c2c1] |
| 235 | 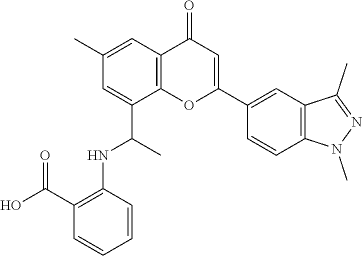 [SMILES: Cc1cc(C(C)Nc2ccccc2C(=O)O)c2oc(-c3ccc4c(c3)c(C)nn4C)cc(=O)c2c1] |
| 236 | 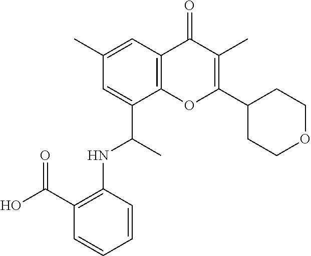 [SMILES: Cc1cc(C(C)Nc2ccccc2C(=O)O)c2oc(C3CCOCC3)c(C)c(=O)c2c1] |
| 237 | 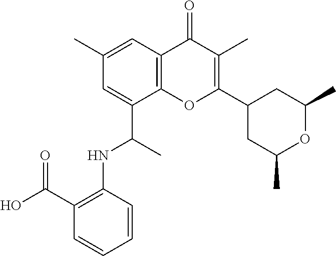 [SMILES: Cc1cc(C(C)Nc2ccccc2C(=O)O)c2oc(C3C[C@@H](C)O[C@@H](C)C3)c(C)c(=O)c2c1] |
| 238 | 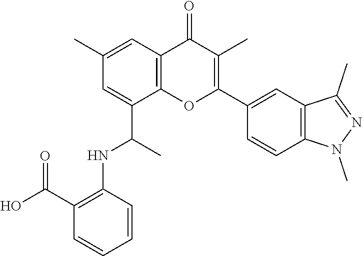 [SMILES: Cc1cc(C(C)Nc2ccccc2C(=O)O)c2oc(-c3ccc4c(c3)c(C)nn4C)c(C)c(=O)c2c1] |
| 239 | 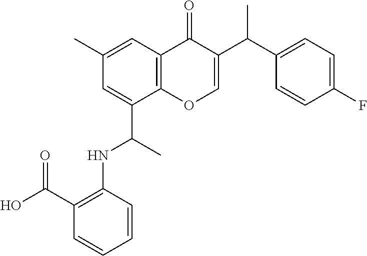 [SMILES: Cc1cc(C(C)Nc2ccccc2C(=O)O)c2occ(C(C)c3ccc(F)cc3)c(=O)c2c1] |
| 240 | 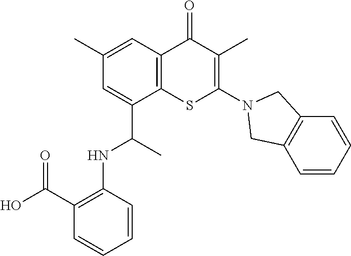 [SMILES: Cc1cc(C(C)Nc2ccccc2C(=O)O)c2sc(N3Cc4ccccc4C3)c(C)c(=O)c2c1] |
| 241 | 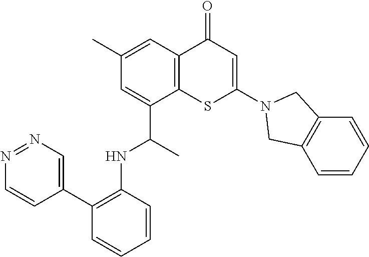 [SMILES: Cc1cc(C(C)Nc2ccccc2-c2ccnnc2)c2sc(N3Cc4ccccc4C3)cc(=O)c2c1] |
| 242 | 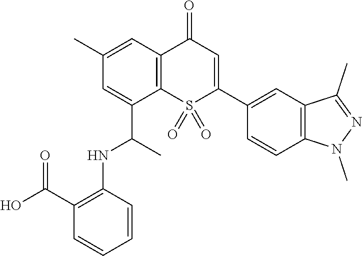 [SMILES: Cc1cc2c(c(C(C)Nc3ccccc3C(=O)O)c1)S(=O)(=O)C(c1ccc3c(c1)c(C)nn3C)=CC2=O] |
| 243 | 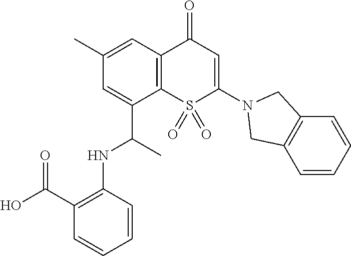 [SMILES: Cc1cc2c(c(C(C)Nc3ccccc3C(=O)O)c1)S(=O)(=O)C(N1Cc3ccccc3C1)=CC2=O] |

### Pharmaceutical Compositions

Some embodiments provide a pharmaceutical composition comprising a compound of Formula (I), Formula (II), or Formula (III), or a pharmaceutically acceptable salt of any of the foregoing, and one or more pharmaceutically acceptable excipients.

### Methods of Treatment

A “PI3Kα inhibitor” as used herein (e.g., compounds of Formula (I), Formula (II), Formula (III), and pharmaceutically acceptable salts of any of the foregoing) includes any compound exhibiting PI3Kα inactivation activity (e.g., inhibiting or decreasing). In some embodiments, a PI3Kα inhibitor can be selective for a PI3Kα having one or more mutations.

The ability of test compounds to act as inhibitors of PI3Kα may be demonstrated by assays known in the art. The activity of the compounds and compositions provided herein as PI3Kα inhibitors can be assayed in vitro, in vivo, or in a cell line. In vitro assays include assays that determine inhibition of the kinase. Alternate in vitro assays quantitate the ability of the inhibitor to bind to the protein kinase and can be measured either by radio labeling the compound prior to binding, isolating the compound/kinase complex and determining the amount of radio label bound, or by running a competition experiment where new compounds are incubated with the kinase bound to known radio ligands.

Potency of a PI3Kα inhibitor as provided herein can be determined by EC₅₀ value. A compound with a lower EC₅₀ value, as determined under substantially similar conditions, is a more potent inhibitor relative to a compound with a higher EC₅₀ value.

Potency of a PI3Kα inhibitor as provided herein can also be determined by IC₅₀ value. A compound with a lower IC₅₀ value, as determined under substantially similar conditions, is a more potent inhibitor relative to a compound with a higher IC₅₀ value. In some embodiments, the substantially similar conditions comprise determining a PI3Kα-dependent phosphorylation level, in vitro or in vivo.

The selectivity between wild type PI3Kα and PI3Kα containing one or more mutations as described herein can also be measured using in vitro assays such as surface plasmon resonance and fluorence-based binding assays, and cellular assays such as the levels of pAKT, a biomarker of PI3Kα activity, and/or proliferation assays where cell proliferation is dependent on mutant PI3Kα kinase activity.

In some embodiments, the compounds provided herein can exhibit potent and selective inhibition of PI3Kα. For example, the compounds provided herein can bind to the helical phosphatidylinositol kinase homology domain catalytic domain of PI3Kα. In some embodiments, the compounds provided herein can exhibit nanomolar potency against a PI3Kα kinase including one or more mutations, for example, the mutations in Table 1.

In some embodiments, the compounds of Formula (I), Formula (II), or Formula (III), or a pharmaceutically acceptable salt of any of the foregoing, can selectively target PI3Kα. For example, a compound of Formula (I), Formula (II), or Formula (III), or a pharmaceutically acceptable salt of any of the foregoing, can selectively target PI3Kα over another kinase or non-kinase target.

In some embodiments, a compound of Formula (I), Formula (II), or Formula (III), or a pharmaceutically acceptable salt of any of the foregoing, can exhibit greater inhibition of PI3Kα containing one or more mutations as described herein (e.g., one or more mutations as described in Table 1) relative to inhibition of wild type PI3Kα. In some embodiments, a compound of Formula (I), Formula (II), or Formula (III), or a pharmaceutically acceptable salt of any of the foregoing can exhibit at least 2-fold, 3-fold, 5-fold, 10-fold, 25-fold, 50-fold or 100-fold greater inhibition of PI3Kα containing one or more mutations as described herein relative to inhibition of wild type PI3Kα. In some embodiments, a compound of Formula (I), Formula (II), or Formula (III), or a pharmaceutically acceptable salt of any of the foregoing, can exhibit up to 1,000-fold greater inhibition of PI3Kα containing one or more mutations as described herein relative to inhibition of wild type PI3Kα. In some embodiments, a compound of Formula (I), Formula (II), or Formula (III), or a pharmaceutically acceptable salt of any of the foregoing, can exhibit up to 10,000-fold greater inhibition of PI3Kα having a combination of mutations described herein relative to inhibition of wild type PI3Kα.

In some embodiments, a compound of Formula (I), Formula (II), or Formula (III), or a pharmaceutically acceptable salt of any of the foregoing, can exhibit from about 2-fold to about 10-fold greater inhibition of PI3Kα containing one or more mutations as described herein relative to inhibition of wild type PI3Kα. In some embodiments, a compound of Formula (I), Formula (II), or Formula (III), or a pharmaceutically acceptable salt of any of the foregoing, can exhibit from about 10-fold to about 100-fold greater inhibition of PI3Kα containing one or more mutations as described herein relative to inhibition of wild type PI3Kα. In some embodiments, a compound of Formula (I), Formula (II), or Formula (III), or a pharmaceutically acceptable salt of any of the foregoing, can exhibit from about 100-fold to about 1,000-fold greater inhibition of PI3Kα containing one or more mutations as described herein relative to inhibition of wild type PI3Kα. In some embodiments, a compound of Formula (I), Formula (II), or Formula (III), or a pharmaceutically acceptable salt of any of the foregoing, can exhibit from about 1000-fold to about 10,000-fold greater inhibition of PI3Kα containing one or more mutations as described herein relative to inhibition of wild type PI3Kα.

Compounds of Formula (I), Formula (II), or Formula (III), or pharmaceutically acceptable salts of any of the foregoing, are useful for treating diseases which can be treated with a PI3Kα inhibitor, such as PI3Kα-associated diseases, e.g., proliferative disorders such as cancers, including hematological cancers and solid tumors (e.g., advanced or metastatic solid tumors).

In some embodiments, the subject has been identified or diagnosed as having a cancer with a dysregulation of a PIK3CA gene, a PI3Kα protein, or expression or activity, or level of any of the same (a PI3Kα-associated cancer), for example, as determined using a regulatory agency-approved, e.g., FDA-approved, assay or kit. In some embodiments, the subject has a tumor that is positive for a dysregulation of a PIK3CA gene, a PI3Kα protein, or expression or activity, or level of any of the same (e.g., as determined using a regulatory agency-approved assay or kit). For example, the subject has a tumor that is positive for a mutation as described in Table 1. The subject can be a subject with a tumor(s) that is positive for a dysregulation of a PIK3CA gene, a PI3Kα protein, or expression or activity, or level of any of the same (e.g., identified as positive using a regulatory agency-approved, e.g., FDA-approved, assay or kit). The subject can be a subject whose tumors have a dysregulation of a PIK3CA gene, a PI3Kα protein, or expression or activity, or a level of the same (e.g., where the tumor is identified as such using a regulatory agency-approved, e.g., FDA-approved, kit or assay). In some embodiments, the subject is suspected of having a PI3Kα-associated cancer. In some embodiments, the subject has a clinical record indicating that the subject has a tumor that has a dysregulation of a PIK3CA gene, a PI3Kα protein, or expression or activity, or level of any of the same (and optionally the clinical record indicates that the subject should be treated with any of the compositions provided herein).

In certain embodiments, compounds of Formula (I), Formula (II), or Formula (III), or pharmaceutically acceptable salts of any of the foregoing, are useful for preventing diseases as defined herein such as cancer. The term “preventing” as used herein means to delay the onset, recurrence or spread, in whole or in part, of the disease as described herein, or a symptom thereof.

The term “PI3Kα-associated disease” as used herein refers to diseases associated with or having a dysregulation of a PIK3CA gene, a PI3Kα protein, or the expression or activity or level of any (e.g., one or more) of the same (e.g., any of the types of dysregulation of a PIK3CA gene, or a PI3Kα protein, or the expression or activity or level of any of the same described herein). Non-limiting examples of a PI3Kα-associated disease include, for example, proliferative disorders such as cancer (e.g., PI3Kα-associated cancer).

The term “PI3Kα-associated cancer” as used herein refers to cancers associated with or having a dysregulation of a PIK3CA gene, a PI3Kα protein, or expression or activity, or level of any of the same. Non-limiting examples of PI3Kα-associated cancer are described herein.

The phrase “dysregulation of a PIK3CA gene, a PI3Kα protein, or the expression or activity or level of any of the same” refers to a genetic mutation (e.g., a mutation in a PIK3CA gene that results in the expression of a PI3Kα that includes a deletion of at least one amino acid as compared to a wild type PI3Kα, a mutation in a PIK3CA gene that results in the expression of PI3Kα with one or more point mutations as compared to a wild type PI3Kα, a mutation in a PIK3CA gene that results in the expression of PI3Kα with at least one inserted amino acid as compared to a wild type PI3Kα, a gene duplication that results in an increased level of PI3Kα in a cell, or a mutation in a regulatory sequence (e.g., a promoter and/or enhancer) that results in an increased level of PI3Kα in a cell), an alternative spliced version of PI3Kα mRNA that results in PI3Kα having a deletion of at least one amino acid in the PI3Kα as compared to the wild type PI3Kα), or increased expression (e.g., increased levels) of a wild type PI3Kα in a mammalian cell due to aberrant cell signaling and/or dysregulated autocrine/paracrine signaling (e.g., as compared to a control non-cancerous cell). As another example, a dysregulation of a PIK3CA gene, a PI3Kα protein, or expression or activity, or level of any of the same, can be a mutation in a PIK3CA gene that encodes a PI3Kα that is constitutively active or has increased activity as compared to a protein encoded by a PIK3CA gene that does not include the mutation. Non-limiting examples of PI3Kα point mutations/substitutions/insertions/deletions are described in Table 1.

The term “wild type” describes a nucleic acid (e.g., a PIK3CA gene or a PI3Kα mRNA) or protein (e.g., a PI3Kα) sequence that is typically found in a subject that does not have a disease related to the reference nucleic acid or protein.

The term “wild type PI3Kα” or “wild-type PI3Kα” describes a normal PI3Kα nucleic acid (e.g., a PIK3CA or PI3Kα mRNA) or protein that is found in a subject that does not have a PI3Kα-associated disease, e.g., a PI3Kα-associated cancer (and optionally also does not have an increased risk of developing a PI3Kα-associated disease and/or is not suspected of having a PI3Kα-associated disease), or is found in a cell or tissue from a subject that does not have a PI3Kα-associated disease, e.g., a PI3Kα-associated cancer (and optionally also does not have an increased risk of developing a PI3Kα-associated disease and/or is not suspected of having a PI3Kα-associated disease).

Provided herein is a method of treating cancer (e.g., a PI3Kα-associated cancer) in a subject in need thereof, comprising administering to the subject a therapeutically effective amount of a compound of Formula (I), Formula (II), or Formula (III), or a pharmaceutically acceptable salt of any of the foregoing, or a pharmaceutical composition thereof. For example, provided herein are methods for treating PI3Kα-associated cancer in a subject in need thereof, comprising a) detecting a dysregulation of PIK3CA gene, a PI3Kα protein, or the expression or activity or level of any of the same in a sample from the subject; and b) administering a therapeutically effective amount of a compound of Formula (I), Formula (II), or Formula (III), or a pharmaceutically acceptable salt of any of the foregoing. In some embodiments, the dysregulation of a PIK3CA gene, a PI3Kα protein, or the expression or activity or level of any of the same includes one or more a PI3Kα protein substitutions/point mutations/insertions. Non-limiting examples of PI3Kα protein substitutions/insertions/deletions are described in Table 1.

salt of any of the foregoingsalt of any of the foregoingsalt of any of the foregoingsalt of any of the foregoingsalt of any of the foregoingsalt of any of the foregoingsalt of any of the foregoingsalt of any of the foregoingsalt of any of the foregoingsalt of any of the foregoingsalts of any of the foregoingsalts of any of the foregoingsalt of any of the foregoingsalt of any of the foregoing

In some embodiments, the compound is a compound of Formula (I), or a pharmaceutically acceptable salt thereof. In some embodiments, the compound is a compound of Formula (II), or a pharmaceutically acceptable salt thereof. In some embodiments, the compound is a compound of Formula (III), or a pharmaceutically acceptable salt thereof.

In some embodiments, the PI3Kα protein substitution/insertion/deletion is selected from the group consisting of E542A, E542G, E542K, E542Q, E542V, E545A, E545D, E545G, E545K, E545Q, M1043I, M1043L, M1043T, M1043V, H1047L, H1047Q, H1047R, H1047Y, G1049R, and combinations thereof. In some embodiments, the PI3Kα protein substitution/insertion/deletion is H1047X, where X is any amino acid other than H. In some embodiments, the PI3Kα protein substitution/insertion/deletion is E542X, where X is any amino acid other than E. In some embodiments, the PI3Kα protein substitution/insertion/deletion is E545X, where X is any amino acid other than E.

In some embodiments, the dysregulation of a PIK3CA gene, a PI3Kα protein, or expression or activity or level of any of the same, includes a splice variation in a PI3Kα mRNA which results in an expressed protein that is an alternatively spliced variant of PI3Kα having at least one residue deleted (as compared to the wild type PI3Kα protein) resulting in a constitutive activity of a PI3Kα protein domain.

In some embodiments, the dysregulation of a PIK3CA gene, a PI3Kα protein, or expression or activity or level of any of the same, includes at least one point mutation in a PIK3CA gene that results in the production of a PI3Kα protein that has one or more amino acid substitutions or insertions or deletions in a PIK3CA gene that results in the production of a PI3Kα protein that has one or more amino acids inserted or removed, as compared to the wild type PI3Kα protein. In some cases, the resulting mutant PI3Kα protein has increased activity, as compared to a wild type PI3Kα protein or a PI3Kα protein not including the same mutation. In some embodiments, the compounds described herein selectively inhibit the resulting mutant PI3Kα protein relative to a wild type PI3Kα protein or a PI3Kα protein not including the same mutation.

In some embodiments of any of the methods or uses described herein, the cancer (e.g., PI3Kα-associated cancer) is selected from a hematological cancer and a solid tumor.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), uterine cancer (including endometrial cancer), lung cancer (including small cell lung cancer (SCLC) and non-small cell lung cancer (NSCLS, including adenocarcinoma lung cancer and squamous cell lung carcinoma)), esophageal squamous cell carcinoma, ovarian cancer, colorectal cancer, esophagastric adenocarcinoma, bladder cancer, head and neck cancer (including head and neck squamous cell cancers such as oropharyngeal squamous cell carcinoma), thyroid cancer, glioma, cervical cancer, lymphangioma, meningioma, melanoma (including uveal melanoma), kidney cancer, pancreatic neuroendocine neoplasms (pNETs), stomach cancer, esophageal cancer, acute myeloid leukemia, relapsed and refractory multiple myeloma, hepatocellular carcinoma, prostate cancer, Malignant Peripheral Nerve Sheath Tumor (MPNST), glioblastoma, cholangiocarcinoma, and pancreatic cancer.

In some embodiments of any of the methods or uses described herein, the cancer (e.g., PI3Kα-associated cancer) is selected from breast cancer (including both HER2⁺ and HER2⁻ breast cancer, ER⁺ breast cancer, and triple negative breast cancer), colon cancer, rectal cancer, colorectal cancer, ovarian cancer, lymphangioma, meningioma, head and neck squamous cell cancer (including oropharyngeal squamous cell carcinoma), melanoma (including uveal melanoma), kidney cancer, pancreatic neuroendocine neoplasms (pNETs), stomach cancer, esophageal cancer, acute myeloid leukemia, relapsed and refractory multiple myeloma, pancreatic cancer, lung cancer (including adenocarcinoma lung cancer and squamous cell lung carcinoma), and endometrial cancer.

In some embodiments of any of the methods or uses described herein, the cancer (e.g., PI3Kα-associated cancer) is selected from breast cancer, SCLC, NSCLC, endometrial cancer, esophageal squamous cell carcinoma, ovarian cancer, colorectal cancer, esophagastric adenocarcinoma, bladder cancer, head and neck cancer, thyroid cancer, glioma, and cervical cancer.

In some embodiments ofany of the methods or uses described herein, the PI3Kα-associated cancer is breast cancer. In some embodiments ofany of the methods or uses described herein, the PI3Kα-associated cancer is colorectal cancer. In some embodiments ofany of the methods or uses described herein, the PI3Kα-associated cancer is endometrial cancer. In some embodiments of any of the methods or uses described herein, the PI3Kα-associated cancer is lung cancer.

In some embodiments of any of the methods or uses described herein, the PI3Kα-associated cancer is selected from the cancers described in Table 1.

TABLE 1
PI3Kα Protein Amino Acid Substitutions/Insertions/Deletions^{A}

| | Non-Limiting | |
|---|---|---|
| Amino Acid | Exemplary | |
| Position | Mutations | Non-Limiting Exemplary PI3Kα Associated Cancer(s) |
| 1043 | M1043I, | Breast Invasive Lobular Carcinoma |
| | M1043L, | Tubular Stomach Adenocarcinoma |
| | M1043T, | Uterine Endometrioid Carcinoma |
| | M1043V | Mucinous Adenocarcinoma of the Colon and Rectum |
| | | Papillary Thyroid Cancer |
| | | Esophageal Squamous Cell Carcinoma |
| | | Colon Adenocarcinoma |
| | | Breast Invasive Ductal Carcinoma |
| | | Bladder Urothelial Carcinoma |
| | | Pancreatic Adenocarcinoma |
| | | Oligodendroglioma |
| | | Uterine Serous Carcinoma/Uterine Papillary Serous |
| | | Carcinoma |
| | | Glioblastoma Multiforme |
| | | Head and Neck Squamous Cell Carcinoma |
| 1044 | N1044I, N1044K, | Uterine Endometrioid Carcinoma |
| | N1044Y | Breast Invasive Ductal Carcinoma |
| 1045 | D1045A, | Uterine Endometrioid Carcinoma |
| | D1045V | Lung Squamous Cell Carcinoma |
| 1047 | H1047L, | Esophageal Squamous Cell Carcinoma |
| | H1047Q, | Uterine Endometrioid Carcinoma |
| | H1047R, H1047Y | Hepatocellular Carcinoma |
| | | Cutaneous Melanoma |
| | | Mucinous Adenocarcinoma of the Colon and Rectum |
| | | Bladder Urothelial Carcinoma |
| | | Cervical Squamous Cell Carcinoma |
| | | Intrahepatic Cholangiocarcinoma |
| | | Uterine Mixed Endometrial Carcinoma |
| | | Breast Invasive Ductal Carcinoma |
| | | Renal Clear Cell Carcinoma |
| | | Uterine Serous Carcinoma/Uterine Papillary Serous |
| | | Carcinoma |
| | | Head and Neck Squamous Cell Carcinoma |
| | | Lung Squamous Cell Carcinoma |
| | | Breast Invasive Lobular Carcinoma |
| | | Breast Invasive Carcinoma (NOS) |
| | | Astrocytoma |
| | | Colon Adenocarcinoma |
| | | Leiomyosarcoma |
| | | Uterine Carcinosarcoma/Uterine Malignant Mixed |
| | | Mullerian Tumor |
| | | Oligodendroglioma |
| | | Serous Ovarian Cancer |
| | | Mucinous Stomach Adenocarcinoma |
| | | Rectal Adenocarcinoma |
| | | Intestinal Type Stomach Adenocarcinoma |
| | | Diffuse Type Stomach Adenocarcinoma |
| | | Prostate Adenocarcinoma |
| | | Lung Adenocarcinoma |
| | | Stomach Adenocarcinoma |
| | | Tubular Stomach Adenocarcinoma |
| | | Adrenocortical Carcinoma |
| | | Undifferentiated Pleomorphic Sarcoma/Malignant Fibrous |
| | | Histiocytoma/High-Grade Spindle Cell Sarcoma |
| | | Glioblastoma Multiforme |
| | | Oligoastrocytoma |
| 1048 | H1048R | Colon Adenocarcinoma |
| | | Renal Clear Cell Carcinoma |
| 1049 | G1049R | Intestinal Type Stomach Adenocarcinoma |
| | | Bladder Urothelial Carcinoma |
| | | Renal Clear Cell Carcinoma |
| | | Breast Invasive Ductal Carcinoma |
| | | Breast Invasive Lobular Carcinoma |
| | | Uterine Endometrioid Carcinoma |
| | | Colon Adenocarcinoma |
| 1052 | T1052K | Hepatocellular Carcinoma |
| | | Colon Adenocarcinoma |
| 1055 | M1055I | Uterine Mixed Endometrial Carcinoma |
| 1058 | I1058M | Uterine Carcinosarcoma/Uterine Malignant Mixed |
| | | Mullerian Tumor |
| 1065 | H1065L | Breast Invasive Lobular Carcinoma |
| 1066 | A1066V | Uterine Mixed Endometrial Carcinoma |
| 1068 | N1068Y, | Pleural Mesothelioma, Epithelioid Type |
| | N1068fs*5 | Dedifferentiated Liposarcoma |
| | (Frame Shift | Head and Neck Squamous Cell Carcinoma |
| | Insertion) | |

^{A}Unless noted otherwise, the mutations of Table 1 are found in cBioPortal database derived from Cerami et al. The cBio Cancer Genomics Portal: An Open Platform for Exploring Multidimensional Cancer Genomics Data. Cancer Discovery. May 2012 2; 401; and Gao et al. Integrative analysis of complex cancer genomics and clinical profiles using the cBioPortal. Sci. Signal. 6, pl1 (2013).
† Velho S, Oliveira C, Ferreira A, Ferreira A C, Suriano G, Schwartz S Jr, Duval A, Carneiro F, Machado J C, Hamelin R, Seruca R. The prevalence of PIK3CA mutations in gastric and colon cancer. Eur J Cancer. 2005 July; 41(11): 1649-54. doi: 10.1016/j.ejca.2005.04.022. PMID: 15994075.

Exemplary Sequence of Human Phosphatidylinositol 4,5-bisphosphate 3-kinase isoform alpha (UniProtKB entry P42336) (SEQ ID NO: 1)

MPPRPSSGEL WGIHLMPPRI LVECLLPNGM IVTLECLREA TLITIKHELF KEARKYPLHQ
LLQDESSYIF VSVTQEAERE EFFDETRRLC DLRLFQPFLK
VIEPVGNREE KILNREIGFA IGMPVCEFDM VKDPEVQDFR RNILNVCKEA
VDLRDLNSPH SRAMYVYPPN VESSPELPKH IYNKLDKGQI IVVIWVIVSP
NNDKQKYTLK INHDCVPEQV IAEAIRKKTR SMLLSSEQLK LCVLEYQGKY
ILKVCGCDEY FLEKYPLSQY KYIRSCIMLG RMPNLMLMAK ESLYSQLPMD
CFTMPSYSRR ISTATPYMNG ETSTKSLWVI NSALRIKILC ATYVNVNIRD
IDKIYVRTGI YHGGEPLCDN VNTQRVPCSN PRWNEWLNYD IYIPDLPRAA
RLCLSICSVK GRKGAKEEHC PLAWGNINLF DYTDTLVSGK MALNLWPVPH
GLEDLLNPIG VTGSNPNKET PCLELEFDWF SSVVKFPDMS VIEEHANWSV
SREAGFSYSH AGLSNRLARD NELRENDKEQ LKAISTRDPL SEITEQEKDF
LWSHRHYCVT IPEILPKLLL SVKWNSRDEV AQMYCLVKDW PPIKPEQAME
LLDCNYPDPM VRGFAVRCLE KYLTDDKLSQ YLIQLVQVLK YEQYLDNLLV
RFLLKKALTN QRIGHFFFWH LKSEMHNKTV SQRFGLLLES YCRACGMYLK
HLNRQVEAME KLINLTDILK QEKKDETQKV QMKFLVEQMR RPDFMDALQG
FLSPLNPAHQ LGNLRLEECR IMSSAKRPLW LNWENPDIMS ELLFQNNEII
FKNGDDLRQD MLTLQIIRIM ENIWQNQGLD LRMLPYGCLS IGDCVGLIEV
VRNSHTIMQI QCKGGLKGAL QFNSHTLHQW LKDKNKGEIY DAAIDLFTRS
CAGYCVATFI LGIGDRHNSN IMVKDDGQLF HIDFGHFLDH KKKKFGYKRE
RVPFVLTQDF LIVISKGAQE CTKTREFERF QEMCYKAYLA IRQHANLFIN
LFSMMLGSGM PELQSFDDIA YIRKTLALDK TEQEALEYFM KQMNDAHHGG
WTTKMDWIFH TIKQHALN

Also provided is a method for inhibiting PI3Kα activity in a cell, comprising contacting the cell with a compound of Formula (I), Formula (II), or Formula (III), or a pharmaceutically acceptable salt of any of the foregoing. In some embodiments, the contacting is in vitro. In some embodiments, the contacting is in vivo. In some embodiments, the contacting is in vivo, wherein the method comprises administering an effective amount of a compound of Formula (I), Formula (II), or Formula (III), or a pharmaceutically acceptable salt of any of the foregoing, to a subject having a cell having aberrant PI3Kα activity. In some embodiments, the cell is a cancer cell. In some embodiments, the cancer cell is any cancer as described herein. In some embodiments, the cancer cell is a PI3Kα-associated cancer cell. As used herein, the term “contacting” refers to the bringing together of indicated moieties in an in vitro system or an in vivo system. For example, “contacting” a PI3Kα protein with a compound provided herein includes the administration of a compound provided herein to an individual or subject, such as a human, having a PI3Kα protein, as well as, for example, introducing a compound provided herein into a sample containing a cellular or purified preparation containing the PI3Kα protein.

Also provided herein is a method of inhibiting cell proliferation, in vitro or in vivo, comprising contacting a cell with an effective amount of a compound of Formula (I), Formula (II), or Formula (III), or a pharmaceutically acceptable salt of any of the foregoing, or a pharmaceutical composition thereof as defined herein.

Further provided herein is a method of increase cell death, in vitro or in vivo, comprising contacting a cell with an effective amount of a compound of Formula (I), Formula (II), or Formula (III), or a pharmaceutically acceptable salt of any of the foregoing, or a pharmaceutical composition thereof as defined herein. Also provided herein is a method of increasing tumor cell death in a subject, comprising administering to the subject an effective compound of Formula (I), Formula (II), or Formula (III), or a pharmaceutically acceptable salt of any of the foregoing, in an amount effective to increase tumor cell death.

In some embodiments, the PI3Kα is human PI3Kα. In some embodiments, the PI3Kα has one or more point mutations in the PIK3CA gene. In some embodiments, the point mutations include a substitution at amino acid position 1047 of a human PI3Kα protein. In some embodiments, the substitution is H1047R.

When employed as pharmaceuticals, the compounds of Formula (I), Formula (II), or Formula (III), including pharmaceutically acceptable salts of any of the foregoing, can be administered in the form of pharmaceutical compositions as described hereinsalt of any of the foregoingsalt of any of the foregoingsalt of any of the foregoingsalt of any of the foregoingsalt of any of the foregoingsalts of any of the foregoing.

### EMBODIMENTS

1. A compound of Formula (I), Formula (II), or Formula (III):

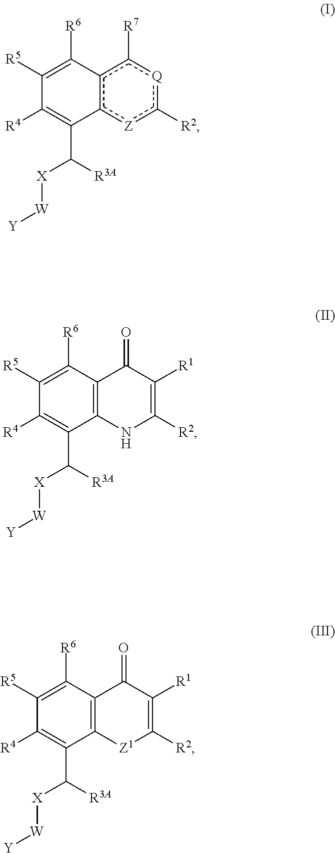

or a pharmaceutically acceptable salt of any of the foregoing, wherein:
Q is NR^{1A} or CR¹;
Z is NR^{Z1} or CR^{Z2}, wherein one or both of Q and Z is N;
Z¹ is S, —S(O₂)—, or O;
each [symbol] represents a single bond or a double bond;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1A} is absent, hydrogen, C1-C6 alkyl, C1-C6 haloalkyl, C1-C6 thioalkyl, C1-C6 haloalkyl, C1-C6 alkoxy, or C1-C6 alkoxyalkyl;
R^{Z1} is absent, hydrogen, cyano, C1-C6 alkyl, or C1-C6 alkyl;
R^{Z2} is hydrogen, C1-C6 alkyl, or C1-C6 haloalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B},

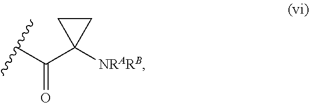

(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G}
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NHR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, C1-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═NR^{7A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or

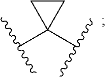 [SMILES: *C1(*)CC1]

W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

2. The compound of Embodiment 1, wherein the compound is a compound of Formula (II).

3. The compound of Embodiment 1, wherein the compound is a compound of Formula (III).

4. The compound of Embodiment 1 or 3, wherein Z¹ is S.

5. The compound of Embodiment 1 or 3, wherein Z¹ is O.

6. The compound of Embodiment 1 or 3, wherein Z¹ is —S(O₂)—.

7. The compound of Embodiment 1, wherein the compound is a compound of Formula (I).

8. The compound of Embodiment 1 or 7, wherein Q is CR¹.

9. The compound of any one of Embodiments 1-8 wherein R¹ is hydrogen.

10. The compound of any one of Embodiments 1-8, wherein R¹ cyano.

11. The compound of any one of Embodiments 1-8, wherein R¹ is C3-C6 cycloalkyl.

12. The compound of any one of Embodiments 1-8, wherein R¹ is C1-C6 alkyl optionally substituted with phenyl optionally substituted with halogen.

13. The compound of any one of Embodiments 1-8, wherein R¹ is C1-C6 alkyl substituted with phenyl optionally substituted with halogen.

14. The compound of any one of Embodiments 1-8, wherein R¹ is C1-C6 alkyl substituted with phenyl substituted with halogen.

15. The compound of any one of Embodiments 1-8, wherein R¹ is C1-C6 alkyl substituted with phenyl.

16. The compound of any one of Embodiments 1-8, wherein R¹ is C1-C6 alkyl.

17. The compound of any one of Embodiments 1-8, wherein R¹ is C1-C6 thioalkyl.

18. The compound of any one of Embodiments 1-8, wherein R¹ is C1-C6 haloalkyl.

19. The compound of any one of Embodiments 1-8, wherein R¹ is C1-C6 alkoxy.

20. The compound of any one of Embodiments 1-8, wherein R¹ is C1-C6 alkoxyalkyl.

21. The compound of any one of Embodiments 1-7, wherein Q is NR^{1A}.

22. The compound of Embodiment 21, wherein R^{1A} is absent.

23. The compound of Embodiment 21, wherein R^{1A} is hydrogen.

24. The compound of Embodiment 21, wherein R^{1A} is C1-C6 alkyl.

25. The compound of Embodiment 24, wherein R^{1A} is C1-C3 alkyl.

26. The compound of Embodiment 25, wherein R^{1A} is methyl.

27. The compound of Embodiment 21, wherein R^{1A} is C1-C6 haloalkyl.

28. The compound of Embodiment 27, wherein R^{1A} is trifluoromethyl.

29. The compound of Embodiment 21, wherein R^{1A} is C1-C6 thioalkyl.

30. The compound of Embodiment 21, wherein R^{1A} is C1-C6 alkoxy.

31. The compound of Embodiment 21, wherein R^{1A} is C1-C6 alkoxyalkyl.

32. The compound of any one of Embodiments 1 or 7-31, wherein Z is NR^{Z1}.

33. The compound of Embodiment 32, wherein R^{Z1} is absent.

34. The compound of Embodiment 32, wherein R^{Z1} is hydrogen.

35. The compound of Embodiment 32, wherein R^{Z1} is C1-C6 alkyl.

36. The compound of Embodiment 32, wherein R^{Z1} is C1-C3 alkyl.

37. The compound of Embodiment 36, wherein R^{Z1} is methyl.

38. The compound of Embodiment 32, wherein R^{Z1} is C1-C6 haloalkyl.

39. The compound of Embodiment 38, wherein R^{Z1} is trifluoromethyl.

40. The compound of Embodiment 32, wherein R^{Z1} is cyano.

41. The compound of any one of Embodiments 1 or 7-31, wherein Z is CR^{Z2}.

42. The compound of Embodiment 41, wherein R^{Z2} is hydrogen.

43. The compound of Embodiment 41, wherein R^{Z2} is C1-C6 alkyl.

44. The compound of Embodiment 43, wherein R^{Z2} is C1-C3 alkyl.

45. The compound of Embodiment 44, wherein R^{Z2} is methyl.

46. The compound of Embodiment 41, wherein R^{Z2} is C1-C6 haloalkyl.

47. The compound of Embodiment 46, wherein R^{Z2} is trifluoromethyl.

48. The compound of any one of Embodiments 1-47, wherein R² is phenyl optionally substituted with 1-3 independently selected R^{2A}.

49. The compound of any one of Embodiments 1-47, wherein R² is 5-10 membered heteroaryl optionally substituted with 1-3 independently selected R^{2A}.

50. The compound of any one of Embodiments 1-47, wherein R² is 4-10 membered heterocyclyl optionally substituted with 1-3 independently selected R^{2A}.

51. The compound of any one of Embodiments 1-47, wherein R² is 4-10 membered cycloalkyl optionally substituted with 1-3 independently selected R^{2A}.

52. The compound of any one of Embodiments 1-51, wherein 1, 2, or 3 of R^{2A} are independently halogen.

53. The compound of any one of Embodiments 1-52, wherein 1, 2, or 3 of R^{2A} are independently cyano.

54. The compound of any one of Embodiments 1-53, wherein 1, 2, or 3 of R^{2A} are independently hydroxyl.

55. The compound of any one of Embodiments 1-54, wherein 1, 2, or 3 of R^{2A} are independently —NR^{A}R^{B}.

56. The compound of any one of Embodiments 1-55, wherein 1, 2, or 3 of R^{2A} are independently —C(═O)NR^{A}R^{B}.

57. The compound of any one of Embodiments 1-56, wherein 1, 2, or 3 of R^{2A} are independently

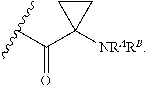

58. The compound of any one of Embodiments 1-57, wherein 1, 2, or 3 of R^{2A} are independently —NHC(═O)R^{C}.

59. The compound of any one of Embodiments 1-58, wherein 1, 2, or 3 of R^{2A} are independently —C(═O)NR^{D}R^{E}.

60. The compound of any one of Embodiments 1-59, wherein 1, 2, or 3 of R^{2A} are independently —C(═O)OR^{F}.

61. The compound of any one of Embodiments 1-60, wherein 1, 2, or 3 of R^{2A} are independently —SO₂R^{F}.

62. The compound of any one of Embodiments 1-61, wherein 1, 2, or 3 of R^{2A} are independently —NHSO₂R^{F}.

63. The compound of any one of Embodiments 1-62, wherein 1, 2, or 3 of R^{2A} are independently —SO₂NR^{F}R^{G}.

64. The compound of any one of Embodiments 1-63, wherein 1, 2, or 3 of R^{2A} are independently —NHC(═O)C1-C6 alkyl optionally substituted with NR^{A}R^{B}.

65. The compound of any one of Embodiments 1-64, wherein 1, 2, or 3 of R^{2A} are independently —NHC(═O)C1-C6 alkyl substituted with NR^{A}R^{B}.

66. The compound of any one of Embodiments 1-65, wherein 1, 2, or 3 of R^{2A} are independently C1-C6 haloalkyl.

67. The compound of any one of Embodiments 1-66, wherein 1, 2, or 3 of R^{2A} are independently C1-C6 hydroxyalkyl.

68. The compound of any one of Embodiments 1-67, wherein 1, 2, or 3 of R^{2A} are independently 5-10 membered heteroaryl optionally substituted with 1-3 substituents independently selected from C1-C6 alkyl and —NR^{A}R^{B}.

69. The compound of any one of Embodiments 1-68, wherein 1, 2, or 3 of R^{2A} are independently 5-10 membered heteroaryl substituted with 1-3 substituents independently selected from C1-C6 alkyl and —NR^{A}R^{B}.

70. The compound of any one of Embodiments 1-69, wherein 1, 2, or 3 of R^{2A} are independently 5-10 membered heteroaryl.

71. The compound of any one of Embodiments 1-70, wherein 1, 2, or 3 of R^{2A} are independently 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

72. The compound of any one of Embodiments 1-71, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B}.

73. The compound of any one of Embodiments 1-72, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G} C1-C6 alkyl substituted with C1-C6 alkoxy, —C(═O)NR^{A}R^{B}, or —NHC(═O)C1-C6 alkyl substituted with —NR^{A}R^{B}.

74. The compound of any one of Embodiments 1-73, wherein 1, 2, or 3 of R^{2A} are independently 4-10 membered heterocyclyl substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl, —C(═O)NR^{A}R^{B}, or —NHC(═O)C1-C6 alkyl.

75. The compound of any one of Embodiments 1-70, wherein 1, 2, or 3 of R^{2A} are independently 4-10 membered heterocyclyl.

76. The compound of any one of Embodiments 1-75, wherein 1, 2, or 3 of R^{2A} are independently C1-C6 alkyl optionally substituted with 1-3 substituents independently selected from hydroxyl, oxo, —NR^{A}R^{B}, —C(═O)NR^{A}R^{B}, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

77. The compound of any one of Embodiments 1-76, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl.

78. The compound of any one of Embodiments 1-77, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, C1-C6 alkoxy, and 4-10 membered heterocyclyl substituted with hydroxyl, C1-C6 alkyl, aralkyl, heteroaralkyl, —C(═O)NR^{A}R^{B}, or —C(═O)C3-C6 cycloalkyl.

79. The compound of any one of Embodiments 1-77, wherein 1, 2, or 3 of R^{2A} are independently C1-C6 alkyl substituted with 1-3 substituents independently selected from hydroxyl, oxo, —NR^{A}R^{B}, —C(═O)NR^{A}R^{B}, and 4-10 membered heterocyclyl.

80. The compound of any one of Embodiments 1-77, wherein 1, 2, or 3 of R^{2A} are independently C1-C6 alkyl.

81. The compound of any one of Embodiments 1-80, wherein 1, 2, or 3 of R^{2A} are independently C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl.

82. The compound of any one of Embodiments 1-81, wherein 1, 2, or 3 of R^{2A} are independently C1-C6 alkoxy substituted with —NR^{A}R^{B} or 4-10 membered heterocyclyl optionally substituted with C1-C6 alkyl, aralkyl, heteroaralkyl, or —C(═O)C3-C6 cycloalkyl.

83. The compound of any one of Embodiments 1-82, wherein 1, 2, or 3 of R^{2A} are independently C1-C6 alkoxy substituted with —NR^{A}R^{B} or 4-10 membered heterocyclyl substituted with C1-C6 alkyl, aralkyl, heteroaralkyl, or —C(═O)C3-C6 cycloalkyl.

84. The compound of any one of Embodiments 1-82, wherein 1, 2, or 3 of R^{2A} are independently C1-C6 alkoxy substituted with —NR^{A}R^{B} or 4-10 membered heterocyclyl.

85. The compound of any one of Embodiments 1-81, wherein 1, 2, or 3 of R^{2A} are independently C1-C6 alkoxy.

86. The compound of any one of Embodiments 1-85, wherein 1, 2, or 3 of R^{2A} are independently C3-C6 cycloalkyl optionally substituted with 4-10 membered heterocyclyl optionally substituted with C1-C6 alkyl.

87. The compound of any one of Embodiments 1-86, wherein 1, 2, or 3 of R^{2A} are independently C3-C6 cycloalkyl substituted with 4-10 membered heterocyclyl optionally substituted with C1-C6 alkyl.

88. The compound of any one of Embodiments 1-87, wherein 1, 2, or 3 of R^{2A} are independently C3-C6 cycloalkyl substituted with 4-10 membered heterocyclyl substituted with C1-C6 alkyl.

89. The compound of any one of Embodiments 1-87, wherein 1, 2, or 3 of R^{2A} are independently C3-C6 cycloalkyl substituted with 4-10 membered heterocyclyl.

90. The compound of any one of Embodiments 1-86, wherein 1, 2, or 3 of R^{2A} are independently C3-C6 cycloalkyl.

91. The compound of any one of Embodiments 1-47, wherein R² is C1-C6 alkoxy optionally substituted with —C(═O)NR^{A}R^{C}.

92. The compound of any one of Embodiments 1-47 or 91, wherein R² is C1-C6 alkoxy substituted with —C(═O)NR^{A}R^{C}.

93. The compound of any one of Embodiments 1-47 or 91, wherein R² is C1-C6 alkoxy.

94. The compound of any one of Embodiments 1-47, R² is C1-C6 alkoxyalkyl optionally substituted with —C(═O)NR^{A}R^{C}.

95. The compound of any one of Embodiments 1-47, R² is C1-C6 alkoxyalkyl

96. The compound of any one of Embodiments 1-95, wherein X is a bond.

97. The compound of any one of Embodiments 1-95, wherein X is CH₂.

98. The compound of any one of Embodiments 1-95, wherein X is CH(CH₃).

99. The compound of any one of Embodiments 1-95, wherein X is C(CH₃)₂.

100. The compound of any one of Embodiments 1-95, wherein X is

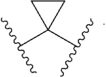 [SMILES: *C1(*)CC1]

101. The compound of any one of Embodiments 1-72, wherein W is O.

102. The compound of any one of Embodiments 1-72, wherein R^{3A} is hydrogen.

103. The compound of any one of Embodiments 1-72, wherein R^{3A} is C1-C6 alkyl.

104. The compound of any one of Embodiments 1-73, wherein R^{3A} is methyl.

105. The compound of any one of Embodiments 1-72, wherein R^{3A} is C1-C6 alkoxy.

106. The compound of any one of Embodiments 1-72, R^{3A} is C1-C6 haloalkyl.

107. The compound of any one of Embodiments 1-72, wherein W is NR^{3B}.

108. The compound of Embodiment 107, wherein one of R^{3A} and R^{3B} is hydrogen and the other of R^{3A} and R^{3B} is C1-C6 alkyl.

109. The compound of Embodiment 107-108, wherein one of R^{3A} and R^{3B} is hydrogen and the other of R^{3A} and R^{3B} is methyl.

110. The compound of Embodiment 107, wherein each of R^{3A} and R^{3B} is hydrogen.

111. The compound of Embodiment 107, wherein each of R^{3A} and R^{3B} is an independently selected C1-C6 alkyl.

112. The compound of Embodiment 107 or 111, wherein each of R^{3A} and RIB is methyl.

113. The compound of Embodiment 107, wherein one of R^{3A} and R^{3B} is hydrogen and the other of R^{3A} and R^{3B} is C1-C6 alkoxy.

114. The compound of Embodiment 107, wherein one of R^{3A} and R^{3B} is C1-C6 alkyl and the other of R^{3A} and R^{3B} is C1-C6 alkoxy.

115. The compound of Embodiment 107, wherein each of R^{3A} and R^{3B} is C1-C6 alkoxy.

116. The compound of Embodiment 107, wherein one of R^{3A} and R^{3B} is hydrogen and the other of R^{3A} and R^{3B} is C1-C6 haloalkyl.

117. The compound of Embodiment 107, wherein one of R^{3A} and R^{3B} is C1-C6 alkyl and the other of R^{3A} and R^{3B} is C1-C6 haloalkyl.

118. The compound of Embodiment 107, wherein each of R^{3A} and R^{3B} is C1-C6 haloalkyl.

119. The compound of Embodiment 107, wherein R^{3A} and R^{3B}, together with the carbon and nitrogen atoms, respectively, to which they are attached together form a 4-8 membered heterocyclyl group.

120. The compound of any one of Embodiments 1-119, wherein Y is phenyl optionally substituted with 1-3 independently selected R^{Y}.

121. The compound of any one of Embodiments 1-119, wherein Y is naphthyl optionally substituted with 1-3 independently selected R^{Y}.

122. The compound of any one of Embodiments 1-119, wherein Y is 5-10 membered heteroaryl optionally substituted with 1-3 independently selected R^{Y}.

123. The compound of any one of Embodiments 1-122, wherein 1, 2, or 3 of R^{Y} is independently halogen.

124. The compound of any one of Embodiments 1-123, wherein 1, 2, or 3 of R^{Y} is hydroxyl.

125. The compound of any one of Embodiments 1-124, wherein 1, 2, or 3 of R^{Y} is cyano.

126. The compound of any one of Embodiments 1-125, wherein 1, 2, or 3 of R^{Y} is independently C1-C6 haloalkyl.

127. The compound of any one of Embodiments 1-126, wherein 1, 2, or 3 of R^{Y} is independently C1-C6 alkoxy.

128. The compound of any one of Embodiments 1-127, wherein 1, 2, or 3 of R^{Y} is independently C1-C6 haloalkoxy.

129. The compound of any one of Embodiments 1-128, wherein 1, 2, or 3 of R^{Y} is independently C1-C6 hydroxyalkyl.

130. The compound of any one of Embodiments 1-129, wherein 1, 2, or 3 of R^{Y} is independently —NHC(═O)R^{C}.

131. The compound of any one of Embodiments 1-130, wherein 1, 2, or 3 of R^{Y} is independently —C(═O)NHR^{Y1}.

132. The compound of any one of Embodiments 1-131, wherein 1, 2, or 3 of R^{Y} is independently —CO₂R^{A}.

133. The compound of any one of Embodiments 1-132, wherein 1, 2, or 3 of R^{Y} is independently —SO₂NR^{F}R^{G}.

134. The compound of any one of Embodiments 1-133, wherein 1, 2, or 3 of R^{Y} is independently —NHSO₂R^{F}.

135. The compound of any one of Embodiments 1-134, wherein 1, 2, or 3 of R^{Y} is independently —S(═O)(═NR^{F})R^{G}.

136. The compound of any one of Embodiments 1-135, wherein 1, 2, or 3 of R^{Y} is independently —SO₂(C1-C6 alkyl).

137. The compound of any one of Embodiments 1-136, wherein 1, 2, or 3 of R^{Y} is independently —C(═O)NR^{A}R^{B}.

138. The compound of any one of Embodiments 1-137, wherein 1, 2, or 3 of R^{Y} is independently 4-6 membered heteroaryl.

139. The compound of any one of Embodiments 1-138, wherein 1, 2, or 3 of R^{Y} is independently heteroaralkyl.

140. The compound of any one of Embodiments 1-139, wherein 1, 2, or 3 of R^{Y} is independently 4-6 membered heterocyclyl.

141. The compound of any one of Embodiments 1-140, wherein 1, 2, or 3 of R^{Y} is independently C1-C6 alkyl optionally substituted with —CO₂R^{A} or 5-6 membered heteroaryl optionally substituted with R^{Y1}.

142. The compound of any one of Embodiments 1-141, wherein 1, 2, or 3 of R^{Y} is independently C1-C6 alkyl substituted with —CO₂R^{A} or 5-6 membered heteroaryl optionally substituted with R^{Y1}.

143. The compound of any one of Embodiments 1-142, wherein 1, 2, or 3 of R^{Y} is independently C1-C6 alkyl substituted with —CO₂R^{A} or 5-6 membered heteroaryl substituted with R^{Y1}.

144. The compound of any one of Embodiments 1-143, wherein 1, 2, or 3 of R^{Y} is independently C1-C6 alkyl substituted with —CO₂R^{A} or 5-6 membered heteroaryl.

145. The compound of any one of Embodiments 1-144, wherein 1, 2, or 3 of R^{Y} is independently C1-C6 alkyl.

146. The compound of any one of Embodiments 1-122 or 141-143, wherein R^{Y1} is —SO₂(C1-C6 alkyl).

147. The compound of any one of Embodiments 1-122 or 141-143, wherein R^{Y1} is C1-C6 alkyl optionally substituted with oxo.

148. The compound of any one of Embodiments 1-145, wherein 1, 2, or 3 of R^{Y1} is hydroxyl.

149. The compound of any one of Embodiments 1-148, wherein R⁴ is hydrogen.

150. The compound of any one of Embodiments 1-148, wherein R⁴ is C1-C6 alkyl.

151. The compound of any one of Embodiments 1-148, wherein R⁴ is acrylamido.

152. The compound of any one of Embodiments 1-151, wherein R⁵ is hydrogen.

153. The compound of any one of Embodiments 1-151, wherein R⁵ is C1-C6 alkyl.

154. The compound of any one of Embodiments 1-151, wherein R⁵ is cyano.

155. The compound of any one of Embodiments 1-151, wherein R⁵ is —NR^{5A}R^{5B}.

156. The compound of any one of Embodiments 1-151, wherein R⁵ is —C(═O)NR^{5A}R^{5B}.

157. The compound of any one of Embodiments 1-151, wherein R⁵ is —NR^{5A}C(═O)R^{5B}.

158. The compound of any one of Embodiments 1-151 or 145-157, wherein one of R^{5A} and R^{5B} is hydrogen and the other of R^{5A} and R^{5B} is C1-C6 alkyl, C2-C6 alkenyl, or C1-C6 hydroxyalkyl.

159. The compound of any one of Embodiments 1-151 or 155-157, wherein one of R^{5A} and R^{5B} is C1-C6 alkyl and the other of R^{5A} and R^{5B} is C1-C6 alkyl, C2-C6 alkenyl, or C1-C6 hydroxyalkyl.

160. The compound of any one of Embodiments 1-151 or 155-157, wherein each of R^{5A} and R^{5B} is hydrogen.

161. The compound of any one of Embodiments 1-151 or 155-157, wherein each of R^{5A} and R^{5B} is an independently selected C1-C6 alkyl.

162. The compound of any one of Embodiments 1-161, wherein R⁶ is hydrogen.

163. The compound of any one of Embodiments 1-161, wherein R⁶ is halogen.

164. The compound of any one of Embodiments 1-161, wherein R⁶ is C1-C6 alkyl.

165. The compound of any one of Embodiments 1-164, wherein R⁷ is hydrogen.

166. The compound of any one of Embodiments 1-164, wherein R⁷ is halogen.

167. The compound of any one of Embodiments 1-164, wherein R⁷ is hydroxyl.

168. The compound of any one of Embodiments 1-164, wherein R⁷ is cyano.

169. The compound of any one of Embodiment 1-164, wherein R⁷ is C1-C6 alkyl.

170. The compound of any one of Embodiment 1-164, wherein R⁷ is C1-C6 alkoxyalkyl.

171. The compound of any one of Embodiment 1-164, wherein R⁷ C1-C6 hydroxyalkyl.

172. The compound of any one of Embodiment 1-164, wherein R⁷ is C1-C6 cyanoalkyl.

173. The compound of any one of Embodiment 1-164, wherein R⁷ is —C(═O)C1-C6 alkyl.

174. The compound of any one of Embodiment 1-164, wherein R⁷ is —NH(CN).

175. The compound of any one of Embodiment 1-164, wherein R⁷ is 5-6 membered heteroaryl.

176. The compound of any one of Embodiments 1-164, wherein R⁷ is —NR^{7A}R^{7B}.

177. The compound of any one of Embodiments 1-164, wherein R⁷ is —NR^{7A}C(═O)R^{7B}.

178. The compound of any one of Embodiment 1-164, wherein R⁷ is —C(═NR^{7A})NR^{7A}R^{7B}.

179. The compound of any one of Embodiment 1-164, wherein R⁷ is 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}.

180. The compound of any one of Embodiment 1-164, wherein each -̅-̅-̅ adjacent to CR⁷ is a single bond, R⁷ is oxo.

181. The compound of any one of Embodiments 1-164, wherein R⁷ is —C(═O)NR^{7A}R^{7B}.

182. The compound of any one of Embodiments 1-164 or 176-179, wherein one of R^{7A} and R^{7B} is hydrogen and the other of R^{1A} and R^{5B} is C2-C6 alkenyl, C2-C6 haloalkenyl, C1-C6 alkyl optionally substituted with oxo, or C1-C6 haloalkyl optionally substituted with oxo.

183. The compound of any one of Embodiments 1-164 or 176-179, wherein one of R^{7A} and R^{7B} is C1-C6 alkyl and the other of R^{7A} and R^{7B} is C2-C6 alkenyl, C2-C6 haloalkenyl, C1-C6 alkyl optionally substituted with oxo, or C1-C6 haloalkyl optionally substituted with oxo.

184. The compound of any one of Embodiments 1-164 or 176-179, wherein each of R^{7A} and R^{7B} is hydrogen.

185. The compound of any one of Embodiments 1-164 or 176-179, wherein each of R^{7A} and R^{7B} is an independently selected C1-C6 alkyl.

186. The compound of any one of Embodiments 1-185, wherein each of R^{A} and R^{B} are independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy.

187. The compound of any one of Embodiments 1-186, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl optionally substituted with hydroxyl or C1-C6 alkoxy.

188. The compound of any one of Embodiments 1-187, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl substituted with hydroxyl or C1-C6 alkoxy.

189. The compound of any one of Embodiments 1-188, wherein one of R^{A} and R^{B} is hydrogen and the other of R^{A} and R^{B} is hydroxyl, C1-C6 alkoxy, C3-C6 cycloalkyl, C2-C6 alkenyl, or C1-C6 alkyl.

190. The compound of any one of Embodiments 1-185, wherein R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl.

191. The compound of any one of Embodiments 1-185 or 190, wherein R^{A} and R^{B} together with the nitrogen atom to which they are attached form a 4-10 membered heterocyclyl substituted with 1-2 substituents independently selected from halogen, C1-C6 alkyl, and —C(═O)C1-C6 alkyl.

192. The compound of any one of Embodiments 1-185 or 190, wherein R^{A} and R^{B} together with the nitrogen atom to which they are attached form a 4-10 membered heterocyclyl.

193. The compound of any one of Embodiments 1-176, wherein each R^{C} is independently C3-C6 cycloalkyl, —C(═O)NR^{Y1}, or a C1-C6 alkyl substituted with —NR^{A}R^{B} or with 4-10 membered heterocyclyl optionally substituted with C1-C6 alkyl or with C1-C6 hydroxylalkyl.

194. The compound of any one of Embodiments 1-193, wherein each R^{C} is independently C3-C6 cycloalkyl, —C(═O)NHR^{Y1}, or a C1-C6 alkyl substituted with —NR^{A}R^{B} or with 4-10 membered heterocyclyl substituted with C1-C6 alkyl or with C1-C6 hydroxylalkyl.

195. The compound of any one of Embodiments 1-193, wherein each R^{C} is independently C3-C6 cycloalkyl, —C(═O)NHR^{Y1}, or a C1-C6 alkyl substituted with —NR^{A}R^{B} or with 4-10 membered heterocyclyl.

196. The compound of any one of Embodiments 1-192, wherein each R^{C} is independently C3-C6 cycloalkyl, —C(═O)NR^{Y1}, or a C1-C6 alkyl.

197. The compound of any one of Embodiments 1-196, wherein one of R^{D} and R^{E} is hydrogen and the other of R^{D} and R^{E} is hydroxyl, C1-C6 alkyl, or C1-C6 alkoxy.

198. The compound of any one of Embodiments 1-196, wherein each of R^{D} and R^{E} is hydrogen.

199. The compound of any one of Embodiments 1-196, wherein each of R^{D} and R^{E} is an independently selected C1-C6 alkyl.

200. The compound of any one of Embodiments 1-196, wherein one of R^{F} and R^{G} is hydrogen and the other of R^{F} and R^{G} is phenyl or C1-C6 alkyl optionally substituted with oxo or —NR^{A}R^{B}.

201. The compound of any one of Embodiments 1-196, wherein one of R^{F} and R^{G} is hydrogen and the other of R^{F} and R^{G} is phenyl or C1-C6 alkyl substituted with oxo or —NR^{A}R^{B} 202. The compound of any one of Embodiments 1-196, wherein one of R^{F} and R^{G} is hydrogen and the other of R^{F} and R^{G} is phenyl or C1-C6 alkyl.

203. The compound of any one of Embodiments 1-196, wherein each of R^{F} and R^{G} is hydrogen.

204. The compound of any one of Embodiments 1-196, wherein each of R^{F} and R^{G} is an independently selected C1-C6 alkyl.

205. A compound selected from the group consisting of the compounds in Table A, Table B, or Table C, or a pharmaceutically acceptable salt of any of the foregoing.

206. A pharmaceutical composition comprising a compound of any one of Embodiments 1-205, or a pharmaceutically acceptable salt of any of the foregoing, and one or more pharmaceutically acceptable excipients.

207. A method for treating cancer in a subject in need thereof, comprising administering to the subject a therapeutically effective amount of a compound of any one of Embodiments 1-205, or a pharmaceutically acceptable salt of any of the foregoing, or a pharmaceutical composition of Embodiment 206.

208. A method for treating cancer in a subject in need thereof, the method comprising (a) determining that the cancer is associated with a dysregulation of a PIK3CA gene, a PI3Kα protein, or expression or activity or level of any of the same, and (b) administering to the subject a therapeutically effective amount of a compound of any one of Embodiments 1-205, or a pharmaceutically acceptable salt of any of the foregoing, or a pharmaceutical composition of Embodiment 206.

209. A method of treating a PI3Kα-associated cancer in a subject, comprising administering to a subject identified or diagnosed as having a PI3Kα-associated cancer a therapeutically effective amount of a compound of any one of Embodiments 1-205 or a pharmaceutically acceptable salt of any of the foregoing, or a pharmaceutical composition of Embodiment 206.

210. A method for inhibiting mutant PI3Kα activity in a mammalian cell, the method comprising contacting the mammalian cell with an effective amount of a compound of any one of Embodiments 1-205, or a pharmaceutically acceptable salt of any of the foregoing.

salt of any of the foregoingsalt of any of the foregoingsalt of any of the foregoingsalt of any of the foregoingsalt of any of the foregoingsalt of any of the foregoingsalt of any of the foregoing

### EXAMPLES

### Preparation of Compounds

The starting materials used for the syntheses are either synthesized or obtained from commercial sources, such as, but not limited to, Sigma-Aldrich, Fluka, Acros Organics, Alfa Aesar, Enamine, Strem, VWR Scientific, and the like. Nuclear Magnetic Resonance (NMR) analysis was conducted using a Bruker AVANCE III HD (300 or 400) MHz spectrometer or Bruker AVANCE NEO 400 MHz spectrometer with an appropriate deuterated solvent. LCMS spectra were obtained on a Shimadzu LCMS-2020 with electrospray ionization in positive ion detection mode with 20ADXR pump, SIL-20ACXR autosampler, CTO-20AC column oven, M20A PDA Detector and LCMS 2020 MS detector.

The general methods for the preparation of the compounds of Formula (I), Formula (II), or Formula (III), have been described in an illustrative manner and are intended to be descriptive, rather than limiting. Thus, it will be appreciated that conditions such as choice of solvent, temperature of reaction, volumes, reaction time may vary while still producing the desired compounds. In addition, it will be appreciated that many of the reagents provided in the following examples may be substituted with other suitable reagents. See, e.g., Smith & March, Advanced Organic Chemistry, 7th Ed. (2013). Such changes and modifications, including without limitation, those relating to the chemical structures, substituents, derivatives, intermediates, syntheses, formulations and/or methods of use provided herein, may be made without departing from the spirit and scope thereof.

### Example 1: Synthesis of Compound 9: 2-((1-(4-hydroxy-6-methyl-2-(pyridin-4-yl)quinazolin-8-yl)ethyl)amino)benzoic acid

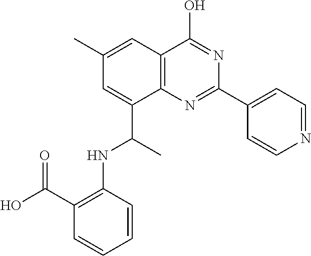 [SMILES: Cc1cc(C(C)Nc2ccccc2C(=O)O)c2nc(-c3ccncc3)nc(O)c2c1]

### Step 1—Synthesis of 8-bromo-6-methyl-2-(pyridin-4-yl)quinazolin-4-ol

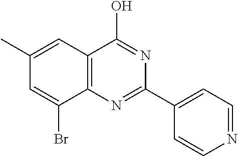 [SMILES: Cc1cc(Br)c2nc(-c3ccncc3)nc(O)c2c1]

A mixture of 2-amino-3-bromo-5-methyl-benzamide (1.8 g, 8.7 mmol) (prepared according to the procedure in WO20075668), CuO (1.32 g, 16.6 mmol) and pyridine-4-carbaldehyde (1.12 g, 10.5 mmol) in dimethylacetamide (DMA, 50 mL) was stirred at 135° C. for 16 hours under O₂ atmosphere. After cooling to room temperature, the reaction was poured into water (100 mL). The suspension was filtered and the filter cake was dried in vacuo to give 8-bromo-6-methyl-2-(pyridin-4-yl)quinazolin-4-ol (360 mg, crude) as a white solid that required no further purification. MS: m/z 318.1 (M+H⁺).

### Step 2—Synthesis of 1-(4-hydroxy-6-methyl-2-(pyridin-4-yl)quinazolin-8-yl)ethan-1-one

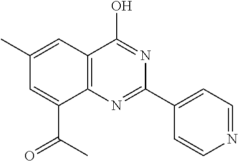 [SMILES: CC(=O)c1cc(C)cc2c(O)nc(-c3ccncc3)nc12]

A mixture of 8-bromo-6-methyl-2-(4-pyridyl)quinazolin-4-ol (190 mg, 601 μmol), tributyl(1-ethoxyvinyl)stannane (543 mg, 1.5 mmol) and Pd(dppf)Cl₂ (44 mg, 60 umol) in dioxane (10 mL) was stirred at 90° C. for 16 hours under N₂ atmosphere. After cooling to room temperature, the reaction mixture was added 1M HCl in water (1 mL) and stirred at room temperature for 0.5 hours. The reaction mixture was added 10 mL 10% KF solution, stirred at room temperature over 2 hours. The mixture was extracted with EtOAc (20 mL×3). The combined organic layers were washed with brine (20 mL), dried over anhydrous Na₂SO₄, filtered and concentrated in vacuo. The crude residue was purified by silica gel chromatography (solvent gradient: 0-50% EtOAc in petroleum ether) to give 1-(4-hydroxy-6-methyl-2-(pyridin-4-yl)quinazolin-8-yl)ethan-1-one (110 mg, 60%) as a white solid. MS: m/z 280.1 (M+H⁺).

### Step 3—Synthesis of 1-(4-(benzyloxy)-6-methyl-2-(pyridin-4-yl)quinazolin-8-yl)ethan-1-one

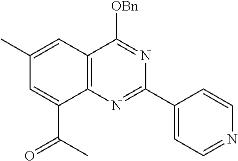 [SMILES: CC(=O)c1cc(C)cc2c(OCc3ccccc3)nc(-c3ccncc3)nc12]

To a solution of 1-(4-hydroxy-6-methyl-2-(pyridin-4-yl)quinazolin-8-yl)ethan-1-one (110 mg, 508 μmol), K₂CO₃ (105 mg, 763 μmol) in DMF (10 mL) was added BnBr (83 mg, 483 μmol). The reaction mixture was heated to 65° C. for 2 hours under N₂ atmosphere. After cooling to room temperature, the reaction was added water (20 mL) and extracted with EtOAc (20 mL×3). The combined organic layers were washed with brine (20 mL×3), dried over anhydrous Na₂SO₄, filtered and concentrated in vacuo. The crude residue was purified by silica gel chromatography (solvent gradient: 0-20% EtOAc in petroleum ether) to give 1-(4-(benzyloxy)-6-methyl-2-(pyridin-4-yl)quinazolin-8-yl)ethan-1-one (180 mg, 44%) as a white solid. MS: m/z 370.1 (M+H⁺).

### Step 4—Synthesis of 1-(4-(benzyloxy)-6-methyl-2-(pyridin-4-yl)quinazolin-8-yl)ethan-1-amine

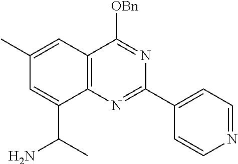 [SMILES: Cc1cc(C(C)N)c2nc(-c3ccncc3)nc(OCc3ccccc3)c2c1]

To a solution of 1-(4-(benzyloxy)-6-methyl-2-(pyridin-4-yl)quinazolin-8-yl)ethan-1-one (180 mg, 327 μmmol), NH₄OAc (252 mg, 3.27 mmol) in MeOH (10 mL) was added NaBH₃CN (62 mg, 980 umol). The reaction was heated to 40° C. for 16 hours under N₂ atmosphere. After cooling to room temperature, the reaction was diluted with water (20 mL), extracted with EtOAc (30 mL×3). The combined organic layers were washed with brine (20 mL), dried over anhydrous Na₂SO₄, filtered and concentrated in vacuo. The crude residue was purified by silica gel chromatography (solvent gradient: 0-10% MeOH in DCM) to give 1-(4-(benzyloxy)-6-methyl-2-(pyridin-4-yl)quinazolin-8-yl)ethan-1-amine (62 mg, 52%) as a white solid. ¹H NMR (400 MHz, DMSO-d₆) δ 8.81 (d, J=6.0 Hz, 2H), 8.41 (d, J=6.4 Hz, 2H), 7.96 (s, 1H), 7.91 (s, 1H), 7.62 (d, J=7.2 Hz, 1H), 7.48-7.42 (m, 1H), 7.38 (d, J=7.2 Hz, 1H), 5.82 (s, 2H), 5.28-5.16 (m, 1H), 2.53 (s, 3H), 1.50 (d, J=6.4 Hz, 3H). MS: m/z 371.1 (M+H⁻).

### Step 5—Synthesis of tert-butyl 2-((1-(4-(benzyloxy)-6-methyl-2-(pyridin-4-yl)quinazolin-8-yl)ethyl)amino)benzoate

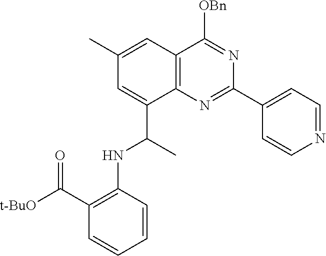 [SMILES: Cc1cc(C(C)Nc2ccccc2C(=O)OC(C)(C)C)c2nc(-c3ccncc3)nc(OCc3ccccc3)c2c1]

A mixture of 1-(4-(benzyloxy)-6-methyl-2-(pyridin-4-yl)quinazolin-8-yl)ethan-1-amine (50 mg, 135 umol), tert-butyl 2-bromobenzoate (103 mg, 2.1 mmol), Cs₂CO₃ (176 mg, 540 umol), Pd₂(dba)₃ (13 mg, 14 umol), Xantphos (16 mg, 27 umol) in dioxane (5 mL) was stirred at 100° C. for 16 hours under N₂ atmosphere. After cooling to room temperature, the reaction was filtered and the filtrate was concentrated in vacuo. The crude residue was purified by silica gel chromatography (solvent gradient: 0-20% EtOAc in petroleum ether) to give the title compound (22 mg, 30%) as a white solid. MS: m/z 547.3 (M+H⁺).

### Step 6—Synthesis of tert-butyl 2-((1-(4-hydroxy-6-methyl-2-(pyridin-4-yl)quinazolin-8-yl)ethyl)amino)benzoate

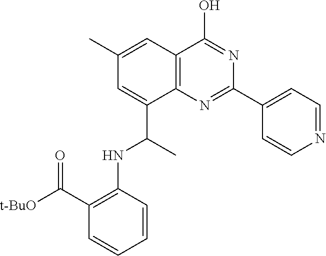 [SMILES: Cc1cc(C(C)Nc2ccccc2C(=O)OC(C)(C)C)c2nc(-c3ccncc3)nc(O)c2c1]

To a solution of tert-butyl 2-((1-(4-(benzyloxy)-6-methyl-2-(pyridin-4-yl)quinazolin-8-yl)ethyl)amino)benzoate (54 mg, 99 umol) in MeOH (5 mL) was added wet Pd/C (10 mg, 10% Pd, 50% wet with water). The reaction was stirred at room temperature for 0.5 hours under H2 atmosphere (15 psi). The reaction was filtered through diatomaceous earth and the filtrate was concentrated in vacuo to afford tert-butyl 2-((1-(4-hydroxy-6-methyl-2-(pyridin-4-yl)quinazolin-8-yl)ethyl)amino)benzoate (12 mg, 27%) as yellow oil that required no further purification. MS: m/z 457.2 (M+H⁺).

### Step 7—Synthesis of 2-((1-(4-hydroxy-6-methyl-2-(pyridin-4-yl)quinazolin-8-yl)ethyl)amino)benzoic acid

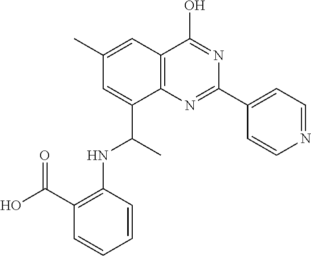 [SMILES: Cc1cc(C(C)Nc2ccccc2C(=O)O)c2nc(-c3ccncc3)nc(O)c2c1]

To a mixture of tert-butyl 2-((1-(4-hydroxy-6-methyl-2-(pyridin-4-yl)quinazolin-8-yl)ethyl)amino)benzoate (12 mg, 26 umol) in DCM (1.5 mL) was added TFA (0.5 mL, 6.9 mmol). The reaction mixture was stirred at room temperature for 16 hours. The mixture was concentrated in vacuo and the residue was purified by reverse phase chromatography (acetonitrile 32%-62%/0.225% formic acid in water) to give 2-((1-(4-hydroxy-6-methyl-2-(pyridin-4-yl)quinazolin-8-yl)ethyl)amino)benzoic acid (2.4 mg, 24%) as a white solid. ¹H NMR (400 MHz, DMSO-d₆) δ 12.78 (s, 1H), 8.81 (d, J=6.0 Hz, 2H), 8.49 (d, J=6.0 Hz, 1H), 8.22 (d, J=6.0 Hz, 2H), 7.88 (s, 1H), 7.81-7.76 (m, 1H), 7.62 (d, J=1.6 Hz, 1H), 7.23-7.15 (m, 1H), 6.56-6.45 (m, 2H), 5.72-5.57 (m, 1H), 2.40 (s, 3H), 1.62 (d, J=6.4 Hz, 3H). MS: m/z 401.2 (M+H⁺).

### Example 2: Synthesis of Compound 20: 2-((1-(7-methyl-3-morpholinoisoquinolin-5-yl)ethyl)amino)benzoic acid

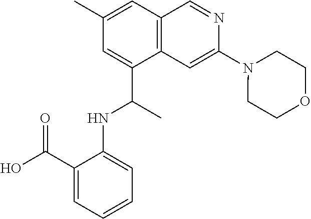 [SMILES: Cc1cc(C(C)Nc2ccccc2C(=O)O)c2cc(N3CCOCC3)ncc2c1]

### Step 1—Synthesis of (3-bromo-5-methylphenyl)methanamine

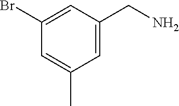

To a solution of 3-bromo-5-methyl-benzonitrile (5 g, 26 mmol) in THE (50 mL) was added borane tetrahydrofuran complex (33 mL, 33 mmol, 1M in THF) at room temperature. The mixture was heated to 70° C. and stirred for 2 hours. After cooling to room temperature, the mixture was quenched with MeOH (20 mL), then the mixture was heated to 70° C. and stirred for 1 hour. After cooling to room temperature, the mixture was concentrated in vacuo. The crude residue was purified by silica gel chromatography (solvent gradient: 0-10% MeOH in DCM) to give (3-bromo-5-methylphenyl)methanamine (3 g, 58%) as yellow oil. ¹H NMR (400 MHz, DMSO-d₆) δ 7.33 (s, 1H), 7.22 (s, 1H), 7.12 (s, 1H), 3.65 (s, 2H), 2.27 (s, 3H). MS: m/z 202.0 (M+H⁺).

### Step 2—Synthesis of N-(3-bromo-5-methylbenzyl)-2,2-dimethoxyacetamide

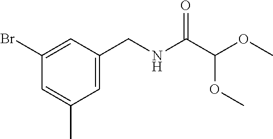 [SMILES: COC(OC)C(=O)NCc1cc(C)cc(Br)c1]

A mixture of (3-bromo-5-methylphenyl)methanamine (3 g, 15 mmol) and methyl 2,2-dimethoxyacetate (2.2 mL, 18 mmol) was heated to 130° C. and stirred for 16 hours. After cooling to room temperature, the reaction mixture was concentrated in vacuo. The crude residue was purified by silica gel chromatography (solvent gradient: 0-5% EtOAc in petroleum ether) to give N-(3-bromo-5-methylbenzyl)-2,2-dimethoxyacetamide (4.3 g, 88%) as yellow oil. ¹H NMR (400 MHz, CDCl₃) δ 7.25 (s, 1H), 7.23 (s, 1H), 7.02 (s, 1H), 6.92-6.81 (m, 1H), 4.76 (s, 1H), 4.41 (d, J=6.0 Hz, 2H), 3.42 (s, 6H), 2.32 (s, 3H). MS: m/z 302.0 (M+H⁺).

### Step 3—Synthesis of 5-bromo-7-methylisoquinolin-3-ol and 7-bromo-5-methylisoquinolin-3-ol

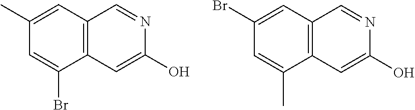

A solution of N-(3-bromo-5-methylbenzyl)-2,2-dimethoxyacetamide (4.3 g, 14 mmol) in conc. H₂SO₄ (12 mL, 230 mmol) was stirred at room temperature for 16 h. The reaction mixture was added to ice water (20 mL) slowly and the mixture was adjusted to pH 7-8 with 1M aqueous NaOH solution, then extracted with EtOAc (60 mL×3). The combined organic layers were washed with brine (50 mL×2), dried over anhydrous Na₂SO₄, filtered and concentrated in vacuo to give 5-bromo-7-methylisoquinolin-3-ol and 7-bromo-5-methylisoquinolin-3-ol (3.1 g, crude, mixture of two isomers, inseparable) as yellow solids that required no further purification. ¹H NMR (400 MHz, DMSO-d₆) δ 10.99 (s, 1H), 8.87 (s, 1H), 8.05-7.84 (m, 1H), 7.76-7.54 (m, 1H), 6.95-6.88 (m, 1H), 2.41 (s, 3H). MS: m/z 239.9 (M+H⁻).

### Step 4—Synthesis of 5-bromo-7-methylisoquinolin-3-yl trifluoromethanesulfonate

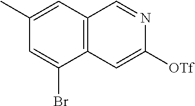 [SMILES: Cc1cc(Br)c2cc(OS(=O)(=O)C(F)(F)F)ncc2c1]

To a solution of 5-bromo-7-methylisoquinolin-3-ol and 7-bromo-5-methylisoquinolin-3-ol (1 g, 4.2 mmol, mixture of two isomers) in DCM (15 mL) was added pyridine (1 mL, 12.6 mmol) and Tf₂O (1.8 g, 6.3 mmol). The mixture was stirred at 0° C. for 2 hours. The reaction was quenched with water (30 mL) and extracted with DCM (60 mL×3). The combined organic layers were washed with brine (50 mL), dried over anhydrous Na₂SO₄, filtered and concentrated in vacuo. The crude residue was purified by silica gel chromatography (solvent gradient: 0-2% EtOAc in petroleum ether) to give 5-bromo-7-methylisoquinolin-3-yl trifluoromethanesulfonate (600 mg, 37%) as a yellow solid, which was further confirmed by NOESY. ¹H NMR (400 MHz, CDCl₃) δ 8.97 (s, 1H), 7.94 (s, 1H), 7.85 (s, 1H), 7.80 (s, 1H), 2.58 (s, 3H). MS: m/z 369.9 (M+H⁺).

### Step 5—Synthesis of 4-(5-bromo-7-methylisoquinolin-3-yl)morpholine

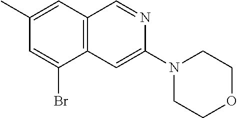 [SMILES: Cc1cc(Br)c2cc(N3CCOCC3)ncc2c1]

A mixture of 5-bromo-7-methylisoquinolin-3-yl trifluoromethanesulfonate (500 mg, 1.4 mmol), Pd₂(dba)₃ (124 mg, 135 μmol), Xantphos (156 mg, 270 μmol), Cs₂CO₃ (1.3 g, 4.1 mmol) and morpholine (130 mg, 1.5 mmol) in dioxane (20 mL) was stirred at 80° C. for 3 hours under N₂ atmosphere. After cooling to room temperature, the reaction was quenched with water (30 mL) and extracted with EtOAc (50 mL×3). The combined organic layers were washed with brine (30 mL), dried over anhydrous Na₂SO₄, filtered and concentrated in vacuo. The crude residue was purified by silica gel chromatography (solvent gradient: 0-25% EtOAc in petroleum ether) to give 4-(5-bromo-7-methylisoquinolin-3-yl)morpholine (170 mg, 37%) as a yellow solid. MS: m/z 309.0 (M+H⁺).

### Step 6—Synthesis of 1-(7-methyl-3-morpholinoisoquinolin-5-yl)ethan-1-one

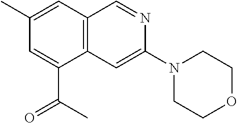 [SMILES: CC(=O)c1cc(C)cc2cnc(N3CCOCC3)cc12]

A mixture of 4-(5-bromo-7-methylisoquinolin-3-yl)morpholine (170 mg, 0.6 mmol), tributyl(1-ethoxyvinyl)stannane (354 uL, 1 mmol) and Pd(dppf)Cl₂ (40 mg, 55 μmol) in dioxane (10 mL) was degassed and purged with N₂ atmosphere for 3 times. The reaction mixture was stirred at 90° C. for 16 hours under N₂ atmosphere. After cooling to room temperature, HCl (2 mL, 1 M) was added. The mixture was stirred at room temperature for 2 hours. The reaction mixture was added 20 mL 10% KF aqueous solution, stirred at room temperature for 2 hours. The mixture was extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-10% EtOAc in petroleum ether) to give 1-(7-methyl-3-morpholinoisoquinolin-5-yl)ethan-1-one (120 mg, 50%) as a yellow solid. ¹H NMR (400 MHz, CDCl₃) δ 8.89 (s, 1H), 8.04 (s, 1H), 7.97 (s, 1H), 7.76 (s, 1H), 3.90-3.88 (m, 4H), 3.62-3.60 (m, 4H), 2.72 (s, 3H), 2.52 (s, 3H). MS: m/z 271.1 (M+H⁺).

### Step 7—Synthesis of 1-(7-methyl-3-morpholinoisoquinolin-5-yl)ethan-1-amine

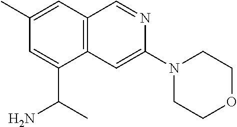 [SMILES: Cc1cc(C(C)N)c2cc(N3CCOCC3)ncc2c1]

To a solution of 1-(7-methyl-3-morpholinoisoquinolin-5-yl)ethan-1-one (110 mg, 407 μmol), NH₄OAc (470 mg, 6 mmol) and AcOH (47 μL, 814 μmol) in MeOH (5 mL). The mixture was stirred at room temperature for 0.5 hours, then NaBH₃CN (77 mg, 1 mmol) was added. The reaction was heated to 60° C. for 16 hours under N₂ atmosphere. After cooling to room temperature, the reaction was diluted with water (20 mL), extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-5% MeOH in DCM) to give 1-(7-methyl-3-morpholinoisoquinolin-5-yl)ethan-1-amine (50 mg, 36%) as a yellow solid. ¹H NMR (400 MHz, DMSO-d₆) δ 8.89 (s, 1H), 7.63 (s, 1H), 7.52 (s, 1H), 7.00 (s, 1H), 4.66 (q, J=6.4 Hz, 1H), 3.78-3.75 (m, 4H), 3.48-3.46 (m, 4H), 2.41 (s, 3H), 1.33 (d, J=6.4 Hz, 3H). MS: m/z 272.3 (M+H⁻).

### Step 8—Synthesis of tert-butyl 2-((1-(7-methyl-3-morpholinoisoquinolin-5-yl)ethyl)amino)benzoate

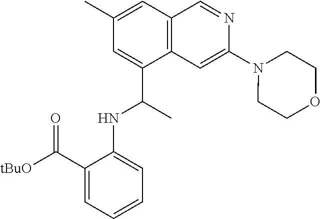 [SMILES: Cc1cc(C(C)Nc2ccccc2C(=O)OC(C)(C)C)c2cc(N3CCOCC3)ncc2c1]

A mixture of 1-(7-methyl-3-morpholinoisoquinolin-5-yl)ethan-1-amine (50 mg, 184 mol), Pd₂(dba)₃ (17 mg, 18 μmol), Xantphos (21 mg, 37 μmol), Cs₂CO₃ (180 mg, 553 μmol) and tert-butyl 2-iodobenzoate (112 mg, 369 μmol) in dioxane (5 mL) was stirred at 110° C. for 16 hours under N₂ atmosphere. After cooling to room temperature, the reaction was diluted with water (15 mL) and extracted with EtOAc (30 mL×2). The combined organic layers were washed with brine (30 mL), dried over anhydrous Na₂SO₄, filtered and concentrated in vacuo. The crude residue was purified by silica gel chromatography (solvent gradient: 0-10% EtOAc in petroleum ether) to give tert-butyl 2-((1-(7-methyl-3-morpholinoisoquinolin-5-yl)ethyl)amino)benzoate (40 mg, 46%) as yellow oil. ¹H NMR (400 MHz, DMSO-d₆) δ 8.94 (s, 1H), 8.18 (d, J=6.4 Hz, 1H), 7.76 (dd, J=8.0, 1.2 Hz, 1H), 7.56 (s, 1H), 7.38 (s, 1H), 7.19-7.15 (m, 1H), 7.11 (s, 1H), 6.54-6.50 (m, 1H), 6.40 (d, J=8.4 Hz, 1H), 5.36-5.29 (m, 1H), 3.78-3.75 (m, 4H), 3.51-3.49 (m, 4H), 2.33 (s, 3H), 1.59 (d, J=6.4 Hz, 3H), 1.57 (s, 9H). MS: m/z 448.1 (M+H⁺).

### Step 9—Synthesis of 2-((1-(7-methyl-3-morpholinoisoquinolin-5-yl)ethyl)amino) benzoic acid

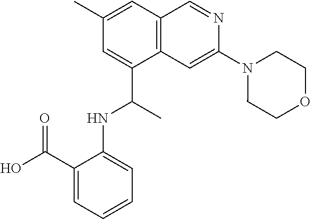 [SMILES: Cc1cc(C(C)Nc2ccccc2C(=O)O)c2cc(N3CCOCC3)ncc2c1]

To a mixture of tert-butyl 2-((1-(7-methyl-3-morpholinoisoquinolin-5-yl)ethyl)amino)benzoate (40 mg, 89 μmol) in DCM (1.5 mL) was added TFA (0.5 mL). The reaction was stirred at room temperature for 16 h. The reaction mixture was concentrated in vacuo. The resulting residue was purified by reverse phase chromatography (acetonitrile 40-70%/0.225% formic acid in water) to give 2-((1-(7-methyl-3-morpholinoisoquinolin-5-yl)ethyl)amino) benzoic acid (16 mg, 44%) as a yellow solid. ¹H NMR (400 MHz, DMSO-d₆) δ 8.93 (s, 1H), 8.46 (s, 1H), 7.81-7.79 (m, 1H), 7.55 (s, 1H), 7.38 (s, 1H), 7.18-7.14 (m, 1H), 7.11 (s, 1H), 6.52-6.48 (m, 1H), 6.36 (d, J=8.4 Hz, 1H), 5.35-5.29 (m, 1H), 3.76 (t, J=4.8 Hz, 4H), 3.50 (t, J=4.8 Hz, 4H), 2.33 (s, 3H), 1.57 (d, J=6.8 Hz, 3H). MS: m/z 392.1 (M+H⁺).

### Example 3: Synthesis of Compound 26: 2-((1-(4-cyano-2-(4,4-dimethylpiperidin-1-yl)-6-methylquinazolin-8-yl)ethyl)amino)benzoic acid

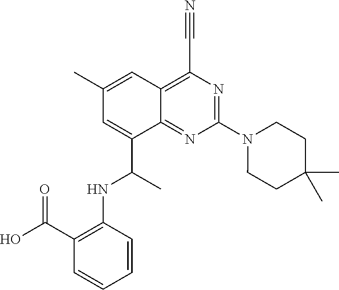 [SMILES: Cc1cc(C(C)Nc2ccccc2C(=O)O)c2nc(N3CCC(C)(C)CC3)nc(C#N)c2c1]

### Step 1—Synthesis of 8-bromo-2-(4,4-dimethylpiperidin-1-yl)-6-methylquinazolin-4(3H)-one

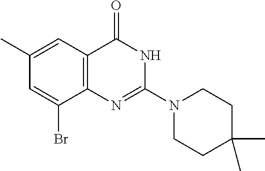

To a mixture of methyl 2-amino-3-bromo-5-methylbenzoate (3 g, 12.3 mmol) (prepared according to the procedure in CN111440161) and 4,4-dimethylpiperidine-1-carbonitrile (2.55 g, 18.5 mmol) in THE (40 mL) was added NaH (983 mg, 24.8 mmol, 60% purity). The reaction mixture was heated to 90° C. for 5 hours under N₂ atmosphere. After cooling to room temperature, the reaction was quenched water (50 mL) and extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-30% EtOAc in petroleum ether) to give 8-bromo-2-(4,4-dimethylpiperidin-1-yl)-6-methylquinazolin-4(3H)-one (1.5 g, 35%) as a white solid. ¹H NMR (400 MHz, DMSO-d₆) δ 11.39 (s, 1H), 7.76 (d, J=2.0 Hz, 1H), 7.69 (d, J=2.0 Hz, 1H), 3.70-3.63 (m, 4H), 2.32 (s, 3H), 1.40-1.33 (m, 4H), 0.96 (s, 6H). MS: m/z 352.0 (M+H⁺).

### Step 2—Synthesis of 8-acetyl-2-(4,4-dimethylpiperidin-1-yl)-6-methylquinazolin-4(3H)-one

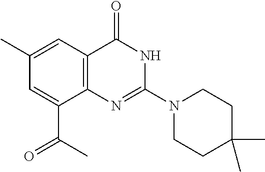

A mixture of 8-bromo-2-(4,4-dimethylpiperidin-1-yl)-6-methylquinazolin-4(3H)-one (650 mg, 1.87 mmol), tributyl(1-ethoxyvinyl)stannane (1.90 g, 5.26 mmol) and Pd(dppf)Cl₂ (272 mg, 0.4 mmol) in dioxane (6 mL) was stirred at 90° C. for 16 hours under N₂ atmosphere. After cooling to room temperature, the reaction mixture was added 1M HCl in water (2 mL) and stirred at room temperature for 0.5 hours. The reaction mixture was added 20 mL 10% KF solution, stirred at room temperature over 2 hours. The mixture was extracted with EtOAc (30 mL×3). The combined organic layers were washed with brine (20 mL), dried over anhydrous Na₂SO₄, filtered and concentrated in vacuo. The crude residue was purified by silica gel chromatography (solvent gradient: 0-50% EtOAc in petroleum ether) to give 8-acetyl-2-(4,4-dimethylpiperidin-1-yl)-6-methylquinazolin-4(3H)-one (450 mg, 51%) as a yellow solid. ¹H NMR (400 MHz, DMSO-d₆) δ 11.45 (s, 1H), 7.90 (s, 1H), 7.63 (s, 1H), 3.66-3.60 (m, 4H), 2.73 (s, 3H), 2.35 (s, 3H), 1.41-1.35 (m, 4H), 0.97 (s, 6H). MS: m/z 314.1 (M+H⁺).

### Step 3—Synthesis of 2-(4,4-dimethylpiperidin-1-yl)-8-(1-hydroxyethyl)-6-methylquinazolin-4(31)-one

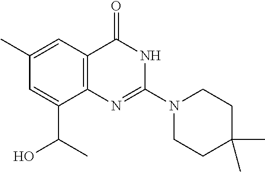

To a solution of 8-acetyl-2-(4,4-dimethylpiperidin-1-yl)-6-methylquinazolin-4(3H)-one (0.6 g, 1.91 mmol) in MeOH (10 mL) was added NaBH₄ (150 mg, 3.96 mmol) at 0° C. The mixture was stirred at 0° C. for 2 hours under N₂ atmosphere. The reaction was quenched with saturated aqueous (sat. aq.) NH₄Cl (3 mL), diluted with water (20 mL), extracted with EtOAc (30 mL×2). The combined organic layers were washed with brine (20 mL), dried over anhydrous Na₂SO₄, filtered and concentrated in vacuo. The crude residue was purified by silica gel chromatography (solvent gradient: 0-10% MeOH in DCM) to give 2-(4,4-dimethylpiperidin-1-yl)-8-(1-hydroxyethyl)-6-methylquinazolin-4(3H)-one (460 mg, 76%) as a yellow solid. ¹H NMR (400 MHz, DMSO-d₆) δ 11.26 (s, 1H), 7.59 (s, 1H), 7.55 (s, 1H), 5.36-5.27 (m, 1H), 5.11 (d, J=4.4 Hz, 1H), 3.62-3.55 (m, 4H), 2.34 (s, 3H), 1.41-1.31 (m, 7H), 0.97 (s, 6H). MS: m/z 316.2 (M+H⁺).

### Step 4—Synthesis of 8-(1-bromoethyl)-2-(4,4-dimethylpiperidin-1-yl)-6-methylquinazolin-4(3H)-one

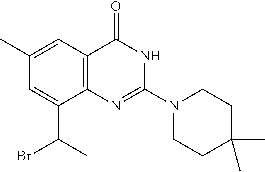

To a solution of 2-(4,4-dimethyl-1-piperidyl)-8-(1-hydroxyethyl)-6-methyl-3H-quinazolin-4-one (460 mg, 1.46 mmol) in DCM (6 mL) was added PBr₃ (592.16 mg, 2.19 mmol) at 0° C. The reaction mixture was stirred at 0° C. for 1 hour under N₂ atmosphere. The mixture was quenched with sat. aq. NaHCO₃ (20 mL), extracted with DCM (30 mL×2). The combined organic layers were washed with brine (30 mL), dried over anhydrous Na₂SO₄, filtered and concentrated in vacuo to give 8-(1-bromoethyl)-2-(4,4-dimethylpiperidin-1-yl)-6-methylquinazolin-4(3H)-one (500 mg, crude) as a yellow solid that required no further purification.

### Step 5—Synthesis of tert-butyl 2-((1-(2-(4,4-dimethylpiperidin-1-yl)-6-methyl-4-oxo-3,4-dihydroquinazolin-8-yl)ethyl)amino)benzoate

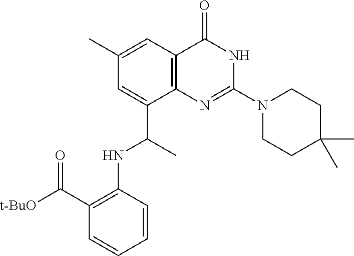 [SMILES: Cc1cc(C(C)Nc2ccccc2C(=O)OC(C)(C)C)c2nc(N3CCC(C)(C)CC3)[nH]c(=O)c2c1]

To a solution of 8-(1-bromoethyl)-2-(4,4-dimethyl-1-piperidyl)-6-methyl-3H-quinazolin-4-one (0.4 g, 1.06 mmol) in DMF (4 mL) was added tert-butyl 2-aminobenzoate (613 mg, 3.17 mmol). The mixture was stirred at 90° C. for 16 hours. After cooling to room temperature, the reaction mixture was quenched with water (20 mL) and extracted with EtOAc (40 mL×2). The combined organic layers were washed with brine (30 mL×3), dried over anhydrous Na₂SO₄, filtered and concentrated in vacuo. The residue was purified by reverse phase chromatography (acetonitrile 85%-100%/0.225% formic acid in water) to give tert-butyl 2-((1-(2-(4,4-dimethylpiperidin-1-yl)-6-methyl-4-oxo-3,4-dihydroquinazolin-8-yl)ethyl)amino)benzoate (30 mg, 7%) as yellow oil. MS: m/z 491.4 (M+H⁺).

### Step 6—Synthesis of tert-butyl 2-((1-(4-cyano-2-(4,4-dimethylpiperidin-1-yl)-6-methylquinazolin-8-yl)ethyl)amino)benzoate

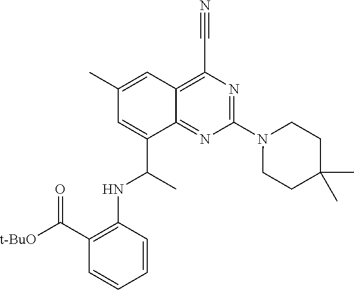 [SMILES: Cc1cc(C(C)Nc2ccccc2C(=O)OC(C)(C)C)c2nc(N3CCC(C)(C)CC3)nc(C#N)c2c1]

To a solution of tert-butyl 2-((1-(2-(4,4-dimethylpiperidin-1-yl)-6-methyl-4-oxo-3,4-dihydroquinazolin-8-yl)ethyl)amino)benzoate (30 mg, 61 μmol) in DMF (1 mL) was added BOP (35 mg, 79 μmol), DBU (14 mg, 92 μmol), NaCN (10 mg, 183 μmol,) and 18-crown-6 (48 mg, 182 μmol). The reaction mixture was stirred at room temperature for 2 hours. The reaction was added water (20 mL) and extracted with EtOAc (30 mL×2). The combined organic layers were washed with brine (30 mL×3), dried over anhydrous Na₂SO₄, filtered and concentrated in vacuo. The crude residue was purified by silica gel chromatography (solvent gradient: 0-5% EtOAc in petroleum ether) to give tert-butyl 2-((1-(4-cyano-2-(4,4-dimethylpiperidin-1-yl)-6-methylquinazolin-8-yl)ethyl)amino)benzoate (18 mg, 59%) as a yellow solid. ¹H NMR (500 MHz, CDCl₃) δ 7.88-7.85 (m, 1H), 7.69 (d, J=1.6 Hz, 1H), 7.61-7.59 (m, 1H), 7.24-7.20 (m, 1H), 6.83-6.77 (m, 2H), 5.65-5.59 (m, 1H), 3.94-3.88 (m, 4H), 2.41 (s, 3H), 1.82 (d, J=5.2 Hz, 3H), 1.60 (s, 9H), 1.48-1.43 (m, 4H) 1.05 (s, 6H). MS: m/z 500.1 (M+H⁺).

### Step 7—Synthesis of 2-((1-(4-cyano-2-(4,4-dimethylpiperidin-1-yl)-6-methylquinazolin-8-yl)ethyl)amino)benzoic acid

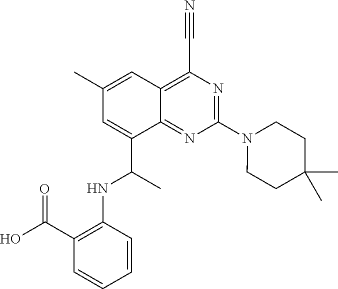 [SMILES: Cc1cc(C(C)Nc2ccccc2C(=O)O)c2nc(N3CCC(C)(C)CC3)nc(C#N)c2c1]

To a solution of tert-butyl 2-((1-(4-cyano-2-(4,4-dimethylpiperidin-1-yl)-6-methylquinazolin-8-yl)ethyl)amino)benzoate (18 mg, 36 umol) in DCM (2 mL) was added TFA (0.5 mL, 6.9 mmol). The reaction mixture was stirred at room temperature for 16 hours. The mixture was concentrated in vacuo. The residue was purified by reverse phase chromatography (acetonitrile 85%-100%/0.225% formic acid in water) to give 2-((1-(4-cyano-2-(4,4-dimethylpiperidin-1-yl)-6-methylquinazolin-8-yl)ethyl)amino)benzoic acid (2.7 mg, 19%) as a yellow solid. ¹H NMR (400 MHz, DMSO-d₆) δ 12.70 (s, 1H), 8.48 (s, 1H), 7.79 (d, J=6.4 Hz, 1H), 7.64 (s, 1H), 7.56 (s, 1H), 7.16 (s, 1H), 6.52-6.48 (m, 1H), 6.40 (s, 1H), 5.43 (s, 1H), 3.89 (s, 4H), 2.39 (s, 3H), 1.60 (d, J=6.4 Hz, 3H), 1.46-1.40 (m, 4H), 1.01 (s, 6H). MS: m/z 444.2 (M+H⁺).

### Example 4: Synthesis of Compound 32: (R)-2-((1-(2-(4-fluorophenyl)-3,6-dimethyl-4-oxo-3,4-dihydroquinazolin-8-yl)ethyl)amino)benzoic acid

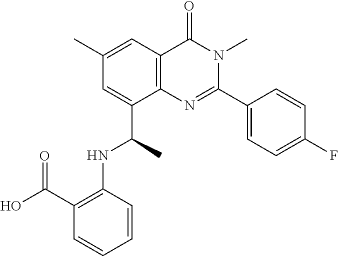 [SMILES: Cc1cc([C@@H](C)Nc2ccccc2C(=O)O)c2nc(-c3ccc(F)cc3)n(C)c(=O)c2c1]

### Step 1—Synthesis of 8-bromo-2-(4-fluorophenyl)-3,6-dimethylquinazolin-4(3H)-one

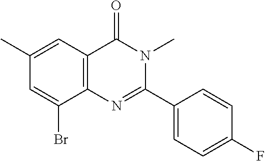 [SMILES: Cc1cc(Br)c2nc(-c3ccc(F)cc3)n(C)c(=O)c2c1]

A mixture of 2-amino-3-bromo-N,5-dimethylbenzamide (2.3 g, 9.5 mmol) (prepared according to the procedure in Eur. J. Med. Chem., 2021, 225, 113764), 4-fluorobenzaldehyde (2 g, 16.1 mmol) and CuO (1.5 g, 18.9 mmol) in DMA (20 mL) was stirred at 135° C. for 16 hours under O₂ atmosphere. After cooling to room temperature, the reaction was poured into water (50 mL), the suspension was filtered and the filter cake was dried in vacuo to give 8-bromo-2-(4-fluorophenyl)-3,6-dimethylquinazolin-4(3H)-one (1.3 g, crude) as a white solid that required no further purification. ¹H NMR (400 MHz, DMSO-d₆) δ 7.99 (s, 1H), 7.95 (s, 1H), 7.79-7.74 (m, 2H), 7.44-7.35 (m, 2H), 3.35 (s, 3H), 2.45 (s, 3H). MS: m/z 347.0 (M+H⁺).

### Step 2—Synthesis of 8-acetyl-2-(4-fluorophenyl)-3,6-dimethylquinazolin-4(3H)-one

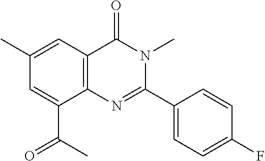 [SMILES: CC(=O)c1cc(C)cc2c(=O)n(C)c(-c3ccc(F)cc3)nc12]

A mixture of 8-bromo-2-(4,4-dimethylpiperidin-1-yl)-6-methylquinazolin-4(3H)-one (1.28 g, 3.69 mmol), tributyl(1-ethoxyvinyl)stannane (4 mL, 11.85 mmol) and Pd(dppf)Cl₂ (270 mg, 0.37 mmol) in dioxane (10 mL) was stirred at 90° C. for 16 hours under N₂ atmosphere. After cooling to room temperature, the reaction mixture was added 1M HCl in water (5 mL) and stirred at room temperature for 0.5 hours. The reaction mixture was added 40 mL 10% KF solution, stirred at room temperature over 2 hours. The mixture was extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-30% EtOAc in petroleum ether) to give 8-acetyl-2-(4-fluorophenyl)-3,6-dimethylquinazolin-4(3H)-one (500 mg, 44%) as a yellow solid. ¹H NMR (400 MHz, DMSO-d₆) δ 8.15 (d, J=1.2 Hz, 1H), 7.84-7.74 (m, 3H), 7.43-7.37 (m, 2H), 3.40 (s, 3H), 2.71 (s, 3H), 2.49 (s, 3H). MS: m/z 311.1 (M+H⁺).

### Step 3—Synthesis of 8-(1-aminoethyl)-2-(4-fluorophenyl)-3,6-dimethylquinazolin-4(3H)-one

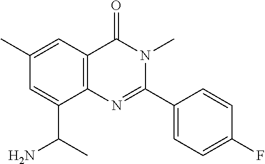 [SMILES: Cc1cc(C(C)N)c2nc(-c3ccc(F)cc3)n(C)c(=O)c2c1]

To a solution of 8-acetyl-2-(4-fluorophenyl)-3,6-dimethylquinazolin-4(3H)-one (180 mg, 0.58 mmol), NH₄OAc (447 mg, 5.8 mmol) in MeOH (10 mL) was added NaBH₃CN (110 mg, 1.74 mmol). The reaction was heated to 40° C. for 16 hours under N₂ atmosphere. After cooling to room temperature, the reaction was diluted with water (20 mL), extracted with EtOAc (30 mL×3). The combined organic layers were washed with brine (30 mL), dried over anhydrous Na₂SO₄, filtered and concentrated in vacuo. The crude residue was purified by silica gel chromatography (solvent gradient: 0-10% MeOH in DCM) to give 8-(1-aminoethyl)-2-(4-fluorophenyl)-3,6-dimethylquinazolin-4(3H)-one (160 mg, 88%) as a white solid. ¹H NMR (400 MHz, DMSO-d₆) δ 7.88 (s, 1H), 7.81-7.77 (m, 3H), 7.43-7.37 (m, 2H), 4.88-4.82 (m, 1H), 3.38 (s, 3H), 2.47 (s, 3H), 1.36 (d, J=6.8 Hz, 3H). MS: m/z 312.2 (M+H⁺).

### Step 4—Synthesis of tert-butyl 2-((1-(2-(4-fluorophenyl)-3,6-dimethyl-4-oxo-3,4-dihydroquinazolin-8-yl)ethyl)amino)benzoate

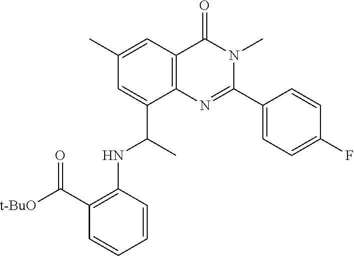 [SMILES: Cc1cc(C(C)Nc2ccccc2C(=O)OC(C)(C)C)c2nc(-c3ccc(F)cc3)n(C)c(=O)c2c1]

A mixture of 8-(1-aminoethyl)-2-(4-fluorophenyl)-3,6-dimethylquinazolin-4(3H)-one (160 mg, 514 μmol), tert-butyl 2-iodobenzoate (391 mg, 1.28 mmol), Pd₂(dba)₃ (47 mg, 51 μmol), Xantphos (60 mg, 103 μmol) and Cs₂CO₃ (502 mg, 1.54 mmol) in dioxane (5 mL) was stirred at 100° C. for 16 hours under N₂ atmosphere. After cooling to room temperature, the reaction was filtered and the filtrate was concentrated in vacuo. The crude residue was purified by silica gel chromatography (solvent gradient: 0-20% EtOAc in petroleum ether) to give tert-butyl 2-((1-(2-(4-fluorophenyl)-3,6-dimethyl-4-oxo-3,4-dihydroquinazolin-8-yl)ethyl)amino)benzoate (180 mg, 71%) as a white solid. ¹H NMR (400 MHz, DMSO-d₆) δ 8.24 (d, J=6.8 Hz, 1H), 7.93-7.84 (m, 3H), 7.73 (dd, J=8.0, 1.6 Hz, 1H), 7.60 (d, J=1.6 Hz, 1H), 7.44-7.38 (m, 2H), 7.22-7.13 (m, 1H), 6.55-6.42 (m, 2H), 5.47-5.36 (m, 1H), 3.41 (s, 3H), 2.40 (s, 3H), 1.59-1.53 (m, 12H). MS: m/z 488.2 (M+H⁺).

### Step 5—Synthesis of 2-((1-(2-(4-fluorophenyl)-3,6-dimethyl-4-oxo-3,4-dihydroquinazolin-8-yl)ethyl)amino)benzoic acid

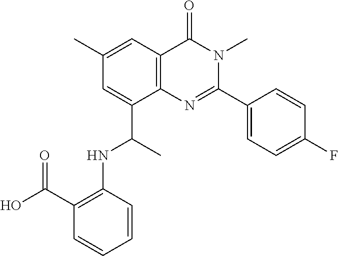 [SMILES: Cc1cc(C(C)Nc2ccccc2C(=O)O)c2nc(-c3ccc(F)cc3)n(C)c(=O)c2c1]

To a mixture of tert-butyl 2-((1-(2-(4-fluorophenyl)-3,6-dimethyl-4-oxo-3,4-dihydroquinazolin-8-yl)ethyl)amino)benzoate (100 mg, 205 μmol) in DCM (2 mL) was added TFA (0.5 mL). The reaction mixture was stirred at room temperature for 16 hours. The mixture was concentrated in vacuo. The residue was purified by reverse phase chromatography (acetonitrile 51%-81%/0.225% formic acid in water) to give 2-((1-(2-(4-fluorophenyl)-3,6-dimethyl-4-oxo-3,4-dihydroquinazolin-8-yl)ethyl)amino)benzoic acid (65 mg, 73%) as a white solid. ¹H NMR (400 MHz, DMSO-d₆) δ 12.69 (s, 1H), 8.52-8.31 (m, 1H), 7.88-7.83 (m, 3H), 7.78 (dd, J=8.0, 1.6 Hz, 1H), 7.57 (d, J=1.6 Hz, 1H), 7.45-7.38 (m, 2H), 7.19-7.13 (m, 1H), 6.52-6.46 (m, 1H), 6.41 (d, J=8.4 Hz, 1H), 5.50-5.43 (m, 1H), 3.41 (s, 3H), 2.39 (s, 3H), 1.53 (d, J=6.8 Hz, 3H). MS: m/z 432.2 (M+H⁺).

### Step 6—Synthesis of (R)-2-((1-(2-(4-fluorophenyl)-3,6-dimethyl-4-oxo-3,4-dihydroquinazolin-8-yl)ethyl)amino)benzoic acid & (S)-2-((1-(2-(4-fluorophenyl)-3,6-dimethyl-4-oxo-3,4-dihydroquinazolin-8-yl)ethyl)amino)benzoic acid

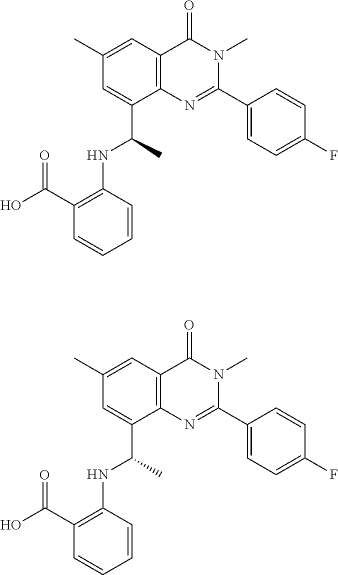 [SMILES: Cc1cc([C@@H](C)Nc2ccccc2C(=O)O)c2nc(-c3ccc(F)cc3)n(C)c(=O)c2c1 ; Cc1cc([C@H](C)Nc2ccccc2C(=O)O)c2nc(-c3ccc(F)cc3)n(C)c(=O)c2c1]

2-((1-(2-(4-fluorophenyl)-3,6-dimethyl-4-oxo-3,4-dihydroquinazolin-8-yl)ethyl)amino) benzoic acid (65 mg, 151 umol) was separated by using chiral SFC (DAICEL CHIRALCEL AD (250 mm*30 mm, 10 um); Supercritical CO₂/EtOH+0.1% NH₃·H₂O=75/25; 70 mL/min) to afford (R)-2-((1-(2-(4-fluorophenyl)-3,6-dimethyl-4-oxo-3,4-dihydroquinazolin-8-yl)ethyl)amino)benzoic acid (19 mg, first peak) and (S)-2-((1-(2-(4-fluorophenyl)-3,6-dimethyl-4-oxo-3,4-dihydroquinazolin-8-yl)ethyl)amino)benzoic acid (16 mg, second peak) both as white solid. Absolute configuration was arbitrarily assigned to each enantiomer. ¹H NMR (400 MHz, DMSO-d₆) δ 8.51-8.39 (m, 1H), 7.88-7.82 (m, 3H), 7.78 (dd, J=8.0, 1.6 Hz, 1H), 7.57 (d, J=2.0 Hz, 1H), 7.45-7.37 (m, 2H), 7.19-7.12 (m, 1H), 6.51-6.45 (m, 1H), 6.40 (d, J=8.4 Hz, 1H), 5.49-5.43 (m, 1H), 3.41 (s, 3H), 2.38 (s, 3H), 1.53 (d, J=6.8 Hz, 3H). MS: m/z 432.2 (M+H⁺).

### Example 5: Synthesis of Compound 33: (S)-2-((1-(2-(1,3-dimethyl-1H-indazol-5-yl)-3,6-dimethyl-4-oxo-3,4-dihydroquinazolin-8-yl)ethyl)amino)benzoic acid

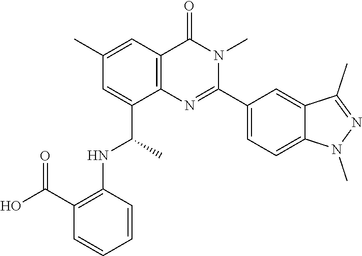 [SMILES: Cc1cc([C@H](C)Nc2ccccc2C(=O)O)c2nc(-c3ccc4c(c3)c(C)nn4C)n(C)c(=O)c2c1]

### Step 1—Synthesis of 1,3-dimethyl-1H-indazole-5-carbaldehyde

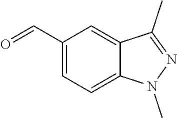

To a stirred solution of 5-bromo-1,3-dimethyl-indazole (2 g, 8.89 mmol) in dry THF (20 mL) was added n-BuLi (4.27 mL, 10.68 mmol, 2.5 M in hexane) at −78° C. under N₂ atmosphere. The resulting reaction mixture was stirred at this temperature for 15 minutes. Then dry DMF (685 uL, 8.89 mmol) was added to the reaction mixture at −78° C. The mixture was stirred at this temperature for 2 hours. After complete consumption of the starting material, the reaction mixture was quenched with water (30 mL), extracted with EtOAc (50 mL×2). The combined organic layers were washed with brine (50 mL), dried over anhydrous Na₂SO₄, filtered and concentrated in vacuo. The crude residue was purified by silica gel chromatography (solvent gradient: 0-30% EtOAc in petroleum ether) to give 1,3-dimethyl-1H-indazole-5-carbaldehyde (1.5 g, 96%) as a white solid. ¹H NMR (400 MHz, DMSO-d₆) δ 10.01 (s, 1H), 8.38 (s, 1H), 7.84 (d, J=8.8 Hz, 1H), 7.70 (d, J=8.8 Hz, 1H), 4.00 (s, 3H), 2.54 (s, 3H). MS: m/z 175.1 (M+H⁺).

### Step 2—Synthesis of 8-bromo-2-(1,3-dimethyl-1H-indazol-5-yl)-3,6-dimethylquinazolin-4(3H)-one

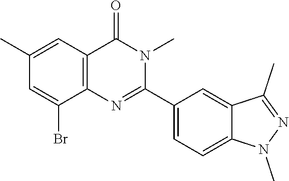 [SMILES: Cc1cc(Br)c2nc(-c3ccc4c(c3)c(C)nn4C)n(C)c(=O)c2c1]

A mixture of 2-amino-3-bromo-N,5-dimethylbenzamide (1.5 g, 6.17 mmol) (prepared according to the procedure in Eur. J Med. Chem., 2021, 225, 113764), 1,3-dimethyl-1H-indazole-5-carbaldehyde (1.29 g, 7.40 mmol) and CuO (982 mg, 12.34 mmol) in DMA (30 mL) was stirred at 135° C. for 16 hours under O₂ atmosphere. After cooling to room temperature, the reaction was poured into water (100 mL), extracted with EtOAc (100 mL×2). The combined organic layers were washed with brine (100 mL×3), dried over anhydrous Na₂SO₄, filtered and concentrated in vacuo. The crude residue was purified by silica gel chromatography (solvent gradient: 0-50% EtOAc in petroleum ether) to give 8-bromo-2-(1,3-dimethyl-1H-indazol-5-yl)-3,6-dimethylquinazolin-4(3H)-one (1.5 g, 61%) as a white solid. ¹H NMR (400 MHz, DMSO-d₆) δ 8.07 (s, 1H), 8.02 (d, J=1.6 Hz, 1H), 7.99 (s, 1H), 7.71 (s, 1H), 7.70 (d, J=1.2 Hz, 1H), 4.03 (s, 3H), 3.40 (s, 3H), 2.53 (s, 3H), 2.47 (s, 3H). MS: m/z 397.1 (M+H⁺).

### Step 3—Synthesis of 8-acetyl-2-(1,3-dimethyl-1H-indazol-5-yl)-3,6-dimethylquinazolin-4(3H)-one

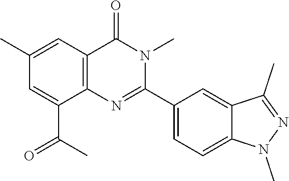 [SMILES: CC(=O)c1cc(C)cc2c(=O)n(C)c(-c3ccc4c(c3)c(C)nn4C)nc12]

A mixture of 8-bromo-2-(1,3-dimethylindazol-5-yl)-3,6-dimethyl-quinazolin-4-one (400 mg, 1.01 mmol), tributyl(1-ethoxyvinyl)stannane (1.09 g, 3.02 mmol), Pd(dppf)Cl₂ (74 mg, 101 μmol) in dioxane (10 mL) was stirred at 90° C. for 16 hours under N₂ atmosphere. After cooling to room temperature, the reaction mixture was added 1M HCl in water (5 mL) and stirred at room temperature for 0.5 hours. The reaction mixture was added 50 mL 10% KF solution, stirred at room temperature over 2 hours. The mixture was extracted with EtOAc (60 mL×3). The combined organic layers were washed with brine (50 mL), dried over anhydrous Na₂SO₄, filtered and concentrated in vacuo. The crude residue was purified by silica gel chromatography (solvent gradient: 0-50% EtOAc in petroleum ether) to give 8-acetyl-2-(1,3-dimethyl-1H-indazol-5-yl)-3,6-dimethylquinazolin-4(3H)-one (200 mg, 51%) as yellow oil. MS: m/z 361.2 (M+H⁺).

### Step 4—Synthesis of 8-(1-aminoethyl)-2-(1,3-dimethyl-1H-indazol-5-yl)-3,6-dimethylquinazolin-4(3H)-one

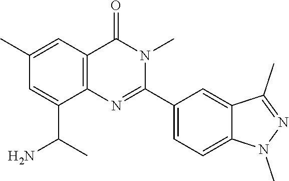 [SMILES: Cc1cc(C(C)N)c2nc(-c3ccc4c(c3)c(C)nn4C)n(C)c(=O)c2c1]

To a solution of 8-acetyl-2-(1,3-dimethylindazol-5-yl)-3,6-dimethyl-quinazolin-4-one (700 mg, 1.94 mmol), NH₄OAc (1.50 g, 19.42 mmol) in MeOH (10 mL) was added NaBH₃CN (336 mg, 5.83 mmol). The reaction was heated to 40° C. for 16 hours under N₂ atmosphere. After cooling to room temperature, the reaction was diluted with water (30 mL), extracted with EtOAc (50 mL×3). The combined organic layers were washed with brine (50 mL), dried over anhydrous Na₂SO₄, filtered and concentrated in vacuo. The crude residue was purified by silica gel chromatography (solvent gradient: 0-10% MeOH in DCM) to give 8-(1-aminoethyl)-2-(1,3-dimethyl-1H-indazol-5-yl)-3,6-dimethylquinazolin-4(3H)-one (250 mg, 36%) as a white solid. ¹H NMR (400 MHz, DMSO-d₆) δ 8.07 (s, 1H), 7.85 (s, 1H), 7.78 (d, J-1.6 Hz, 1H), 7.73-7.67 (m, 2H), 4.85-4.67 (m, 1H), 4.02 (s, 3H), 3.42 (s, 3H), 2.52 (s, 3H), 2.47 (s, 3H), 2.11 (s, 2H), 1.31 (d, J=6.4 Hz, 3H). MS: m/z 362.3 (M+H⁺).

### Step 5—Synthesis of tert-butyl 2-((1-(2-(1,3-dimethyl-1H-indazol-5-yl)-3,6-dimethyl-4-oxo-3,4-dihydroquinazolin-8-yl)ethyl)amino)benzoate

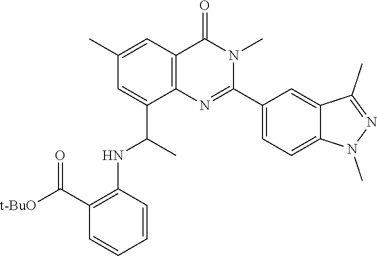 [SMILES: Cc1cc(C(C)Nc2ccccc2C(=O)OC(C)(C)C)c2nc(-c3ccc4c(c3)c(C)nn4C)n(C)c(=O)c2c1]

A mixture of 8-(1-aminoethyl)-2-(1,3-dimethylindazol-5-yl)-3,6-dimethyl-quinazolin-4-one (250 mg, 692 μmol), tert-butyl 2-iodobenzoate (421 mg, 1.38 mmol), Pd₂(dba)₃ (63 mg, 69 μmol), Xantphos (80 mg, 138 μmol) and Cs₂CO₃ (676 mg, 2.08 mmol) in dioxane (10 mL) was stirred at 100° C. for 3 hours under N₂ atmosphere. After cooling to room temperature, the reaction was filtered and the filtrate was concentrated in vacuo. The crude residue was purified by silica gel chromatography (solvent gradient: 0-20% EtOAc in petroleum ether) to give tert-butyl 2-((1-(2-(1,3-dimethyl-1H-indazol-5-yl)-3,6-dimethyl-4-oxo-3,4-dihydroquinazolin-8-yl)ethyl)amino)benzoate (350 mg, 94%) as a white solid. ¹H NMR (400 MHz, DMSO-d₆) δ 8.23 (d, J=6.8 Hz, 1H), 8.14 (s, 1H), 7.88 (d, J=0.8 Hz, 1H), 7.80-7.77 (m, 1H), 7.74-7.71 (m, 2H), 7.59 (d, J=1.6 Hz, 1H), 7.21-7.15 (m, 1H), 6.54-6.40 (m, 2H), 5.51-5.43 (m, 1H), 3.47 (s, 3H), 2.53 (s, 3H), 2.40 (s, 3H), 1.99 (s, 3H), 1.57 (d, J=6.8 Hz, 3H), 1.52 (s, 9H). MS: m/z 538.2 (M+H⁺).

### Step 6—Synthesis of 2-((1-(2-(1,3-dimethyl-1H-indazol-5-yl)-3,6-dimethyl-4-oxo-3,4-dihydroquinazolin-8-yl)ethyl)amino)benzoic acid

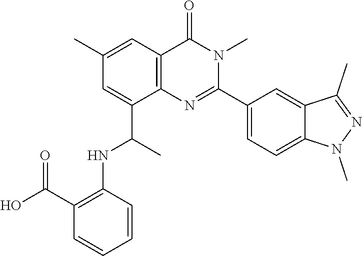 [SMILES: Cc1cc(C(C)Nc2ccccc2C(=O)O)c2nc(-c3ccc4c(c3)c(C)nn4C)n(C)c(=O)c2c1]

To a mixture of tert-butyl 2-((1-(2-(1,3-dimethyl-1H-indazol-5-yl)-3,6-dimethyl-4-oxo-3,4-dihydroquinazolin-8-yl)ethyl)amino)benzoate (350 mg, 651 μmol) in DCM (3 mL) was added TFA (0.5 mL, 6.9 mmol). The reaction mixture was stirred at room temperature for 16 hours. The mixture was concentrated in vacuo. The residue was purified by reverse phase chromatography (acetonitrile 51%-81%/0.225% formic acid in water) to give 2-((1-(2-(1,3-dimethyl-1H-indazol-5-yl)-3,6-dimethyl-4-oxo-3,4-dihydroquinazolin-8-yl)ethyl)amino)benzoic acid (150 mg, 41%) as a white solid. ¹H NMR (400 MHz, DMSO-d₆) δ 12.68 (s, 1H), 8.40 (s, 1H), 8.15 (s, 1H), 7.88 (s, 1H), 7.78 (s, 1H), 7.78-7.75 (m, 1H), 7.74-7.71 (m, 1H), 7.57 (d, J=1.6 Hz, 1H), 7.17 (s, 1H), 6.55-6.39 (m, 2H), 5.51-5.49 (m, 1H), 4.02 (s, 3H), 3.46 (s, 3H), 2.53 (s, 3H), 2.39 (s, 3H), 1.54 (d, J=6.8 Hz, 3H). MS: m/z 482.3 (M+H⁺).

### Step 7—Synthesis of (R)-2-((1-(2-(1,3-dimethyl-1H-indazol-5-yl)-3,6-dimethyl-4-oxo-3,4-dihydroquinazolin-8-yl)ethyl)amino)benzoic acid & (S)-2-((1-(2-(1,3-dimethyl-1H-indazol-5-yl)-3,6-dimethyl-4-oxo-3,4-dihydroquinazolin-8-yl)ethyl)amino)benzoic acid

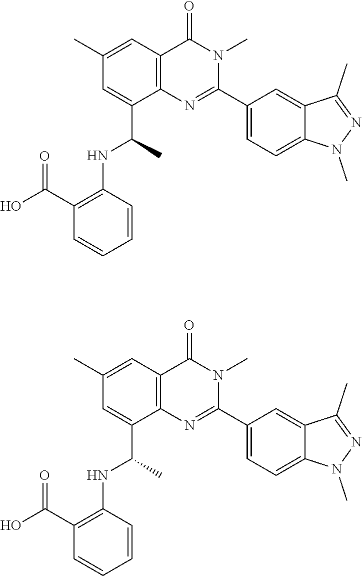 [SMILES: Cc1cc([C@@H](C)Nc2ccccc2C(=O)O)c2nc(-c3ccc4c(c3)c(C)nn4C)n(C)c(=O)c2c1 ; Cc1cc([C@H](C)Nc2ccccc2C(=O)O)c2nc(-c3ccc4c(c3)c(C)nn4C)n(C)c(=O)c2c1]

2-((1-(2-(1,3-dimethyl-1H-indazol-5-yl)-3,6-dimethyl-4-oxo-3,4-dihydroquinazolin-8-yl)ethyl)amino)benzoic acid (150 mg, 312 μmol) was separated by using chiral SFC (DAICEL CHIRALCEL AD (250 mm*30 mm, 10 um); Supercritical CO₂/EtOH+0.1% NH₃·H₂O=55/45; 70 mL/min) to afford (R)-2-((1-(2-(1,3-dimethyl-1H-indazol-5-yl)-3,6-dimethyl-4-oxo-3,4-dihydroquinazolin-8-yl)ethyl)amino)benzoic acid (60 mg, first peak) and (S)-2-((1-(2-(1,3-dimethyl-1H-indazol-5-yl)-3,6-dimethyl-4-oxo-3,4-dihydroquinazolin-8-yl)ethyl)amino)benzoic acid (59 mg, second peak) both as white solid. Absolute configuration was arbitrarily assigned to each enantiomer. Second peak: ¹H NMR (400 MHz, DMSO-d₆) δ 8.51-8.35 (m, 1H), 8.15 (s, 1H), 7.88 (s, 1H), 7.82-7.70 (m, 3H), 7.57 (d, J=1.8 Hz, 1H), 7.23-7.10 (m, 1H), 6.54-6.40 (m, 2H), 5.53-5.42 (m, 1H), 4.03 (s, 3H), 3.47 (s, 3H), 2.53 (s, 3H), 2.39 (s, 3H), 1.54 (d, J=6.8 Hz, 3H). MS: m/z 482.2 (M+H⁺).

### Example 6: Synthesis of Compound 62: 2-((1-(4-ethoxy-2-(isoindolin-2-yl)-6-methylquinazolin-8-yl)ethyl)amino)benzoic acid

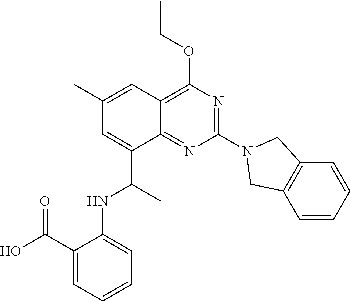 [SMILES: CCOc1nc(N2Cc3ccccc3C2)nc2c(C(C)Nc3ccccc3C(=O)O)cc(C)cc12]

### Step 1—Synthesis of methyl 2-amino-3-bromo-5-methylbenzoate

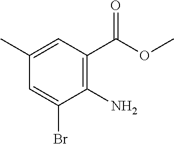

To a solution of 2-amino-3-bromo-5-methyl-benzoic acid (30 g, 130.4 mmol) in MeOH (100 mL) was slowly added concentrated H₂SO₄ (30 mL, 562.8 mmol) dropwise with stirring at room temperature. After the addition, the reaction was heated to 50° C. and stirred for 16 h. After cooling to room temperature, the reaction solution was slowly poured into ice water (60 mL). The mixture was adjusted to pH 7 with aqueous NaOH (2M), then extracted with EtOAc (200 mL×3). The combined organic layers were washed with brine (200 mL), dried over anhydrous Na₂SO₄, filtered and concentrated in vacuo. The crude residue was purified by silica gel chromatography (solvent gradient: 0-30% EtOAc in petroleum ether) to give the title compound (30 g, 94% yield) as a yellow solid. ¹H NMR (400 MHz, DMSO-d₆) δ 7.59 (s, 1H), 7.51 (s, 1H), 6.47 (s, 2H), 3.81 (s, 3H), 2.17 (s, 3H).

### Step 2—Synthesis of 8-bromo-2-(isoindolin-2-yl)-6-methylquinazolin-4(3H)-one

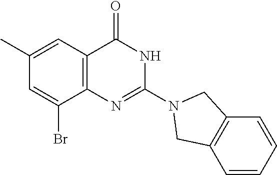 [SMILES: Cc1cc(Br)c2nc(N3Cc4ccccc4C3)[nH]c(=O)c2c1]

To a solution of methyl 2-amino-3-bromo-5-methyl-benzoate (15 g, 61.45 mmol) and isoindoline-2-carbonitrile (13.29 g, 92.18 mmol) in THF (30 mL) was added NaH (4.92 g, 122.91 mmol) with stirring at room temperature. The mixture was heated to 70° C. for 3 h under N₂ atmosphere. After cooling to room temperature, the reaction mixture was quenched with sat. aq. NH₄Cl (40 mL), then extracted with EtOAc (100 mL×3). The combined organic layers were washed with brine (100 mL), dried over anhydrous Na₂SO₄, filtered and concentrated in vacuo. The crude residue was purified by silica gel chromatography (solvent gradient: 0-30% EtOAc in petroleum ether) to give the title compound (11 g, 50%) as a yellow solid. ¹H NMR (400 MHz, DMSO-d₆) δ 11.49 (s, 1H), 7.79 (s, 1H), 7.75 (s, 1H), 7.43-7.40 (m, 2H), 7.34-7.31 (m, 2H), 4.90 (s, 4H), 2.34 (s, 3H).

### Step 3—Synthesis of 8-acetyl-2-(isoindolin-2-yl)-6-methylquinazolin-4(3H)-one

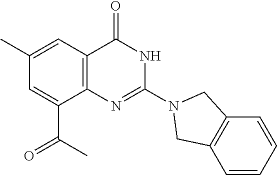 [SMILES: CC(=O)c1cc(C)cc2c(=O)[nH]c(N3Cc4ccccc4C3)nc12]

A mixture of 8-bromo-2-(isoindolin-2-yl)-6-methylquinazolin-4(3H)-one (24.0 g, 67.4 mmol), tributyl(1-ethoxyvinyl)stannane (37.8 g, 104.6 mmol), Pd(dppf)Cl₂ (2.46 g, 3.37 mmol) in dioxane (300 mL) was stirred at 90° C. for 16 h under N₂ atmosphere. After cooling to room temperature, the mixture was quenched with 200 mL of 10% KF aqueous solution and stirred for 30 min. The mixture was extracted with EtOAc (300 mL×2). The combined organic layers were washed with brine (300 mL), dried over Na₂SO₄, filtered and concentrated in vacuo to give a crude product (23 g) as a yellow solid. The crude product was dissolved in dioxane (300 mL), then added aqueous HCl (1M, 40 mL). The mixture was stirred at room temperature for 2 h. The reaction mixture was filtered to give the title compound (21 g, crude) as a gray solid that required no further purification. ¹H NMR (400 MHz, DMSO-d₆) δ 8.07 (s, 1H), 7.54-7.30 (m, 5H), 4.98 (s, 4H), 2.84 (s, 3H), 2.42 (s, 3H). MS: m/z 320.2 (M+H⁺).

### Step 4—Synthesis of 8-(1-hydroxyethyl)-2-(isoindolin-2-yl)-6-methylquinazolin-4(3H₁)-one

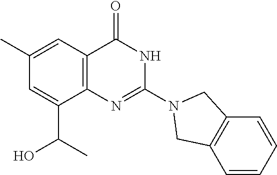 [SMILES: Cc1cc(C(C)O)c2nc(N3Cc4ccccc4C3)[nH]c(=O)c2c1]

To a solution of 8-acetyl-2-(isoindolin-2-yl)-6-methylquinazolin-4(3H)-one (16.0 g, 50.1 mmol) in MeOH (30 mL) was added NaBH₄ (3.61 g, 95.4 mmol) at 0° C. The mixture was stirred at room temperature for 2 h under N₂ atmosphere. The mixture was quenched with sat. aq. NH₄Cl (30 mL) at 0° C., diluted with water (30 mL). The mixture was stirred at 0° C. for 10 min. The suspension solution was filtered, the filter cake was washed with water (30 mL×2) and dried in vacuo to give the title compound (14 g, crude) as a yellow solid that required no further purification. ¹H NMR (400 MHz, DMSO-d₆) δ 10.62 (s, 1H), 7.62 (s, 1H), 7.55 (s, 1H), 7.44-7.39 (m, 2H), 7.34-7.30 (m, 2H), 5.45-5.33 (m, 1H), 5.23 (s, 1H), 4.87 (s, 4H), 2.34 (s, 3H), 1.41 (d, J=6.0 Hz, 3H). MS: m/z 322.2 (M+H⁺).

### Step 5—Synthesis of 8-(1-bromoethyl)-2-(isoindolin-2-yl)-6-methylquinazolin-4(3H)-one

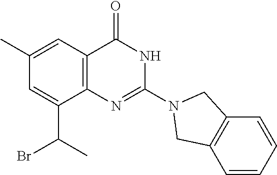 [SMILES: Cc1cc(C(C)Br)c2nc(N3Cc4ccccc4C3)[nH]c(=O)c2c1]

To a solution of 8-(1-hydroxyethyl)-2-(isoindolin-2-yl)-6-methylquinazolin-4(3H)-one (1.1 g, 3.4 mmol) in DCM (33 mL) was slowly added PBr₃ (926 mg, 3.4 mmol) at 0° C. under N₂ atmosphere. The mixture was stirred at 0° C. for 1 h. After the reaction was completed, the reaction mixture was concentrated to give the title compound (1.31 g, crude) that required no further purification.

### Step 6—Synthesis of tert-butyl 2-((1-(2-(isoindolin-2-yl)-6-methyl-4-oxo-3,4-dihydroquinazolin-8-yl)ethyl)amino)benzoate

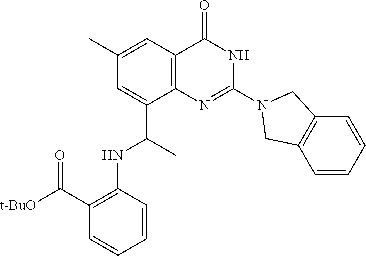 [SMILES: Cc1cc(C(C)Nc2ccccc2C(=O)OC(C)(C)C)c2nc(N3Cc4ccccc4C3)[nH]c(=O)c2c1]

The solution of 8-(1-bromoethyl)-2-(isoindolin-2-yl)-6-methylquinazolin-4(3H)-one (1.31 g, 3.4 mmol) in dioxane (11 mL) was adjusted to pH 8 with DIPEA at 0° C. The mixture was added tert-butyl 2-aminobenzoate (1.32 g, 6.8 mmol) and heated to 90° C. for 16 h under N₂ atmosphere. After cooling to room temperature, the reaction mixture was concentrated and the residue was purified by silica gel chromatography (solvent gradient: 0-50% EtOAc in petroleum ether) to give the title compound (1 g, 40%) as a yellow solid. ¹H NMR (400 MHz, DMSO-d₆) δ 11.37 (s, 1H), 8.34-8.27 (m, 1H), 7.75-7.70 (m, 2H), 7.66-7.62 (m, 1H), 7.41-7.40 (m, 2H), 7.34-7.32 (m, 2H), 7.25-7.17 (m, 1H), 6.61-6.55 (m, 1H), 6.50-6.44 (m, 1H), 5.42-5.35 (m, 1H), 5.02-4.90 (m, 4H), 2.28 (s, 3H), 1.61 (d, J=6.8 Hz, 3H), 1.53 (s, 9H). MS: m/z 497.3 (M+H⁺).

### Step 7—Synthesis of tert-butyl 2-((1-(4-ethoxy-2-(isoindolin-2-yl)-6-methylquinazolin-8-yl)ethyl)amino)benzoate

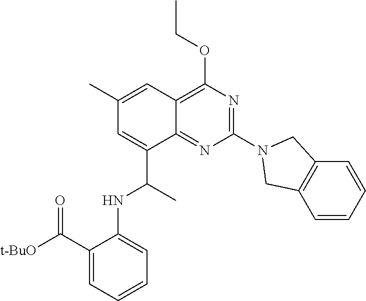 [SMILES: CCOc1nc(N2Cc3ccccc3C2)nc2c(C(C)Nc3ccccc3C(=O)OC(C)(C)C)cc(C)cc12]

To a solution of tert-butyl 2-((1-(4-hydroxy-2-(isoindolin-2-yl)-6-methylquinazolin-8-yl)ethyl)amino)benzoate (30 mg, 60 umol) in DMF (3 mL) was added K₂CO₃ (25 mg, 0.18 mmol) and iodoethane (10 mg, 66 μmol). The mixture was stirred at room temperature for 3 h. After the reaction was completed, the reaction was added water (20 mL) and extracted with EtOAc (20 mL×3). The combined organic layers were washed with brine (20 mL×3), dried over anhydrous Na₂SO₄, filtered and concentrated in vacuo. The crude residue was purified by silica gel chromatography (solvent gradient: 0-10% EtOAc in petroleum ether) to give the title compound (23 mg, 73%) as yellow oil, which was further confirmed by 2D NMR (¹³C NMR). ¹H NMR (400 MHz, DMSO-d₆) δ 8.40 (d, J=7.6 Hz, 1H), 7.75-7.70 (m, 1H), 7.60 (s, 1H), 7.49-7.45 (m, 3H), 7.35-7.31 (m, 2H), 7.22-7.15 (m, 1H), 6.60 (d, J=8.4 Hz, 1H), 6.49-6.45 (m, 1H), 5.51-5.47 (m, 1H), 5.02-4.89 (m, 4H), 4.64 (q, J=7.2 Hz, 2H), 2.33 (s, 3H), 1.67 (d, J=6.8 Hz, 3H), 1.53 (s, 9H), 1.48 (t, J=7.2 Hz, 3H). MS: m/z 525.3 (M+H⁺).

### Step 8—Synthesis of 2-((1-(4-ethoxy-2-(isoindolin-2-yl)-6-methylquinazolin-8-yl)ethyl)amino)benzoic acid

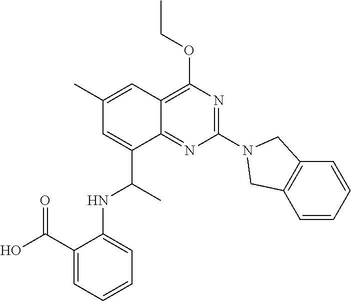 [SMILES: CCOc1nc(N2Cc3ccccc3C2)nc2c(C(C)Nc3ccccc3C(=O)O)cc(C)cc12]

To a mixture of tert-butyl 2-((1-(4-ethoxy-2-(isoindolin-2-yl)-6-methylquinazolin-8-yl)ethyl)amino)benzoate (70 mg, 80 umol) in DCM (3 mL) was added TFA (0.5 mL, 6.9 mmol).

The reaction mixture was stirred at room temperature for 16 h. The mixture was concentrated and the residue was purified by reverse phase chromatography (acetonitrile 75%-100%/0.225% formic acid in water) to give the title compound (7 mg, 19%) as a brown solid. ¹H NMR (400 MHz, DMSO-d₆) δ 12.63 (s, 1H), 8.63 (s, 1H), 7.77 (d, J=8.4 Hz, 1H), 7.60 (s, 1H), 7.50-7.43 (m, 3H), 7.36-7.33 (m, 2H), 7.23-7.16 (m, 1H), 6.56 (d, J=8.8 Hz, 1H), 6.47 (t, J=7.6 Hz, 1H), 5.54-5.50 (m, 1H), 5.11-4.87 (m, 4H), 4.63 (q, J=7.2 Hz, 2H), 2.33 (s, 3H), 1.65 (d, J=6.4 Hz, 3H), 1.48 (t, J=7.2 Hz, 3H). MS: m/z 469.1 (M+H⁺).

### Example 7: Synthesis of Compound 63: 2-((1-(4-(3-hydroxyazetidin-1-yl)-2-(isoindolin-2-yl)-6-methylquinazolin-8-yl)ethyl)amino)benzoic acid

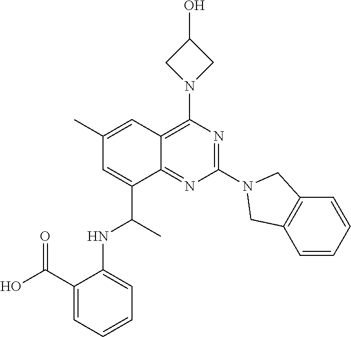 [SMILES: Cc1cc(C(C)Nc2ccccc2C(=O)O)c2nc(N3Cc4ccccc4C3)nc(N3CC(O)C3)c2c1]

### Step 1—Synthesis of tert-butyl 2-((1-(4-(3-hydroxyazetidin-1-yl)-2-(isoindolin-2-yl)-6-methylquinazolin-8-yl)ethyl)amino)benzoate

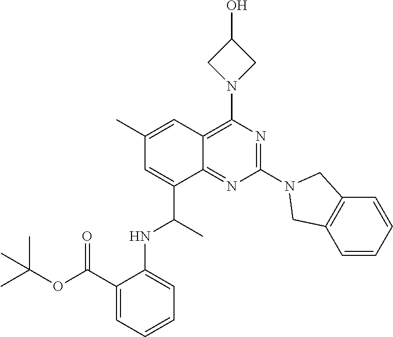 [SMILES: Cc1cc(C(C)Nc2ccccc2C(=O)OC(C)(C)C)c2nc(N3Cc4ccccc4C3)nc(N3CC(O)C3)c2c1]

To a solution of tert-butyl 2-((1-(4-hydroxy-2-(isoindolin-2-yl)-6-methylquinazolin-8-yl)ethyl)amino)benzoate (150 mg, 302 μmol) in acetonitrile (6 mL) was added BOP (200 mg, 453 mol) and DBU (138 mg, 906 μmol). The mixture was stirred at room temperature for 10 min, azetidin-3-ol (44 mg, 604 μmol,) was added. The final mixture was stirred at room temperature for 2 h. The reaction mixture was concentrated in vacuo and the residue was purified by silica gel chromatography (solvent gradient: 0-5% MeOH in DCM) to give the title compound (110 mg, 81%) as a white solid. MS: m/z 552.1 (M+H⁺).

### Step 2—Synthesis of 2-((1-(4-(3-hydroxyazetidin-1-yl)-2-(isoindolin-2-yl)-6-methylquinazolin-8-yl)ethyl)amino)benzoic acid

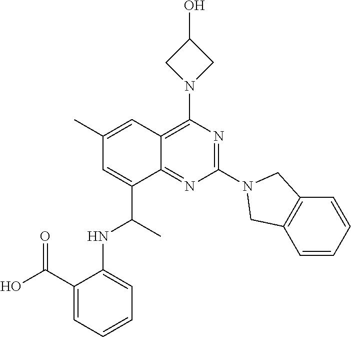 [SMILES: Cc1cc(C(C)Nc2ccccc2C(=O)O)c2nc(N3Cc4ccccc4C3)nc(N3CC(O)C3)c2c1]

To a mixture of tert-butyl 2-((1-(4-(3-hydroxyazetidin-1-yl)-2-(isoindolin-2-yl)-6-methylquinazolin-8-yl)ethyl)amino)benzoate (70 mg, 80 umol) in DCM (2 mL) was added TFA (0.5 mL, 6.9 mmol). The reaction mixture was stirred at room temperature for 16 h. The mixture was concentrated and the residue was purified by reverse phase chromatography (acetonitrile 26%-56%/0.225% formic acid in water) to give the title compound (11 mg, 56%) as a white solid. ¹H NMR (400 MHz, DMSO-d₆) δ 7.78-7.73 (m, 1H), 7.65-7.53 (m, 1H), 7.47-7.39 (m, 3H), 7.35-7.28 (m, 3H), 7.20-7.12 (m, 1H), 6.53 (d, J=8.0 Hz, 1H), 6.47-6.43 (m, 1H), 5.81-5.77 (m, 1H), 5.57-5.48 (m, 1H), 4.98-4.81 (m, 4H), 4.72-4.65 (m, 2H), 4.23-4.13 (m, 2H), 2.29 (s, 3H), 1.61 (d, J=6.4 Hz, 3H). MS: m/z 496.1 (M+H⁺).

### Example 8: Preparation of (S)-2-((1-(2-(isoindolin-2-yl)-6-methyl-4-oxo-4H-thiochromen-8-yl)ethyl)amino)benzoic acid (Compound 159) and (S)-2-((1-(2-(isoindolin-2-yl)-6-methyl-4-oxo-4H-thiochromen-8-yl)ethyl)amino)benzoic acid (Compound 157)

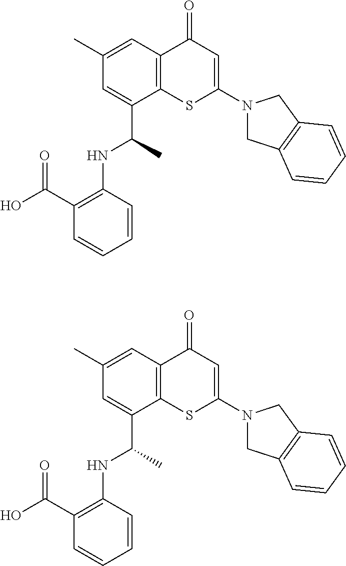 [SMILES: Cc1cc([C@@H](C)Nc2ccccc2C(=O)O)c2sc(N3Cc4ccccc4C3)cc(=O)c2c1 ; Cc1cc([C@H](C)Nc2ccccc2C(=O)O)c2sc(N3Cc4ccccc4C3)cc(=O)c2c1]

### Step 1: Synthesis of 1-(3-bromo-2-fluoro-5-methylphenyl)ethan-1-ol

To a solution of 3-bromo-2-fluoro-5-methyl-benzaldehyde (3 g, 13.8 mmol) in THF (20 mL) was added MeMgBr (3 M in THF, 9.22 mL) at 0° C. The reaction was stirred at 0° C. for 2 h under a N₂ atmosphere. The mixture was quenched with sat. aq. NH₄Cl (20 mL), diluted with water (20 mL), extracted with EtOAc (30 mL×2). The combined organic layers were washed with brine (20 mL), dried over anhydrous Na₂SO₄, filtered and concentrated in vacuo. The crude residue was purified by silica gel chromatography (solvent gradient: 0-20% EtOAc in petroleum ether) to give the title compound (2.5 g, 78%) as a yellow solid. ¹H NMR (400 MHz, CDCl₃): δ 7.25-7.12 (m, 2H), 5.08 (q, J=6.4 Hz, 1H), 1.97 (s, 3H), 1.42 (d, J=6.4 Hz, 3H).

### Step 2: Synthesis of 1-(3-bromo-2-fluoro-5-methylphenyl)ethan-1-one

To a solution of 1-(3-bromo-2-fluoro-5-methylphenyl)ethan-1-ol (2.8 g, 12.01 mmol) in DCM (20 mL) was added Dess-Martin (7.6 g, 18 mmol). The reaction mixture was stirred at room temperature for 2 h. The mixture was quenched with sat. aq. Na₂S₂O₃ (20 mL), diluted with water (20 mL), extracted with DCM (30 mL×2). The combined organic layers were washed with brine (20 mL), dried over anhydrous Na₂SO₄, filtered and concentrated in vacuo. The crude residue was purified by silica gel chromatography (solvent gradient: 0-20% EtOAc in petroleum ether) to give the title compound (2.6 g, 93%) as a yellow solid. ¹H NMR (400 MHz, CDCl₃) δ=7.59-7.39 (m, 2H), 2.64 (d, J=5.2 Hz, 3H), 2.34 (s, 3H).

### Step 3: Synthesis of 8-bromo-2-mercapto-6-methyl-4H-thiochromen-4-one

To a solution of 1-(3-bromo-2-fluoro-5-methylphenyl)ethan-1-one (3.1 g, 13.42 mmol) in DMF (12 mL) and PhMe (8 mL) was added NaH (1.07 g, 26.8 mmol, 60% purity) and CS₂ (2.04 g, 26.8 mmol). The mixture was stirred at room temperature for 2 h. The reaction was quenched with sat. aq. NH₄Cl (20 mL), diluted with water (20 mL), extracted with EtOAc (30 mL×2). The combined organic layers were washed with brine (20 mL), dried over anhydrous Na₂SO₄, filtered and concentrated in vacuo to give the title compound (3 g, 78%) as a yellow solid. MS: m/z 572.8 (2M+H⁺).

### Step 4: Synthesis of 8-bromo-2-(ethylthio)-6-methyl-4H-thiochromen-4-one

To a solution of 8-bromo-2-mercapto-6-methyl-4H-thiochromen-4-one (2.5 g, 8.7 mmol) in DCM (20 mL) was added EtI (2.72 g, 17.41 mmol) and Et₃N (2.4 mL, 17.41 mmol). The mixture was stirred at room temperature for 2 h. The reaction was concentrated in vacuo. The crude residue was purified by silica gel chromatography (solvent gradient: 0-50% EtOAc in petroleum ether) to give the title compound (2.5 g, 91%) as a yellow solid. ¹H NMR (400 MHz, DMSO-d₆): δ 8.21-8.08 (m, 1H), 8.01-7.88 (m, 1H), 6.99 (s, 1H), 3.30-3.24 (m, 2H), 2.45-2.41 (m, 3H), 1.36-1.29 (m, 3H). MS: m/z 315.0 (M+H⁺).

### Step 5: Synthesis of 8-bromo-2-(ethylsulfonyl)-6-methyl-4H-thiochromen-4-one

To a solution of 8-bromo-2-(ethylthio)-6-methyl-4H-thiochromen-4-one (2.3 g, 7.20 mmol) in AcOH (15 mL) was added H₂O₂ (1.80 g, 15.84 mmol). The mixture was stirred at 60° C. for 2 h. After cooling to room temperature, the mixture was filtered. The crude filter was concentrated in vacuo to give title compound (2.4 g, crude) as a yellow solid. MS: m/z 348.9 (M+H⁺).

### Step 6: Synthesis of 8-bromo-2-(isoindolin-2-yl)-6-methyl-4H-thiochromen-4-one

To a solution of 8-bromo-2-(ethylsulfonyl)-6-methyl-4H-thiochromen-4-one (1.2 g, 3.46 mmol) in t-BuOH (10 mL) was added isoindoline (823 mg, 6.9 mmol). The mixture was stirred at 130° C. for 36 h. After cooling to room temperature, the reaction was concentrated in vacuo. The crude residue was purified by silica gel chromatography (solvent gradient: 0-50% EtOAc in petroleum ether) to give the title compound (1.2 g, 93%) as a black solid. ¹H NMR (400 MHz, DMSO-d₆): δ 8.14 (s, 1H), 7.83 (s, 1H), 7.45-7.42 (m, 2H), 7.38-7.34 (m, 2H), 5.89 (s, 1H), 4.88 (s, 4H), 2.41 (s, 3H). MS: m/z 371.8 (M+H⁺).

### Step 7: Synthesis of 8-acetyl-2-(isoindolin-2-yl)-6-methyl-4H-thiochromen-4-one

To a solution of 8-bromo-2-(isoindolin-2-yl)-6-methyl-4H-thiochromen-4-one (1 g, 2.69 mmol) in dioxane (10 mL) was added Pd(PPh₃)₂Cl₂ (188 mg, 268 μmol) and tributyl(1-ethoxyvinyl)stannane (1.94 g, 5.37 mmol). The mixture was stirred at 100° C. for 16 h under N₂ atmosphere. After cooling to room temperature. Aq. HCl (1M, 2 mL) was added and the mixture was stirred at 50° C. for additional 3 h. After cooling to room temperature, the mixture was concentrated in vacuo. The crude residue was purified by silica gel chromatography (solvent gradient: 0-30% EtOAc in petroleum ether) to give the title compound (800 mg, 88%) as colorless oil. ¹H NMR (400 MHz, DMSO-d₆): δ 8.43 (s, 1H), 8.29 (s, 1H), 7.45-7.42 (m, 2H), 7.37-7.34 (m, 2H), 5.76-5.75 (m, 1H), 4.83 (s, 4H), 2.72 (s, 3H), 2.47 (s, 3H). MS: m/z 336.1 (M+H⁺).

### Step 8: Synthesis of 8-(1-hydroxyethyl)-2-(isoindolin-2-yl)-6-methyl-4H-thiochromen-4-one

To a solution of 8-acetyl-2-(isoindolin-2-yl)-6-methyl-4H-thiochromen-4-one in MeOH (5 mL) was added NaBH₄ (45 mg, 1.2 mmol) at 0° C. The mixture was stirred at 0° C. for 2 h under N₂ atmosphere. The reaction was quenched with sat. aq. NH₄Cl (2 mL), diluted with water (20 mL), extracted with EtOAc (10 mL×2). The combined organic layers were washed with brine (20 mL), dried over anhydrous Na₂SO₄, filtered and concentrated in vacuo. The crude residue was purified by silica gel chromatography (solvent gradient: 0-50% EtOAc in petroleum ether) to give the title compound (0.18 g, 89%) as a yellow solid. ¹H NMR (400 MHz, CD₃OD): δ 8.16, (s, 1H), 8.13 (s, 1H), 7.44-7.40 (m, 2H), 7.35-7.32 (m, 2H), 6.03 (s, 1H), 5.49 (s, 4H), 4.77-4.74 (m, 1H), 2.41 (s, 3H), 1.61-1.54 (m, 3H). MS: m/z 338.2 (M+H⁺).

### Step 9: Synthesis of 8-(1-bromoethyl)-2-(isoindolin-2-yl)-6-methyl-4H-thiochromen-4-one

To a solution of 8-(1-hydroxyethyl)-2-(isoindolin-2-yl)-6-methyl-4H-thiochromen-4-one (150 mg, 444 umol) in DCM (3 mL) was added PBr₃ (120 mg, 666 umol) at 0° C. The mixture was stirred at 0° C. for 1 h under N₂ atmosphere. The reaction was concentrated in vacuo to give the title compound (2.25 g, crude) as a yellow solid. MS: m/z 400.1 (M+H⁺).

### Step 10: Synthesis of tert-butyl 2-((1-(2-(isoindolin-2-yl)-6-methyl-4-oxo-4H-thiochromen-8-yl)ethyl)amino)benzoate

To a solution of 8-(1-bromoethyl)-2-(isoindolin-2-yl)-6-methyl-4H-thiochromen-4-one (50 mg, 125 umol) in DMF (2 mL) was added tert-butyl 2-aminobenzoate (48 mg, 250 umol). The mixture was stirred at 100° C. for 2 h. The reaction was concentrated in vacuo. The crude residue was purified by silica gel chromatography (solvent gradient: 0-50% EtOAc in petroleum ether) to give the title compound (25 mg, 39%) as a white solid. ¹H NMR (400 MHz, DMSO-d₆): δ 8.29 (d, J=5.6 Hz, 1H), 8.05 (m, 1H), 7.78 (d, J=6.8 Hz, 1H), 7.51 (s, 1H), 7.45-7.32 (m, 4H), 7.25-7.18 (m, 1H), 6.61-6.52 (m, 1H), 6.32 (d, J=8.4 Hz, 1H), 5.90 (s, 1H), 5.04-4.93 (m, 1H), 4.91-4.80 (m, 4H), 2.34 (s, 3H), 1.66 (d, J=6.8 Hz, 3H), 1.60 (s, 9H). MS: m/z 513.1 (M+H⁺).

### Step 11: Synthesis of 2-((1-(2-(isoindolin-2-yl)-6-methyl-4-oxo-4H-thiochromen-8-yl)ethyl)amino)benzoic acid

To a solution of tert-butyl 2-((1-(2-(isoindolin-2-yl)-6-methyl-4-oxo-4H-thiochromen-8-yl)ethyl)amino)benzoate (25 mg, 49 umol) in DCM (2 mL) was added TFA (1 mL). The mixture was stirred at room temperature for 16 h. The reaction was concentrated in vacuo. The crude residue was purified by silica gel chromatography (solvent gradient: 0-10% MeOH in DCM (1% AcOH)) to give the title compound (20 mg, 89%) as a white solid. ¹H NMR (400 MHz, DMSO-d₆): δ 12.85 (s, 1H), 8.50 (d, J=5.2 Hz, 1H), 8.11 (s, 1H), 7.88-7.78 (m, 1H), 7.50-7.47 (m, 1H), 7.47-7.42 (m, 2H), 7.40-7.34 (m, 2H), 7.23-7.21 (m, 1H), 6.62-6.48 (m, 1H), 6.35-6.24 (m, 1H), 5.92 (s, 1H), 5.03-4.95 (m, 1H), 4.93-4.83 (m, 4H), 2.34 (s, 3H), 1.63 (d, J=6.8 Hz, 1H). MS: m/z 457.1 (M+H⁺).

### Step 12: Synthesis of (R)-2-((1-(2-(isoindolin-2-yl)-6-methyl-4-oxo-4H-thiochromen-8-yl)ethyl)amino)benzoic acid (Compound 159) and (S)-2-((1-(2-(isoindolin-2-yl)-6-methyl-4-oxo-4H-thiochromen-8-yl)ethyl)amino)benzoic acid (Compound 157)

2-((1-(2-(isoindolin-2-yl)-6-methyl-4-oxo-4H-thiochromen-8-yl)ethyl)amino)benzoic acid (20 mg, 43 umol) was separated by using chiral SFC (DAICEL CHIRALCEL J (250 mm*30 mm, 10 um); Supercritical CO₂/EtOH+0.1% NH₃H₂O=55/45; 60 ml/min) to afford (R)-2-((1-(2-(isoindolin-2-yl)-6-methyl-4-oxo-4H-thiochromen-8-yl)ethyl)amino)benzoic acid (8 mg, first peak) and (S)-2-((1-(2-(isoindolin-2-yl)-6-methyl-4-oxo-4H-thiochromen-8-yl)ethyl)amino)benzoic acid (7 mg, second peak) both as white solid. Absolute configuration was arbitrarily assigned to each enantiomer.

Compound 159: ¹H NMR (400 MHz, DMSO-d₆): δ 12.87 (s, 1H), 8.59 (s, 1H), 8.06 (s, 1H), 7.83 (d, J=5.6 Hz, 1H), 7.50-7.47 (m, 1H), 7.47-7.42 (m, 2H), 7.40-7.34 (m, 2H), 7.22-7.20 (m, 1H), 6.62-6.48 (m, 1H), 6.35-6.24 (m, 1H), 5.93 (s, 1H), 5.03-4.95 (m, 1H), 4.93-4.83 (m, 4H), 2.34 (s, 3H), 1.63 (d, J=6.8 Hz, 1H). MS: m/z 457.1 (M+H⁺).

### Example 9: Preparation of (R)-2-((1-(6-methyl-4-oxo-2-(pyridin-4-yl)-4H-pyrano[2,3-c]pyridin-8-yl)ethyl)amino)benzoic acid trifluoroacetate (Compound 158)

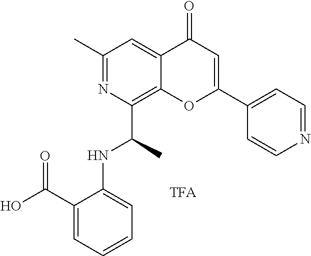 [SMILES: Cc1cc2c(=O)cc(-c3ccncc3)oc2c([C@@H](C)Nc2ccccc2C(=O)O)n1]

### Step 1: Synthesis of 2,4-dibromo-3-(methoxymethoxy)-6-methylpyridine

To a solution of 2,4-dibromo-6-methyl-pyridin-3-ol (3.4 g, 12.7 mmol) in DCM (30 mL) was added DIEA (3.33 mL, 19.1 mmol) and bromo(methoxy)methane (1.25 mL, 15.3 mmol) dropwise at room temperature. After the addition, the reaction was stirred at room temperature for 2 h. The reaction was quenched with water (20 mL), extracted with DCM (30 mL×2). The combined organic layers were washed with brine (20 mL), dried over anhydrous Na₂SO₄ and filtered. The filtrate was concentrated in vacuo. The crude residue was purified by silica gel chromatography (solvent gradient: 0-5% ethyl acetate in petroleum ether) to give the title compound (3.7 g, 93%) as colorless oil. ¹H NMR (400 MHz, CDCl₃): δ 7.34 (s, 1H), 5.19 (s, 2H), 3.73 (s, 3H), 2.50 (s, 3H).

### Step 2: Synthesis of 1-(2-bromo-3-(methoxymethoxy)-6-methylpyridin-4-yl)ethan-1-ol

To a solution of 2,4-dibromo-3-(methoxymethoxy)-6-methylpyridine (3.7 g, 11.9 mmol) in THF (40 mL) was added n-BuLi (2.5 M, 5.71 mL) dropwise at −78° C. under N₂ atmosphere. After the addition, the mixture was stirred at −78° C. for 10 min. Then acetaldehyde (5 M, 7.14 mL) was added to the mixture dropwise. The mixture was stirred at room temperature for 16 h. The mixture was quenched with sat. aq. NH₄Cl (30 mL) and extracted with EtOAc (30 mL×2). The combined organic layers were washed with brine (20 mL), dried over anhydrous Na₂SO₄ and filtered. The filtrate was concentrated in vacuo. The crude residue was purified by silica gel chromatography (solvent gradient: 0-20% ethyl acetate in petroleum ether) to give the title compound (1.67 g, 51%) as yellow oil. ¹H NMR (400 MHz, CDCl₃): δ 7.22 (s, 1H), 5.22-5.16 (m, 2H), 5.10 (d, J=6.4 Hz, 1H), 3.64 (s, 3H), 2.86 (d, J=3.6 Hz, 1H), 2.52 (s, 3H), 1.51 (d, J=6.4 Hz, 3H). MS: m/z 276.1 (M+H⁺).

### Step 3: Synthesis of 1-(2-bromo-3-(methoxymethoxy)-6-methylpyridin-4-yl)ethan-1-one

To a solution of 1-(2-bromo-3-(methoxymethoxy)-6-methylpyridin-4-yl)ethan-1-ol (1.67 g, 6.05 mmol) in DCM (30 mL) was added Dess-Martin (3.08 g, 7.26 mmol) in portions at 0° C. After the addition, the mixture was stirred at room temperature for 16 h. The mixture was quenched with sat. aq. Na₂S₂O₃ (20 mL) and washed with sat. aq. NaHCO₃ (100 mL). The mixture was extracted with DCM (100 mL×2). The combined organic layers were washed with brine (100 mL), dried over anhydrous Na₂SO₄ and filtered. The filtrate was concentrated in vacuo. The crude residue was purified by silica gel chromatography (solvent gradient: 0-12% ethyl acetate in petroleum ether) to give the title compound (1.4 g, 84%) as colorless oil. ¹H NMR (400 MHz, CDCl₃): δ 7.13 (s, 1H), 5.09 (s, 2H), 3.51 (s, 3H), 2.61 (s, 3H), 2.55 (s, 3H). MS: m/z 274.1 (M+H⁺).

### Step 4: Synthesis of 1-(2-bromo-3-hydroxy-6-methylpyridin-4-yl)ethan-1-one

To a solution of 1-(2-bromo-3-(methoxymethoxy)-6-methylpyridin-4-yl)ethan-1-one (1 g, 3.65 mmol) in DCM (5 mL) was added HCl (5 mL, 4M in dioxane). After the addition, the reaction mixture was stirred at room temperature for 2 h. The mixture was concentrated to remove the solvent. The residue was dissolved in EtOAc (30 mL), washed with saturated NaHCO₃ (20 mL), brine (20 mL), dried over Na₂SO₄ and filtered. The filtrate was concentrated in vacuo. The crude residue was purified by silica gel chromatography (solvent gradient: 0-12% EtOAc in petroleum ether) to give the title compound (294 mg, 35%) as a yellow solid. ¹H NMR (400 MHz, CDCl₃): δ 12.03 (s, 1H), 7.34 (s, 1H), 2.69 (s, 3H), 2.55 (s, 3H). MS: m/z 230.0 (M+H⁺).

### Step 5: Synthesis of 1-(2-bromo-3-hydroxy-6-methylpyridin-4-yl)-3-(pyridin-4-yl)propane-1,3-dione

To a solution of 1-(2-bromo-3-hydroxy-6-methylpyridin-4-yl)ethan-1-one (1 g, 4.35 mmol) in THF (15 mL) was added LiHMDS (1 M, 13.9 mL) at −78° C. The reaction mixture was stirred at 0° C. for 1 hour. Then the mixture was cooled to −78° C. and to the mixture was added methyl pyridine-4-carboxylate (0.62 mL, 5.22 mmol) dropwise. The mixture was stirred at room temperature for 16 h. The reaction was quenched with aq. HCl (1M, 10 mL) and extracted with EtOAc (30 mL×2). The combined organic layers were washed with brine (20 mL), dried over anhydrous Na₂SO₄ and filtered. The filtrate was concentrated in vacuo to give the title compound (1.34 g, crude) as a yellow solid that required no further purification. MS: m/z 337.0 (M+H⁺).

### Step 6: Synthesis of 8-bromo-6-methyl-2-(pyridin-4-yl)-4H-pyrano[2,3-c]pyridin-4-one

To a mixture of 1-(2-bromo-3-hydroxy-6-methylpyridin-4-yl)-3-(pyridin-4-yl)propane-1,3-dione (1.34 g, 4.00 mmol) in AcOH (10 mL) was added H₂SO₄ (0.42 mL, 8.00 mmol). After the addition, the mixture was stirred at 80° C. for 3 h. The mixture was cooled to room temperature and poured into ice-water (30 mL). The mixture was basified with sat. aq. NaHCO₃ to pH˜7, extracted with EtOAc (50 mL×2). The combined organic layers were washed with brine (20 mL), dried over anhydrous Na₂SO₄ and filtered. The filtrate was concentrated in vacuo. The crude residue was purified by silica gel chromatography (solvent gradient: 0-50% EtOAc in petroleum ether) to give the title compound (696 mg, 55%) as a brown solid. ¹H NMR (400 MHz, DMSO-d₆): δ 8.90-8.83 (m, 2H), 8.09-8.03 (m, 2H), 7.78 (s, 1H), 7.45 (s, 1H), 2.60 (s, 3H). MS: m/z 317.0 (M+H⁺).

### Step 7: Synthesis of 8-(1-ethoxyvinyl)-6-methyl-2-(pyridin-4-yl)-4H-pyrano[2,3-c]pyridin-4-one

A mixture of tributyl(1-ethoxyvinyl)stannane (1.64 g, 4.54 mmol), 8-bromo-6-methyl-2-(pyridin-4-yl)-4H-pyrano[2,3-c]pyridin-4-one (696 mg, 2.19 mmol), Pd(dppf)Cl₂ (160 mg, 219 umol) in dioxane (10 mL) was degassed with N₂ for 3 times and stirred at 100° C. for 16 h. The mixture was cooled to room temperature. The mixture was poured into 10% KF solution (20 mL) and stirred for 30 min. The mixture was extracted with EtOAc (30 mL×2). The combined organic layers were washed with brine (20 mL), dried over anhydrous Na₂SO₄ and filtered. The filtrate was concentrated in vacuo. The crude residue was purified with silica gel chromatography (solvent gradient: 0-60% EtOAc in petroleum ether) to give the title compound (545 mg, 80%) as a brown solid. ¹H NMR (400 MHz, CDCl₃): δ 8.87-8.84 (m, 2H), 8.87-8.84 (m, 1H), 7.88-7.83 (m, 3H), 6.99 (s, 1H), 4.91 (d, J=2.8 Hz, 1H), 4.74 (d, J=2.8 Hz, 1H), 4.17-4.11 (m, 2H), 2.72 (s, 3H), 1.49 (t, J=7.2 Hz, 3H). MS: m/z 309.1 (M+H⁺).

### Step 8: Synthesis of 8-acetyl-6-methyl-2-(pyridin-4-yl)-4H-pyrano[2,3-c]pyridin-4-one

To a solution of 8-(1-ethoxyvinyl)-6-methyl-2-(pyridin-4-yl)-4H-pyrano[2,3-c]pyridin-4-one in THE (10 mL) was added aq. HCl (1M, 3 mL). After the addition, the reaction mixture was stirred at room temperature for 3 h. The mixture was basified with sat. aq. NaHCO₃ to pH 7. Then the mixture was extracted with EtOAc (20 mL×2). The combined organic layers were washed with brine (20 mL), dried over anhydrous Na₂SO₄ and filtered. The filtrate was concentrated to give the title compound (495 mg, 99%) as a yellow solid. ¹H NMR (400 MHz, DMSO-d₆): δ 8.88-8.85 (m, 2H), 8.15-8.12 (m, 2H), 8.04 (s, 1H), 7.47 (s, 1H), 2.74 (s, 3H), 2.68 (s, 3H). MS: m/z 281.1 (M+H⁺).

### Step 9: Synthesis of 8-(1-aminoethyl)-6-methyl-2-(pyridin-4-yl)-4H-pyrano[2,3-c]pyridin-4-one

A mixture of 8-acetyl-6-methyl-2-(pyridin-4-yl)-4H-pyrano[2,3-c]pyridin-4-one (100 mg, 356 umol) and NH₄OAc (275 mg, 3.57 mmol) in MeOH (4 mL) was stirred at room temperature for 1 h. Then to the mixture was added NaBH₃CN (67.3 mg, 1.07 mmol). The resulting mixture was stirred at 60° C. for 4 h. The mixture was concentrated in vacuo. The crude residue was purified by silica gel chromatography (solvent gradient: 0-5% MeOH in DCM) to give the title compound (62 mg, 62%) as a yellow solid. ¹H NMR (400 MHz, DMSO-d₆): δ 8.90-8.82 (m, 2H), 8.27 (s, 2H), 8.12-8.06 (m, 2H), 7.84 (s, 1H), 7.44 (s, 1H), 5.30-5.22 (m, 1H), 2.68 (s, 3H), 1.61 (d, J=6.8 Hz, 3H). MS: m/z 282.1 (M+H⁺).

### Step 10: Synthesis of tert-butyl 2-((1-(6-methyl-4-oxo-2-(pyridin-4-yl)-4H-pyrano[2,3-c]pyridin-8-yl)ethyl)amino)benzoate

A mixture of 8-(1-aminoethyl)-6-methyl-2-(pyridin-4-yl)-4H-pyrano[2,3-c]pyridin-4-one (80 mg, 284 umol), tert-butyl 2-iodobenzoate (173 mg, 569 umol), Pd₂(dba)₃ (26 mg, 28.4 umol), Xantphos (33 mg, 56.9 umol) and Cs₂CO₃ (278 mg, 853 umol) in dioxane (2 mL) was degassed with N₂ for 3 times. Then the reaction mixture was stirred at 100° C. for 16 h. The mixture was cooled to room temperature, quenched with water (20 mL). The mixture was extracted with EtOAc (20 mL×2). The combined organic layers were washed with brine (20 mL×2), dried over anhydrous Na₂SO₄ and filtered. The filtrate was concentrated in vacuo. The crude residue was purified by silica gel chromatography (solvent gradient: 0-60% EtOAc in petroleum ether) to give the title compound (114 mg, 88%) as a yellow solid. ¹H NMR (400 MHz, DMSO-d₆): δ 8.88-8.82 (m, 2H), 8.63 (d, J=7.6 Hz, 1H), 8.15-8.09 (m, 2H), 7.76-7.72 (m, 1H), 7.72 (s, 1H), 7.39 (s, 1H), 7.32 (s, 1H), 6.94 (d, J=8.4 Hz, 1H), 6.58-6.54 (m, 1H), 5.56-5.44 (m, 1H), 2.62 (s, 3H), 1.68 (d, J=6.4 Hz, 3H), 1.51 (s, 9H). MS: m/z 458.0 (M+H⁺).

### Step 11: Synthesis of tert-butyl (R)-2-((1-(6-methyl-4-oxo-2-(pyridin-4-yl)-4H-pyrano[2,3-c]pyridin-8-yl)ethyl)amino)benzoate and tert-butyl (S)-2-((1-(6-methyl-4-oxo-2-(pyridin-4-yl)-4H-pyrano[2,3-c]pyridin-8-yl)ethyl)amino)benzoate

Tert-butyl 2-((1-(6-methyl-4-oxo-2-(pyridin-4-yl)-4H-pyrano[2,3-c]pyridin-8-yl)ethyl)amino)benzoate (80 mg, 174.86 umol) was separated by prep.SFC (column: DAICEL CHIRALPAK IC(250 mm*30 mm, 10 um), Supercritical CO₂/EtOH+0.1% NH₄OH=40/60, 80 mL/min) to give tert-butyl (R)-2-((1-(6-methyl-4-oxo-2-(pyridin-4-yl)-4H-pyrano[2,3-c]pyridin-8-yl)ethyl)amino)benzoate (27 mg, 34%) and tert-butyl (S)-2-((1-(6-methyl-4-oxo-2-(pyridin-4-yl)-4H-pyrano[2,3-c]pyridin-8-yl)ethyl)amino)benzoate (27 mg, 34%) both as white solid. Absolute configuration was arbitrarily assigned to each enantiomer. MS: m/z 402.0 (M+H⁺).

### Step 12: Synthesis of (R)-2-((1-(6-methyl-4-oxo-2-(pyridin-4-yl)-4H-pyrano[2,3-c]pyridin-8-yl)ethyl)amino)benzoic acid trifluoroacetate (Compound 158)

To a solution of tert-butyl tert-butyl (R)-2-((1-(6-methyl-4-oxo-2-(pyridin-4-yl)-4H-pyrano[2,3-c]pyridin-8-yl)ethyl)amino)benzoate (27 mg, 59.01 umol) in DCM (1 mL) was added TFA (1 mL). After the addition, the reaction mixture was stirred at room temperature for 16 h. The mixture was concentrated to remove the solvent. The residue was triturated in MeOH (3 mL). The mixture was basified with 1M LiOH to pH 8 and acidified with formic acid to pH 5. To the mixture was added water (2 mL). The mixture was extracted with EtOAc (20 mL×3). The combined organic layers were washed with brine (20 mL), dried over anhydrous Na₂SO₄ and filtered. The filtrate was concentrated in vacuo. The crude residue was purified by silica gel chromatography (solvent gradient: 0-5% MeOH in DCM) to give the title compound (14 mg, 59%) as a white solid. ¹H NMR (400 MHz, DMSO-d₆): δ 9.00 (s, 1H), 8.84 (d, J=5.2 Hz, 2H), 8.15-8.05 (m, 2H), 7.83-7.76 (m, 1H), 7.72 (s, 1H), 7.39 (s, 1H), 7.35-7.28 (m, 1H), 6.92 (d, J=8.8 Hz, 1H), 6.57-6.53 (m, 1H), 5.55-5.44 (m, 1H), 2.62 (s, 3H), 1.65 (d, J=6.4 Hz, 3H). MS: m/z 402.0 (M+H⁺).

Compounds of Formula (III) in Table C can be prepared analogously to Example 8 and 9.

Compounds of Formula (I) in Table A can be prepared analogously to Examples 1-7

Compounds of Formula (I) in Table D below were prepared analogously to Examples 1-7.

TABLE D

| Cmpd. No. | Characterization Data |
|---|---|
| 5 | LC-MS: (M + H)+ found 455.1 |
| 6 | LC-MS: (M + H)+ found 441.1 |
| 8 | LC-MS: (M + H)+ found 419.2 |
| 10 | LC-MS: (M + H)+ found 419.2 |
| 11 | LC-MS: (M + H)+ found 435.3 |
| 17 | LC-MS: (M + H)+ found 409.1 |
| 18 | LC-MS: (M + H)+ found 409.1 |
| 19 | LC-MS: (M + H)+ found 409.1 |
| 23 | LC-MS: (M + H)+ found 462.1 |
| 24 | LC-MS: (M + H)+ found 468.2 |
| 32 | LC-MS: (M + H)+ found 432.1 |
| 33 | LC-MS: (M + H)+ found 482.1 |
| 34 | LC-MS: (M + H)+ found 602.3 |
| 36 | LC-MS: (M + H)+ found 425.3 |
| 46 | LC-MS: (M + H)+ found 449.1 |
| 51 | LC-MS: (M + H)+ found 440.2 |
| 57 | LC-MS: (M + H)+ found 392.1 |
| 61 | LC-MS: (M + H)+ found 444.3 |
| 63 | LC-MS: (M + H)+ found 496.1 |
| 65 | LC-MS: (M + H)+ found 496.1 |
| 69 | LC-MS: (M + H)+ found 481.3 |
| 71 | LC-MS: (M + H)+ found 496.1 |
| 73 | LC-MS: (M + H)+ found 467.3 |
| 74 | LC-MS: (M + H)+ found 407.1 |
| 76 | LC-MS: (M + H)+ found 431.2 |
| 78 | LC-MS: (M + H)+ found 454.1 |
| 81 | LC-MS: (M + H)+ found 424.3 |
| 84 | LC-MS: (M + H)+ found 451.1 |
| 86 | LC-MS: (M + H)+ found 481.1 |
| 87 | LC-MS: (M + H)+ found 481.1 |
| 89 | LC-MS: (M + H)+ found 424.3 |
| 128 | LC-MS: (M + H)+ found 456.1 |
| 131 | LC-MS: (M + H)+ found 391.1 |
| 132 | LC-MS: (M + H)+ found 407.2 |
| 133 | LC-MS: (M + H)+ found 481.2 |
| 134 | LC-MS: (M + H)+ found 481.3 |
| 135 | LC-MS: (M + H)+ found 429.1 |
| 136 | LC-MS: (M + H)+ found 455.1 |
| 137 | LC-MS: (M + H)+ found 415.2 |
| 138 | LC-MS: (M + H)+ found 440.1 |
| 139 | LC-MS: (M + H)+ found 414.1 |
| 140 | LC-MS: (M + H)+ found 463.4 |
| 141 | LC-MS: (M + H)+ found 414.2 |
| 142 | LC-MS: (M + H)+ found 450.1 |
| 143 | LC-MS: (M + H)+ found 440.1 |
| 144 | LC-MS: (M + H)+ found 496.1 |
| 145 | LC-MS: (M + H)+ found 455.1 |
| 146 | LC-MS: (M + H)+ found 497.1 |
| 147 | LC-MS: (M + H)+ found 469.1 |

### Biochemical Assays

PI3Kα cellular assay experimental procedure:
SKBR3 or T47D cells are seeded in DMEM containing 10% FBS at 25 k cells/well into 96-well cell culture format. Cells are incubated overnight at 37° C. in a 5% CO2 incubator and the following day cell media is aspirated, adherent cells are washed 1× with room temperature PBS prior to serum-free media application. Cells are returned to 37° C. 5% CO2 incubator and incubated a further 16 hrs. Compounds are added to serum starved adherent cells with a top dose of 10,000 nM and 3× multiple dose reductions for a minimum dose of 0.5 nM diluted in DMSO. Cell/compound incubation continues for 1 hr in a 37° C., 5% CO2 incubator prior to 10 minute PIK3CA stimulation with 20 ng/ml EGF. Cells treated with 0.1% DMSO and 20 ng/mL EGF are employed as negative control, cells treated with 10 uM Alpelisib and 20 ng/mL EGF are employed as a positive control. After 10 mins, plates are removed from incubator and cells are lysed with buffer and shaking 45 minutes. 20 μL of lysate is transferred to an opti-384 plate and 2.5 μl of Phospho-AKT d2 antibody with 2.5 μl of Phospho-AKT Eu Cryptate solution in the detection buffer are added to each well. 384 well plate is left overnight at room temperature before reading HTRF on an Envision plate reader.

The biological activity of certain compounds using the assays described above is shown in Table 2. The ranges are as follows:
for T47D pAKT IC₅₀ (nM): A denotes <750 nM; B denotes 750 nM≤IC₅₀<2,000 nM; C denotes ≥2,000 nM. ND denotes value not determined with that assay for the specified compound;
for T47D (H1047R) selectivity over SKBR3 (WT): A denotes >20-fold; B denotes 20-fold≥value>5-fold; C denotes ≤5-fold. ND denotes value not determined with that assay for the specified compound;
for PI3K H1047R ADP-Glo (PI/PS) IC₅₀ (nM): A denotes <1,000 nM; B denotes 1,000 nM≤IC₅₀<10,000 nM; C denotes ≥10,000 nM. ND denotes value not determined with that assay for the specified compound; and
for PI3K H1047R ADP-Glo (PI/PS) IC₅₀: H1047R Selectivity: A denotes >20-fold; B denotes 20-fold≥value>5-fold; C denotes ≤5-fold. ND denotes value not determined with that assay for the specified compound.

TABLE 2
Biological Activity of Selected Compounds

| | T47D pAKT S473 | T47D pAKT S473 | PI3K H1047R | |
|---|---|---|---|---|
| | HTRF IC50 | HTRF IC₅₀ (H1047R): | ADP-Glo | PI3K H1047R |
| | (H1047R): | T47D (H1047R) | (PI/PS) IC₅₀: | ADP-Glo (PI/PS) |
| Cmpd. | Average | selectivity | Average | IC₅₀: H1047R |
| No. | IC₅₀ (nM) | over SKBR3 (WT) | IC₅₀ (nM) | Selectivity |
| 1 | C | C | ND | ND |
| 2 | C | C | ND | ND |
| 3 | C | C | ND | ND |
| 4 | C | C | ND | ND |
| 5 | B | B | C | ND |
| 6 | C | C | ND | ND |
| 7 | C | C | ND | ND |
| 8 | A | A | A | B |
| 9 | A | B | ND | ND |
| 10 | A | A | A | A |
| 11 | B | B | ND | ND |
| 12 | C | C | ND | ND |
| 17 | A | B | ND | ND |
| 18 | C | C | ND | ND |
| 19 | B | B | B | B |
| 21 | C | C | ND | ND |
| 23 | C | C | ND | ND |
| 24 | A | A | ND | ND |
| 26 | C | C | C | C |
| 30 | B | B | ND | ND |
| 32 | A | A | A | A |
| 33 | A | A | A | B |
| 34 | C | C | ND | ND |
| 35 | C | C | ND | ND |
| 36 | B | B | ND | ND |
| 37 | A | A | A | A |
| 38 | A | A | A | A |
| 39 | C | C | ND | ND |
| 40 | A | A | A | A |
| 41 | C | C | C | ND |
| 46 | C | C | ND | ND |
| 51 | A | A | A | A |
| 57 | A | A | A | A |
| 58 | C | C | C | ND |
| 59 | B | B | ND | ND |
| 60 | C | C | C | ND |
| 61 | A | A | ND | ND |
| 63 | B | B | ND | ND |
| 65 | B | B | ND | ND |
| 69 | B | B | ND | ND |
| 71 | C | C | ND | ND |
| 73 | A | A | A | A |
| 74 | A | A | A | A |
| 75 | B | B | C | ND |
| 76 | A | A | A | A |
| 77 | A | A | A | A |
| 78 | A | A | A | A |
| 79 | C | C | C | ND |
| 80 | C | C | C | ND |
| 81 | A | A | A | A |
| 82 | A | A | A | A |
| 83 | C | C | C | C |
| 84 | A | A | A | A |
| 85 | C | B | B | C |
| 86 | A | A | ND | ND |
| 87 | A | A | ND | ND |
| 88 | A | A | A | A |
| 89 | A | A | ND | ND |
| 128 | A | A | ND | ND |
| 131 | A | A | ND | ND |
| 132 | A | A | ND | ND |
| 133 | A | A | ND | ND |
| 134 | A | A | ND | ND |
| 135 | A | A | ND | ND |
| 136 | A | A | ND | ND |
| 137 | A | A | ND | ND |
| 138 | A | A | ND | ND |
| 139 | B | B | ND | ND |
| 140 | A | B | ND | ND |
| 141 | A | B | ND | ND |
| 142 | A | A | ND | ND |
| 143 | A | A | ND | ND |
| 144 | B | B | ND | ND |
| 145 | A | B | ND | ND |
| 146 | A | A | ND | ND |
| 147 | C | C | ND | ND |

What is claimed is:

1. A compound of Formula (I), Formula (II), or Formula (III): 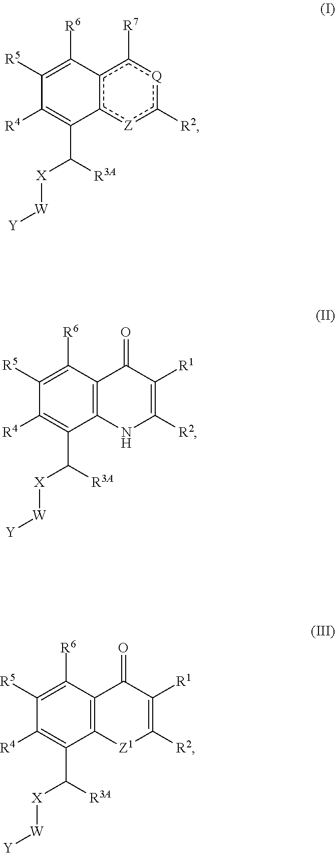
or a pharmaceutically acceptable salt of any of the foregoing, wherein:
Q is NR^{1A} or CR¹;
Z is NR^{Z1} or CR^{Z2}, wherein one or both of Q and Z is N;
Z¹ is S, —S(O₂)—, or O;
each [symbol] represents a single bond or a double bond;
R¹ is hydrogen, cyano, C3-C6 cycloalkyl, C1-C6 alkyl optionally substituted with phenyl optionally substituted with halogen, C1-C6 thioalkyl, C1-C6 haloalkyl, C1-C6 alkoxy, or C1-C6 alkoxyalkyl;
R^{1A} is absent, hydrogen, C1-C6 alkyl, C1-C6 haloalkyl, C1-C6 thioalkyl, C1-C6 haloalkyl, C1-C6 alkoxy, or C1-C6 alkoxyalkyl;
R^{Z1} is absent, hydrogen, cyano, C1-C6 alkyl, or C1-C6 alkyl;
R^{Z2} is hydrogen, C1-C6 alkyl, or C1-C6 haloalkyl;
R² is phenyl optionally substituted with 1-4 independently selected R^{2A}, 5-10 membered heteroaryl optionally substituted with 1-4 independently selected R^{2A}, 4-10 membered heterocyclyl optionally substituted with 1-4 independently selected R^{2A}, 4-10 membered cycloalkyl optionally substituted with 1-4 independently selected R^{2A}, or C1-C6 alkoxy optionally substituted with —C(═O)NR^{A}R^{C};
each R^{2A} is independently selected from:
(i) halogen,
(ii) cyano,
(iii) hydroxyl,
(iv) —NR^{A}R^{B},
(v) —C(═O)NR^{A}R^{B}, 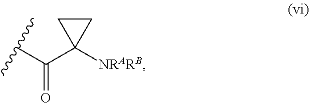
(vii) —NHC(═O)R^{C},
(viii) —C(═O)NR^{D}R^{E},
(ix) —C(═O)OR^{F},
(x) —SO₂R^{F},
(xi) —NHSO₂R^{F},
(xii) —SO₂NR^{F}R^{G},
(xiii) —NHC(═O)C1-C6 alkyl optionally substituted with NR^{A}R^{B},
(xiv) C1-C6 haloalkyl,
(xv) C1-C6 hydroxyalkyl,
(xvi) 5-10 membered heteroaryl optionally substituted with 1-3 substituents independently selected from C1-C6 alkyl and —NR^{A}R^{B},
(xvii) 4-10 membered heterocyclyl optionally substituted with 1-3 substituents independently selected from hydroxyl, C1-C6 haloalkyl, —C(═O)C1-C6 alkyl, —SO₂(C1-C6 alkyl), —SO₂NR^{F}R^{G}, C1-C6 alkyl optionally substituted with C1-C6 alkoxy, —C(═O)NR^{A}R^{B}, or —NHC(═O)C1-C6 alkyl optionally substituted with —NR^{A}R^{B},
(xviii) C1-C6 alkyl optionally substituted with 1-3 substituents independently selected from hydroxyl, oxo, —NR^{A}R^{B}, —C(═O)NR^{A}R^{B}, C1-C6 alkoxy, and 4-10 membered heterocyclyl optionally substituted with hydroxyl, C1-C6 alkyl, aralkyl, heteroaralkyl, —C(═O)NR^{A}R^{B}, or —C(═O)C3-C6 cycloalkyl,
(xix) C1-C6 alkoxy optionally substituted with —NR^{A}R^{B} or 4-10 membered heterocyclyl optionally substituted with C1-C6 alkyl, aralkyl, heteroaralkyl, or —C(═O)C3-C6 cycloalkyl, and
(xx) C3-C6 cycloalkyl optionally substituted with 4-10 membered heterocyclyl optionally substituted with C1-C6 alkyl;
each R^{A} and R^{B} is independently selected from hydrogen, hydroxyl, C1-C6 alkoxy, C3-C6 cycloalkyl, C2-C6 alkenyl, and C1-C6 alkyl optionally substituted with hydroxyl or C1-C6 alkoxy, or R^{A} and R^{B} together with the nitrogen atom to which they are attached form a 4-10 membered heterocyclyl optionally substituted with 1-2 substituents independently selected from halogen, C1-C6 alkyl, and —C(═O)C1-C6 alkyl;
each R^{C} is independently selected from C3-C6 cycloalkyl, —C(═O)NR^{Y1}, or a C1-C6 alkyl optionally substituted with —NR^{A}R^{B} or with 4-10 membered heterocyclyl optionally substituted with C1-C6 alkyl or with C1-C6 hydroxylalkyl;
each R^{D} and R^{E} is independently selected from hydrogen, hydroxyl, C1-C6 alkyl, and C1-C6 alkoxy;
each R^{3A} and R^{3B} is independently selected from hydrogen, C1-C6 alkyl, C1-C6 alkoxy, and C1-C6 haloalkyl, or R^{3A} and R^{3B}, together with the carbon and nitrogen atoms, respectively, to which they are attached together form a 4-8 membered heterocyclyl group;
R⁴ is hydrogen, C1-C6 alkyl, or acrylamido;
R⁵ is hydrogen, C1-C6 alkyl, cyano, —NR^{5A}R^{5B}, —NR^{5A}C(═O)R^{5B}, or —C(═O)NR^{5A}R^{5B};
R^{5A} and R^{5B} are independently selected from hydrogen, CJ-C6 alkyl, C2-C6 alkenyl, and C1-C6 hydroxyalkyl;
R⁶ is hydrogen, halogen, or C1-C6 alkyl;
R⁷ is hydrogen, halogen, hydroxyl, cyano, C1-C6 alkyl, C1-C6 alkoxy, C1-C6 alkoxyalkyl, C1-C6 hydroxyalkyl, C1-C6 cyanoalkyl, —C(═O)C1-C6 alkyl, —NH(CN), 5-6 membered heteroaryl, —NR^{7A}R^{7B}, —NR^{7A}C(═O)R^{7B}, —C(═R^{A})NR^{7A}R^{7B}, or —C(═O)NR^{7A}R^{7B}, 4-10 membered heterocyclyl optionally substituted with 1-3 substituents independently selected from hydroxyl, C1-C6 haloalkyl, —C(═O)C1-C6 alkyl, C1-C6 alkyl optionally substituted with C1-C6 alkoxy, —C(═O)NR^{7A}R^{7B}, or —NHC(═O)C1-C6 alkyl optionally substituted with —NR^{7A}R^{7B}, or when each [symbol] adjacent to CR⁷ is a single bond, R⁷ can be oxo;
each R^{7A} and R^{7B} are independently selected from hydrogen, C2-C6 alkenyl, C2-C6 haloalkenyl, C1-C6 alkyl optionally substituted with oxo, or C1-C6 haloalkyl optionally substituted with oxo;
X is a bond, CH₂, CH(CH₃), C(CH₃)₂, or 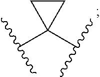 [SMILES: *C1(*)CC1]
W is NR^{3B} or O;
Y is phenyl optionally substituted with 1-3 independently selected R^{Y}, naphthyl optionally substituted with 1-3 independently selected R^{Y}, or 5-10 membered heteroaryl optionally substituted with 1-3 independently selected R^{Y};
each R^{Y} is independently selected from: halogen, cyano, hydroxyl, C1-C6 haloalkyl optionally substituted with hydroxyl, C1-C6 alkoxy, C1-C6 haloalkoxy, C1-C6 hydroxyalkyl, —NHC(═O)R^{C}, —C(═O)NHR^{Y1}, —CO₂R^{A}, —SO₂NR^{F}R^{G}, —NHSO₂R^{F}, —S(═O)(═NR^{F})R^{G}, —SO₂(C1-C6 alkyl), —C(═O)NR^{A}R^{B}, 5-6 membered heteroaryl, heteroaralkyl, 4-6 membered heterocyclyl optionally substituted with R^{Y1}, and C1-C6 alkyl optionally substituted with —CO₂R^{A} or 4-6 membered heteroaryl optionally substituted with R^{Y1};
R^{Y1} is —SO₂(C1-C6 alkyl), hydroxyl, or C1-C6 alkyl optionally substituted with oxo; and
each R^{F} and R^{G} is independently selected from hydrogen, phenyl, and C1-C6 alkyl optionally substituted with oxo or —NR^{A}R^{B}.

2. The compound of claim 1, wherein the compound is a compound of Formula (II).

3. The compound of claim 1, wherein the compound is a compound of Formula (III).

4. The compound of claim 1 or 3, wherein Z¹ is S.

5. The compound of claim 1 or 3, wherein Z¹ is O.

6. The compound of claim 1 or 3, wherein Z¹ is —S(O₂)—.

7. The compound of claim 1, wherein the compound is a compound of Formula (I).

8. The compound of claim 1 or 7, wherein Q is CR¹.

9. The compound of any one of claims 1-8, wherein R² is phenyl optionally substituted with 1-3 independently selected R^{2A}.

10. The compound of any one of claims 1-8, wherein R² is 5-10 membered heteroaryl optionally substituted with 1-3 independently selected R^{2A}.

11. The compound of any one of claims 1-8, wherein R² is 4-10 membered heterocyclyl optionally substituted with 1-3 independently selected R^{2A}.

12. The compound of any one of claims 1-8, wherein R² is 4-10 membered cycloalkyl optionally substituted with 1-3 independently selected R^{2A}.

13. The compound of any one of claims 1-8, wherein R² is C1-C6 alkoxy optionally substituted with —C(═O)NR^{A}R^{C}.

14. The compound of any one of claims 1-8, R² is C1-C6 alkoxyalkyl

15. The compound of any one of claims 1-14, wherein X is a bond.

16. The compound of any one of claims 1-14, wherein X is CH₂.

17. The compound of any one of claims 1-14, wherein X is CH(CH₃).

18. The compound of any one of claims 1-14, wherein X is C(CH₃)₂.

19. The compound of any one of claims 1-14, wherein X is

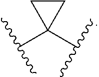 [SMILES: *C1(*)CC1]

20. The compound of any one of claims 1-19, wherein W is O.

21. The compound of any one of claims 1-19, wherein W is NR^{3B}.

22. The compound of any one of claims 1-21, wherein Y is phenyl optionally substituted with 1-3 independently selected R^{Y}.

23. The compound of any one of claims 1-21, wherein Y is naphthyl optionally substituted with 1-3 independently selected R^{Y}.

24. The compound of any one of claims 1-21, wherein Y is 5-10 membered heteroaryl optionally substituted with 1-3 independently selected R^{Y}.

25. The compound of any one of claims 1-24, wherein R^{Y} is halogen.

26. The compound of any one of claims 1-24, wherein R^{Y} is hydroxyl.

27. The compound of any one of claims 1-24, wherein R^{Y} is cyano.

28. The compound of any one of claims 1-24, wherein R^{Y} is C1-C6 haloalkyl.

29. The compound of any one of claims 1-24, wherein R^{Y} is C1-C6 alkoxy.

30. The compound of any one of claims 1-24, wherein R^{Y} is C1-C6 haloalkoxy.

31. The compound of any one of claims 1-24, wherein R^{Y} is C1-C6 hydroxyalkyl.

32. The compound of any one of claims 1-24, wherein R^{Y} is —NHC(═O)R^{C}.

33. The compound of any one of claims 1-24, wherein R^{Y} is —C(═O)NHR^{Y1}.

34. The compound of any one of claims 1-24, wherein R^{Y} is —CO₂R^{A}.

35. The compound of any one of claims 1-24, wherein R^{Y} is —SO₂NR^{F}R^{G}.

36. The compound of any one of claims 1-24, wherein R^{Y} is —NHSO₂R^{F}.

37. The compound of any one of claims 1-24, wherein R^{Y} is —S(═O)(═NR^{F})R^{G}.

38. The compound of any one of claims 1-24, wherein R^{Y} is —SO₂(C1-C6 alkyl).

39. The compound of any one of claims 1-24, wherein R^{Y} is —C(═O)NR^{A}R^{B}.

40. The compound of any one of claims 1-24, wherein R^{Y} is 4-6 membered heteroaryl.

41. The compound of any one of claims 1-24, wherein R^{Y} is heteroaralkyl.

42. The compound of any one of claims 1-24, wherein R^{Y} is 4-6 membered heterocyclyl.

43. The compound of any one of claims 1-24, wherein R^{Y} is C1-C6 alkyl optionally substituted with —CO₂R^{A} or 5-6 membered heteroaryl optionally substituted with R^{Y1}.

44. The compound of any one of claims 1-24, wherein R^{Y} is C1-C6 alkyl substituted with —CO₂R^{A} or 5-6 membered heteroaryl optionally substituted with R^{Y1}.

45. The compound of any one of claims 1-24, wherein R^{Y} is C1-C6 alkyl substituted with —CO₂R^{A} or 5-6 membered heteroaryl substituted with R^{Y1}.

46. The compound of any one of claims 1-24, wherein R^{Y} is C1-C6 alkyl substituted with —CO₂R^{A} or 5-6 membered heteroaryl.

47. The compound of any one of claims 43-45, wherein R^{Y1} is —SO₂(C1-C6 alkyl).

48. The compound of any one of claims 43-45, wherein R^{Y1} is C1-C6 alkyl optionally substituted with oxo.

49. The compound of any one of claims 43-45, wherein R^{Y1} is hydroxyl.

50. A compound selected from the group consisting of the compounds in Table A, or a pharmaceutically acceptable salt of any of the foregoing.

51. A compound selected from the group consisting of the compounds in Table B, or a pharmaceutically acceptable salt of any of the foregoing.

52. A compound selected from the group consisting of the compounds in Table C, or a pharmaceutically acceptable salt of any of the foregoing.

53. A pharmaceutical composition comprising a compound of any one of claims 1-52, or a pharmaceutically acceptable salt of any of the foregoing, and one or more pharmaceutically acceptable excipients.

54. A method for treating cancer in a subject in need thereof, comprising administering to the subject a therapeutically effective amount of a compound of any one of claims 1-52, or a pharmaceutically acceptable salt of any of the foregoing, or a pharmaceutical composition of claim 53.

55. A method for treating cancer in a subject in need thereof, the method comprising (a) determining that the cancer is associated with a dysregulation of a PIK3CA gene, a PI3Kα protein, or expression or activity or level of any of the same; and (b) administering to the subject a therapeutically effective amount of a compound of any one of claims 1-52, or a pharmaceutically acceptable salt of any of the foregoing, or a pharmaceutical composition of claim 53.

56. A method of treating a PI3Kα-associated cancer in a subject, comprising administering to a subject identified or diagnosed as having a PI3Kα-associated cancer a therapeutically effective amount of a compound of any one of claims 1-52 or a pharmaceutically acceptable salt of any of the foregoing, or a pharmaceutical composition of claim 53.

57. A method for inhibiting mutant PI3Kα activity in a mammalian cell, the method comprising contacting the mammalian cell with an effective amount of a compound of any one of claims 1-52, or a pharmaceutically acceptable salt of any of the foregoing.
